(19) United States
(12) Patent Application Publication
(10) Pub. No.: US 2026/0240994 A1
(43) Pub. Date: Aug. 20, 2026

(54) ENGINEERED MESENCHYMAL STROMAL CELLS

(71) Applicant: Mayo Foundation for Medical Education and Research, Rochester, MN (US)

(72) Inventors: Saad J. Kenderian, Rochester, MN (US); Olivia Sirpilla, Rochester, MN (US)

(21) Appl. No.: 19/126,728

(22) PCT Filed: Nov. 2, 2023
(86) PCT No.: PCT/US2023/078478

§ 371 Date: May 2, 2025

Related U.S. Application Data
(60) Provisional application No. 63/421,832, filed on Nov. 2, 2022.

(51) Int. Cl. A61K 40/11

(52) U.S. Cl. CPC A61K 40/11

(57) ABSTRACT

This document relates to methods and materials for using engineered mesenchymal stromal cells (MSCs) to treat a mammal (e.g., a human) in need of immunosuppression (e.g., a human having or at risk of developing one or more autoimmune diseases (e.g., graft-versus-host disease (GVHD)). For example, CAR-MSCs (e.g., MSCs expressing a chimeric antigen receptor (CAR) having the ability to bind to a tissue-specific antigen) having (e.g., engineered to have) an elevated level of one or more immunosuppressive polypeptides, an elevated level of one or more regenerative polypeptides, and/or an elevated level of one or more trafficking polypeptides are provided. Methods for administering one or more CAR-MSCs provided herein to a mammal (e.g., a human) in need of immunosuppression (e.g., a human having or at risk of developing one or more autoimmune diseases such as GVHD) to treat the mammal are also provided.

### CROSS-REFERENCE TO RELATED APPLICATIONS

This application claims the benefit of U.S. Patent Application Ser. No. 63/421,832, filed on Nov. 2, 2022. The disclosure of the prior application is considered part of, and is incorporated by reference in, the disclosure of this application.

### SEQUENCE LISTING

This application contains a Sequence Listing that has been submitted electronically as an XML file named “07039-2165WO1.xml.” The XML file, created on Oct. 31, 2023, is 160,000 bytes in size. The material in the XML file is hereby incorporated by reference in its entirety.

### TECHNICAL FIELD

This document relates to methods and materials for using engineered mesenchymal stromal cells (MSCs) to treat a mammal (e.g., a human) in need of immunosuppression (e.g., a human having or at risk of developing one or more autoimmune diseases (e.g., graft-versus-host disease (GVHD)). For example, this document provides CAR-MSCs (e.g., MSCs expressing a chimeric antigen receptor (CAR) having the ability to bind to a tissue-specific antigen) having (e.g., engineered to have) an elevated level of one or more immunosuppressive polypeptides, an elevated level of one or more regenerative polypeptides, and/or one or more trafficking polypeptides. Such CAR-MSCs can exert an immunosuppressive effect (e.g., can reduce or eliminate an immune response such as an overactive immune response) in a targeted tissue. This document also provides methods for administering one or more CAR-MSCs having an elevated level of one or more immunosuppressive polypeptides, an elevated level of one or more regenerative polypeptides, and/or an elevated level of one or more trafficking polypeptides to a mammal (e.g., a human) in need of immunosuppression (e.g., a human having or at risk of developing one or more autoimmune diseases such as GVHD) to treat the mammal.

### BACKGROUND INFORMATION

MSCs are an attractive cell therapy candidate due to their trophic abilities allowing for homing to sites of inflammation (Shi et al., Nat. Rev. Nephrol., 14:493-507 (2018)) and immunomodulatory functions supporting immune homeostasis (Uccelli et al., Nat. Rev. Immunol., 8:726-736 (2008)). However, reliable therapeutic efficacy has not been demonstrated in human clinical trials, making MSCs the most studied experimental cell therapy platform in the world with no resultant US FDA-approved therapies (Murata et al., Bone Marrow Transplant., 56:2355-2366 (2021)).

MSC therapeutic efficacy in humans is limited by several factors including 1) suboptimal immunosuppression and 2) insufficient homing to target sites once administered. Strategies to individually address these shortcomings with cytokine modulation (Guess et al. Stem Cells Transl. Med., 6:1868-1879 (2017)), manipulation of MSC extracellular vesicles (Harrell et al., Cells, 8(2019)), and genetic modification (Sarkar et al., Blood 118: e184-191 (2011)), have been attempted, but have yet to simultaneously enhance both immunosuppression and homing capacity.

### SUMMARY

This document provides methods and materials involved in treating a mammal (e.g., a human) in need of immunosuppression (e.g., a human having or at risk of developing one or more autoimmune diseases such as GVHD). For example, one or more CAR-MSCs having (e.g., engineered to have) an elevated level of one or more immunosuppressive polypeptides, an elevated level of one or more regenerative polypeptides, and/or an elevated level of one or more trafficking polypeptides can be administered to a mammal to induce an immunosuppressive response (e.g., to reduce or eliminate an inflammatory immune response) in a targeted tissue within the mammal. In some cases, one or more CAR-MSCs having (e.g., engineered to have) an elevated level of one or more immunosuppressive polypeptides, an elevated level of one or more regenerative polypeptides, and/or an elevated level of one or more trafficking polypeptides can be administered (e.g., by adoptive transfer) to a mammal (e.g., a human) having (or at risk of developing) one or more autoimmune diseases (e.g., GVHD) to treat the mammal. As demonstrated herein, CAR-MSCs having elevated levels of a nuclear factor kappa B subunit 1 (NFκB1) polypeptide, a Jun proto-oncogene (JUN) polypeptide, a transcription factor RelB (RELB) polypeptide, an interferon regulatory factor 1 (IRF1) polypeptide, a tumor necrosis factor (TNF) a polypeptide, an interleukin (IL)-10 polypeptide, a fibroblast growth factor (FGF)-2 polypeptide, a granulocyte colony stimulating factor (G-CSF) polypeptide, a granulocyte-macrophage colony-stimulating factor (GM-CSF) polypeptide, an eotaxin polypeptide, a galectin 9 (Gal-9) polypeptide, a programmed cell death protein 1 (PD-1) polypeptide, a T-cell immunoglobin mucin-3 (TIM-3) polypeptide, a CXC chemokine receptor (CXCR) 3 polypeptide, and/or a CXCR4 polypeptide exhibited both enhanced immunosuppression and enhanced homing capacity to sites of inflammation. Also as demonstrated herein, CAR-MSCs having elevated levels of a cytotoxic T-lymphocyte-associated-protein 4 (CTLA4) polypeptide, a toll-like receptor (TLR) 3 polypeptide, a TLR4 polypeptide, a TLR9 polypeptide, and/or a TNF receptor 2 (TNFR2) polypeptide exhibited both enhanced immunosuppression and enhanced homing capacity to sites of inflammation.

In general, one aspect of this document features a MSC including (1) exogenous nucleic acid encoding a CAR targeting an epithelial-specific antigen, where the MSC expresses the CAR, and (2) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, a CXCR4 polypeptide, a CTLA4 polypeptide, a TLR3 polypeptide, a TLR4 polypeptide, a TLR9 polypeptide, and a TNFR2 polypeptide. The MSC can be a human MSC. The MSC can be an adipose derived-MSC. The epithelial-specific antigen can be E-cadherin (Ecad). The polypeptide can be the NFκB1 polypeptide, the JUN polypeptide, the RELB polypeptide, or the IRF1 polypeptide. The MSC can include exogenous nucleic acid encoding the NFκB1 polypeptide, where the MSC expresses the NFκB1 polypeptide. The MSC can include exogenous nucleic acid encoding the JUN polypeptide, where the MSC expresses the JUN polypeptide. The MSC can include exogenous nucleic acid encoding the RELB polypeptide, where the MSC expresses the RELB polypeptide. The MSC can include exogenous nucleic acid encoding the IRF1 polypeptide, where the MSC expresses the IRF1 polypeptide. The polypeptide can be the CXCR3 polypeptide or the CXCR4 polypeptide. The MSC can include exogenous nucleic acid encoding the CXCR3 polypeptide, where the MSC expresses the CXCR3 polypeptide. The MSC can include exogenous nucleic acid encoding the CXCR4 polypeptide, where the MSC expresses the CXCR4 polypeptide. The polypeptide can be the PD-1 polypeptide, the Gal-9 polypeptide, or the TIM-3 polypeptide. The MSC can include exogenous nucleic acid encoding the PD-1 polypeptide, where the MSC expresses the PD-1 polypeptide. The MSC can include exogenous nucleic acid encoding the Gal-9 polypeptide, where the MSC expresses the Gal-9 polypeptide. The MSC can include exogenous nucleic acid encoding the TIM-3 polypeptide, where the MSC expresses the TIM-3 polypeptide. The polypeptide can be the TNFα polypeptide, the IL-10 polypeptide, or the FGF-2 polypeptide. The MSC can include exogenous nucleic acid encoding the TNFα polypeptide, where the MSC expresses the TNFα polypeptide. The MSC can include exogenous nucleic acid encoding the IL-10 polypeptide, where the MSC expresses the IL-10 polypeptide. The MSC can include exogenous nucleic acid encoding the FGF-2 polypeptide, where the MSC expresses the FGF-2 polypeptide. The CAR can include a heavy chain comprising the CDRs set forth in SEQ ID NO: 1, and a light chain comprising the CDRs set forth in SEQ ID NO:2. The heavy chain can include an amino acid sequence set forth in SEQ ID NO:1, and where the light chain can include an amino acid sequence set forth in SEQ ID NO:2. The CAR can include a heavy chain comprising the CDRs set forth in SEQ ID NO:3, and a light chain comprising the CDRs set forth in SEQ ID NO:4. The heavy chain can include an amino acid sequence set forth in SEQ ID NO:3, and where the light chain can include an amino acid sequence set forth in SEQ ID NO:4.

In another aspect, this document features compositions including a MSC including (1) exogenous nucleic acid encoding a CAR targeting an epithelial-specific antigen, where the MSC expresses the CAR, and (2) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, a CXCR4 polypeptide, a CTLA4 polypeptide, a TLR3 polypeptide, a TLR4 polypeptide, a TLR9 polypeptide, and a TNFR2 polypeptide.

In another aspect, this document features methods for treating a mammal having GVHD. The methods can include, or consist essentially of, administering to a mammal having GVHD a MSC including (1) exogenous nucleic acid encoding a CAR targeting an epithelial-specific antigen, where the MSC expresses the CAR, and (2) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, a CXCR4 polypeptide, a CTLA4 polypeptide, a TLR3 polypeptide, a TLR4 polypeptide, a TLR9 polypeptide, and a TNFR2 polypeptide. The mammal can be a human. The symptom of the GVHD can be reduced at least 10 percent. The number of Tregs within the mammal can be increased at least 10 percent.

In another aspect, this document features methods for suppressing an immune response within a mammal. The methods can include, or consist essentially of, administering to a mammal a MSC including (1) exogenous nucleic acid encoding a CAR targeting an epithelial-specific antigen, where the MSC expresses the CAR, and (2) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, a CXCR4 polypeptide, a CTLA4 polypeptide, a TLR3 polypeptide, a TLR4 polypeptide, a TLR9 polypeptide, and a TNFR2 polypeptide. The mammal can be a human. The number of activated T cells within the mammal can be reduced at least 10 percent. The number of Tregs within the mammal can be increased at least 10 percent.

In another aspect, this document features methods for reducing the number of activated T cells within a mammal. The methods can include, or consist essentially of, administering to a mammal a MSC including (1) exogenous nucleic acid encoding a CAR targeting an epithelial-specific antigen, where the MSC expresses the CAR, and (2) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, a CXCR4 polypeptide, a CTLA4 polypeptide, a TLR3 polypeptide, a TLR4 polypeptide, a TLR9 polypeptide, and a TNFR2 polypeptide. The mammal can be a human. The number of activated T cells within the mammal can be reduced at least 10 percent. The number of Tregs within the mammal can be increased at least 10 percent.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIGS. 1A-1J show that MSCs are stably transduced to express CAR and that EcCAR-MSCs exhibit superior T cell suppression compared to untransduced (UTD) MSCs. FIG. 1A is a representative flow cytometry plot depicting the expression of CAR19 as detected by goat anti-mouse IgG (y-axis) following transduction of MSCs with VSV-γ lentiviral particles with increasing concentration of protamine sulfate enhancer (50 μg/mL and 100 μg/mL). Data are representative of >3 independent experiments using 5 different biological MSC donors. FIG. 1B is a histogram showing CAR expression of anti-Ecad CAR-MSC (EcCAR-MSC), anti-CD103 T cell integrin CAR-MSC (CD103CAR-MSC), and anti-CD19 CAR MSC (CD19CAR-MSC) positive control as detected by flow cytometry using goat anti-mouse IgG antibody. Data shown are mean+/−s.d. representative of at >6 independent experiment using 5 MSC donors. FIG. 1C is a graph showing CAR expression of EcCAR-MSC compared to UTD-MSC by flow cytometry 2 days and 11 days following transduction with multiple cell passages. Data shown are mean+/−standard deviation (s.d.) representative of 3 independent experiments using 3 MSC donors. Statistical analysis was performed by 2-way ANOVA (****p≤0.0001) (n=3 per group). FIG. 1D is a histogram depicting >90% expression of Ecad-CAR (EcCAR) on MSCs across 3 different primary biological donors. Data are representative of >10 independent experiments using 5 different biological MSC donors. The x axis represents the level of detection of CAR by goat anti-mouse within the APC channel of the flow cytometry instrument. FIG. 1E is a graph of flow cytometry-based quantification of absolute CD³⁺ cells following a co-culture of EcCAR-MSCs, UTD-MSCs, or no MSCs with activated T cells at 1:5 and 1:10 MSC: T cell ratio. Data shown are mean+/−s.d. using 3 MSC donors. Statistical analysis was performed by 2-way ANOVA (*p≤0.05, ****p<0.0001) (n=3 per group). FIG. 1F is a graph of flow cytometry-based quantification of absolute CD³⁺ cells following a co-culture of EcCAR-MSCs, UTD-MSCs, or no MSCs with activated T cells, in the presence of media alone or soluble Ecad at 250 ng/mL. Data shown are mean+/−s.d. of representative experiments using 5 MSC donors. Statistical analysis was performed by 2-way ANOVA (no significant (ns)=p≥0.05, **p≤0.01) (n=2 per group). FIG. 1G is a graph of flow cytometry-based quantification of absolute CD³⁺ cells following co-culture of EcCAR-MSCs, UTD-MSCs, or no MSCs with activated T cells, in the presence of the irradiated Ecad positive cell line MCF-7, enhanced T cell suppression with antigen specific activation. Data shown are mean+/−s.d. of representative experiments using 3 different biological MSC donors. Statistical analysis was performed by 2-way ANOVA (ns=p≥0.05, *p≤0.05) (n=2 per group). FIG. 1H is a graph showing the stemness immuno-phenotype of UTD-MSCs and EcCAR-MSCs measured with flow cytometry after staining MSCs for CD105, CD90, CD73, CD34, CD45, and CD14. Data shown are mean+/−s.d. of 4 independent experiments from 4 biological MSC donors. Statistics were performed by 2-way ANOVA (ns=p≥0.05) (n=3 per group). FIGS. 1I-1J contain graphs of bulk RNAseq analysis of upregulated (FIG. 1I) and downregulated (FIG. 1J) pathways in EcCAR-MSCs as compared to UTD-MSC by CellMarker Augmented gene set enrichment analysis. Significant genes were selected by >1 or <−1 log fold change and <0.05 adjusted p values via differential expression analysis of 3 biological replicates per group. Calculated −log₁₀ p value displayed on x-axis to represent statistically significant enrichment for all pathways −log₁₀ (p<0.05). Data representing analysis of 3 different biological MSC donors per differential expression analysis group.

FIGS. 2A-2H show that EcCAR-MSCs suppress T cell activity in tumor and GVHD models. FIG. 2A is a schematic of an exemplary method for evaluating the effect of EcCAR-MSCs on a Nalm6 xenograft tumor model. Immunocompromised NOD-SCID-y-(NSG) mice were engrafted with luciferase⁺ CD19⁺ Nalm6 cells (1×10⁶ intravenously (i.v.)). Bioluminescent imaging was performed 5 days later to confirm engraftment. All mice were then treated with CD19-targeted CAR T (CART19) cells (1×10⁶ cells i.v.) and irradiated Ecad⁺ cell line MCF-7. Mice were additionally randomized to treatment with UTD-MSCs or EcCAR-MSCs (1×10⁶ cells intraperitoneally (i.p.)). Mice were then followed biweekly for bioluminescent imaging and monitored for survival. FIG. 2B is a graph of tumor flux following CART19 infusion in Nalm6 model, comparing EcCAR-MSCs, UTD-MSCs, and no treatment. Data shown are mean+/−standard error of the mean (SEM) with statistical analysis by 2-way ANOVA (**p≤0.01, ***p≤0.001, ns=p≥0.05) (n=3-4 mice per group). FIG. 2C is a graph of survival outcomes of EcCAR-MSC compared to UTD-MSC and no MSC control. Statistical analysis was performed by Kaplan-Meier Simple survival analysis (*p≤0.05, ns=p≥0.05) (n=3-4 mice per group). FIG. 2D is a schematic of an exemplary method for evaluating the effect of EcCAR-MSCs in a GVHD Xenograft mouse model. Human PBMCs were injected (25-30×10⁶ cells i.v.) into NSG mice along with either EcCAR-MSCs or UTD-MSCs (1×10⁶ cells i.p. on days 0, 14, and 28). Mice were then monitored for weight loss, the development of clinical signs of GVHD (scored based on weight loss, diarrhea, posture, activity, fur texture, and skin integrity), and survival. FIG. 2E is a graph of percent weight change from baseline in GVHD xenografts following treatment with UTD-MSCs, EcCAR-MSCs, or no treatment. Data shown are mean+/−s.d. with statistical analysis performed by 2-way ANOVA (****p≤0.0001, ns=p≥0.05) (n=5 mice per group). FIG. 2F contains representative mouse images (right) and a graph (left) of GVHD clinical scoring results from day 50 on following treatment with EcCAR-MSC, UTD-MSC, or no MSCs, with. Data shown are mean+/−s.d. with statistical analysis performed by 2-way ANOVA **p≤0.01, ns=p≥0.05) (n=5 mice per group). FIG. 2G is a graph of a mouse peripheral blood assessment comparing absolute number of human CD³⁺ T cells between EcCAR-MSC and UTD-MSC treatment groups by flow cytometry 2 weeks following first MSC injection. Data shown are mean+/−s.d. with statistical analysis performed by unpaired t-test (**p≤0.01) (n=5 mice per group). FIG. 2H is a graph of survival outcomes following treatment with EcCAR-MSCs compared to UTD-MSCs and no MSC control. Statistical analysis was performed by Kaplan-Meier Simple survival analysis (**p≤0.01, ns=p≥0.05) (n≥5 mice per group). Tumor figures (FIGS. 2A-2C) are representative of 2 independent experiments on 2 different tumor models and 2 different biological MSC donors. GVHD figures (FIGS. 2D-2H) are representative of 3 independent experiments on 3 different biological PBMC donors and 3 different biological MSC donors performed on both male and female NSG mice.

FIGS. 3A-3E show that RNAseq analyses revealed antigen specific activation of EcCAR-MSCs leading to enrichment of immunosuppressive pathways. FIG. 3A is a heat map depicting the distinct gene expression profile of EcCAR-MSC+Ecad samples highlighting the functional impact of CAR antigen-specific stimulation through hierarchical clustering with unstimulated EcCAR-MSC, stimulated UTD-MSC+Ecad, and unstimulated UTD-MSC samples. Displaying gene fold counts normalized across all samples (padj≤0.05) (n=3 MSC donors per condition). FIG. 3B is a PCA depicting RNAseq transcript profiles of 6 samples from 3 biological MSC replicates indicating clustering solely by donor in unstimulated UTD-MSCs and stimulated UTD-MSCs. FIG. 3C is a PCA of unstimulated and stimulated EcCAR-MSCs revealing unique clustering by stimulation group indicating CAR based functional activation. FIG. 3D uses Ingenuity Pathway Analysis (IPA) to reveal upregulated canonical pathways in stimulated vs. unstimulated EcCAR-MSCs. Dashed line marking significantly enriched pathways (p≤0.05) (n=3 donors per group). FIG. 3E contains graphs of a normalized gene count comparisons across unstimulated UTD-MSC, stimulated UTD-MSC, unstimulated EcCAR-MSC, and stimulated EcCAR-MSC showing activation of CAR (CD28)-linked transcription factor genes NFκB1, JUN, RELB, and IRF1 in stimulated EcCAR-MSCs. Data shown are mean+/−s.d. of normalized gene counts across groups. Statistical analysis was performed by 1-way ANOVA (**p≤0.01, ***p≤0.001, ****p≤0.0001) (n=3 donors per group).

FIGS. 4A-4I show that EcCAR-MSCs support enhanced immunosuppression through increased secretion of cytokines, T cell mediation, and upregulation of inhibitory surface markers. FIG. 4A contains graphs of serum cytokines levels in pg/mL of IL-10 polypeptides, TNFα polypeptides, G-CSF polypeptides, Eotaxin polypeptides, and FGF-2 polypeptides in EcCAR-MSC treated mice as compared to UTD-MSC, and untreated xenografts by multiplex assay 17 days following administration. Data shown are mean+/−SEM with statistical analysis performed by ordinary 1-way ANOVA (*p≤0.05, **p≤0.01, ***p≤0.001, ****p≤0.0001) (n=4-6 samples per group). FIG. 4B is a graph of human T cell subsets in EcCAR-MSC vs. UTD-MSC treated mice from GVHD xenografts. Quantification of human CD4+ and CD8⁺ T cell suppression are depicted by absolute cell counts within peripheral mouse blood 2 weeks following first MSC injection by flow cytometry. Data shown are mean+/−s.d. of the absolute number of cell subsets. Statistical analysis was performed by 2-way ANOVA (*p≤0.05, ***p≤0.001) (n=5 per group). FIG. 4C is a graph of human CD4⁺ to CD8⁺ T cell proportions by flow cytometry in mice treated with EcCAR-MSCs compared to UTD-MSCs in GVHD xenografts. Data shown are mean+/−s.d. of percent of cell subsets. Statistical analysis was performed by 2-way ANOVA (**p≤0.01) (n=5 per group). FIG. 4D is a graph correlating percent weight change in GVHD mice from baseline day 0 to day 31 showing significant amelioration of weight loss and prevention of GVHD in EcCAR-MSC treated mice only. Data shown are mean+/−SEM of percent weight change. Statistical analysis was performed by 2-way ANOVA (***p≤0.001) (n=5 per group). FIG. 4E is a graph of human CD4″, CD25+, and CD127 Treg-like cell subset 4 weeks following PBMC administration with MSC treatment. Flow cytometry analysis revealed a significant increase in Treg % within EcCAR-MSC treated mice as compared to UTD-MSC. Data shown are mean+/−s.d. of cell subset percentages Statistical analysis was performed by unpaired t-test (***p≤0.001) (n=4 per group). FIG. 4F is a T-distributed stochastic neighbor embedding (tSNE) plot of surface marker expression by flow cytometry time of flight (CyTOF) of Ecad stimulated and unstimulated EcCAR-MSCs and UTD-MSCs. Ecad stimulation was induced via Ecad⁺ irradiated cell line MCF7 for 24 hours prior to CyTOF analysis. tSNE plot of surface marker landscape within samples with unsupervised clustering revealed the presence of 4 major cell populations: Ecad⁺ Cell Line, UTD-MSC, EcCAR-MSCs, and Ecad stimulated EcCAR-MSCs. FIG. 4G is a heat map displaying cluster surface characterization with CXCR3 and PD1 markers uniquely characterizing stimulated EcCAR-MSC population cluster. FIG. 4H is a graph of the percent of the expression of inhibitory receptor surface markers PD-1 and Gal-9 analyzed by flow cytometry in UTD-MSCs and EcCAR-MSCs stimulated with soluble Ecad and cocultured with PBMCs for 5 days before surface marker assessment. FIG. 4I is a graph of the percent of the expression of migratory chemokine surface markers CXCR3 and CXCR4 analyzed by flow cytometry in UTD-MSCs and EcCAR-MSCs stimulated with soluble Ecad and cocultured with PBMCs for 5 days before surface marker assessment. Data shown in FIGS. 4H-4I are mean+/−s.d. % surface expression representing 3 MSC donors. Statistical analysis was performed by 2-way ANOVA (**p≤0.01, ****p≤0.0001) (n=3 per group).

FIGS. 5A-5G show EcCAR-MSC homing and safety profiles within in vivo canine and murine models. FIG. 5A is a schematic of an exemplary method for CAR-MSC manufacturing, safety, and homing analysis for in vivo healthy canine models. FIG. 5B contains an exemplary immunohistochemical (IHC) analysis of a transverse colon histology stained for Ecad 3 days following EcCAR-MSC administration indicating homing of human EcCAR-MSCs to canine Ecad⁺ rich tissues. Human CD105 positive antibody (top) and canine Ecad positive antibody (bottom) appear colocalized via IHC analysis at various magnifications in canine colon tissue. FIG. 5C contains graphs of complete blood count levels following the i.p. administration of EcCAR-MSCs. This includes, from left to right, white blood cells (WBCs), monocytes, lymphocytes, neutrophils, and platelets with short term (3 day) and long term (28 day) blood level monitoring following in vivo EcCAR-MSC injection as compared to baseline. Data shown are mean+/−s.d. of blood composition with statistical analysis performed by 2-way ANOVA (ns=p≥0.05 between groups) (n=3 per experimental group). FIG. 5D contains graphs, from left to right, of total protein, BUN, creatinine, albumin, and alkaline phosphatase levels with short term (3 day) and long term (28 day) monitoring following in vivo EcCAR-MSC injection. Data shown are mean+/−s.d. of blood composition with statistical analysis performed by 2-way ANOVA (ns=p≥0.05). FIG. 5E is a schematic of an exemplary method for determining the expansion kinetics and persistence of CAR-MSCs in vivo using a luciferase positive MSC xenograft model. Luciferase expressing UTD-MSC or CAR-MSCs were injected into human PBMC-primed NSG mice with or without irradiated Ecad⁺ MCF7 cells as a form of CAR stimulation. Mice were followed by serial bioluminescent imaging on daily basis. FIG. 5F is a graph of the flux of bioluminescent imaging as a measure of MSC expansion and clearance kinetics following administration of EcCAR-MSC or UTD-MSC with or without Ecad stimulation. FIG. 5G is an exemplary image of luciferase-positive MSC expansion and clearance in mice from 3 to 24 days following administration. Data shown are mean+/−standard deviation (s.d.) with statistical analysis performed by 2-way ANOVA (ns=p≥0.05) (n=5 per group).

FIG. 6 shows the canine/human cross reactivity of a luciferase canine Ecad⁺ MCKD cell line co-cultured with human versus mouse versus canine directed Ecad CAR T cells. Bioluminescent imaging was taken after 24 hours of co-culturing. UTD=untransduced.

FIG. 7 shows a schematic of an exemplary EcCAR-MSC construct where hmcECAD.6 represents human, mouse, and canine cross-reactive Ecad antigen binding domain.

FIG. 8A is a schematic of an exemplary method for evaluating the effect of EcCAR-MSCs on JeKo1 cell xenograft tumor model. JeKo1 cells were injected (1×10⁶ cells i.v.) into NSG mice along with either EcCar-MSCs or UTD-MSCs (1×10⁶ cells i.p. on days 0, 14, and 28). Mice were then monitored for weight loss, the development of clinical signs of GVHD (scored based on weight loss, diarrhea, posture, activity, fur texture, and skin integrity), and survival. FIG. 8B is a graph of the tumor flux (photons/sec) of NSG mice injected with JeKo1 cells.

FIG. 9A is a graph of differentially expressed genes in unstimulated EcCAR-MSC vs. UTD-MSC, Ecad-stimulated vs. unstimulated EcCAR-MSCs, and Ecad-stimulated vs. unstimulated UTD-MSCs. Data display unregulated and downregulated gene counts within comparisons with adj. p value<0.01 and ±1−log fold change. Transcriptional alterations induced by Ecad stimulation of CAR-MSCs included 2362 significant genes vs. EcCAR-MSC alone and 3032 significant genes vs. Ecad stimulated UTD-MSCs. Transcriptional alterations induced by CAR transduction included 606 significant genes. Transcriptional alterations induced by Ecad stimulation included 206 significant genes. FIG. 9B shows that Ingenuity Pathway Analysis (IPA) revealed upregulated canonical pathways in unstimulated EcCAR-MSCs vs. UTD-MSCs. The dashed line marks enriched pathways (p≤0.05) (n=3 MSC donors per group).

FIG. 10 contains graphs of, from left to right, MDC polypeptides, GRO polypeptides, GM-CSF polypeptides, MCP-3 polypeptides, and Flt-3 polypeptides in EcCAR-MSC treated mice as compared to UTD-MSC, and untreated xenografts by multiplex assay 17 days following administration. Data shown are mean+/−SEM with statistical analysis performed by ordinary 1-way ANOVA (*p≤0.05, **p≤0.01, ***p≤0.001, ****p≤0.0001) (n=4-6 samples per group).

FIG. 11 is a heat map of the clustering of surface marker expression in populations of cells identified by CyTOF constituting cell clusters from FIG. 4F.

FIG. 12A displays bodyweight changes in healthy canines following administration of EcCAR-MSCS as compared to control. No significant differences in body weight changes were found between groups. Data showing mean±s.d. of % weight change from baseline with statistical analysis performed by ordinary 1-way ANOVA (ns=p≥0.05). FIG. 12B contains data displaying transverse colonic tissue sections of canines through H&E staining 28 days following administration at 20× and 40× magnification following treatment with human EcCAR-MSC (left) or control (right).

FIG. 13 is a histogram showing CAR expression on MSCs containing variable intracellular signaling domains including 4-1BB, CD28, TLR, and IFNγ signaling domains as detected by flow cytometry using CAR⁺ FMC63 antibody. Data shown are mean+/−s.d. representative of at >6 independent experiment using 5 MSC donors.

FIG. 14 contains a schematic of exemplary mechanisms for enhancing immunosuppression and trafficking of CAR-MSCs.

FIGS. 15A-15F show that MSCs are stably transduced to express CAR and maintain stem-like features following transduction and stimulation. FIG. 15A is a representative flow cytometry plot depicting CAR19 expression following lentiviral transduction of MSCs with increasing concentrations of protamine sulfate enhancer (50 μg/mL and 100 μg/mL, as indicated) compared to untransduced (UTD) MSCs. Data are representative using 6 different biological MSC donors. FIG. 15B is a histogram showing CAR expression of anti-Ecad CAR and anti-CD19 CAR positive control as detected by flow cytometry. Data shown are mean±standard deviation (s.d.) of 6 different biological MSC donors. FIG. 15C is a graph showing CAR expression on EcCAR-MSC compared to UTD-MSC by flow cytometry 2 days and 11 days following transduction and ex vivo expansion. Data shown are mean±s.d. representative of 3 independent experiments using 3 MSC donors. Statistical analysis was performed by 2-way ANOVA (****p≤0.0001). FIG. 15D is a histogram showing EcCAR expression on MSCs across 3 different primary biological MSC donors. Data are representative of ≥10 independent experiments using 6 different MSC donors. FIG. 15E is a graph showing the stem phenotype of EcCAR-MSC and UTD-MSC measured with flow cytometry after staining MSCs for CD105, CD90, CD73, CD34, CD45, and CD14 surface markers. Data shown are mean±s.d. results of 3 independent experiments of 4 biological MSC donors. Statistics were performed by 2-way ANOVA (ns=p≥0.05). FIG. 15F depicts two graphs of bulk RNAseq analysis of upregulated (left panel) and downregulated (right panel) phenotypic enrichments of EcCAR-MSCs as compared to UTD-MSCs by CellMarker Augmented gene set enrichment analysis. Significant genes were selected by ≥1 or ≤−1 log fold change and ≤0.05 adjusted p values (padj) via differential expression analysis of 3 individual MSC donors. Dashed line across x axes represent statistically significant enrichment for all pathways −log (p≤0.05).

FIGS. 16A-16G show that EcCAR-MSCs demonstrate superior antigen-specific suppression of primary T cells in vitro. FIG. 16A is a histogram displaying establishment of Ecad⁺ NALM6 with matched Ecad⁻ NALM6, making the Ecad cell-based stimulation appropriate for use in in vitro suppression assays and in vivo tumor models. FIG. 16B is a graph of the absolute numbers of CD³⁺ T cells following coculture with EcCAR-MSCs or UTD-MSCs with increasing concentrations of soluble Ecad (Ong/mL, 250 ng/mL, and 1000 ng/mL). Data shown are mean±s.d. of 3 independent experiments using 3 individual MSC donors. Statistics were performed by 2-way ANOVA (***p≤0.001). FIG. 16C is a graph of the stem phenotype of EcCAR-MSC with increasing (0, 250, or 1000 ng/ml) soluble Ecad stimulation measured with flow cytometry after staining MSCs for CD73, CD90, CD105, CD14, CD34, and CD45. Data shown are mean±s.d. using 4 different MSC donors. Statistics were performed using a 2-way ANOVA (ns=p≥0.05). FIGS. 16D, 16E, and 16F are graphs of the absolute numbers of CD³⁺ T cells of MSCs from 3 different donors following co-culture with EcCAR-MSCs or UTD-MSCs derived with or without Ecad⁺ or Ecad⁻ cell-based stimulation. Data shown are mean±s.d. Statistics were performed using a 2-way ANOVA (n=2-3 replicates per donor) (*p≤0.05, **p≤0.01). FIG. 16G is a graph of the stem phenotype of EcCAR-MSCs or UTD-MSCs following co-culture alone or with Ecad* or Ecad cell-based stimulation measured by flow cytometry after staining MSCs for CD73, CD90, CD105, CD14, CD34, and CD45. Data shown are mean±s.d. results from n=3 MSC donors. Statistics were performed using a 2-way ANOVA (ns=p≥0.05).

FIGS. 17A-17G show that EcCAR-MSCs induce superior immunosuppression in tumor and GvHD Xenograft models. FIG. 17A is a schematic of an exemplary method for evaluating the effect of Ecad⁺ in a Nalm6 xenograft tumor model. Immunocompromised NOD-SCID-γ^{-/-} (NSG) mice were engrafted with luciferase (luc)+Ecad⁺ or luc⁺ Ecad-NALM6 cells (1×10⁶ cells i.v.) and treated with CD19-targeted CAR T (CART19) (1×10⁶ cells i.v.). Mice were then randomized to receive untransduced MSCs (UTD-MSCs) or EcCAR-MSCs (1×10⁶ cells i.p.) and monitored for tumor burden by bioluminescent imaging (BLI) and monitored for survival and health. FIG. 17B is a graph of Ecad⁺ NALM6 tumor flux following CART19 infusion in the Nalm6 model, comparing UTD-MSCs and EcCAR-MSCs. Data shown are median±standard error of the mean (SEM) with statistical analysis using a 2-way ANOVA (***p≤0.001; n=4-5 mice per group, 2 independent experiments). FIG. 17C is a graph of Ecad⁻ NALM6 tumor flux following CART19 infusion in the Nalm6 model, comparing UTD-MSCs and EcCAR-MSCs. Data shown are median±SEM with statistical analysis using a 2-way ANOVA (ns=p≥0.05; n=4-5 mice per group; 2 independent experiments). FIG. 17D is a schematic of an exemplary method for evaluating the effect of EcCAR-MSCs in a human PBMC-induced GvHD xenograft model. Human peripheral blood mononuclear cells (PBMCs) were injected (25-30×10⁶ cells i.v) into NSG mice that had been randomized to receive treatment with EcCAR-MSCs or UTD-MSCs (1×10⁶ cells i.p.). Mice were monitored for weight loss, the development of clinical GvHD symptoms (scored based on weight loss, diarrhea, posture, activity, fur texture, and skin integrity), and survival. FIG. 17E is a graph of the percent weight change from baseline in GvHD xenografts following treatment with UTD-MSCs, EcCAR-MSCs, or no treatment. Data shown are mean±SEM with statistical analysis using a 2-way ANOVA (ns=p≥0.05, *p≤0.05, ****p≤0.0001; n=5 mice per group, 3 independent experiments). FIG. 17F is a graph of the GvHD clinical scoring severity (left) following treatment with EcCAR-MSCs, UTD-MSCs, or no MSCs, with representative mouse images (right). Data shown are mean±SEM with statistical analysis using a 2-way ANOVA (ns=p≥0.05, *p≤0.05; n=5 mice per group, 3 independent experiments). FIG. 17G is a graph of the survival outcomes following treatment with EcCAR-MSCs compared to UTD-MSCs and no MSC control. Statistical analysis used a Kaplan-Meier survival analysis (ns=p≥0.05, *p≤0.05, **p≤0.01; n=5 mice per group).

FIGS. 18A-18G shows that EcCAR-MSCs display antigen-specific activation and trafficking to Ecad⁺ colonic target tissue in acute GvHD xenograft models. FIG. 18A is a schematic of an exemplary method of for evaluating the effect of Ecad⁺ in an acute GvHD xenograft model. Immunocompromised NOD-SCID-γ^{-/-} (NSG) mice were first irradiated at 250 cGy to further prime an inflammatory environment. Human PBMCs were injected (10-15×10⁶ cells i.v) into mice along with luc⁺ GFP⁻ Ecad-CAR-MSCs or luc⁺ GFP⁺ CD19-CAR-MSCs (1×10⁶ cells i.p.). Mice were monitored for long-term weight loss, the development of clinical GvHD, and survival. Satellite mice were isolated one week following MSC injection for assessment of MSC localization in colonic target organs by bioluminescent imaging and immunofluorescent staining. FIG. 18B is a graph of the percent weight change from baseline in acute GvHD xenografts following treatment with Ecad-CAR-MSCs, CD19-CAR-MSCs, or no treatment. Data shown are mean±SEM with statistical analysis using a 2-way ANOVA (**p≤0.01, ***p≤0.001; n=4-5 mice per group, 3 independent experiments). FIG. 18C is a graph of survival outcomes following treatment with Ecad-CAR-MSCs compared to CD19-CAR-MSCs and no MSC control. Statistical analysis used a Kaplan-Meier survival analysis (*p≤0.05; n=4-5 mice per group). FIG. 18D is a graph of the absolute number of human CD³⁺ T cells in peripheral blood comparing Ecad-CAR-MSC and CD19-CAR-MSC treated mice by flow cytometry 2 weeks following MSC injection. Data shown are mean±s.d. with statistical analysis using an ordinary 1-way ANOVA (*p≤0.05; n=4-5 mice per group, 2 independent experiments). FIG. 18E shows representative bioluminescent imaging across luc⁺ CD19-CAR-MSC and Ecad-CAR-MSC treated mice (left) with percent MSC flux to colon relative to total flux detected across all organs (right). Data shown are mean+/−s.d. with statistical analysis using unpaired t-test (*p≤0.05; n=4-5 mice per group, 2 independent experiments). FIG. 18F is a graph of immunofluorescent-based quantification of EcCAR-MSC compared to CD19-CAR-MSC localization to Ecad⁺ colon tissue. Data were determined by the percent of MSC⁺ colonic crypts per focal image. Data shown are mean±s.d. with statistical analysis using and ordinary 1-way ANOVA (**p≤0.01; n=3 mice per group). FIG. 18G is a series of representative immunofluorescent image of mouse colonic tissue isolated from acute GvHD xenograft models 7 days following GFP⁺ MSC administration. Comparisons are between CD19-CAR-MSC and Ecad-CAR-MSC (2^{nd} row) colocalization with E-cadherin⁺ (3^{rd} row) colonic regions. Cell nuclei are stained with DAPI with each color channel displayed to make merged image. Images obtained at 40× magnification capturing 1x crop area.

FIGS. 19A-19F show that activation of antigen-specific immunosuppressive signaling pathways identified in EcCAR-MSCs. FIG. 19A is a heat map depicting the distinct gene expression profile of stimulated EcCAR-MSC+Ecad samples highlighting the functional impact of CAR antigen-specific stimulation through hierarchical clustering with unstimulated EcCAR-MSC, stimulated UTD-MSC+Ecad, and unstimulated UTD-MSC samples. Data shown are gene fold counts normalized across all samples (padj≤0.05) (n=3 MSC donors per condition). FIG. 19B is a graph of a principal Component Analysis (PCA) of gene expression profiles across 6 samples cluster by derived MSC donor in stimulated compared to unstimulated UTD-MSCs. FIG. 19C is a graph of a PCA of gene expression profiles of stimulated compared to unstimulated EcCAR-MSCs and reveals unique clustering by stimulation group. FIG. 19D is a schematic summary of an analysis using Ingenuity Pathway Analysis (IPA) machine learning algorithm illustrating factors activated when stimulated EcCAR-MSCs compared to unstimulated EcCAR-MSCs. Factors including upstream regulators, canonical pathways, and biological functions were combined to predict meaningful functional impacts. Analysis revealed that apoptosis of leukocytes (center) to be the most significantly enriched (padj≤0.0001) functional pathway directly associated with all 17 activated molecules predicted in the dataset including CD28 CAR signaling molecule. FIG. 19E is a graphical representation of the IPA of FIG. 16D revealing upregulated canonical pathways in stimulated EcCAR-MSCs compared to unstimulated EcCAR-MSCs. Dashed line across x axes represent statistically significant enrichment for all pathways-log (p≤0.05) (n=3 MSC donors per group). FIG. 19F is a series of graphs of normalized gene count comparisons across unstimulated UTD-MSCs, stimulated UTD-MSCs, unstimulated EcCAR-MSCs, and stimulated EcCAR-MSCs. CD28-linked transcription factor genes (NFκB1, JUN, RELB, and IRF1) and downstream effector genes (TRAF1, TLR3, FYN) in stimulated EcCAR-MSC group only. Data shown are mean±s.d. Gene counts are normalized across groups. Statistical analysis used a 1-way ANOVA (ns=p≥0.05, *p≤0.05, **p≤0.01, ***p≤0.001, ****p≤0.0001).

FIGS. 20A-20H show that EcCAR-MSC stimulation resulted in increased cytokine secretion, surface marker expression, and subsequent T cell modulation. FIG. 20A is a heatmap displaying upregulated cytokines in Ecad⁺ cell line stimulation compared to Ecad-cell line stimulation on T cells alone, UTD-MSCs, and EcCAR-MSCs by multiplexed cytokine assay. Data shown are degree of cytokine fold-change as measured by multiplexed assay normalized by smallest (0) and largest (100) values per cytokine (n=2 technical replicates per assay). FIG. 20B is a series of graphs of inhibitory surface marker expression across UTD-MSCs and EcCAR-MSCs with Ecad⁺ cell line stimulation as compared to Ecad-cell line stimulation. Data shown are mean±s.d. results with statistical analysis using a 2-way ANOVA (***p≤0.001, ****p≤0.0001; n=3 replicates per donor). FIG. 20C is a series of graphs of enriched serum cytokines (IL-10, TNFα, G-CSF, eotaxin, and FGF-2 by pg/mL) at 2 weeks in EcCAR-MSC-treated mice from tumor xenografts. Data shown are mean±SEM with statistical analysis using an ordinary 1-way ANOVA (*p≤0.05, **p≤0.01, ***p≤0.001, ****p≤0.0001; n=4-6 mice per group). FIG. 20D is a graph of the percent weight change compared to baseline showing prevention of GvHD-induced weight loss following single dose treatment of EcCAR-MSCs compared to UTD-MSCs. Displaying mean±SEM of percent weight change. Statistical analysis used a 2-way ANOVA (***p≤0.001; n=5 mice per group). FIG. 20E is a graph of the absolute number of human CD³⁺ T cells in mouse peripheral blood in EcCAR-MSC and UTD-MSC treatment groups as measured by flow cytometry 2 weeks following first MSC injection. Data shown are mean±s.d. with statistical analysis using an unpaired t-test (**p≤0.01; n=5 mice per group). FIG. 20F is a graph of human CD4⁺ and CD8⁺ T cells quantified in mouse peripheral blood 2 weeks following EcCAR-MSC treatment. Data shown are mean±s.d. cell counts. Statistical analysis used a 2-way ANOVA (*p≤0.05, ***p≤0.001; n=5 mice per group). FIG. 20G is a graph showing an alteration in human CD4⁺ to CD8⁺ T cell proportions in mice treated with EcCAR-MSCs compared to treatment with UTD-MSCs. Data shown are mean±s.d. of percent cell subsets. Statistical analysis used a 2-way ANOVA (**p≤0.01; n=5 mice per group). FIG. 20H is a graph of human CD4⁺CD25⁺CD127⁻ Treg cell subsets 4 weeks following MSC treatment in GvHD xenograft mice. Data shown are mean±s.d. % of cells. Statistical analysis used an unpaired t-test (***p≤0.001; n=4 mice per group).

FIGS. 21A-21G show that the CD28 signaling domain within EcCAR-MSCs increases immunosuppressive efficacy. FIG. 21A is a schematic of an exemplary method of EcCAR-MSC construct designs used to investigate CAR-MSC mechanism of action. Constructs contained the full CD28ζ, CD28 alone, CD3ζ alone, and null (no signaling domain) EcCAR-MSCs. FIG. 21B is a histogram of CAR expression on MSCs for CD28ζ, CD28, CD3ζ, and null EcCAR-MSC constructs detected by flow cytometry. Displaying CAR % expression representing ≥5 independent experiments on 4 independent MSC donors. FIG. 21C is a series of graphs showing the degree of CD3⁺ T cell suppression following Ecad⁺ cell line stimulation across each EcCAR-MSC signaling domain subtype or T cells alone in culture with Ecad⁺ and matched Ecad cell line. Data shown are mean±s.d. representative of 4 primary T cell donors. Statistical analysis used a 1-way ANOVA (ns=p≥0.05, *p≤0.05, **p≤0.01, ***p≤0.001; n=3 technical replicates per donor). FIG. 21D is a schematic of a representative method to test the effect of various CAR-MSC signaling domains in an acute GvHD xenograft mouse model. NSG mice were irradiated at 250 cGy to prime inflammatory environment. Human PBMCs were injected (10-15×10⁶ cells i.v) into mice along with one of the following MSC groups: CD28ζ, CD28, CD3ζ, or null EcCAR-MSCs, UTD-MSCs (1×10⁶ cells i.p.), or “No MSC” treatment. Mice were monitored for long-term weight loss, the development of clinical GvHD symptoms, and survival. FIG. 21E is a graph of the percent weight change from baseline in the acute GvHD xenografts following treatment with each of the MSC groups or no treatment. Data shown are mean±SEM with statistical analysis using a 2-way ANOVA (ns=p≥0.05, *p≤0.05, **p≤0.01; n=5-6 mice per group, 2 independent experiments). FIG. 21F is a graph of the GvHD clinical scoring severity of symptom progression following treatment with alternative MSC signaling domains. Data shown are mean±SEM with statistical analysis using a 2-way ANOVA (*p≤0.05; n=5-6 mice per group, 2 independent experiments). FIG. 21G is a graph of the survival outcomes of acute GvHD xenograft mouse model mice following treatment with each EcCAR-MSC subtype. Data depicting trends in enhanced survival, but without statistical significance, are included with prospective p value measurements. Statistical analysis used a Kaplan-Meier survival analysis (ns=p≥0.05, *p≤0.05, **p≤0.01; n=5-6 mice per group).

FIGS. 22A-22I show EcCAR-MSC safety and clearance profiles across tissues. FIG. 22A is a schematic for a representative method to determine the effect of EcCAR-MSCs in a MSC persistence model. Either luc⁺ GFP⁺ UTD-MSCs or luc⁺ GFP⁺ CAR-MSCs were injected into human PBMC-primed NSG mice with or without CAR stimulation through coadministration of irradiated Ecad⁺ cells. Mice were followed by serial biweekly bioluminescent imaging (BLI). FIG. 22B is a representative image of luciferase⁺ MSC expansion and clearance in mice from 3 to 24 days following administration. FIG. 22C is a graph of the MSC expansion and clearance kinetics following administration of EcCAR-MSCs or UTD-MSCs with or without additional Ecad stimulation as measured by flux of BLI. Data shown are mean BLI with statistical analysis using a 2-way ANOVA (ns=p≥0.05; n=4-5 mice per group). FIG. 22D is a graph of the absolute number of keratinocytes following 24-hour coculture with UTD-MSCs or EcCAR-MSCs. Data shown are mean #s.d. with statistical analysis using a paired t-test (ns=p≥0.05; n=3 replicates per donor). FIG. 22E is a graph of Ecad expression identified on keratinocytes following 24-hour coculture with UTD-MSCs or EcCAR-MSCs. Data shown are mean %±s.d. with statistical analysis using a paired t-test (ns=p≥0.05; n=3 replicates per donor). FIG. 22F is a heatmap displaying upregulated cytokines across UTD-MSCS, EcCAR-MSCs, and keratinocytes at baseline and following 24-hour coculture conditions. Data shown are cytokine levels measured by multiplexed assay normalized by smallest (0) and largest (100) values per cytokine (n=2 technical replicates per assay). FIG. 22G is a graph of the absolute number of bronchial cells following 24-hour coculture with varying ratios of UTD-MSCs or EcCAR-MSCs. Shown data are mean±s.d. with statistical analysis using a paired t-test (ns=p≥0.05; n=3 technical replicates). FIG. 22H is a graph of immunofluorescent-based quantification of EcCAR-MSC localization compared to CD19-CAR-MSC localization in Ecad⁺ lung tissue. Data was determined by the % of MSC⁺ regions per focal image. Data shown are mean±s.d. with statistical analysis using an ordinary 1-way ANOVA (ns=p≥0.05; n=3 mice per group). FIG. 22I is a representative image of immunofluorescence imaging of mouse bronchial (lung) tissue isolated from acute GvHD xenograft model 7 days following GFP⁺ MSC administration. Comparisons are between colocalization of Ecad⁺ (2^{nd} column) to bronchial regions in CD19-CAR-MSC and Ecad-CAR-MSC (last column) treated mice. Cell nuclei are stained with DAPI (3^{rd} column) with each color channel displayed to make merged image. Images obtained at 40× magnification capturing 1x crop area.

FIGS. 23A-23D show EcCAR construct design and manufacturing. FIG. 23A is a representative sequence of an optimized anti-Ecad scFv clone sequence (hmcECAD.6) identified through phage display generation with Ecad selection. hmcECAD was selected with affinity to human, mouse, and canine cross-reactivity and cloned into EcCAR construct to generate EcCAR-MSCs. FIG. 23B is a representative schematic of a full EcCAR-MSC construct containing hmcCAD heavy and light chains for binding to Ecad, CD28 hinge and transmembrane domains for passage through MSC cell membrane, and CD28 intracellular signaling domain for immunosuppressive activation with CD33 costimulation as regularly employed for CAR construct stability. FIG. 23C is a graph of phage ELISA results used to test the relative affinity of hmcECAD.6 scFv clone to mouse Ecad as compared to FLAG tag positive control. Data shown are all technical replicates (n=3) with statistical analysis using a 2-way ANOVA (ns=p≥0.05). FIG. 23D is a graph showing dose-dependent killing of human and mouse Ecad⁺ tumors (MCF7 and 4T1, respectively) by CART cells containing hmcECAD.6 scFv construct. Data shown are all technical replicates (n=3) with statistical analysis using a 2-way ANOVA (**p≤0.01, ****p≤0.0001).

FIGS. 24A-24D show an exemplary method and confirmation of the generation of CAR-MSCS with verification of stemness. FIG. 24A is a schematic of an exemplary method for EcCAR-MSC generation in culture following thawing from cryogenic storage systems. Briefly, following in vitro outgrowth in T175 flasks, MSCs are digested and quantified for transduction with optimized CAR plasmid-containing lentiviral vector delivery system and protamine sulfate enhancer. CAR % confirmation is completed by day 5 via flow cytometric analysis of goat anti-mouse antibody. FIG. 24B is a graph of the stem phenotype of EcCAR-MSC with and without Ecad⁺ cell line stimulation measured by flow cytometry after staining with surface markers and with additional positive control measurements using primary naïve T cells. Data shown are mean±s.d. from 3 individual MSC donors. Statistical analysis used a 2-way ANOVA (****p≤0.0001). FIG. 24C is a representative image of UTD-MSC and CAR-MSC morphological comparisons at baseline and with the addition of matched Ecad and Ecad⁺ cell line as a means of CAR antigen-specific stimulation. FIG. 24D is series of histograms characterizing MSC stem phenotype by positive CD105, CD90, and CD73 and negative CD34, CD45, and CD14 surface markers. Positive gates were established by the fluorescence minus one (FMO) method for negative control (top) for positive sample quantification of UTD-MSCs (middle) and EcCAR-MSCs (bottom).

FIGS. 25A-25G show the effect of EcCAR-MSCs on CART effector functions in in vivo tumor model. FIG. 25A is a graph comparing of Ecad⁻ NALM6 and Ecad⁺ NALM6 tumor flux measurements across control mice without CART19 cell infusion. Data shown are mean±SEM with statistical analysis using a 2-way ANOVA (ns=p≥0.05; n=4-5 mice per group). FIGS. 25B and 25C are graphs of the relative levels of luc⁺ Ecad⁻ NALM6 (FIG. 25B) and luc⁺ Ecad⁺ NALM6 (FIG. 25C) as measured by luminescence following 24-hour in vitro coculture with UTD-MSCs or EcCAR-MSCs at varying MSC to NALM6 ratios. Data shown are mean±s.d. with statistical analysis using a 2-way ANOVA (ns=p≥0.05; n=2 replicates per group). FIG. 25D is a representative schematic of a method of evaluating CART19 in NALM6 and JeKo-1 tumor models. NSG mice engrafted with luciferase⁺ CD19⁺ Nalm6 or JeKo-1 cells (1×10⁶ i.v.) and treated with CART19 (1×10⁶ cells i.v.) and irradiated Ecad⁺ cell line. Mice were then randomized to receive UTD-MSCs or EcCAR-MSCs (1×10⁶ cells i.p.) and monitored biweekly for bioluminescent imaging and survival. FIG. 25E is a graph of tumor flux following CART19 infusion in Jeko-1 tumor model, comparing EcCAR-MSCs, UTD-MSCs, or no MSC treatment. Data shown are mean±SEM with statistical analysis by 2-way ANOVA (**p≤0.01; n=3-4 mice per group). FIG. 25F is a graph of tumor flux measurements across MSC administration groups following CART19 infusion in NALM6 model. Data shown are mean±SEM with statistical analysis using a 2-way ANOVA (ns=p≥0.05, ***p≤0.001; n=3-4 mice per group, 2 independent experiments). FIG. 25g is a graph of the survival outcomes of mice following treatment with EcCAR-MSCs compared to UTD-MSCs and no MSC control groups. Statistical analysis use a Kaplan-Meier survival analysis (ns=p≥0.05, *p≤0.05; n=3-4 mice per group, 2 independent experiments).

FIGS. 26A-26B show exemplary flow cytometry gating strategies. FIG. 26A is an exemplary gating strategy used for identification of positive MSC surface markers as verified by FMO and internal controls. MSC populations are identified by size, single cells are selected through diagonal gating, live MSCs are stained with live/dead fixable aqua fluorophore to exclude dead cells, and positive cell markers (CAR⁺ % shown) are identified based on positive gating (verified by internal and FMO controls) of cellular stain of interest. FIG. 26B is an exemplary gating strategy used for identification of luciferase⁺ and CAR⁺ MSCs for assessment in in vivo clearance experiments. MSCs were transduced with both luciferase-expressing transgene as well as CAR construct and compared to luciferase⁺ MSC and UTD-MSC controls. Results indicate successful transduction of luciferase and CAR with >50% double luciferase⁺ CAR⁺ for intraperitoneal injection in vivo.

FIGS. 27A-27E show bulk RNAseq pathway analysis and cytokine secretion. FIG. 27A is a graph showing a summary of differentially expressed genes in unstimulated EcCAR-MSCs compared to UTD-MSCs, Ecad-stimulated compared to unstimulated EcCAR-MSCs, Ecad-stimulated compared to unstimulated UTD-MSCs, and Ecad-stimulated EcCAR-MSCs compared to UTD-MSCs. Data shown are gene counts of significantly unregulated and downregulated genes within comparisons with adj. p value<0.01 and ±1-log fold change. Transcriptional alterations induced by Ecad stimulation of CAR-MSCs included 2362 significant genes compared to EcCAR-MSC alone and 3032 significant genes compared to Ecad stimulated UTD-MSCs. Transcriptional alterations induced by CAR transduction included 606 significant genes. Transcriptional alterations induced by Ecad stimulation included 206 significant genes. FIG. 27B is a graph of an Ingenuity Pathway Analysis (IPA) revealing upregulated canonical pathways in unstimulated EcCAR-MSCs compared to UTD-MSCs. Dashed line across x axes represent statistically significant enrichment for all pathways −log (p≤0.05) (n=3 MSC donors per group). FIGS. 27C and 27D are graphical summaries of the IPA of FIG. 27B illustrating significant entities activated in unstimulated EcCAR-MSCs vs. UTD-MSCs (FIG. 27C) and significant entities activated in stimulated EcCAR-MSCs compared to UTD-MSCs (FIG. 27D). Canonical pathways and activated molecules were used to predict meaningful functional impacts between datasets. FIG. 27E is a graph showing serum cytokine elevations found in peripheral blood from EcCAR-MSC-treated tumor xenograft mice as compared to UTD-MSC and control. Cytokines include macrophage-derived chemokine (MDC), growth related alpha protein (GRO), granulocyte macrophage colony-stimulating factor (GM-CSF), monocyte chemotactic protein 3 (MCP-3), and FMS-related tyrosine kinase 3 ligand (Flt-3L) in pg/mL. Data showing mean±s.d. with statistical analysis determined by multiple t tests (*p≤0.05, **p≤0.01, ****p≤0.0001; n=4-6 mice per group).

FIGS. 28A-28E show EcCAR-MSC safety profiles as determined using an in vivo canine model. FIG. 28A is a schematic for an exemplary method of CAR-MSC manufacturing and safety analysis in a healthy canine model. EcCARs with cross reactivity to human, mouse, and canine Ecad were lentivirally transduced into human MSCs and expanded in vitro for subsequent i.p. injection into healthy canine subjects. Subgroups were monitored for hematological and organ toxicity for 28 days. FIG. 28B is a series of graph of the complete blood count levels displayed as a determinant of hematopoietic safety following administration of EcCAR-MSCs. This includes white blood cells, monocytes, lymphocytes, neutrophils, and platelets with short term (3 day) and long term (28 day) blood level monitoring following in vivo EcCAR-MSC injection as compared to baseline. Displaying mean±s.d. of blood composition with statistics by 1-way ANOVA (ns=p≥0.05; n=3 subjects per experimental group). FIG. 28C is a series of graphs of total protein, BUN, creatinine, albumin, and alkaline phosphatase levels depicted for safety confirmation with short term (3 day) and long term (28 day) monitoring following in vivo EcCAR-MSC injection. Data shown are mean±s.d. with statistical analysis using a 1-way ANOVA (ns=p≥0.05; n=3 subjects per experimental group). FIG. 28D is a graph of bodyweight changes in healthy canines following administration of EcCAR-MSCS as compared to control. No significant differences in body weight changes were found between groups. Data shown are mean±s.d. of % weight change from baseline with statistical analysis performed by ordinary 1-way ANOVA (ns=p≥0.05), (ns=p≥0.05; n=3 subjects per experimental group).

FIG. 28E is a representative image of transverse colonic tissue sections of canines through H&E staining 28 days following administration at 20× and 40× magnification following treatment with human EcCAR-MSCs (left) or control (right).

FIG. 29 is a graph depicting the percentage of EcCAR positivity following transduction of mouse adipose-derived MSCs as determined by flow cytometry. Data shown are mean+/−s.d. representative of 3 technical mouse cell line replicates.

### DETAILED DESCRIPTION

This document provides methods and materials involved in treating a mammal (e.g., a human) in need of immunosuppression (e.g., a human having or at risk of developing one or more autoimmune diseases such as GVHD). In some cases, one or more CAR-MSCs having (e.g., engineered to have) an elevated level of one or more immunosuppressive polypeptides, an elevated level of one or more regenerative polypeptides, and/or an elevated level of one or more trafficking polypeptides can be administered to a mammal to induce an immunosuppressive response (e.g., to reduce or eliminate an inflammatory immune response) in a targeted tissue within the mammal. For example, a CAR-MSC can express a CAR that can target an epithelial-specific antigen (e.g., Ecad), also referred to as CDH1)) to target the MSC to epithelial tissues, and also can have (e.g., can be engineered to have) an elevated level of one or more immunosuppressive polypeptides, an elevated level of one or more regenerative polypeptides, and/or an elevated level of one or more trafficking polypeptides to enhance immunosuppression within the targeted epithelial tissues. In some cases, a CAR-MSC can express a CAR that can target a neural-specific antigen (e.g., myelin oligodendrocyte glycoprotein (MOG)) to target the MSC to neural tissues, and also can have (e.g., can be engineered to have) an elevated level of one or more immunosuppressive polypeptides, an elevated level of one or more regenerative polypeptides, and/or an elevated level of one or more trafficking polypeptides to enhance immunosuppression within the targeted neural tissues. For example, one or more CAR-MSCs having (e.g., engineered to have) an elevated level of one or more immunosuppressive polypeptides, an elevated level of one or more regenerative polypeptides, and/or an elevated level of one or more trafficking polypeptides can be administered (e.g., by adoptive transfer) to a mammal (e.g., a human) having (or at risk of developing) an inflammatory disease or condition to treat one or more autoimmune diseases (e.g., GVHD) in a targeted tissue (e.g., an inflamed tissue) within the mammal.

The term “elevated level” as used herein with respect to a level of a polypeptide (e.g., an immunosuppressive polypeptide or a trafficking polypeptide) refers to any level that is higher than a reference level of that polypeptide. The term “reference level” as used herein with respect to a polypeptide (e.g., an immunosuppressive polypeptide or a trafficking polypeptide) refers to the level of that polypeptide typically observed in a MSC (e.g., a CAR-MSC) not engineered to have an elevated level of that polypeptide as described herein.

A MSC described herein (e.g., a CAR-MSC having an elevated level of one or more immunosuppressive polypeptides, an elevated level of one or more regenerative polypeptides, and/or an elevated level of one or more trafficking polypeptides) can be any appropriate MSC. Examples of MSCs that can be used as described herein include, without limitation, MSCs derived from adipose, MSCs derived from bone marrow, MSCs derived from placental tissue, MSCs derived from dental pulp tissue, MSCs derived from an umbilical cord, MSCs derived from cord blood, MSCs derived from Wharton's jelly, MSCs derived from dermis, MSCs derived from olfactory mucosa, MSCs derived from peripheral blood, and MSCs derived from an amniotic membrane. For example, a CAR-MSC having (e.g., engineered to have) an elevated level of one or more immunosuppressive polypeptides, an elevated level of one or more regenerative polypeptides, and/or an elevated level of one or more trafficking polypeptides can be an adipose derived-MSC.

A CAR-MSC provided herein (e.g., a CAR-MSC having an elevated level of one or more immunosuppressive polypeptides, an elevated level of one or more regenerative polypeptides, and/or an elevated level of one or more trafficking polypeptides) can express any appropriate CAR. A CAR can include (a) an antigen-binding domain, (b) a transmembrane domain, and (c) one or more signaling domains.

An antigen-binding domain of a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be any appropriate antigen-binding domain. In some cases, an antigen-binding domain can include an antibody or a fragment thereof that targets an antigen (e.g., a tissue-specific antigen such as an epithelial-specific antigen or a neural-specific antigen). Examples of antigen-binding domains include, without limitation, an antigen-binding fragment (Fab), a variable region of an antibody heavy (VH) chain, a variable region of a light (VL) chain, and a single chain variable fragment (scFv). In some cases, an antigen-binding domain can target (e.g., can target and bind to) a tissue-specific antigen (e.g., an epithelial-specific antigen or a neural-specific antigen). For example, a CAR-MSC described herein can express (e.g., can be engineered to express) a CAR that can bind to a tissue-specific antigen (e.g., an antigen present on cells within a tissue with minimal, or no, expression on other cell types).

In some cases, a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be engineered to express a CAR that can target (e.g., can target and bind to) an antigen (e.g., a cell surface antigen) expressed by epithelial cells (e.g., an epithelial-specific antigen or an epithelial antigen) in a mammal in need of immunosuppression (e.g., a human having or at risk of developing one or more autoimmune diseases such as GVHD) within an epithelial tissue. An epithelial-specific antigen can be any appropriate epithelial-specific antigen. An epithelial-specific antigen can be expressed on any appropriate type of epithelial cell (e.g., gastrointestinal tract cells such as colon cells and rectal cells, skin cells, lung cells, liver cells, genital tract cells, and urinary tract cells). In some cases, an epithelial-specific antigen can be a cell adhesion molecule (CAM). In some cases, an epithelial-specific antigen can be an integrin (e.g., a gut integrin). Examples of epithelial-specific antigens include, without limitation, Ecad, CD103, αEβ7, and α4β7. For example, a CAR-MSC engineered to target epithelial tissues can bind to Ecad. In some cases, a CAR-MSC can be engineered to express a CAR-Ecad to target Ecad expressed by epithelial cells in a mammal. In some cases, a CAR expressed by a CAR-MSC provided herein can include an Ecad binding domain. For example, a CAR expressed by a CAR-MSC provided herein can include an anti-Ecad scFv that includes a heavy chain comprising the CDRs set forth in SEQ ID NO:1, and a light chain comprising the CDRs set forth in SEQ ID NO:2. For example, a CAR expressed by a CAR-MSC provided herein can have an anti-Ecad scFv that includes a heavy chain that comprises, consists essentially of, or consists of an amino acid sequence set forth in SEQ ID NO: 1 and includes a light chain that comprises, consists essentially of, or consists of an amino acid sequence set forth in SEQ ID NO:2 (see, e.g., Example 2). In some cases, a CAR expressed by a CAR-MSC provided herein can include a CD103 binding domain. For example, a CAR expressed by a CAR-MSC provided herein can include an anti-CD103 scFv that includes a heavy chain comprising the CDRs set forth in SEQ ID NO:3, and a light chain comprising the CDRs set forth in SEQ ID NO:4. For example, a CAR expressed by a CAR-MSC provided herein can have an anti-CD103 scFv that includes a heavy chain that comprises, consists essentially of, or consists of an amino acid sequence set forth in SEQ ID NO:3 and includes a light chain that comprises, consists essentially of, or consists of an amino acid sequence set forth in SEQ ID NO:4 (see, e.g., Example 2).

In some cases, a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be engineered to express a CAR that can target (e.g., can target and bind to) an antigen (e.g., a cell surface antigen) expressed by neural cells (e.g., a neural-specific antigen or a neural antigen) in a mammal in need of immunosuppression (e.g., a human having or at risk of developing one or more autoimmune diseases such as multiple sclerosis) within a neural tissue. When an antigen is a neural-specific antigen, the neural-specific antigen can be any appropriate neural-specific antigen. A neural-specific antigen can be expressed on any appropriate type of neural cell (e.g., sensory neurons, motor neurons, interneurons, glial cells, and neural sheath cells). A neural-specific antigen can be expressed on a neural cell in the central nervous system (CNS) and/or in the peripheral nervous system (PNS). In some cases, a neural-specific antigen can be a transmembrane protein. In some cases, a neural-specific antigen can target the myelin. Examples of neural-specific antigens include, without limitation, MOG and AMPA (e.g., an antigen on one or more AMPA receptor polypeptides). For example, a CAR-MSC engineered to target neural tissues can bind to MOG. In some cases, a CAR-MSC can be engineered to express a CAR-MOG to target MOG expressed by neural cells in a mammal having, or at risk of developing, multiple sclerosis. In some cases, a CAR expressed by a CAR-MSC provided herein can include a MOG antigen-binding domain. For example, a CAR expressed by a CAR-MSC provided herein can include an anti-MOG scFv that includes a heavy chain comprising the CDRs set forth in SEQ ID NO:5, and a light chain comprising the CDRs set forth in SEQ ID NO:6. For example, a CAR expressed by a CAR-MSC provided herein can have an anti-MOG scFv that includes a heavy chain that comprises, consists essentially of, or consists of an amino acid sequence set forth in SEQ ID NO: 5 and includes a light chain that comprises, consists essentially of, or consists of an amino acid sequence set forth in SEQ ID NO:6 (see, e.g., Example 2). In some cases, a CAR expressed by a CAR-MSC provided herein can include an AMPA antigen-binding domain. For example, a CAR expressed by a CAR-MSC provided herein can include an anti-AMPA scFv that includes a heavy chain comprising the CDRs set forth in SEQ ID NO:7, and a light chain comprising the CDRs set forth in SEQ ID NO:8. For example, a CAR expressed by a CAR-MSC provided herein can have an anti-AMPA scFv that includes a heavy chain that comprises, consists essentially of, or consists of an amino acid sequence set forth in SEQ ID NO: 7 and includes a light chain that comprises, consists essentially of, or consists of an amino acid sequence set forth in SEQ ID NO:8 (see, e.g., Example 2).

A transmembrane domain of a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be any appropriate transmembrane domain. Examples of transmembrane domains that can be used as described herein include, without limitation, NKG2D transmembrane domains, CD8a transmembrane domains, CD28 transmembrane domains, IgG4 transmembrane domains, TNFR transmembrane domains, and TLR transmembrane domains. In some cases, a transmembrane domain that is included in a CAR expressed by a CAR-MSC provided herein can be linked (e.g., covalently linked) to an adaptor polypeptide. In some cases, a CAR expressed by a CAR-MSC provided herein can include a CD28 transmembrane domain. For example, a CAR expressed by a CAR-MSC provided herein can include a CD28 transmembrane domain that comprises, consists essentially of, or consists of the amino acid sequence set forth in SEQ ID NO:9 (see, e.g., Example 2).

A signaling domain of a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can include any appropriate one or more signaling domains. For example, a CAR expressed by a CAR-MSC provided herein can be designed to include one, two, three, or four signaling domains. When a CAR expressed by a CAR-MSC provided herein provided herein includes more than one (e.g., two, three, or four) signaling domains, the CAR can include any appropriate combination of signaling domains. In some cases, a CAR expressed by a CAR-MSC provided herein can be designed to include one or more signaling domains normally found within an immune cell (e.g., a lymphocyte such as a TIL, a T cell, or a NK cell). In some cases, a CAR expressed by a CAR-MSC provided herein can be designed to include one or more signaling domains normally found within an MSC (e.g., an MSC with an immunosuppressive phenotype). In some cases, a CAR expressed by a CAR-MSC provided herein can be designed to include one or more regenerative signaling domains (e.g., to stimulate tissue regeneration). Examples of signaling domains that can be used as described herein include, without limitation, CD3zeta signaling domains, CD28 signaling domains, 4-1BB signaling domains, OX40 signaling domains, TLR3 signaling domains, TLR4 signaling domains, TIR3 signaling domains, TIR4 signaling domains, IFNγ signaling domains, brain derived neurotrophic factor (BDNF) signaling domains, vascular endothelial derived growth factor receptor (VEGFR2) signaling domains, TLR3/TIR3 signaling domains, TLR4/TIR4 signaling domains, TLR9/TIR9 signaling domains, IFNGR1 signaling domains, IL10RA signaling domains, and TNER2 signaling domains. In some cases, a CAR expressed by a CAR-MSC provided herein can be designed to be a first generation CAR having a CD3 signaling domain. In some cases, a CAR expressed by a CAR-MSC provided herein can be designed to be a second generation CAR having a CD28 signaling domain followed by a CD3ζ signaling domain. In some cases, a CAR expressed by a CAR-MSC provided herein can be designed to be a third generation CAR having (a) a CD28 signaling domain followed by (b) a CD27 signaling domain, an OX40 signaling domains, or a 4-1BB signaling domain followed by (c) a CD3ζ signaling domain. In some cases, a CAR expressed by a CAR-MSC provided herein can include a CD28 signaling domain. For example, a CAR expressed by a CAR-MSC provided herein can include a CD28 signaling domain that comprises, consists essentially of, or consists of the amino acid sequence set forth in SEQ ID NO: 10 (see, e.g., Example 2) and a CD3ζ signaling domain that comprises, consists essentially of, or consists of the amino acid set forth in SEQ ID NO:11 (see, e.g., Example 2).

In some cases, a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can include one or more additional components. Examples of additional components that can be included in a CAR expressed by a CAR-MSC provided herein include, without limitation, linkers, hinge domains, and detectable markers. When an antigen receptor (e.g., a CAR) provided herein includes a detectable marker, the detectable marker can be any appropriate detectable marker. Examples of detectable markers that can be included in a CAR expressed by a CAR-MSC provided herein include, without limitation, bioluminescent polypeptides (e.g., luciferase polypeptides), fluorescent polypeptides (e.g., green fluorescent polypeptides (GFPs)), sodium iodine symporter (NIS), SSTR2 polypeptides, PSMA polypeptides, hdCK polypeptides, and eDHFR polypeptides.

In some cases, a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can include an anti-Ecad scFv, a CD28 transmembrane domain, a CD28 signaling domain, and a CD3ζ signaling domain. For example, a CAR expressed by a CAR-MSC provided herein can include an anti-Ecad scFv having a heavy chain comprising the CDRs set forth in SEQ ID NO:1, and a light chain comprising the CDRs set forth in SEQ ID NO: 2 (see, e.g., Example 2), a CD28 transmembrane domain that comprises, consists essentially of, or consists of the amino acid sequence set forth in SEQ ID NO:9 (see, e.g., Example 2), a CD28 signaling domain that comprises, consists essentially of, or consists of the amino acid sequence set forth in SEQ ID NO: 10 (see, e.g., Example 2), and a CD3ζ signaling domain that comprises, consists essentially of, or consists of the amino acid sequence set forth in SEQ ID NO:11 (see, e.g., Example 2).

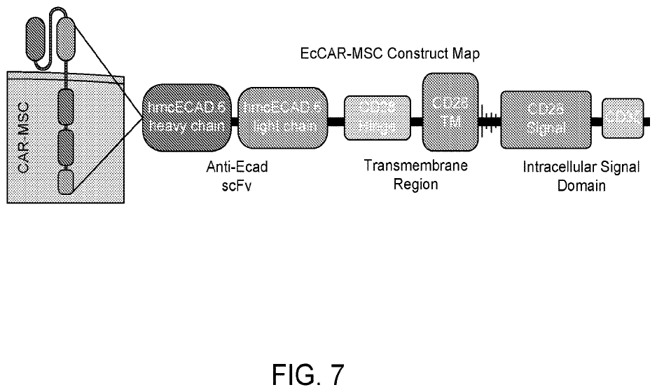

Any appropriate method can be used to express a CAR described herein (e.g., a CAR targeting a tissue-specific antigen such as an epithelial-specific antigen or a neural-specific antigen) on the surface of a CAR-MSC provided herein (e.g., a CAR-MSC having an elevated level of one or more immunosuppressive polypeptides, an elevated level of one or more regenerative polypeptides, and/or an elevated level of one or more trafficking polypeptides). For example, a nucleic acid encoding a CAR can be introduced into a MSC. In some cases, a nucleic acid encoding a CAR can be introduced into a MSC by transduction (e.g., viral transduction) or transfection. In some cases, a nucleic acid encoding a CAR described herein can be introduced ex vivo into one or more MSCs. For example, ex vivo engineering of MSCs to express a CAR described herein can include transducing isolated MSCs with a vector (e.g., viral vector such as a lentiviral vector, a retroviral vector, an adenoviral vector, or an adeno-associated viral (AAV) vector) encoding a CAR. In cases where MSCs are engineered ex vivo to express a CAR, the MSCs can be obtained from any appropriate source (e.g., a mammal such as the mammal to be treated or a donor mammal, or a cell line). In some cases, CAR-MSCs can be prepared as described herein (see, e.g., FIG. 7 and Example 1). For example, a CAR-Ecad can be expressed on a MSC to direct the MSC to epithelial tissues by introducing one or more constructs containing a nucleic acid encoding the CAR (e.g., a CAR targeting Ecad) into the MSC. For example, a CAR-MOG can be expressed on a MSC to direct the MSC to neural tissues by introducing one or more constructs containing a nucleic acid encoding the CAR (e.g., a CAR targeting MOG) into the MSC.

In some cases, a CAR expressed by a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be a CAR as described elsewhere. See, for example, International Patent Application Publication No. WO 2021/092577 at, for example, page 18, line 16 to page 20, line 25, and page 21, line 29 to page 25, line 11.

A CAR-MSC provided herein (e.g., a CAR-MSC having an elevated level of one or more immunosuppressive polypeptides, an elevated level of one or more regenerative polypeptides, and/or one and elevated level of one or more trafficking polypeptides) can express any appropriate one or more immunosuppressive polypeptides, one or more regenerative polypeptides, and/or an elevated level of one or more trafficking polypeptides. In some cases, a polypeptide (e.g., an immunosuppressive polypeptide or a trafficking polypeptide) expressed by a CAR-MSC provided herein can be an exogenous polypeptide. In some cases, a polypeptide (e.g., an immunosuppressive polypeptide or a trafficking polypeptide) expressed by a CAR-MSC provided herein can be part of a signaling pathway (e.g., a TNFα signaling pathway, a TLR signaling pathway, and an IL-10 signaling pathway). In some cases, a polypeptide (e.g., an immunosuppressive polypeptide or a trafficking polypeptide) expressed by a CAR-MSC provided herein can be associated with T cell suppression. In some cases, a polypeptide (e.g., an immunosuppressive polypeptide or a trafficking polypeptide) expressed by a CAR-MSC provided herein can be a kinase (e.g., a tyrosine kinase). In some cases, a polypeptide (e.g., an immunosuppressive polypeptide or a trafficking polypeptide) expressed by a CAR-MSC provided herein can be a transcription factor. In some cases, a polypeptide (e.g., an immunosuppressive polypeptide or a trafficking polypeptide) expressed by a CAR-MSC provided herein can be chemokine (e.g., a homing chemokine). In some cases, a polypeptide (e.g., an immunosuppressive polypeptide or a trafficking polypeptide) expressed by a CAR-MSC provided herein can be a cytokine (e.g., an anti-inflammatory cytokine). In some cases, a polypeptide (e.g., an immunosuppressive polypeptide or a trafficking polypeptide) expressed by a CAR-MSC provided herein can be an immunomodulatory cell surface polypeptide. In some cases, a polypeptide (e.g., an immunosuppressive polypeptide or a trafficking polypeptide) expressed by a CAR-MSC provided herein can be involved in cellular migration (e.g., transendothelial migration).

Examples of immunosuppressive polypeptides and trafficking polypeptides that a CAR-MSC provided herein can express (e.g., can be designed to express) include, without limitation, NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, CXCR4 polypeptides, CTLA4 polypeptides, TLR3 polypeptides, TLR4 polypeptides, TLR9 polypeptides, and TNFR2 polypeptides.

Any appropriate method can be used to increase a level of one or more immunosuppressive polypeptides, to increase a level of one or more regenerative polypeptides, and/or to increase a level of one or more trafficking polypeptides in a CAR-MSC provided herein. In some cases, a nucleic acid (e.g., an exogenous nucleic acid) encoding a polypeptide (e.g., an immunosuppressive polypeptide or a trafficking polypeptide) can be introduced into a CAR-MSC. For example, an exogenous nucleic acid encoding one or more immunosuppressive polypeptides, one or more regenerative polypeptides, and/or one or more trafficking polypeptides can be introduced into a MSC by transduction (e.g., viral transduction) or transfection. In some cases, a nucleic acid (e.g., an endogenous nucleic acid) encoding one or more immunosuppressive polypeptides, one or more regenerative polypeptides, and/or one or more trafficking polypeptides within a CAR-MSC can be modified to increase a level of expression of one or more immunosuppressive polypeptides, one or more regenerative polypeptides, and/or one or more trafficking polypeptides in the CAR-MSC. For example, an endogenous nucleic acid encoding an immunosuppressive polypeptide, a regenerative polypeptide, or a trafficking polypeptide within a MSC can be modified by gene-editing techniques (e.g., clustered regularly interspaced short palindromic repeats (CRISPR)/CRISPR-associated nucleases (Cas) systems, transcription activator-like effector nucleases (TALENs) systems, zinc finger nuclease (ZFN) systems, and base pair editing) to increase the level of expression of one or more immunosuppressive polypeptides, to increase a level of expression of one or more regenerative polypeptides, and/or to increase the level of one or more trafficking polypeptides in the CAR-MSC.

In some cases, a CAR-MSC provided herein (e.g., a CAR-MSC having an elevated level of one or more immunosuppressive polypeptides, an elevated level of one or more regenerative polypeptides, and/or an elevated level of one or more trafficking polypeptides) can be engineered to have an elevated level of one or more immunosuppressive polypeptides, an elevated level of one or more regenerative polypeptides, and/or an elevated level of one or more trafficking polypeptides ex vivo. For example, ex vivo engineering of CAR-MSCs to have an elevated level of one or more immunosuppressive polypeptides, an elevated level of one or more regenerative polypeptides, and/or an elevated level of one or more trafficking polypeptides can include transducing isolated CAR-MSCs with a vector (e.g., viral vector such as a lentiviral vector, a retroviral vector, an adenoviral vector, or an AAV vector) encoding the polypeptide(s). For example, ex vivo engineering of CAR-MSCs to have an elevated level of one or more immunosuppressive polypeptides, an elevated level of one or more regenerative polypeptides, and/or an elevated level of one or more trafficking polypeptides can include transducing isolated CAR-MSCs with one or more lentiviral vectors encoding gene-editing components.

Examples of NFκB1 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the National Center for Biotechnology Information (NCBI) databases at, for example, accession no. NM 001165412 (version NM_001165412.1), accession no. NM_001319226 (version NM_001319226.2), and accession no. NM_001382625 (version NM_001382625.1). In some cases, a NFκB1 polypeptide that can be elevated in a CAR-MSC provided herein can have an amino acid sequence set forth in SEQ ID NO: 12 (see, e.g., Example 2).

Examples of JUN polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_002228 (version NM_002228.4), accession no. AL136985 (version AL136985.11), and accession no. CH471059 (version CH471059.2). In some cases, a JUN polypeptide that can be elevated in a CAR-MSC provided herein can have an amino acid sequence set forth in SEQ ID NO:13 (see, e.g., Example 2).

Examples of RELB polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, NM_001411087 (version NM_001411087.1) and accession no. NM_006509 (version NM_006509.4). In some cases, a RELB polypeptide that can be elevated in a CAR-MSC provided herein can have an amino acid sequence set forth in SEQ ID NO: 14 (see, e.g., Example 2).

Examples of IRF1 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001354924 (version NM_001354924.1), accession no. NM_001354925 (version NM_001354925.1), and accession no. NM_002198 (version NM_002198.3). In some cases, a IRF1 polypeptide that can be elevated in a CAR-MSC provided herein can have an amino acid sequence set forth in SEQ ID NO: 15 (see, e.g., Example 2).

Examples of TNFα polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_000594 (version NM_000594.4). In some cases, a TNFα polypeptide that can be elevated in a CAR-MSC provided herein can have an amino acid sequence set forth in SEQ ID NO: 16 (see, e.g., Example 2).

Examples of IL-10 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_000572 (version NM_000572.3) and accession no. NM_001382624 (version NM_001382624.1). In some cases, a IL-10 polypeptide that can be elevated in a CAR-MSC provided herein can have an amino acid sequence set forth in SEQ ID NO:17 (see, e.g., Example 2).

Examples of FGF-2 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001361665 (version NM_001361665.2) and accession no. NM_002006 (version NM_002006.6). In some cases, a FGF-2 polypeptide that can be elevated in a CAR-MSC provided herein can have an amino acid sequence set forth in SEQ ID NO:18 (see, e.g., Example 2).

Examples of G-CSF polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_000759 (version NM_000759.4), accession no. NM_001178147 (version NM_001178147.2), and accession no. NM_172219 (version NM_172219.3). In some cases, a G-CSF polypeptide that can be elevated in a CAR-MSC provided herein can have an amino acid sequence set forth in SEQ ID NO:19 (see, e.g., Example 2).

Examples of GM-CSF polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_000758 (version NM_000758.4), accession no. AC003959 (version AC003959.1), and accession no. DQ789232 (version DQ789232.1). In some cases, a GM-CSF polypeptide that can be elevated in a CAR-MSC provided herein can have an amino acid sequence set forth in SEQ ID NO:20 (see, e.g., Example 2).

Examples of eotaxin polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_002986 (version NM_002986.3), accession no. AC003959 (version AC003959.1), and accession no. CH471062 (version CH471062.2). In some cases, an eotaxin polypeptide that can be elevated in a CAR-MSC provided herein can have an amino acid sequence set forth in SEQ ID NO:21 (see, e.g., Example 2).

Examples of Gal-9 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001330163 (version NM_001330163.2), accession no. NM_002308 (version NM_002308.4), and accession no. NM_009587 (version NM_009587.3). In some cases, a Gal-9 polypeptide that can be elevated in a CAR-MSC provided herein can have an amino acid sequence set forth in SEQ ID NO:22 (see, e.g., Example 2).

Examples of PD-1 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_005018 (version NM_005018.3), accession no. AF363458 (version AF363458.1), and accession no. EF064716 (version EF064716.1). In some cases, a PD-1 polypeptide that can be elevated in a CAR-MSC provided herein can have an amino acid sequence set forth in SEQ ID NO:23 (see, e.g., Example 2).

Examples of TIM-3 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_032782 (version NM_032782.5), accession no. CQ834184 (version CQ834184.1), and accession no. AC011377 (version AC011377.6). In some cases, a TIM-3 polypeptide that can be elevated in a CAR-MSC provided herein can have an amino acid sequence set forth in SEQ ID NO:24 (see, e.g., Example 2).

Examples of CXCR3 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001142797 (version NM_001142797.2), accession no. NM_001504 (version NM_001504.2), and accession no. AB032738 (version AB032738.1). In some cases, a CXCR3 polypeptide that can be elevated in a CAR-MSC provided herein can have an amino acid sequence set forth in SEQ ID NO:25 (see, e.g., Example 2).

Examples of CXCR4 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001008540 (version NM_001008540.2), accession no. NM_001348056 (version NM_001348056.2), and accession no. NM_001348059 (version NM 001348059.2). In some cases, a CXCR4 polypeptide that can be elevated in a CAR-MSC provided herein can have an amino acid sequence set forth in SEQ ID NO:26 (see, e.g., Example 2).

Examples of CTLA4 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001037631 (version NM_001037631.2), NM_001037631 (version NM_001037631.3), and NM_005214 (version NM_005214.4 and NM_005214.5). In some cases, a CTLA4 polypeptide that can be elevated in a CAR-MSC provided herein can have an amino acid sequence set forth in SEQ ID NO:91 (see, e.g., Example 2).

Examples of TLR3 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_003265 (version NM_003265.2 and NM_003265.3), NM_133484 (NM_133484.2), and NM_126166 (version NM_126166.4). In some cases, a TLR3 polypeptide that can be elevated in a CAR-MSC provided herein can have an amino acid sequence set forth in SEQ ID NO:92 (see, e.g., Example 2).

Examples of TLR4 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_003266 (version NM_003266.4), NM_138557 (version NM_138557.3) and NM_138554 (version NM_138554.4). In some cases, a TLR4 polypeptide that can be elevated in a CAR-MSC provided herein can have an amino acid sequence set forth in SEQ ID NO: 93 (see, e.g., Example 2).

Examples of TLR9 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_017442 (version NM_017442.3 and NM_017442.4) and NM_031178 (version NM_031178.2). In some cases, a TLR9 polypeptide that can be elevated in a CAR-MSC provided herein can have an amino acid sequence set forth in SEQ ID NO:94 (see, e.g., Example 2).

Examples of TNFR2 polypeptides that can be elevated in a CAR-MSC provided herein (e.g., a CAR-MSC having an elevated level one or more immunosuppressive polypeptides, an elevated level of one or more regenerative polypeptides, and/or an elevated level of one or more trafficking polypeptides) include those set forth in the NCBI databases at, for example, accession no. NM_001066 (version NM_001066.2) and NM_011610 (version NM_011610.3). In some cases, a TNFR2 polypeptide that can be elevated in a CAR-MSC provided herein can have an amino acid sequence set forth in SEQ ID NO:95 (see, e.g., Example 2).

This document also provides materials and methods for treating mammals in need of immunosuppression (e.g., a human having or at risk of developing one or more autoimmune diseases such as GVHD). In some cases, a mammal (e.g., a human) in need of immunosuppression can have (or be at risk of developing) GVHD. For example, one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) can be administered (e.g., by adoptive transfer) to a mammal having one or more autoimmune diseases to reduce the severity of the immune response within the mammal. Any appropriate method can be used to identify a mammal as being in need of immunosuppression. Once identified as being in need of immunosuppression, one or more CAR-MSCs provided herein can be administered to the mammal (e.g., a human) as described herein to reduce the immune response within the mammal by, for example, 10, 20, 30, 40, 50, 60, 70, 80, 90, 95, or more percent.

In some cases, when treating a mammal (e.g., a human) in need of immunosuppression (e.g., a human having or at risk of developing one or more autoimmune diseases such as GVHD) as described herein, the treatment can be effective to reduce inflammation within the mammal (e.g., within a targeted tissue within the mammal). For example, the methods and materials described herein can be used to reduce inflammation within a mammal having one or more autoimmune disorder (e.g., GVHD) by, for example, 10, 20, 30, 40, 50, 60, 70, 80, 90, 95, or more percent.

In some cases, when treating a mammal (e.g., a human) in need of immunosuppression (e.g., a human having or at risk of developing one or more autoimmune diseases such as GVHD) as described herein, the treatment can be effective to increase the number of regulatory T cells (Tregs) within the mammal (e.g., within a targeted tissue within the mammal). For example, the methods and materials described herein can be used to increase the number of Tregs present within a mammal in need of immunosuppression (e.g., a human having or at risk of developing one or more autoimmune diseases such as GVHD) by, for example, 10, 20, 30, 40, 50, 60, 70, 80, 90, 95, or more percent. In some cases, the number of Tregs present within a mammal does not decrease.

In some cases, when treating a mammal (e.g., a human) in need of immunosuppression (e.g., a human having or at risk of developing one or more autoimmune diseases such as GVHD) as described herein, the treatment can be effective to reduce the number of activated T cells (e.g., CD4⁺ T cells and/or CD8⁺ T cells) within the mammal (e.g., within a targeted tissue within the mammal). For example, the methods and materials described herein can be used to reduce the number of activated T cells (e.g., CD4⁺ T cells and/or CD8⁺ T cells) present within a mammal in need of immunosuppression (e.g., a human having or at risk of developing one or more autoimmune diseases such as GVHD) by, for example, 10, 20, 30, 40, 50, 60, 70, 80, 90, 95, or more percent. In some cases, the number of activated T cells (e.g., CD4⁺ T cells and/or CD8⁺ T cells) present within a mammal does not increase.

In some cases, when treating a mammal (e.g., a human) in need of immunosuppression (e.g., a human having or at risk of developing one or more autoimmune diseases such as GVHD) as described herein, the treatment can be effective to reduce the rate of T cell proliferation (e.g., activated T cell proliferation) within the mammal (e.g., within a targeted tissue within the mammal). For example, the methods and materials described herein can be used to reduce rate of T cell proliferation within a mammal in need of immunosuppression (e.g., a human having or at risk of developing one or more autoimmune diseases such as GVHD) by, for example, 10, 20, 30, 40, 50, 60, 70, 80, 90, 95, or more percent. In some cases, the rate of T cell proliferation within a mammal does not increase.

Any appropriate mammal in need of immunosuppression (e.g., a human having or at risk of developing one or more autoimmune diseases such as GVHD) can be treated as described herein. Examples of mammals that can be in need of immunosuppression and can be treated as described herein include, without limitation, humans, non-human primates (e.g., monkeys), dogs, cats, horses, cows, pigs, sheep, mice, and rats. For example, one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) can be administered (e.g., by adoptive transfer) to a human having one or more autoimmune diseases to treat the human.

Any appropriate one or more autoimmune diseases (e.g., GVHD) can be treated as described herein. Examples of autoimmune disease include, without limitation, GVHD, inflammatory bowel diseases (e.g., ulcerative colitis and Crohn's disease), hepatitis, bronchiolitis obliterans, pneumonitis, encephalitis, multiple sclerosis, rheumatoid arthritis, lupus, and psoriasis. In some cases, an autoimmune disease can be GVHD (e.g., acute GVHD or chronic GVHD). GVHD can be allogeneic GVHD (allo-GVHD) or autologous GVHD (auto-GVHD). GVHD can be any stage of GVHD. In some cases, GVHD can be associated with (e.g., following) a transplant. A transplant can be an allogeneic transplant or an autologous transplant. When treating a mammal having (or at risk of developing) GVHD, the mammal can have undergone any type of transplant (e.g., an allogeneic transplant such as bone marrow transplants, stem cell transplants, and organ transplants such as kidney transplants and liver transplants).

In some cases, a mammal can be identified as having or at risk of developing one or more autoimmune diseases (e.g., GVHD). Any appropriate method can be used to identify a mammal as having or at risk of developing one or more autoimmune diseases (e.g., GVHD). In some cases, a mammal can be identified as having (or as being at risk of developing) GVHD. Any appropriate method for identifying a mammal as having (or as being at risk of developing) GVHD can be used. Once identified as having (or as being at risk of developing) GVHD, the mammal can be administered (e.g., by adoptive transfer) or instructed to self-administer one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) to treat the GVHD within the mammal.

In some cases, one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) can be administered (e.g., by adoptive transfer) to a mammal having or at risk of developing GVHD to reduce the severity of GVHD within the mammal. In some cases, reducing the severity of GVHD in a mammal can include reducing or eliminating one or more symptoms of GVHD (e.g., skin rashes, immune-mediated pneumonitis, intestinal inflammation, sloughing of the intestinal mucosal membrane, severe diarrhea, abdominal pain, nausea, vomiting, and/or elevated bilirubin levels). In some cases, reducing the severity of GVHD in a mammal can include reducing the stage of GVHD. The stage of GVHD can be evaluated as described elsewhere (see, e.g., Jacobsohn et al., Orphanet. J. Rare Dis., 2:35 (2007)).

Any appropriate method can be used to administer one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) to a mammal (e.g., a human) in need of immunosuppression (e.g., a human having or at risk of developing one or more autoimmune diseases such as GVHD). Examples of methods of administering MSCs provided herein to a mammal can include, without limitation, injection (e.g., intravenous, intraperitoneal, intramuscular, subcutaneous injection, or local injection into an area of inflammation). For example, a composition including one or more CAR-MSCs provided herein can be administered to a human by intravenous injection.

In some cases, one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) can be the sole active ingredient for treating a mammal (e.g., a human) in need of immunosuppression (e.g., a human having or at risk of developing one or more autoimmune diseases such as GVHD). For example, a composition including one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) can include the one or more CAR-MSCs provided herein as the sole active agent to suppress an immune response within a mammal (e.g., a human) in need of immunosuppression (e.g., a human having or at risk of developing one or more autoimmune diseases such as GVHD).

In some cases, one or more CAR-MSCs provided herein (e.g., one or more CAR-MSCs having an elevated level of one or more immunosuppressive polypeptides, an elevated level of one or more regenerative polypeptides, and/or an elevated level of one or more trafficking polypeptides) can be administered in combination with one or more additional agents that can be used to treat a mammal (e.g., a human) in need of immunosuppression (e.g., a human having or at risk of developing one or more autoimmune diseases such as GVHD). In some cases, an agent that can be used to suppress an immune response can be an anti-inflammatory. In some cases, an agent that can be used to suppress an immune response can be an immunosuppressant. Examples of agents that can be used in combination with one or more CAR-MSCs provided herein include, without limitation, corticosteroids (e.g., prednisone and methylprednisolone), azathioprine, mercaptopurine, cyclosporine, infliximab, adalimumab, golimumab, and vedolizumab. In some cases, one or more CAR-MSCs provided herein can be administered at substantially the same time as one or more additional agents. For example, a composition including one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides) also can include one or more additional agents that can be used to suppress an immune response within a mammal. In some cases, one or more CAR-MSCs provided herein can be administered first, and the one or more additional agents administered second, or vice versa.

In some cases, one or more CAR-MSCs provided herein (e.g., one or more CAR-MSCs having an elevated level of one or more immunosuppressive polypeptides, an elevated level of one or more regenerative polypeptides, and/or an elevated level of one or more trafficking polypeptides) can be used to treat a mammal (e.g., a human) having a disease or disorder that is not characterized by inflammation. For example, a CAR-MSC provided herein can be used to target a diseased tissue within a mammal (e.g., a human that is not in need of immunosuppression) to treat that diseased tissue. In some cases, a CAR-MSC provided herein can be used to target a degenerative tissue within a mammal (e.g., a human that is not in need of immunosuppression) to treat that tissue degeneration. For example, a CAR-MSC can express a CAR that can target a neural-specific antigen (e.g., AMPA) to target the MSC to neural tissues within a mammal (e.g., a mammal having a neurodegenerative diseases such as amyotrophic lateral sclerosis (ALS)) and includes a regenerative signaling domain (e.g., a BDNF signaling domain), and also can have (e.g., can be engineered to have) an elevated level of one or more immunosuppressive polypeptides, an elevated level of one or more regenerative polypeptides, and/or an elevated level of one or more trafficking polypeptides to treat the mammal. In some cases, a CAR-MSC provided herein can be used to target a fibrotic tissue within a mammal (e.g., a human that is not in need of immunosuppression) to treat that fibrotic tissue. For example, a CAR-MSC can express a CAR that can target a cartilage-specific antigen (e.g., fibroblast activating protein (FAP) or signaling lymphocyte activation molecule-7 (SLAMF7, also referred to as CS-1)) to target the MSC to fibrotic tissues within a mammal (e.g., a mammal having one or more fibrotic diseases (e.g., cardiac fibrosis, hepatic fibrosis, pulmonary fibrosis, genital fibrosis, or skin fibrosis), and also can have (e.g., can be engineered to have) an elevated level of one or more immunosuppressive polypeptides, an elevated level of one or more regenerative polypeptides, and/or an elevated level of one or more trafficking polypeptides to treat the mammal. In some cases, a CAR-MSC provided herein can be used to target a cardiac tissue within a mammal (e.g., a human that is not in need of immunosuppression) to treat cardiac disease. For example, a CAR-MSC can express a CAR that can target a cardiac-specific antigen (e.g., HER2) to target the MSC to cardiac tissues within a mammal (e.g., a mammal having cardiac inflammation and/or cardiac degeneration) and includes a regenerative signaling domain (e.g., a VEGFR2 signaling domain), and also can have (e.g., can be engineered to have) an elevated level of one or more immunosuppressive polypeptides, an elevated level of one or more regenerative polypeptides, and/or an elevated level of one or more trafficking polypeptides to treat the mammal.

This document also provides kits containing one or more materials described herein. In some cases, a kit can include one or more CAR-MSCs provided herein (e.g., one or more CAR-MSCs having an elevated level one or more immunosuppressive polypeptides, an elevated level of one or more regenerative polypeptides, and/or an elevated level of one or more trafficking polypeptides). For example, one or more CAR-MSCs provided herein can be combined with packaging material to form a kit. For example, one or more constructs (e.g., nucleic acid constructs) described herein (e.g., a construct encoding a CAR that can bind a tissue-specific antigen such as an epithelial-specific antigen and a construct encoding one or more immunosuppressive polypeptides, one or more regenerative polypeptides, and/or one or more trafficking polypeptides) can be combined with packaging material to form a kit. The packaging material included in such a kit typically contains instructions or a label describing how the composition can be used, for example, in an adoptive transfer to treat a mammal in need of immunosuppression (e.g., a human having or at risk of developing one or more autoimmune diseases such as GVHD) as described herein. In some cases, materials provided in kits described herein can be used for treating mammals (e.g., humans) having (or at risk of developing) GVHD as described herein. In some cases, the packaging material included in such a kit can contain instructions and/or a label describing how a composition described herein can be used. For example, a kit can contain instructions and/or a label describing how a composition described herein can be used to express one or more CARs and how to express one or more immunosuppressive polypeptides, one or more regenerative polypeptides, and/or one or more trafficking polypeptides in MSCs to engineer a CAR-MSC provided herein (e.g., a CAR-MSC having an elevated level one or more immunosuppressive polypeptides, an elevated level of one or more regenerative polypeptides, and/or an elevated level of one or more trafficking polypeptides). In some cases, the packaging material included in such a kit can contain instructions and/or a label describing how the CAR-MSCs provided herein can be used. For example, the packaging material included in such a kit can contain instructions and/or a label describing how the CAR-MSCs provided herein can be used in adoptive transfer to treat a mammal in need of immunosuppression (e.g., a having or at risk of developing one or more autoimmune diseases such as GVHD) as described herein. In some cases, a kit (e.g., a kit containing instructions and/or a label describing how the CAR-MSCs provided herein can be used in adoptive transfer) can include materials for use in an adoptive transfer procedure.

The invention will be further described in the following examples, which do not limit the scope of the invention described in the claims.

### EXAMPLES

### Example 1: Chimeric Antigen Receptor Engineering of Adipose Derived Mesenchymal Stromal Cells (CAR-MSCs) for Enhanced Immunosuppression

Current MSC therapy can be ineffective due to suboptimal homing and immunosuppressive capacity following therapeutic administration. MSCs were lentivirally transduced to stably express CAR constructs and generate CAR-MSCs. This Example describes the design of CAR-MSCs expressing an Ecad-specific scFv to generate anti-Ecad CAR-MSC (EcCAR-MSC). The incorporation of a CD28 signaling domain in EcCAR-MSC resulted in improved T cell suppression in both tumor and GVHD models.

### Materials and Methods

### Cell Lines, Primary PBMC/T Cells, and Primary MSCs

Primary human adipose derived (Ad)-MSCs were obtained and cultured with StemXVivo mesenchymal stromal cell expansion media (R&D Systems, Minneapolis, MN). Human epithelial breast cancer cell line, MCF7, was obtained from ATCC (CRL-3006, Manassas, VA, USA) and cultured in D10 (DMEM Gibco, Gaithersburg, MD, US) with 10% fetal bovine serum (FBS, Millipore Sigma, Ontario, Canada) and 1% Penicillin-Streptomycin-Glutamine (Gibco, Gaithersburg, MD, US). MCF7 was irradiated and confirmed for Ecad positivity by flow cytometry.

Mantle cell lymphoma cell line JeKo-1 and acute lymphoblastic leukemia cell line Nalm6 were purchased from ATCC. JeKo-1 and Nalm6 cells were cultured in R10 (RPMI 1640, Gibco, Gaithersburg, MD, US) with 10% or 20% FBS (Millipore Sigma, Ontario, Canada), respectively, and 1% penicillin-streptomycin-glutamine (Gibco, Gaithersburg, MD, US). For in vivo experiment use, cell lines were transduced with luciferase-ZsGreen lentivirus (Addgene, Cambridge, MA, USA). Cell lines were cultured up to 10 passages, and fresh aliquots were thawed every 7-8 weeks. Cell lines were authenticated by the manufacturer and routinely checked for phenotype by flow cytometry. Cell lines were tested monthly for mycoplasma. Peripheral blood mononuclear cells (PBMC) were isolated from de-identified normal donor blood apheresis cones using SepMate tubes (STEMCELL Technologies, Vancouver, Canada). T cells were separated with negative selection magnetic beads using EasySep™ Human T Cell 80 Isolation Kit (STEMCELL Technologies).

Primary T cells and PBMCs were cultured in T cell medium containing X-VIVO 15 (Lonza, Walkersville, MD, USA), 10% human serum albumin (Innovative Research, Novi, MI, USA), and 1% penicillin-streptomycin-glutamine (Gibco, Gaithersburg, MD, USA) before selection for in vitro coculture. Freshly isolated human PBMCs were injected via intravenous administration for in vivo experiments.

### CAR Design and Virus Production

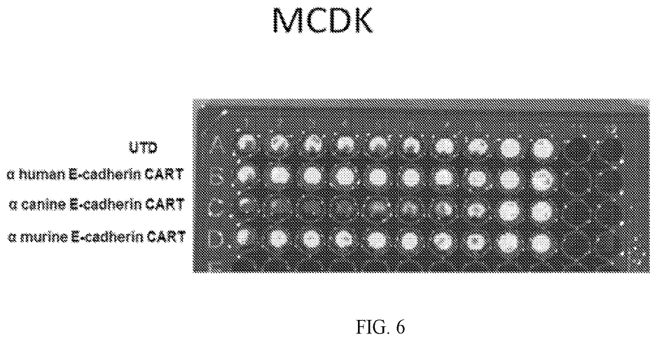

MSCs were all transduced via lentiviral vector encoding specifically designed CAR construct under an EF1alpha promoter. Optimization of transduction was performed with a CART19 construct. The CAR19 used contained a CD19-directed scFv derived from clone FMC-63 fused to 41BB and CD3ζ signaling domains (FMC63-41BB-ζ) A VSV-g-pseudotyped 2nd generation lentivirus was generated by loading the lentivirus with the CAR19 transgene via lipofectamine transfection and transfecting into HEK-293T cells, followed by standard procedures for harvesting, concentration, and functional titration of lentivirus. CAR constructs were then designed to include a scFv against human/canine Ecad (FIG. 6) with a CD28 intracellular signaling domain to induce MSC immunomodulatory activation (FIG. 7). A CAR plasmid was generated, and sequence validated by Addgene (Watertown, MA, USA). Pre-seeded Ad-MSCs (concentration of 250,000 cells/well) were transduced in a 6-well plate with lentiviral particles at a multiplicity of infection (MOI) of 3 with various concentrations of protamine sulfate (25 μg/μL, 50 μg/μL, or 100 μg/μL) for optimization of CAR expression and MSC proliferation.

### T Cell Suppression Assay

The ability of CAR-MSCs to inhibit activated T cells was tested using a T cell suppression assay. Untransduced (UTD)-MSCs or EcCAR-MSCs (suppressors) were co-cultured with activated T cells (effectors) in the presence or absence of soluble Ecad (stimulator) to provide antigen-specific stimulation to the CAR-MSCs. T cells were isolated from PBMCs of normal donors using negative selection magnetic beads. Isolated T cells were non-specifically activated using CD3 stimulating beads at a 3:1 bead to T cell ratio in T cell medium containing X-VIVO 15 (Lonza, Walkersville, MD, USA), 10% human serum albumin (Innovative Research, Novi, MI, USA), and 1% penicillin-streptomycin-glutamine (Gibco, Gaithersburg, MD, USA). After 24 hours, stimulated T cells (effectors) were co-cultured with UTD-MSCs or EcCAR-MSCs (suppressors) alone or with soluble Ecad protein (stimulator) to activate EcCAR-MSCs specifically through the CAR in StemXVivo serum free mesenchymal stromal cell expansion media (R&D Systems, Minneapolis, MN, USA). Cells were co-cultured at 1:10 MSC to T cell ratios with 100,000 T cells per 96 well plate as indicated. Cells were then harvested and analyzed by flow cytometry after different co-culture timeline as indicated in individual experiments.

### Multichannel Flow Cytometry

Staining for flow cytometry was performed. Briefly, MSCs were isolated and grown in culture, then transduced with CAR to create CAR-MSCs to be co-cultured with T cell/PBMC immune cells in 96 well plates for 24 hours and 6 well plates for longer term culture. Following desired co-culture durations, adherent MSC cells were detached with Accutase at 10 mL per 75 cm² surface area and incubated for 10-15 minutes at 37° C. Following detachment, all well contents were spun, washed in flow buffer (PBS, 2% FBS v/v, and 1% sodium azide v/v), and stained with desired antibody mixes in the dark for 15 minutes. Following final wash steps, cells were analyzed for desired surface markers and positivity was determined through negative gating by Fluorescence Minus One (FMO) control wells. Absolute cell count numbers were obtained using volumetric measurement. Cells were gated on the following: SSC vs. FSC plots to for cell separation by size and complexity, FSC-H vs. FSC-A plots exclude doublets, SSC vs. V500 channel live/dead fixable aqua stain to exclude dead cells, followed by cell subset characterization based on predesigned antibody panels optimized and used to stain samples.

Anti-human antibodies were purchased from Biolegend, eBioscience, or BD Biosciences (San Diego, CA, USA). Samples were prepared for flow cytometry as described elsewhere (see, e.g., Teshima et al., Biol Blood Marrow Transplant 22:11-16 (2016)). All antibodies used to stain samples are listed in Table 1. Flow cytometry was performed on three-laser CytoFLEX (Beckman Coulter, Chaska, MN, USA) where cells were gated by singlet discrimination, and live cells were determined by Live/Dead Aqua staining (L34966, Thermo Fisher Scientific, Waltham, MA, USA). All gating analyses were performed with FlowJo X10.0.7r2 software (Ashland, OR, USA) and Kaluza Analysis software (Indianapolis, Indiana, USA).

TABLE 1
Antibodies.

| Antibody Name | Clone | Vendor | Catalog # |
|---|---|---|---|
| PE/Cyanine7 anti-human Galectin-9 Antibody | 9M1-3 | BioLegend | 348916 |
| PE anti-human CD105 Antibody | 43A3 | BioLegend | 323205 |
| PerCP/Cyanine5.5 anti-human CD90 (Thy1) | SE10 | BioLegend | 328117 |
| Antibody | | | |
| CD73 Monoclonal Antibody (AD2), PE, | AD2 | Invitrogen | 25073942 |
| eBioscience ™ | | | |
| APC/Cyanine7 anti-human CD183 (CXCR3) | G025H7 | BioLegend | 353722 |
| Antibody | | | |
| CD184 (CXCR4) Monoclonal Antibody (2B11), | 2B11 | Life | 17999182 |
| APC, eBioscience ™ | | Technologies | |
| Brilliant Violet 650 ™ anti-human CD3 Antibody | OKT3 | BioLegend | 317324 |
| CD279 (PD-1) Monoclonal Antibody (MIH4), | MIH4 | eBioscience | 11996942 |
| FITC, eBioscience ™ | | | |
| Brilliant Violet 650 ™ anti-human CD3 Antibody | OKT3 | BioLegend | 317324 |
| APC anti-human CD4 Antibody | OKT4 | BioLegend | 317416 |
| Brilliant Violet 421 ™ anti-human CD45 | HI30 | BioLegend | 304032 |
| Antibody | | | |
| APC/Cyanine7 anti-mouse CD45 Antibody | 30-F11 | BioLegend | 103116 |
| Brilliant Violet 605 ™ anti-human CD25 | BC96 | BioLegend | 302632 |
| Antibody | | | |
| FITC Mouse Anti-Human CD127 | HIL7RM21 | BD Biosciences | 561697 |
| CD279 (PD-1) Monoclonal Antibody (eBioJ105 | eBioJ105 | Life | 25279942 |
| (J105)), PE-Cyanine7, eBioscience ™ | | Technologies | |
| Goat anti-Mouse IgG (H + L) Cross-Adsorbed | | Invitrogen | A21235 |
| Secondary Antibody, Alexa Fluor ™ 647 | | | |
| Biotin-Protein L | | GenScript | M00097 |
| PE Streptavidin | | BioLegend | 405203 |

### In Vivo Mouse Studies

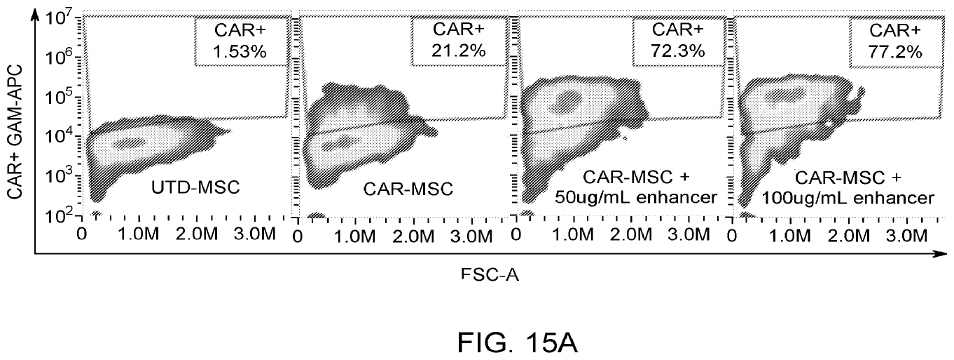

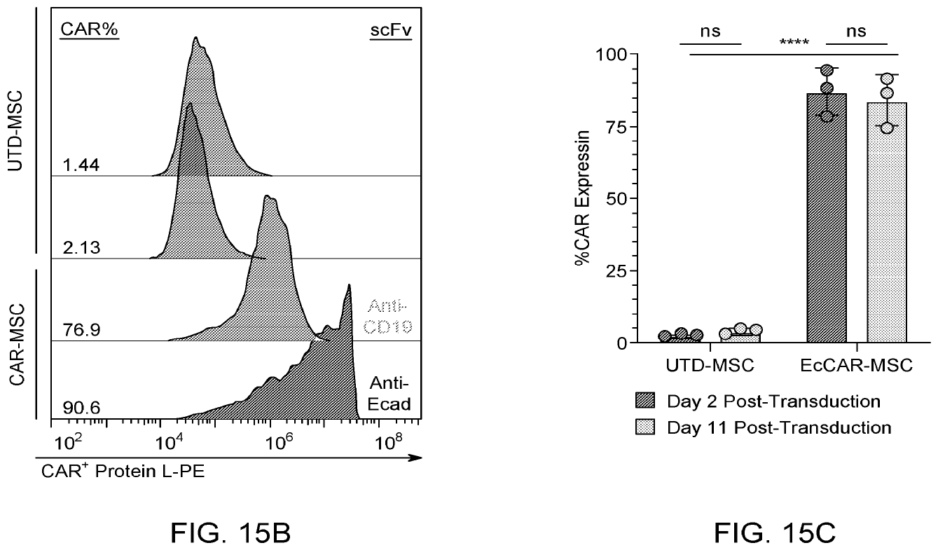

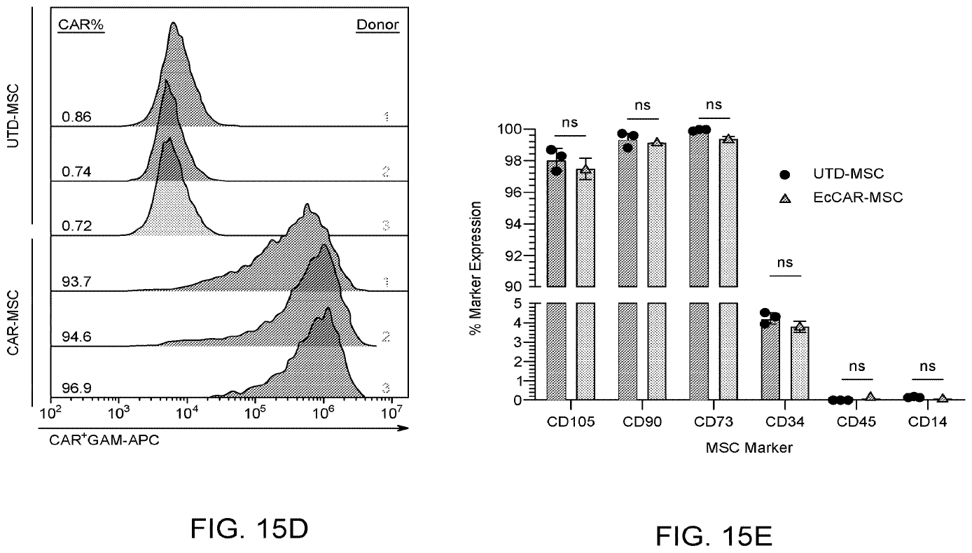

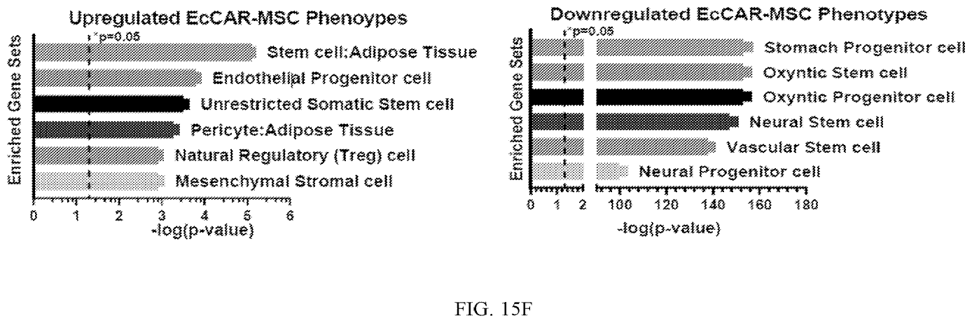

Female and male immunocompromised NOD-SCID-γ^{-/-} (NSG) mice were obtained from Jackson Laboratories at 6-8 weeks old. All cells were injected in 100 μL-200 μLs of PBS via syringe through tail vein injection or intraperitoneal injection. Mice were imaged with a bioluminescent imager using an IVIS® Lumina S5 Imaging System (PerkinElmer, Hopkinton, MA, USA) to confirm engraftment of luciferase positive tumor model CD19⁺ Nalm6/JeKo1 cells in the cancer xenograft model or luciferase positive CAR-MSCs in the MSC persistence xenograft model. Imaging was performed 10 minutes after the intraperitoneal injection of 10 μL/g D-luciferin (15 mg/mL, Gold Biotechnology, St. Louis, MO, USA). In tumor mouse models, NSG mice were engrafted with the CD19⁺ luciferase⁺ JeKo-1 or Nalm6 (1×10⁶ cells given i.v.). Engraftment was confirmed by bioluminescent imaging 1-2 weeks after injection. All mice then received irradiated Ecad⁺ MCF-7 cells (5×10⁶ cells given i.p. to stimulate the EcCAR-MSCs) and CART19 cells (1×10⁶ cells given i.v. as a strategy to treat the CD19⁺ tumor). Mice were then randomized based on bioluminescent imaging as a measure of tumor burden to receive UTD-MSCs, EcCAR-MSCs, or no additional treatment (FIG. 15). Serial bioluminescent imaging was subsequently performed to assess residual disease and determine antitumor activity of CART19 cells.

In GVHD mouse models, GVHD was induced in NSG mice with allogeneic human

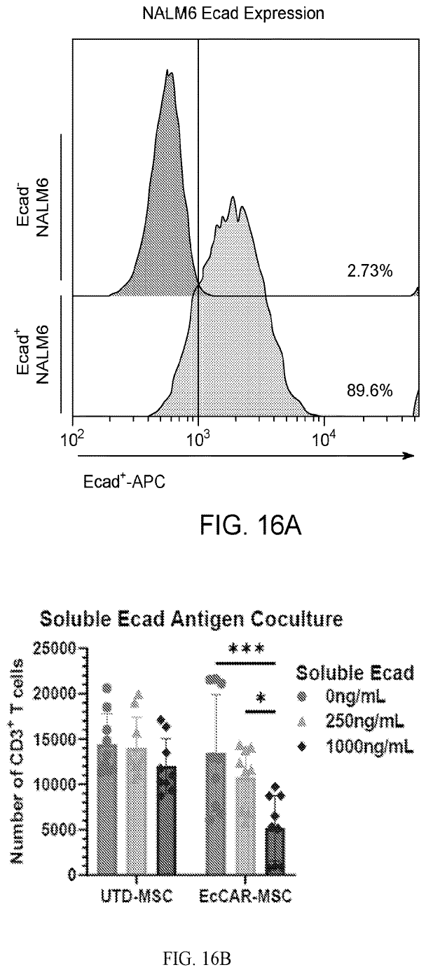

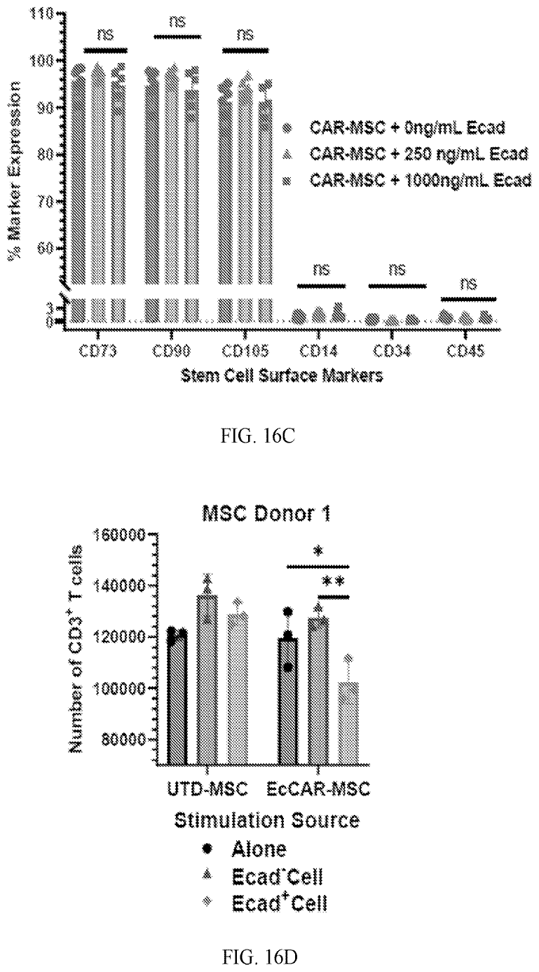

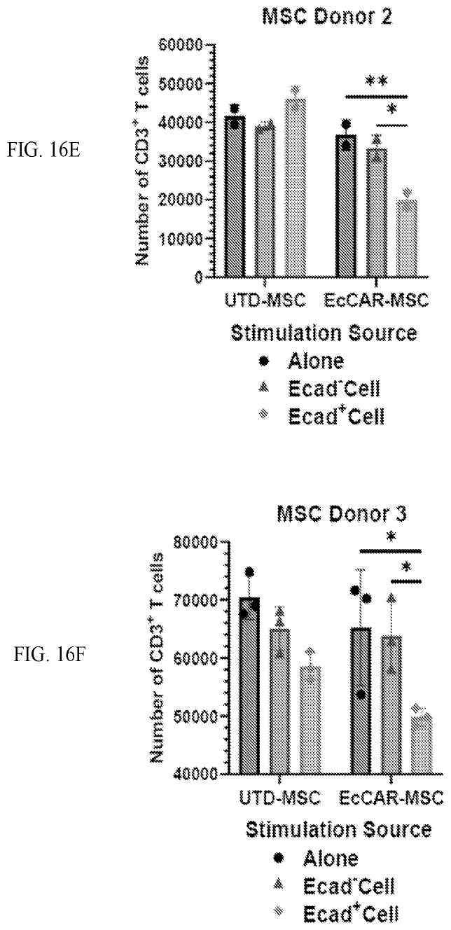

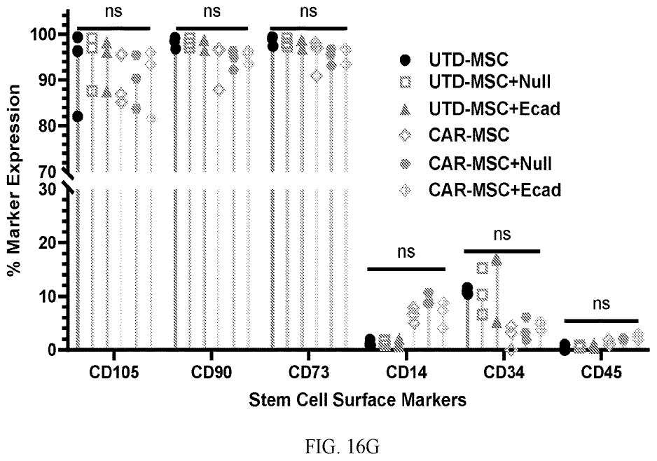

PBMCs (20-30×10⁶ cells i.v.) and treated with either 1) UTD-MSCs or 2) CAR-MSCs (1×10⁶ cells i.p. on days 10 and 20) as well as 3) no MSC control mice (FIG. 16). Body weight and clinical GVHD scoring (incorporating body weight, posture, diarrhea, activity, fur condition, and skin integrity) were monitored in all experiments for GVHD progression in each experimental group (n=5). Mouse blood was collected by tail vein bleed (around 100 μL) with 70 μL of blood used for flow cytometry analysis. RBC lysis was applied using 1:10 BD FACS Lyse buffer (BD Biosciences, San Jose, CA, USA). Cells were then washed in flow buffer and incubated with their specific antibody mix at room temperature in the dark before flow analysis using CytoFLEX (Beckman Coulter, Chaska, MN, USA) was performed. The remainder of the blood was centrifuged at 13,000 rpm for 10 minutes to separate serum for cytokine analysis.

### Canine Studies

The Ecad-targeted CAR was based on a human/canine cross-reactive Ecad-directed scFv. This scFv was generated using phage display, and cross-reactivity with both dog and human Ecad was confirmed (FIG. 7). EcCAR-MSCs were tested in healthy beagles. Subjects were injected i.p. with EcCAR-MSCs (1×10⁶ cells/kg). Subjects were followed daily, weights were monitored, and serial blood sampling was performed on colon biopsies on day 3 and examined by IHC for human CD105⁺ cells to assess MSC homing to canine Ecad⁺ cells.

### RNA Isolation, Sequencing, and Analysis

EcCAR-MSC or UTD-MSC cells were transduced accordingly in culture with or without recombinant human Ecad Fc chimera (BioLegend, San Diego, CA, USA) at 250 ng/ml as a form of CAR specific stimulation for 24 hours. Following in vitro culture, MSCs were detached for RNA isolation using a QIAGEN miRNeasy micro kit (Catalog no. 217084, QIAGEN, Germantown, MD, USA). Bulk RNAseq was performed on MSCs from 3 different biological donor replicates in both UTD-MSC and EcCAR-MSC group to ensure rigor of results.

Total RNA was prepped with a SMARTer stranded total RNA-seq kit v2, Pico input mammalian (Takara, Mountain View, CA, USA). Total RNA (three samples per lane) was sequenced on an Illumina HiSeq 4000 (Illumina, San Diego, CA, USA). Library preparation and sequencing were performed. Quality check was performed with FastQC v0.11.8 on sample generated FastQC files. Cutadapt v1.18 was used to trim and remove adapter sequences. Output files were confirmed for adaptor removal and quality using FastQC v0.11.8.

Paired end reads from trimmed FastQ files were mapped to the latest human reference genome (GRCh38) downloaded from the NCBI databases. Genome index files were built and aligned with STAR v2.5.4b. Generic expression counts for each gene were generated with HTSeq (Python 3.6.5). Gene counts were normalized (geometric mean) and differential expression analysis was calculated with DESeq2 (R v3.6.1, R-project.org/) using adjusted p values<0.05 as statistical cut offs. Heatmaps were created using pheatmap (cran.r-project.org/web/packages/pheatmap/index.html) with PCAs generated with ggplot2 tools (cran.r-project.org/web/packages/ggplot2/index.html).

Gene set enrichment analyses were performed using Enrichr. Protein-protein interaction networks were generated using QIAGEN Ingenuity Pathway Analysis.

### Cytokine Assays

Cytokine assays were performed on mouse serum samples collected 15 days following MSC treatment or control treatment. Debris was removed from serum by centrifugation at 10,000×g for 5 minutes. Serum was diluted 1:2 with serum matrix before plating and following the manufacturer's protocol for Milliplex Human Cytokine/Chemokine MAGNETIC BEAD Premixed 38 Plex Kit (HCYTMAG-60K-PX38, Millipore Sigma, Ontario, Canada). Data were collected using a Luminex (Millipore Sigma, Ontario, Canada) and analyzed with Belysa Immunoassay Curve Fitting Software (Millipore Sigma, Ontario, Canada) and Microsoft Excel (Microsoft, Redmond, WA, USA). Significant findings were determined and reported with Prism Graph Pad (La Jolla, CA, USA).

### Statistical Analysis

In vitro and in vivo experiments were performed using technical and biological replicates to allow for appropriate statistical analysis. 2-way or 1-way ANOVA analyses were used to evaluate the differences between in vitro EcCAR-MSC and UTD-MSC surface marker expression by percent expression and T cell suppression capabilities by absolute T cell count evaluations. ANOVA analyses were also used to determine significant difference between in vivo EcCAR-MSC treated mice and UTD-MSC treated mice through IVIS imaging of luciferase positive tumors and luciferase positive MSCs, weight comparisons, blood composition by flow, and GVHD clinical score comparisons. Kaplan Meier survival analysis was used to determine significance of difference in survival outcomes in both tumor and GVHD Xenograft Models with Cox regression analyses used to adjust for confounders (e.g., sex). To make multiple comparisons between each individual group in each of the previously described analyses (above), Tukey's multiple comparisons test was used as a supplement to ANOVA based analyses. For comparison between two groups, a two tailed unpaired Student's t test was used in place of ANOVA. RNA-seq data was processed with DeSeq2 program where raw counts were normalized across the samples (geometric mean), and Benjamini-Hochberg procedure was used for multiple hypothesis correction. All relevant statistically significant comparisons were demarked with asterisks corresponding to levels of significance below p<0.05 indicating a 95% confidence interval (where *p<0.05, **p<0.005, ***p<0.0005, and ****p<0.0001). End data points without asterisks represent not significant (ns) differences between groups. Relevant data depicts the mean of all data points with either standard deviation (s.d.) or standard error of the mean (SEM) used to determine error bars. Prism Graph Pad (La Jolla, CA, USA) and Microsoft Excel (Microsoft, Redmond, WA, USA) were used to analyze the experimental data.

### Results

### MSCs are Successfully Transduced to Stably Express CAR.

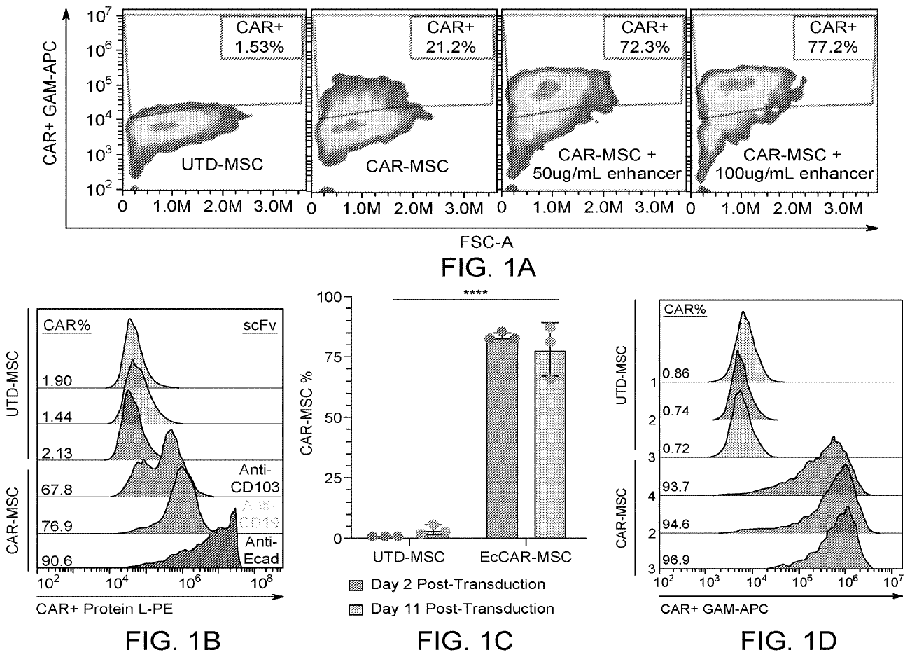

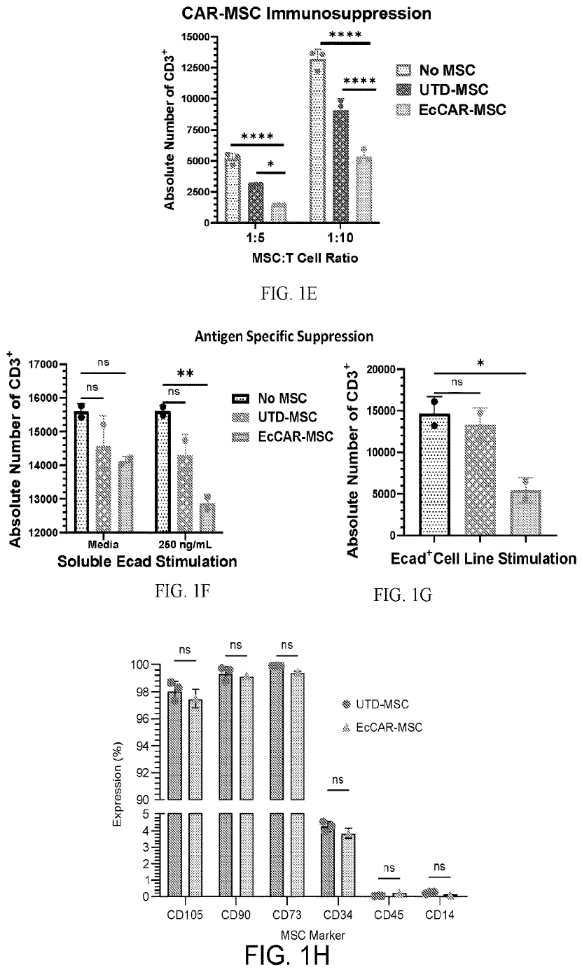

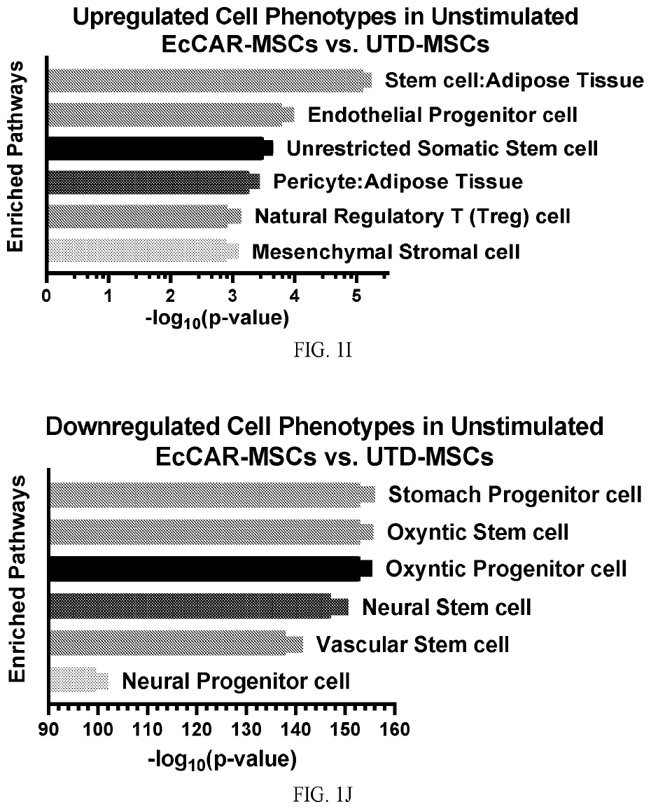

Using polycation enhancer augmented lentiviral vectors, efficient transduction with stable expression of CAR into MSCs was established. Transduction using a CAR19 lentiviral vector was optimized as a proof of concept for CAR-MSC creation. The delivery of CAR-containing lentiviral particles was optimized for efficiency and cell survival with protamine sulfate enhancer. Using protamine sulfate enhancer and lentiviral MOI of 3, consistent CAR transduction efficiency of >70% was achieved (FIG. 1A). Following transduction optimization using CAR19, alternative CAR designs were developed for CAR-MSC based autoimmune disease therapy. The primary CAR-MSC construct was directed to Ecad, a ligand expressed on inflamed intestinal epithelial cells involved in GVHD. As T cell migration is mediated by the interaction of CD103 (αE integrin) on the T cell surface with Ecad, which is universally expressed on all epithelial cells, it was determined whether redirecting MSCs to Ecad via CAR would protect host epithelial tissues by enhancing MSC specificity and activation at sites of inflammation in GVHD and other gut autoimmune diseases. Using phage display, several Ecad directed scFvs were generated and a human and canine cross reactive scFv was identified to enable testing in canine models (FIG. 6). Using this clone, an Ecad-specific CAR construct was generated (FIG. 7) and transduced into MSCs creating anti-Ecad CAR-MSCs (EcCAR-MSCs) with >90% transduction efficiency (FIG. 1B). MSCs were derived from 5 different biological MSC donors (Table 2).

TABLE 2
MSC donor profiles.

| MSC Donor | Gender | Age | Race | Sample |
|---|---|---|---|---|
| D1 | Male | 48 | White | Normal |
| D2 | Female | 26 | White | Pectus Excavatum |
| D3 | Male | 26 | White | Pectus Excavatum |
| D4 | Male | 25 | White | Normal |
| D5 | Male | 28 | White | Normal |

To determine if the immunosuppressive effects of CAR-MSCs could be enhanced through inclusion of specific intracellular signaling domains within the CAR, CD28 signaling was incorporated into the CAR-MSC construct design using a CD28 co-stimulated CAR backbone yielding a CAR targeting Ecad using a CD28ζ CAR backbone (EcCAR-MSCs) (FIG. 7).

It was also demonstrated that stable CAR expression in EcCAR-MSCs over multiple ex vivo passages (>3) was possible, and that >90% transduction efficiency was achieved across multiple MSC cell donors (FIGS. 1C-1D, see Table 3 for a description of GVHD clinical scoring system). To test the ability of EcCAR-MSCs to suppress activated T cells, T cell suppression assays were performed. UTD-MSCs or EcCAR-MSCs (suppressors) were co-cultured with activated T cells (effectors). EcCAR-MSCs induced superior suppression of T cells compared to UTD-MSCs in a dose-dependent manner (FIG. 1E). The antigen-specific activation of EcCAR-MSCs was tested by measuring T cell suppression by EcCAR-MSCs in the presence or absence of Ecad protein to stimulate the anti-Ecad scFv on the CAR. To ensure reproducibility, both soluble Ecad and Ecad⁺ MCF7 cell lines were used as a source of Ecad antigen. Both soluble and cell-based antigen-specific stimulation of EcCAR-MSCs resulted in enhanced suppression of T cells (FIGS. 1G-1G).

TABLE 3
GVHD clinical scoring system.

| | Weight | | | | Fur | Skin |
|---|---|---|---|---|---|---|
| Grade | Loss | Diarrhea | Posture | Activity | Texture | Integrity |
| 0 | <10% | No | Normal | Normal | Normal | Normal |
| 1 | 10%-<25% | Yes | Hunched | Reduced | Rufflings | Flaked |
| 2 | ≥25% | Yes | Severely | Inactive | Severe | Obviously |
| | | | hunched | | Rufflings | denuded |

MSC Engineering does not Induce Differentiation, and CAR-MSCs Retain Stemness.

EcCAR-MSC stem phenotype and pathway enrichment was interrogated to identify unwanted differentiation induced by CAR-MSC engineering. Flow cytometry was used to study EcCAR-MSC stem phenotype (CD105⁺, CD73⁺, CD90⁺, CD34⁻, CD14⁻, and CD45⁻), based on the minimum criteria for MSC stem maintenance set by the International Society of Cell Therapy (see, e.g., Dominici, et al., Cytotherapy 8, 315-317 (2006)). No significant differences between CAR-MSCs and UTD-MSCs were found (FIG. 1H).

Differential gene expression analysis by bulk RNA sequencing (RNAseq) comparing EcCAR-MSCs to UTD-MSCs was performed. Gene set enrichment analysis (GSEA) using CellMarker Augmented gene set revealed enrichment of adipose derived stem cell and unrestricted somatic stem cell subsets in EcCAR-MSCs (FIG. 1I). Conversely, differentiated cell lineages such as smooth muscle, neural or vascular differentiation pathways were downregulated through GSEA (FIG. 1J). These results indicate maintained stem phenotype following CAR-MSC engineering with limited differentiation induced by transduction or CAR expression.

### EcCAR-MSCs Exhibit Superior Immunosuppression in Tumor and GVHD Models In Vivo.

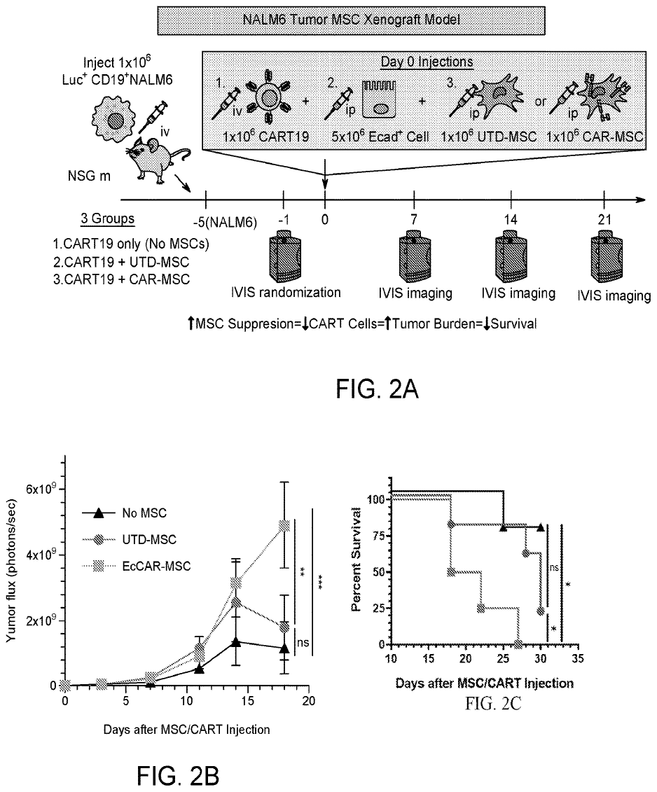

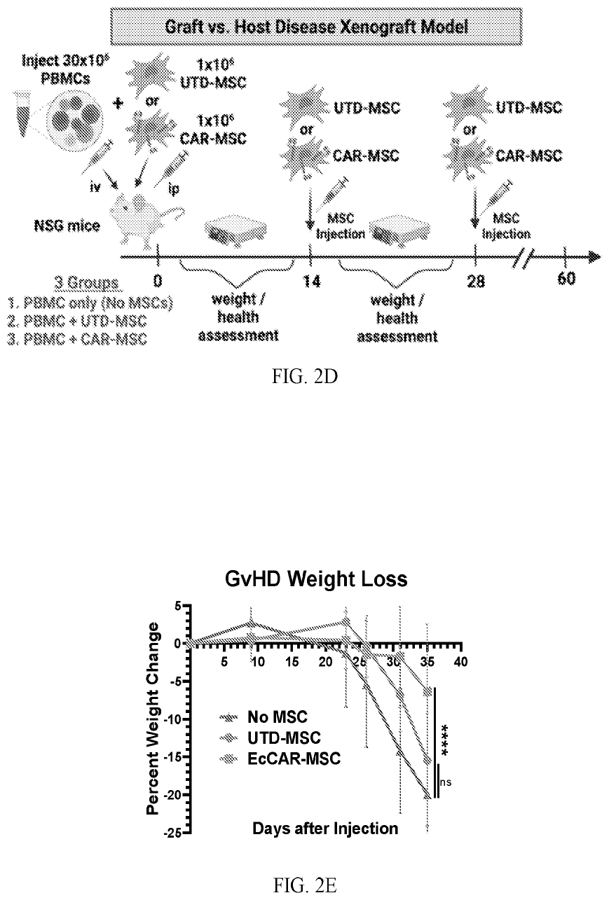

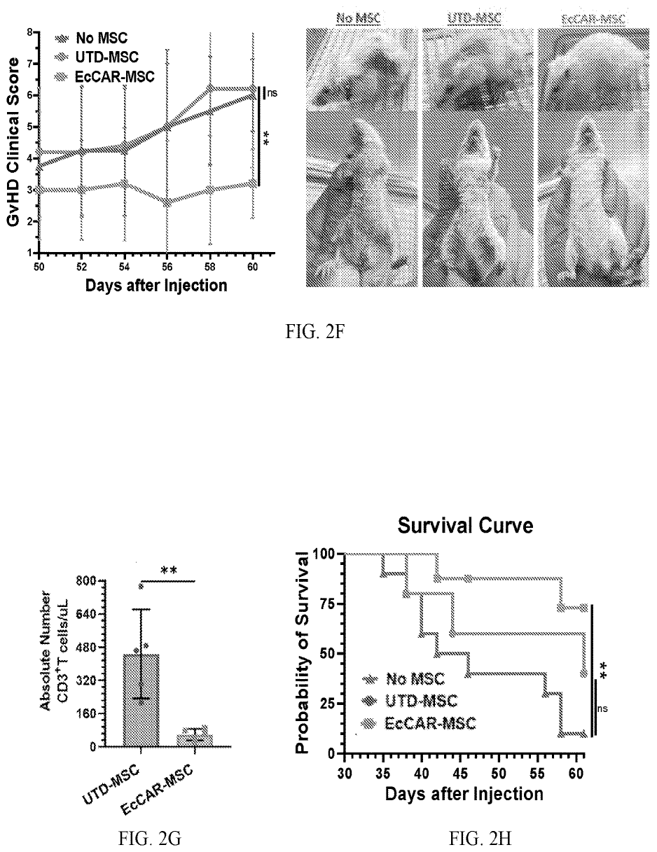

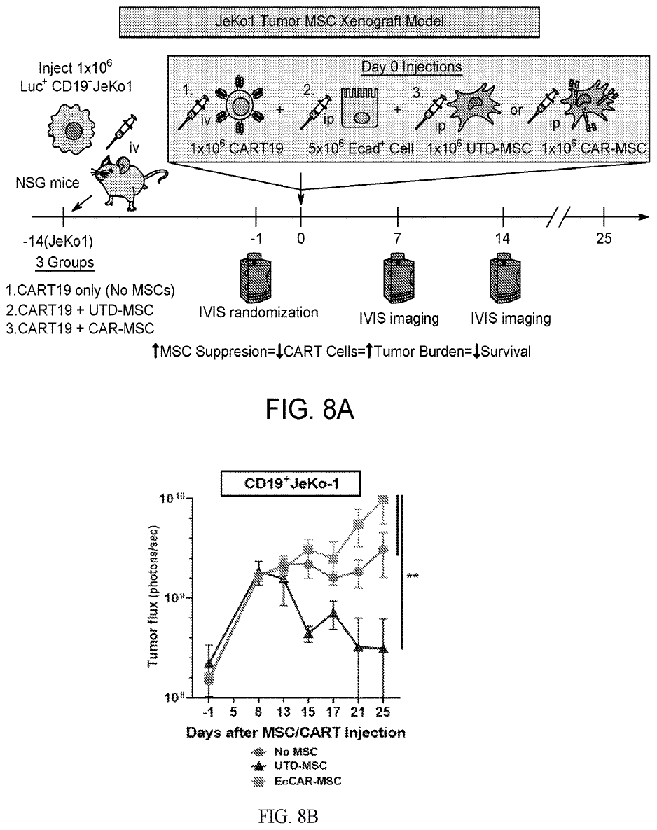

To study the immunosuppressive functions of CAR-MSCs in vivo, multiple tumor and GVHD models were used to ensure reproducibility and rigor of results. For tumor xenograft models, CD19⁺ mantle cell lymphoma JeKo-1 and CD19⁺ acute lymphoblastic leukemia Nalm6 were used to test if CAR-MSCs suppressed the potent antitumor activity of CART19 cells (FIG. 2A). Xenograft models were established through i.v. administration of either luciferase⁺ JeKo-1 or luciferase⁺ Nalm6 cells into immunocompromised NSG mice. Bioluminescent imaging was performed one week later to confirm engraftment, and mice were randomized to treatment with CART19 alone, CART19 in combination with UTD-MSCs, or CART19 in combination with EcCAR-MSCs. Tumor burdens were assessed through biweekly bioluminescent imaging. Mice engrafted with Nalm6 and treated with CART19 plus EcCAR-MSCs displayed a high tumor burden (FIG. 2B) and reduced overall survival compared to mice treated with CART19 plus UTD-MSCs or CART19 alone (FIG. 2C). Similar results were demonstrated using Jeko-1 xenografts treated with CART19 plus EcCAR-MSCs or UTD-MSCs (FIGS. 8A-8B). Together, these data demonstrate superior effector T cell suppression by EcCAR-MSCs through increasing tumor burden and decreasing survival outcomes.

A xenograft GVHD model was then generated through the i.v. administration of human PBMCs into NSG mice. In addition to PBMCs, mice were given PBS control, UTD-MSCs, or EcCAR-MSCs as GVHD treatment via intraperitoneal (i.p.) injection. Additional doses of MSCs or control treatment were administered i.p. every two weeks (FIG. 2D). Mice were monitored for the development of clinical GVHD symptoms (e.g., diarrhea, motor function, posture, fur integrity, and skin integrity, body weight changes, and overall survival (see Table 3 for a description of GVHD clinical scoring system)). Peripheral blood was collected 14 days after the first dose of MSC treatment and analyzed for human T cell proliferation. EcCAR-MSC treatment led to prevention of weight loss (FIG. 2E), amelioration of clinical GVHD (FIG. 2F), suppression of T cell proliferation (FIG. 2G), and improved overall survival (FIG. 2H) compared to treatment with UTD-MSCs Together, these results indicate enhanced T cell suppression by EcCAR-MSCs, leading to the prevention of GVHD.

### EcCAR-MSCs Display Increases in Critical Transcription Factors, Suppressive Cytokines, and Inhibitory Surface Marker Expression Inducing an Immunosuppressive Milieu.

To elucidate the mechanisms of enhanced EcCAR-MSC immunosuppression, CAR-MSCs were surveyed for unique transcriptional activation, cytokine secretion profiles, and surface markers characterizing their phenotype.

To determine the impact of antigen specific CAR-MSC stimulation on the MSC transcriptome, RNAseq was performed on the following conditions: UTD-MSC (unstimulated), UTD-MSC (stimulated with soluble Ecad), EcCAR-MSC (unstimulated) and EcCAR-MSC (stimulated with soluble Ecad, to stimulate CAR-MSC through the CAR).

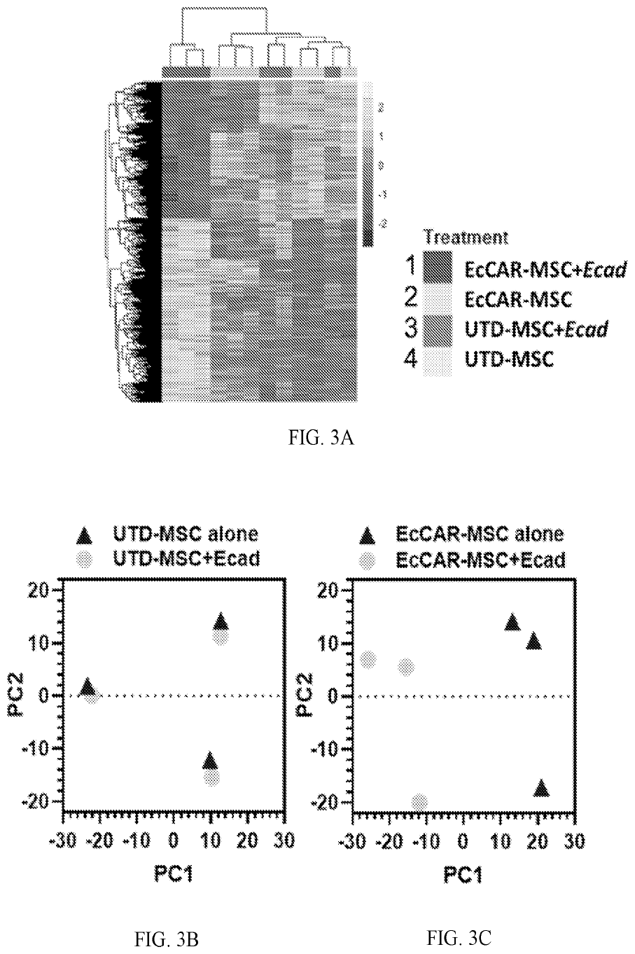

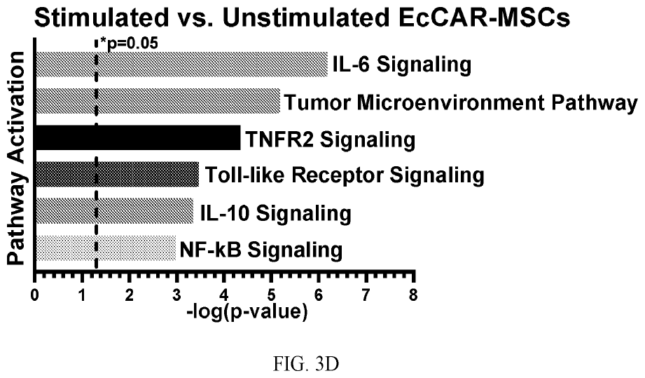

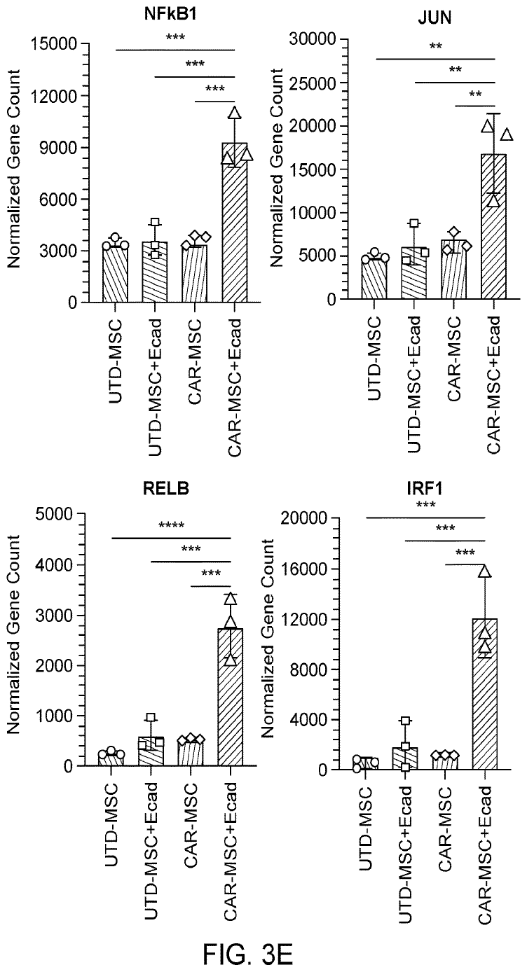

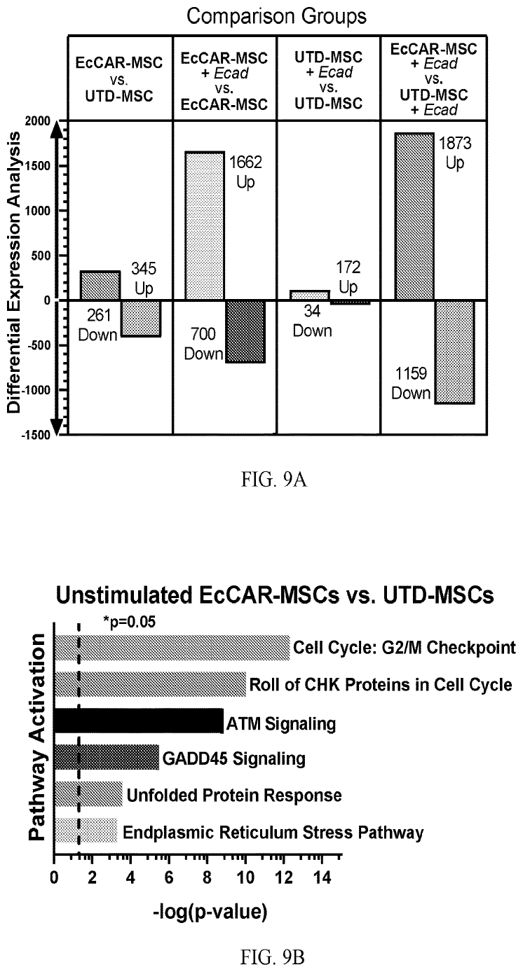

To elucidate pathway activation intrinsic to EcCAR-MSC and stimulated EcCAR-MSC, comprehensive differential gene expression analysis was performed with the following comparisons: 1) EcCAR-MSC (unstimulated) vs. UTD-MSC (unstimulated) to account for intrinsic differences induced by EcCAR transduction and insertion, 2) EcCAR-MSC (stimulated with soluble Ecad) vs. EcCAR-MSC (unstimulated) to account for differences induced by antigen specific stimulation of EcCAR-MSC through the CAR's scFv, 3) UTD-MSC (stimulated with soluble Ecad) vs. UTD-MSC (unstimulated) to serve as a control for potential Ecad-based stimulation in the absence of Ecad-specific CAR scFv. Unstimulated EcCAR-MSCs and UTD-MSCs (comparison 1) revealed distinct pathway activation with significant upregulation (at least 1.5-fold) of 355 genes in EcCAR-MSCs (FIG. 3A and FIG. 9).

Differential gene expression analysis between Ecad-stimulated EcCAR-MSCs and unstimulated EcCAR-MSCs revealed more robust transcriptional activation with 1996 genes upregulated at least 1.5-fold (FIG. 3A). This upregulation of nearly 6 times as many genes as comparison 1 highlights the functional capacity of CAR-based stimulation within the MSC (FIG. 9). PCAs of expression profiles between Ecad-stimulated and unstimulated CAR-MSCs revealed clustering based on stimulation status (FIG. 3B), while Ecad-stimulated and unstimulated UTD-MSCs (comparison 3) clustered solely by MSC biological donor (FIG. 3C). GSEA of upregulated pathways characterized by CAR stimulation was more robustly linked to NFκB-mediated TNFα signaling pathways and IL-10 anti-inflammatory signaling pathways (FIG. 3D).

These pathways are related to canonical CD28 signaling through intracellular proteins binding to CD28 src-like domains, inducing nuclear activation of downstream functional cascades. Comparisons between unstimulated EcCAR-MSCs and UTD-MSCs were indicative of changes in signaling pathways inherent to the inclusion of CAR signaling domains and CAR transduction rather than stimulation of the CAR through its scFv.

Transcriptomic analyses also demonstrated elevation of transcription factors NFκB1, JUN, RELB, and IRF1 in Ecad stimulated EcCAR-MSC (FIG. 3E).

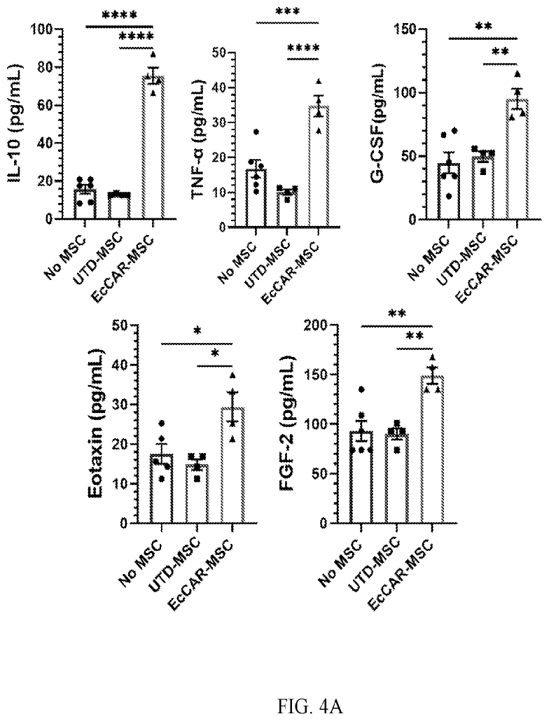

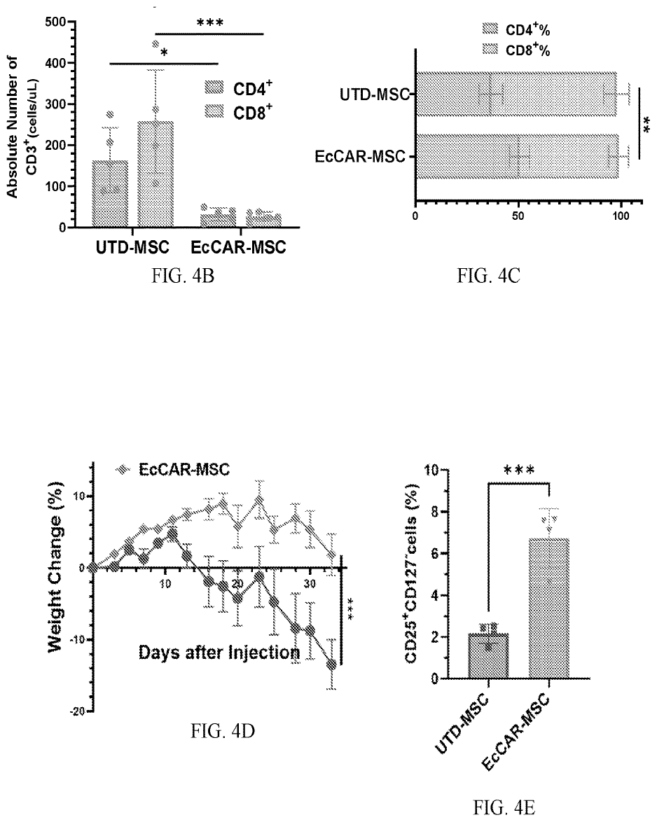

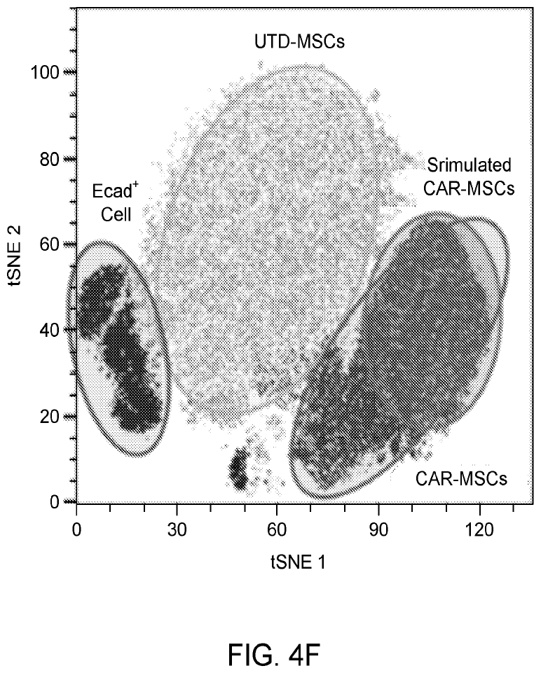

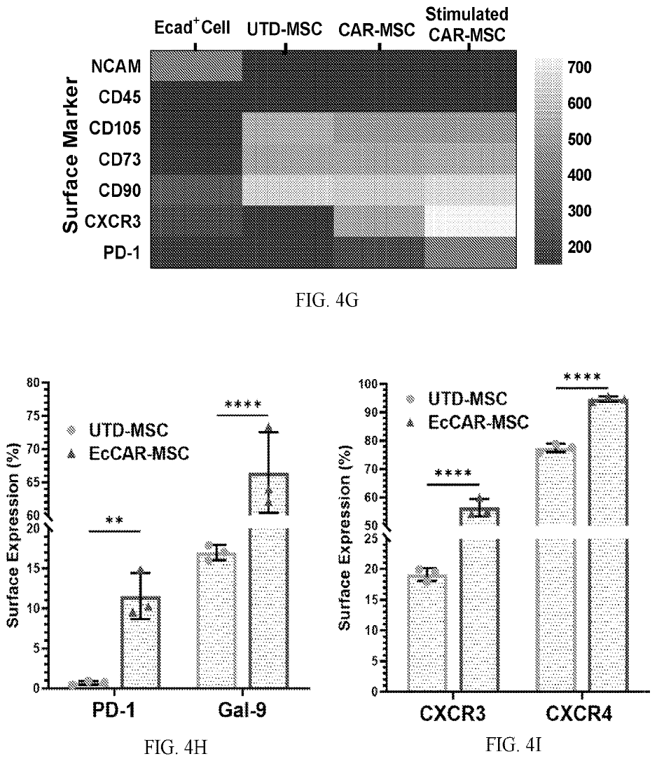

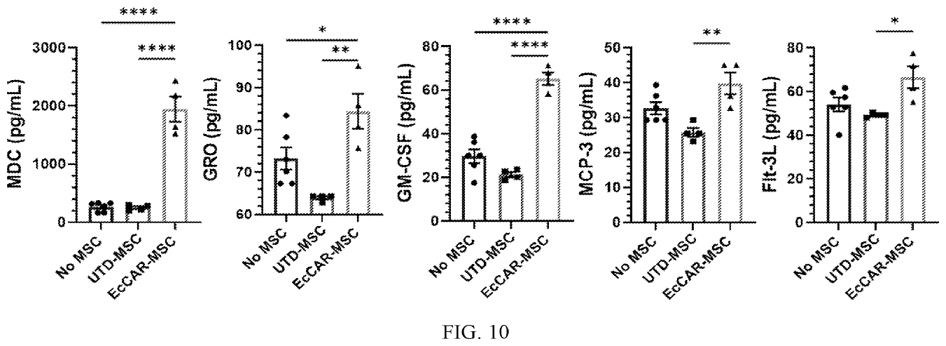

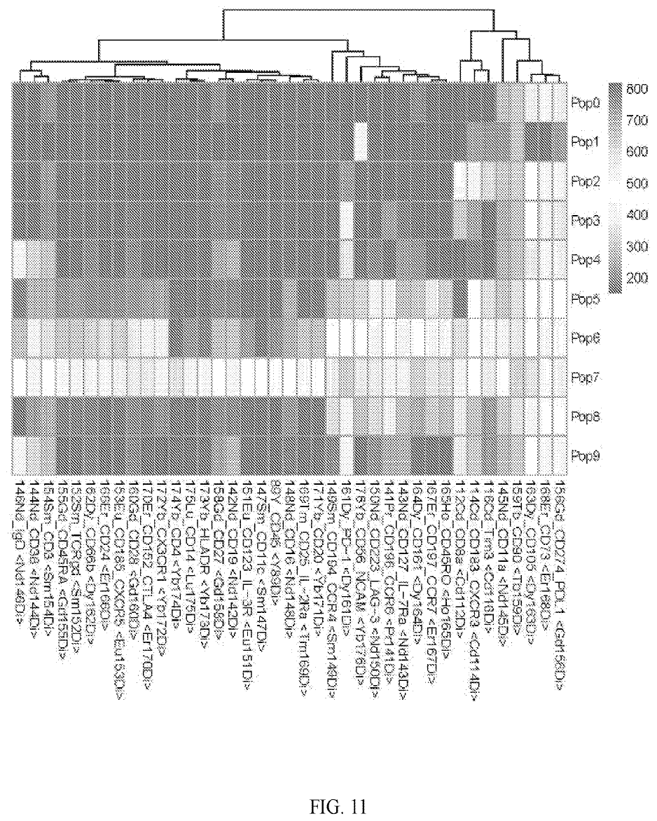

To validate transcriptional findings, human cytokines were measured in the serum of UTD-MSC- and EcCAR-MSC-treated mice. Analysis of serum cytokine composition 17 days following MSC administration supported RNAseq data with significant elevations of TNFα and IL-10 cytokines in EcCAR-MSC-treated mice compared to UTD-MSC-treated mice. Fibroblast Growth Factor 2 (FGF-2) was also elevated the serum of EcCAR-MSC-treated mice (FIG. 4A and 10).

To characterize MSC impact on human T cell subset composition in the mouse studies, peripheral blood was collected 14 and 31 days following UTD-MSC or EcCAR-MSC treatment in the GVHD xenograft model where weight loss was ameliorated following treatment with EcCAR-MSC (see FIG. 2D). Analysis by flow cytometry revealed significant CD4⁺ and CD8⁺ human T cell suppression in the EcCAR-MSC treated group, correlating to sustained weight measurements (FIGS. 4B-4C). Analysis of CD4⁺ to CD8⁺ ratios revealed a propensity towards increased CD4⁺ T cell subsets and/or diminished CD8⁺ cytotoxic T cell subsets in EcCAR-MSC-treated mice as compared to UTD-MSC-treated mice (FIG. 4D). Long-term analysis of blood on day 31 following MSC administration revealed a significant increase in percentage of Treg (human CD4⁺, CD25⁺, CD127⁻) cells in EcCAR-MSC-treated groups (FIG. 4E).

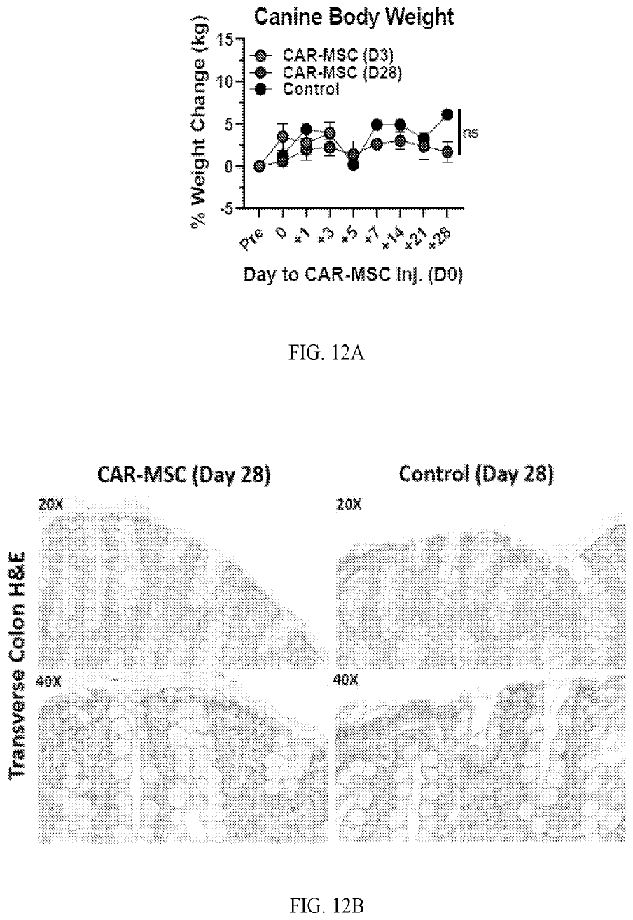

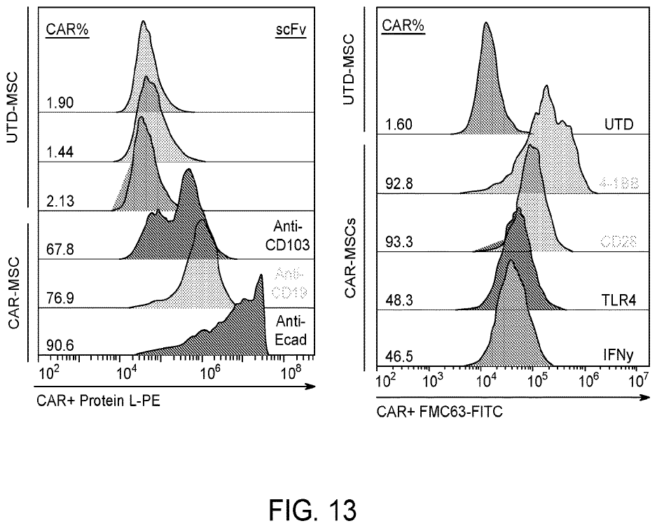

EcCAR-MSC surface marker expression was surveyed with CyTOF, which supported preservation of stem phenotype and revealed alterations in immunosuppressive surface markers and chemokines. Unlike UTD-MSCs, resting and Ecad-stimulated EcCAR-MSCs clustered into separate populations by surface marker-based t-distributed stochastic neighbor embedding statistical analysis indicating functional CAR antigen specific stimulation (FIGS. 4F-4G and 11). To test whether such distinct immunosuppressive surface marker profiles were maintained on EcCAR-MSCs following coculture with effector cells, additional surface marker characterization was performed on PBMC-cocultured MSCs through flow cytometry. EcCAR-MSCs and UTD-MSCs were stimulated with soluble Ecad and cocultured with PBMCs for 5 days before surface marker assessment. These results indicated distinct EcCAR-MSC surface marker upregulation of inhibitory receptors PD-1 and Galectin-9 (Gal-9) (FIG. 4H). CXCR3 and CXCR4 homing chemokines were also elevated on stimulated EcCAR-MSCs (FIG. 4I). This chemokine enhancement was supported by CXCR Chemokine Receptor Binding and Regulation of Leukocyte Chemotactic pathway enrichment in EcCAR-MSC from RNAseq analysis (FIG. 12).

Collectively, these mechanistic assays indicated enhanced immunosuppressive functions intrinsic to EcCAR-MSC creation, but especially upon CAR stimulation.

### EcCAR-MSCs Home to Target Sites and are Safe in Canine Models.

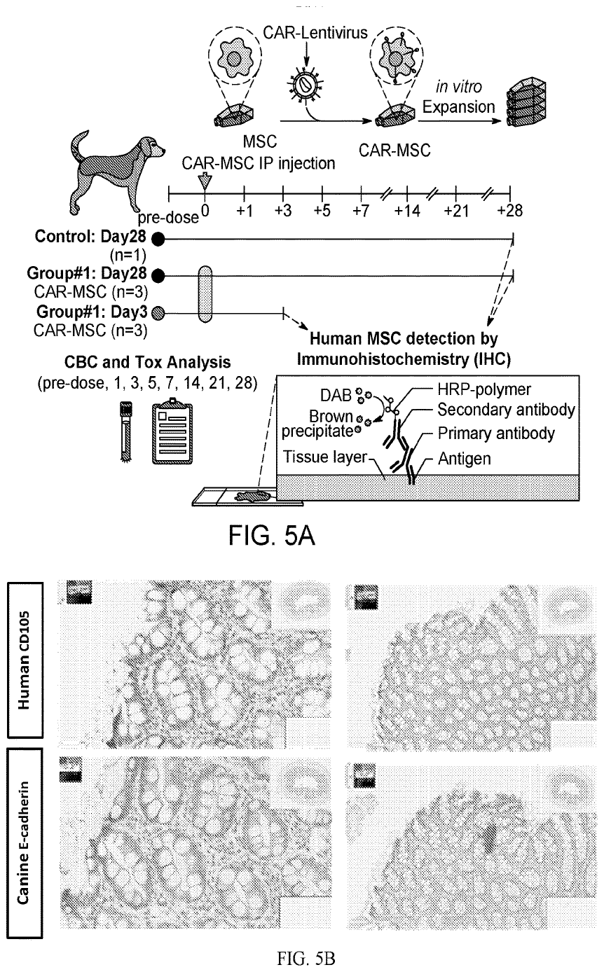

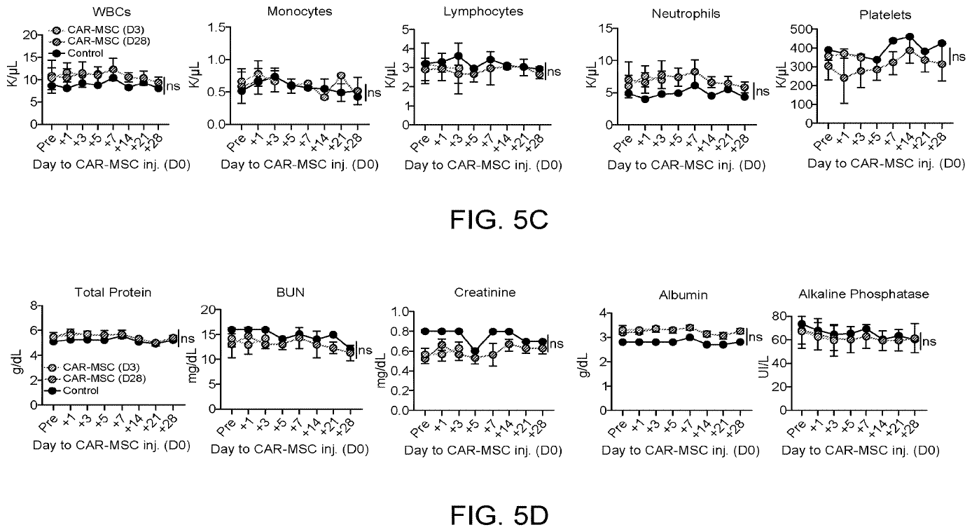

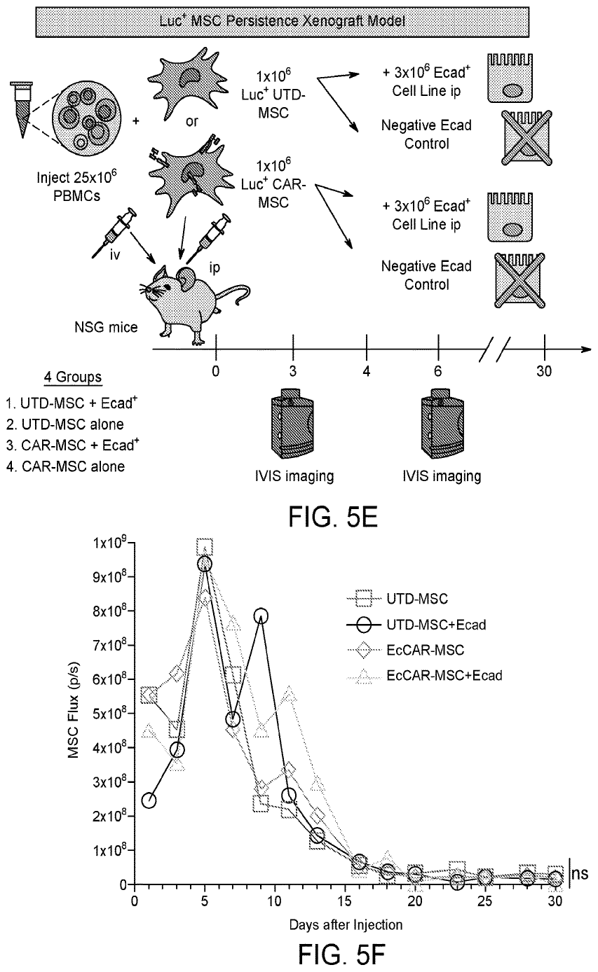

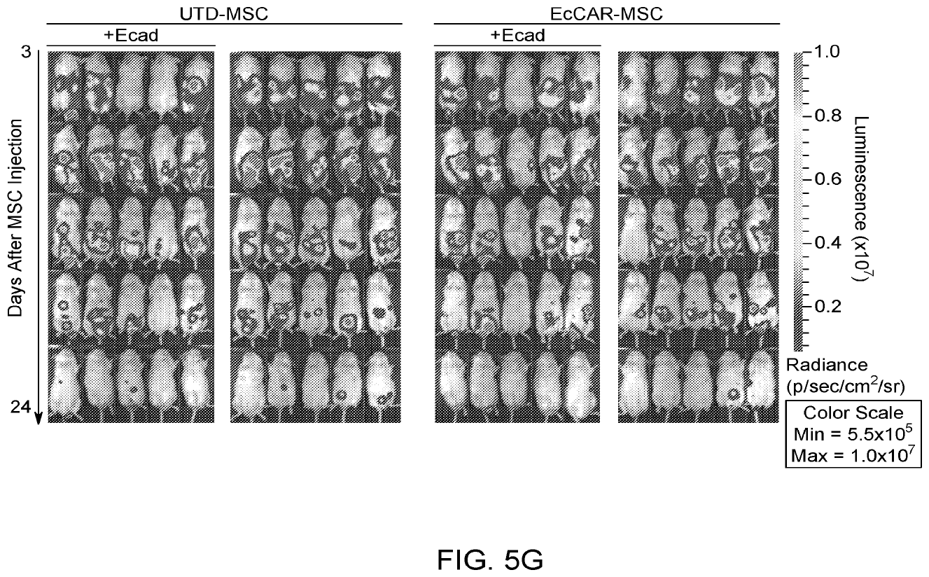

Canine models were used to ensure both homing capacity and safety of EcCAR-MSC administration using the canine cross-reactive EcCAR-MSCs (FIG. 7). CAR-MSCs were designed with human and canine cross reactive anti-Ecad scFv to create EcCAR-MSC. EcCAR-MSCs were manufactured into human MSCs via lentiviral transduction and expanded in vitro until intraperitoneal injection into healthy canine subjects. EcCAR-MSC scFv-mediated homing to canine Ecad⁺ cells was assessed through immunohistochemistry analysis. Subgroups were monitored for hematological and organ toxicity through blood assessment throughout the study. Healthy beagles were administered EcCAR-MSCs through i.p. injection. Colon biopsies were collected 3 and 28 days post-EcCAR-MSC administration evaluating canine Ecad⁺ ligand and human CD105⁺ MSCs cells by immunohistochemistry (FIG. 5A). Human CD105⁺ MSCs were present in colon tissues, overlapping with tissue regions of high canine Ecad expression, indicating target site homing (FIGS. 5B and 13). EcCAR-MSC toxicity was next assessed through serial measurements of complete blood counts, liver functions, kidney functions, and body weight. The administration of EcCAR-MSCs was not associated with weight loss, hematopoietic toxicity (FIG. 5D), or organ toxicity (FIG. 5E), indicating a strong safety profile.

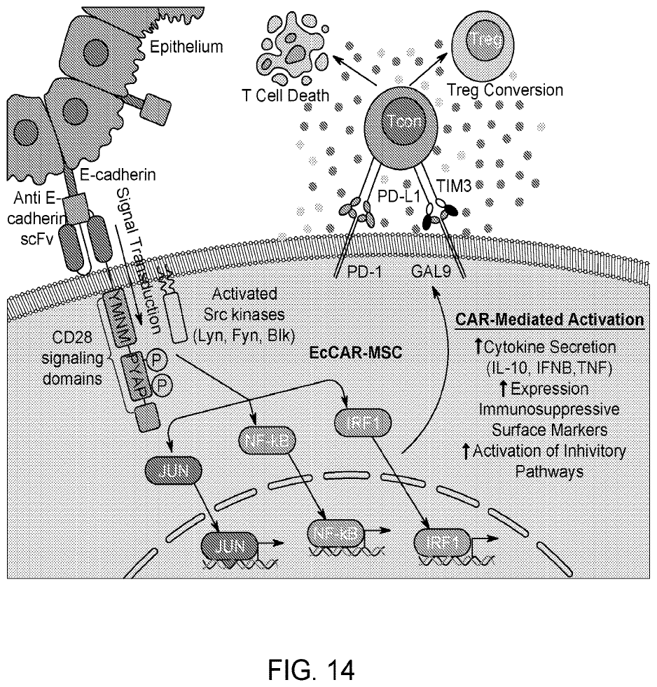

To study the expansion kinetics and persistence for further safety characterization of CAR-MSCs in vivo, luciferase⁺ CAR-MSCs were generated to monitor expansion, persistence, and clearance of EcCAR-MSCs by serial bioluminescent imaging with and without antigen-specific stimulation through the co-administration of irradiated Ecad⁺ MCF-7 cells (FIG. 14). Human PBMCs were administered to NSG mice, similar to the GVHD xenograft model. Mice were then treated with 1) luciferase⁺ UTD-MSCs alone, 2) luciferase⁺ UTD-MSCs plus irradiated Ecad⁺ MCF-7 cells, 3) luciferase⁺ EcCAR-MSCs alone, or 4) luciferase⁺ EcCAR-MSCs plus the irradiated Ecad⁺ MCF-7 cells (FIG. 5G). Mice were then monitored with serial bioluminescent imaging every 2-3 days to detect MSC expansion and persistence. These studies revealed no difference between the clearance time (~25 days) of UTD-MSCs and EcCAR-MSCs, further supporting the safety profile of EcCAR-MSCs (FIG. 5H-5I).

### Example 2: Exemplary Amino Acid Sequences

Exemplary scFv Sequences

CDR sequences are indicated by bold and underlined font.

Exemplary Anti-Ecad scFv (Also Referred to as Anti-hmcECAD.6)

Heavy chain
(SEQ ID NO: 1)
QVQLQESGPGLVKPSETLSLTCTVSGGSVSSYYWSWIRQPPGKGLEWIGHIYYSGNT
NYNPSLKSRVTISVDTSKNQFSLKLSSVTAADTAVYFCARDRWNYYDSSPGYYYYY
GMDVWGQGTTVTVSS
Light chain
(SEQ ID NO: 2)
LPVLTQPPSASGTPGQRVTISCSGSSSNIGSNYVYWYQQLPGTAPKLLIYRNNQRPSG
VPDRFSGSKSGTSASLAISGLQSEDEADYYCASWDTSLRAWVFGGGTKLTVLG
Exemplary anti-CD103 scFv
Heavy chain
(SEQ ID NO: 3)
QVQLQESGPGLVKPSETLSLTCTVSGGSVSSYYWSWIRQPPGKGLEWIGHIYYSGNT
NYNPSLKSRVTISVDTSKNQFSLKLSSVTAADTAVYFCARDRWNYYDSSPGYYYYY
GMDVWGQGTTVTVSS
Light chain
(SEQ ID NO: 4)
DIQMTQSPSSLSASVGDRVTITCRASQGIRNDLGWYQQKPGKAPKRLIFAASHLQS
GVPSRFSGSGSGTEFTLTISSLQPEDFATYYCQQHNSSPFTFGPGTRVDIK
Exemplary anti-MOG scFv
Heavy chain
(SEQ ID NO: 5)
EVKLHESGAGLVKPGASVEISCKATGYTFSSFWIEWVKQRPGHGLEWIGEILPGRG
RTNYNEKFKGKATFTAETSSNTAYMQLSSLTSEDSAVYYCATGNTMVNMPYWGQ
GTTVTVSSGGGGSGGGGSGGGGS
Light chain
(SEQ ID NO: 6)
DIELTQSPSSLAVSAGEKVTMSCKSSOSLLNSGNQKNYLAWYQQKPGLPPKLLIYG
ASTRESGVPDRFTGSGSGTDFTLTISSVQAEDLAVYYCQNDHSYPLTFGAGTKLEIK
Exemplary anti-AMPA scFv
Heavy chain
(SEQ ID NO: 7)
MKLPVLLVVLLLFTSPASSSEVQLQESGPSLVKPSQTLSLTCSVTGDSITSGFWNWLR
KFPGNKLEYLGYINYSGSTYYNPSLKSRISFTRDTSKNQYYLHLNSVTAEDTATYYC
ASWVLRDWGQGTTLTVSSARPTAPSVYPLAPVCGDTTGSSVTLGCLVKGYFPEPVT
LTWNSGSLSSGVHTFPAVLQSDLYTLSSSVTVTSSTWPSQSITCNVAHPASSTKVDKK
IEPRGPTIKPCPPCKCPAPNLLGGPSVFIFPPKIKDVLMISLSPIVTCVVVDVSEDDPDV
QISWFVNNVEVHTAQTQTHREDYNSTLRVVSALPIQHQDWMSGKEFKCKVNNKDL
PAPIERTISKPKGSVRAPQVYVLPPPEEEMTKKQVTLTCMVTDFMPEDIYVEWTNNG
KTELNYKNTEPVLDSDGSYFMYSKLRVEKKNWVERNSYSCSVVHEGLHNHHTTKS
FSRTPGK
Light chain
(SEQ ID NO: 8)
MTSTLPFSPQVSTPRSKFATMEFQTQVLMSLLLCMSGAAADVVMTQTPLTLSVTIGQ
PASISCKSSQSLLDSDGKTYLNWLLQRPGQSPKRLIYLVSKLDSGVPDRFTGSGSGT
DFTLKISRVEAEDLGVYYCWQGTHFPQTFGGGTKLEIKRARADAAPTVSIFPPSSEQ
LTSGGASVVCFLNNFYPKDINVKWKIDGSERQNGVLNSWTDQDSKDSTYSMSSTLT
LTKDEYERHNSYTCEATHKTSTSPIVKSFNRNEC
Exemplary anti-HER2 scFv
(SEQ ID NO: 27)
EVQLVESGGGLVQPGGSLRLSCAASGFNIKDTYIHWVRQAPGKGLEWVARIYPTN
GYTRYADSVKGRFTISADTSKNTAYLQMNSLRAEDTAVYYCSRWGGDGFYAMDY
WGQGTLVTVSSASTKGPSVFPLAPSSKSTSGGTAALGCLVKDYFPEPVTVSWNSGAL
TSGVHTFPAVLQSSGLYSLSSVVTVPSSSLGTQTYICNVNHKPSNTKVDKKVEP
Exemplary anti-FAP scFv
Heavy chain
(SEQ ID NO: 28)
QVQLQQSGAELARPGASVNLSCKASGYTFTNNGINWLKQRTGQGLEWIGEIYPRST
NTLYNEKFKGKATLTADRSSNTAYMELRSLTSEDSAVYFCARTLTAPFAFWGQGT
LVTVSA
Light chain
(SEQ ID NO: 29)
QIVLTQSPAIMSASPGEKVTMTCSASSGVNFMHWYQQKSGTSPKRWIFDTSKLASG
VPARFSGSGSGTSYSLTISSMEAEDAATYYCQQWSFNPPTFGGGTKLEIKR
Exemplary anti-CS1 scFv
Heavy chain
(SEQ ID NO: 30)
MGWSSIILFLVATATGVHSQVQLQQPGAELVRPGASVKLSCKASGYSFTTYWMNW
VKQRPGQGLEWIGMIHPSDSETRLNQKFKDKATLTVDKSSSTA
Light chain
(SEQ ID NO: 31)
DIVMTQSQKSMSTSVGDRVSITCKASQDVITGVAWYQQKPGQSPKLLIYSASYRYT
GVPDRFTGSGSGTDFTFTISNVQAEDLAVYYCQQHYSTPLTFGAGTKLELK

### Exemplary Transmembrane Domain Sequences

Exemplary CD28 transmembrane domain
(SEQ ID NO: 9)
FWVLVVVGGVLACYSLLVTVAFIIFWV
Exemplary CD8 transmembrane domain
(SEQ ID NO: 32)
IYIWAPLAGTCGVLLLSLVITLYC
Exemplary TLR3 transmembrane domain
(SEQ ID NO: 72)
FFMINTSILLIFIFIVLLIHF
Exemplary TLR4 transmembrane domain
(SEQ ID NO: 73)
IGVSVLSVLVVSVVAVLVY
Exemplary IFNGR1 transmembrane domain
(SEQ ID NO: 96)
SLWIPVVAALLLFLVLSLVFI
Exemplary IL10RA transmembrane domain
(SEQ ID NO: 97)
VIIFF AFVLLLSGALAYCLAL
Exemplary TLR9 transmembrane domain
(SEQ ID NO: 98)
FALSLLAVALGLGVPMLHHLC
Exemplary TNFR2 transmembrane domain
(SEQ ID NO: 99)
FALPVGLIVGVTALGLLIIGVVNCVIMTQV

### Exemplary Signaling Domain Sequences

Exemplary 4-1BB signaling domain
(SEQ ID NO: 89)
KRGRKKLLYIFKQPFMRPVQTTQEEDGCSCRFPEEEEGGCEL
Exemplary TLR3 signaling domain
(SEQ ID NO: 74)
EGWRISFYWNVSVHRVLGFKEIDRQTEQFEYAAYIIHAYKDKDWVWEHFSSMEKED
QSLKFCLEERDFEAGVFELEAIVNSIKRSRKIIFVITHHLLKDPLCKRFKVHHAVQQAI
EQNLDSIILVFLEEIPDYKLNHALCLRRGMFKSHCILNWPVQKERIGAFRHKLQVALG
SKNSVH
Exemplary TIR3 signaling domain
(SEQ ID NO: 75)
WRISFYWNVSVHRVLGFKEIDRQTEQFEYAAYIIHAYKDKDWVWEHFSSMEKEDQS
LKFCLEERDFEAGVFELEAIVNSIKRSRKIIFVITHHLLKDPLCKRFKVHHAVQQAIEQ
NLDSIILVFLEEIPDYKLNHALCLRRGMFKSHCILNWPVQKERIGAFRHKLQVALGSK
NSVHRVKFSRSADAPAYKQGQNQLYNELNLGRREEYDVLDKRRGRDPEMGGKPRR
KNPQEGLYNELQKDKMAEAYSEIGMKGERRRGKGHDGLYQGLSTATKDTYDALH
MQALPPR
Exemplary TLR4 signaling domain
(SEQ ID NO: 76)
KFYFHLMLLAGCIKYGRGE
Exemplary TIR4 signaling domain
(SEQ ID NO: 77)
NIYDAFVIYSSQDEDWVRNELVKNLEEGVPPFQLCLHYRDFIPGVAIAANIIHEGFHK
SRKVIVVVSQHFIQSRWCIFEYEIAQTWQFLSSRAGIIFIVLQKVEKTLLRQQVELYRL
LSRNTYLEWEDSVLGRHIFWRRLRKALLDGKSWNPEGTVGTGCNWQEATSI
Exemplary TLR4/TIR4 signaling domain
(SEQ ID NO: 100)
KFYFHLMLLAGCIKYGRGENIYDAFVIYSSQDEDWVRNELVKNLEEGVPPFQLCLHY
RDFIPGVAIAANIIHEGFHKSRKVIVVVSQHFIQSRWCIFEYEIAQTWQFLSSRAGIIFIV
LQKVEKTLLRQQVELYRLLSRNTYLEWEDSVLGRHIFWRRLRKALLDGKSWNPEGT
VGTGCNWQEATSI
Exemplary IFNGR1 signaling domain
(SEQ ID NO: 101)
CFYIKKINPLKEKSIILPKSLISVVRSATLETKPESKYVSLITSYQPFSLEKEVVCEEPLS
PATVPGMHTEDNPGKVEHTEELSSITEVVTTEENIPDVVPGSHLTPIERESSSPLSSNQ
SEPGSIALNSYHSRNCSESDHSRNGFDTDSSCLESHSSLSDSEFPPNNKGEIKTEGQELI
TVIKAPTSFGYDKPHVLVDLLVDDSGKESLIGYRPTEDSKEFS
Exemplary IFNγ signaling domain
(SEQ ID NO: 78)
MKYTSYILAFQLCIVLGSLGCYCQDPYVKEAENLKKYFNAGHSDVADNGTLFLGILK
NWKEESDRKIMQSQIVSFYFKLFKNFKDDQSIQKSVETIKEDMNVKFFNSNKKKRDD
FEKLTNYSVTDLNVQRKAIHELIQVMAELSPAAKTGKRKRSQMLFRG
Exemplary IL10RA signaling domain
(SEQ ID NO: 102)
QLYVRRRKKLPSVLLFKKPSPFIFISQRPSPETQDTIHPLDEEAFLKVSPELKNLDLHGS
TDSGFGSTKPSLQTEEPQFLLPDPHPQADRTLGNREPPVLGDSCSSGSSNSTDSGICLQ
EPSLSPSTGPTWEQQVGSNSRGQDDSGIDLVQNSEGRAGDTQGGSALGHHSPPEPEV
PGEEDPAAVAFQGYLRQTRCAEEKATKTGCLEEESPLTDGLGPKFGRCLVDEAGLH
PPALAKGYLKQDPLEMTLASSGAPTGQWNQPTEEWSLLALSSCSDLGISDWSFAHD
LAPLGCVAAPGGLLGSFNSDLVTLPLISSLQSSE
Exemplary TLR9/TIR9 signaling domain
(SEQ ID NO: 103)
GWDLWYCFHLCLAWLPWRGRQSGRDEDALPYDAFVVFDKTQSAVADWVYNELR
GQLEECRGRWALRLCLEERDWLPGKTLFENLWASVYGSRKTLFVLAHTDRVSGLLR
ASFLLAQQRLLEDRKDVVVLVILSPDGRRSRYVRLRQRLCRQSVLLWPHQPSGQRSF
WAQLGMALTRDNHHFYNRNFCQGPTAE
Exemplary TNFR2 signaling domain
(SEQ ID NO: 104)
KKKPLCLQREAKVPHLPADKARGTQGPEQQHLLITAPSSSSSSLESSASALDRRAPTR
NQPQAPGVEASGAGEARASTGSSDSSPGGHGTQVNVTCIVNVCSSSDHSSQCSSQAS
STMGDTDSSPSESPKDEQVPFSKEECAFRSQLETPETLLGSTEEKPLPLGVPDAGMKP
S
Exemplary CD28 signaling domain
(SEQ ID NO: 10)
RSKRSRLLHSDYMNMTPRRPGPTRKHYQPYAPPRDFAAYRS
Exemplary CD35 signaling domain
(SEQ ID NO: 11)
RVKFSRSADAPAYKQGQNQLYNELNLGRREEYDVLDKRRGRDPEMGGKPRRKNPQ
EGLYNELQKDKMAEAYSEIGMKGERRRGKGHDGLYQGLSTATKDTYDALHMQAL
PPR
Exemplary BDNF signaling domain
(SEQ ID NO: 33)
KLARHSKFGMKGPASVISNDDDSASPLHHISNGSNTPSSSEGGPDAVIIGMTKIPVIEN
PQYFGITNSQLKPDTFVQHIKRHNIVLKRELGEGAFGKVFLAECYNLCPEQDKILVAV
KTLKDASDNARKDFHREAELLTNLQHEHIVKFYGVCVEGDPLIMVFEYMKHGDLN
KFLRAHGPDAVLMAEGNPPTELTQSQMLHIAQQIAAGMVYLASQHFVHRDLATRN
CLVGENLLVKIGDFGMSRDVYSTDYYRVGGHTMLPIRWMPPESIMYRKFTTESDVW
SLGVVLWEIFTYGKQPWYQLSNNEVIECITQGRVLQRPRTCPQEVYELMLGCWQRE
PHMRKNIKGIHTLLQNLAKASPVYLDILG
Exemplary VEGFR2 signaling domain
(SEQ ID NO: 79)
LKLGKPLGRGAFGQVIEADAFGIDKTATCRTVAVKMLKEGATHSEHRALMSELKILI
HIGHHLNVVNLLGACTKPGGPLMVIVEFCKFGNLSTYLRSKRNEFVPYKTKGARFR
QGKDYVGAIPVDLKRRLDSITSSQSSASSGFVEEKSLSDVEEEEAPEDLYKDFLTLEH
LICYSFQVAKGMEFLASRKCIHRDLAARNILLSEKNVVKICDFGLARDIYKDPDYVR
KGDARLPLKWMAPETIFDRVYTIQSDVWSFGVLLWEIFSLGASPYPGVKIDEEFCRR
LKEGTRMRAPDYTTPEMYQTMLDCWHGEPSQRPTFSELVEHLGN

### Exemplary Hinge Domain Sequences

Exemplary CD8 hinge domain
(SEQ ID NO: 34)
TTTPAPRPPTPAPTIASQPLSLRPEACRPAAGGAVHTRGLDFACD
Exemplary CD28 hinge domain
(SEQ ID NO: 35)
LEPKSCDKTHTCPPCPDPK

### Exemplary Leader Sequences

Exemplary leader polypeptide
(SEQ ID NO: 36)
MALPVTALLLPLALLLHAARP

### Exemplary Linker Sequences

Exemplary linker polypeptide
(SEQ ID NO: 37)
EGKSSGSGSESKAS

### Exemplary Polypeptide Sequences

Exemplary NFκB1 polypeptide
(SEQ ID NO: 12)
MAEDDPYLGRPEQMFHLDPSLTHTIFNPEVFQPQMALPTADGPYLQILEQPKQRGFR
FRYVCEGPSHGGLPGASSEKNKKSYPQVKICNYVGPAKVIVQLVINGKNIHLHAHSL
VGKHCEDGICTVTAGPKDMVVGFANLGILHVTKKKVFETLEARMTEACIRGYNPGL
LVHPDLAYLQAEGGGDRQLGDREKELIRQAALQQTKEMDLSVVRLMFTAFLPDSTG
SFTRRLEPVVSDAIYDSKAPNASNLKIVRMDRTAGCVTGGEEIYLLCDKVQKDDIQIR
FYEEEENGGVWEGFGDFSPTDVHRQFAIVFKTPKYKDINITKPASVFVQLRRKSDLET
SEPKPFLYYPEIKDKEEVQRKRQKLMPNFSDSFGGGSGAGAGGGGMFGSGGGGGGT
GSTGPGYSFPHYGFPTYGGITFHPGTTKSNAGMKHGTMDTESKKDPEGCDKSDDKN
TVNLFGKVIETTEQDQEPSEATVGNGEVTLTYATGTKEESAGVQDNLFLEKAMQLA
KRHANALFDYAVTGDVKMLLAVQRHLTAVQDENGDSVLHLAIIHLHSQLVRDLLE
VTSGLISDDIINMRNDLYQTPLHLAVITKQEDVVEDLLRAGADLSLLDRLGNSVLHL
AAKEGHDKVLSILLKHKKAALLLDHPNGDGLNAIHLAMMSNSLPCLLLLVAAGAD
VNAQEQKSGRTALHLAVEHDNISLAGCLLLEGDAHVDSTTYDGTTPLHIAAGRGST
RLAALLKAAGADPLVENFEPLYDLDDSWENAGEDEGVVPGTTPLDMATSWQVFDIL
NGKPYEPEFTSDDLLAQGDMKQLAEDVKLQLYKLLEIPDPDKNWATLAQKLGLGIL
NNAFRLSPAPSKTLMDNYEVSGGTVRELVEALRQMGYTEAIEVIQAASSPVKTTSQA
HSLPLSPASTRQQIDELRDSDSVCDSGVETSFRKLSFTESLTSGASLLTLNKMPHDYG
QEGPLEGKI
Exemplary JUN polypeptide
(SEQ ID NO: 13)
MAEDDPYLGRPEQMFHLDPSLTHTIFNPEVFQPQMALPTADGPYLQILEQPKQRGFR
FRYVCEGPSHGGLPGASSEKNKKSYPQVKICNYVGPAKVIVQLVINGKNIHLHAHSL
VGKHCEDGICTVTAGPKDMVVGFANLGILHVTKKKVFETLEARMTEACIRGYNPGL
LVHPDLAYLQAEGGGDRQLGDREKELIRQAALQQTKEMDLSVVRLMFTAFLPDSTG
SFTRRLEPVVSDAIYDSKAPNASNLKIVRMDRTAGCVTGGEEIYLLCDKVQKDDIQIR
FYEEEENGGVWEGFGDFSPTDVHRQFAIVFKTPKYKDINITKPASVFVQLRRKSDLET
SEPKPFLYYPEIKDKEEVQRKRQKLMPNFSDSFGGGSGAGAGGGGMFGSGGGGGGT
GSTGPGYSFPHYGFPTYGGITFHPGTTKSNAGMKHGTMDTESKKDPEGCDKSDDKN
TVNLFGKVIETTEQDQEPSEATVGNGEVTLTYATGTKEESAGVQDNLFLEKAMQLA
KRHANALFDYAVTGDVKMLLAVQRHLTAVQDENGDSVLHLAIIHLHSQLVRDLLE
VTSGLISDDIINMRNDLYQTPLHLAVITKQEDVVEDLLRAGADLSLLDRLGNSVLHL
AAKEGHDKVLSILLKHKKAALLLDHPNGDGLNAIHLAMMSNSLPCLLLLVAAGAD
VNAQEQKSGRTALHLAVEHDNISLAGCLLLEGDAHVDSTTYDGTTPLHIAAGRGST
RLAALLKAAGADPLVENFEPLYDLDDSWENAGEDEGVVPGTTPLDMATSWQVFDIL
NGKPYEPEFTSDDLLAQGDMKQLAEDVKLQLYKLLEIPDPDKNWATLAQKLGLGIL
NNAFRLSPAPSKTLMDNYEVSGGTVRELVEALRQMGYTEAIEVIQAASSPVKTTSQA
HSLPLSPASTRQQIDELRDSDSVCDSGVETSFRKLSFTESLTSGASLLTLNKMPHDYG
QEGPLEGKI
Exemplary RELB polypeptide
(SEQ ID NO: 14)
MLRSGPASGPSVPTGRAMPSRRVARPPAAPELGALGSPDLSSLSLAVSRSTDELEIIDE
YIKENGFGLDGGQPGPGEGLPRLVSRGAASLSTVTLGPVAPPATPPPWGCPLGRLVS
PAPGPGPQPHLVITEQPKQRGMRFRYECEGRSAGSILGESSTEASKTLPAIELRDCGG
LREVEVTACLVWKDWPHRVHPHSLVGKDCTDGICRVRLRPHVSPRHSFNNLGIQCV
RKKEIEAAIERKIQLGIDPYNAGSLKNHQEVDMNVVRICFQASYRDQQGQMRRMDP
VLSEPVYDKKSTNTSELRICRINKESGPCTGGEELYLLCDKVQKEDISVVFSRASWEG
RADFSQADVHRQIAIVFKTPPYEDLEIVEPVTVNVFLQRLTDGVCSEPLPFTYLPRDH
DSYGVDKKRKRGMPDVLGELNSSDPHGIESKRRKKKPAILDHFLPNHGSGPFLPPSA
LLPDPDFFSGTVSLPLEPPGGPDLLDDGFAYDPTAPTLFTMLDLLPPAPPHASAVVC
SGGAGAVVGETPGPEPLTLDSYQAPGPGDGGTASLVGSNMFPNHYREAAFGGGLLS
PGPEAT
Exemplary IRF1 polypeptide
(SEQ ID NO: 15)
YPYDVPDYAGTELGSTMASWSHPQFEKGGGSGGGSGGGSWSHPQFEKAADITSLYK
KAGSTMPITRMRMRPWLEMQINSNQIPGLIWINKEEMIFQIPWKHAAKHGWDINKD
ACLFRSWAIHTGRYKAGEKEPDPKTWKANFRCAMNSLPDIEEVKDQSRNKGSSAVR
VYRMLPPLTKNQRKERKSKSSRDAKSKAKRKSCGDSSPDTFSDGLSSSTLPDDHSSY
TVPGYMQDLEVEQALTPALSPCAVSSTLPDWHIPVEVVPDSTSDLYNFQVSPMPSTS
EATTDEDEEGKLPEDIMKLLEQSEWQPTNVDGKGYLLNEPGVQPTSVYGDFSCKEEP
EIDSPGGDIGLSLQRVFTDLKNMDATWLDSLLTPVRLPSIQAIPCAPLDPAFLYKVVT
Exemplary TNFα polypeptide
(SEQ ID NO: 16)
MSTESMIRDVELAEEALPKKTGGPQGSRRCLFLSLFSFLIVAGATTLFCLLHFGVIGPQ
REEFPRDLSLISPLAQAVRSSSRTPSDKPVAHVVANPQAEGQLQWLNRRANALLANG
VELRDNQLVVPSEGLYLIYSQVLFKGQGCPSTHVLLTHTISRIAVSYQTKVNLLSAIK
SPCQRETPEGAEAKPWYEPIYLGGVFQLEKGDRL
Exemplary IL-10 polypeptide
(SEQ ID NO: 17)
MHSSALLCCLVLLTGVRASPGQGTQSENSCTHFPGNLPNMLRDLRDAFSRVKTFFQ
MKDQLDNLLLKESLLEDFKGYLGCQALSEMIQFYLEEVMPQAENQDPDIKAHVNSL
GENLKTLRLRLRRCHRFLPCENKSKAVEQVKNAFNKLQEKGIYKAMSEFDIFINYIE
AYMTMKIRN
Exemplary FGF-2 polypeptide
(SEQ ID NO: 18)
GGWLYHHSAGPAGRWRQRRVPAGPF*GSEAPVLQKRRLFPAHSPGWPCGRCA*EK
RPAHQTATASGGTWCSEHQGCMR*PLSGDERRRPPAGEQMRHRRMFLLRTPGVQQ
LQHVSLPQIHLLVRGSETHRPVQTGLQNRPWPESDPVPADVREIL
Exemplary G-CSF polypeptide
(SEQ ID NO: 19)
MTPLGPASSLPQSFLLKCLEQVRKIQGDGAALQEKLCATYKLCHPEELVLLGHSLGIP
WAPLSSCPSQALQLAGCLSQLHSGLFLYQGLLQALEGISPELGPTLDTLQLDVADFA
TTIWQQMEELGMAPALQPTQGAMPAFASAFQRRAGGVLVASHLQSFLEVSYRVLR
HLAQP
Exemplary GM-CSF polypeptide
(SEQ ID NO: 20)
MWLQSLLLLGTVACSISAPARSPSPSTQPWEHVNAIQEARRLLNLSRDTAAEMNETV
EVISEMFDLQEPTCLQTRLELYKQGLRGSLTKLKGPLTMMASHYKQHCPPTPETSCA
TQIITFESFKENLKDFLLVIPFDCWEPVQELPTFLYKVVGSTSGSGKPGSGEGSTKG
Exemplary Eotaxin polypeptide
(SEQ ID NO: 21)
MKVSAALLWLLLIAAAFSPQGLAGPASVPTTCCFNLANRKIPLQRLESYRRITSGKCP
QKAVIFKTKLAKDICADPKKKWVQDSMKYLDQKSPTPKP
Exemplary Gal-9 polypeptide
(SEQ ID NO: 22)
AFSGSQAPYLSPAVPFSGTIQGGLQDGLQITVNGTVLSSSGTRFAVNFQTGFSGNDIA
FHFNPRFEDGGYVVCNTRQNGSWGPEERKTHMPFQKGMPFDLCFLVQSSDFKVMV
NGILFVQYFHRVPFHRVDTISVNGSVQLSYISFQPPGVWPANPAPITQTVIHTVQSAPG
QMFSTPAIPPMMYPHPAYPMPFITTILGGLYPSKSILLSGTVLPSAQRFHINLCSGNHIA
FHLNPRFDENAVVRNTQIDNSWGSEERSLPRKMPFVRGQSFSVWILCEAHCLKVAV
DGQHLFEYYHRLRNLPTINRLEVGGDIQLTHVQT
Exemplary PD1 polypeptide
(SEQ ID NO: 23)
MQIPQAPWPVVWAVLQLGWRPGWFLDSPDRPWNPPTFSPALLVVTEGDNATFTCSF
SNTSESFVLNWYRMSPSNQTDKLAAFPEDRSQPGQDCRFRVTQLPNGRDFHMSVVR
ARRNDSGTYLCGAISLAPKAQIKESLRAELRVTERRAEVPTAHPSPSPRPAGQFQTLV
VGVVGGLLGSLVLLVWVLAVICSRAARGTIGARRTGQPLKEDPSAVPVFSIVYASLD
FQWREKTPEPPVPCVPEQTEYATIVFPSGMGTSSPARRGSADGPRSAQPLRPEDGHCS
WPLTRLQDIKLAVPRARDPPVAT
Exemplary TIM-3 polypeptide
(SEQ ID NO: 24)
MFSHLPFDCVLLLLLLLLTRSSEVEYRAEVGQNAYLPCFYTPAAPGNLVPVCWGKG
ACPVFECGNVVLRTDERDVNYWTSRYWLNGDFRKGDVSLTIENVTLADSGIYCCRI
QIPGIMNDEKFNLKLVIKPAKVTPAPTLQRDFTAAFPRMLTTRGHGPAETQTLGSLPD
INLTQISTLANELRDSRLANDLRDSGATIRIGIYIGAGICAGLALALIFGALIFKWYSHS
KEKIQNLSLISLANLPPSGLANAVAEGIRSEENIYTIEENVYEVEEPNEYYCYVSSRQQ
PSQPLGCRFAMP
Exemplary CXCR3 polypeptide
(SEQ ID NO: 25)
MVLEVSDHQVLNDAEVAALLENFSSSYDYGENESDSCCTSPPCPQDFSLNFDRAFLP
ALYSLLFLLGLLGNGAVAAVLLSRRTALSSTDTFLLHLAVADTLLVLTLPLWAVDA
AVQWVFGSGLCKVAGALFNINFYAGALLLACISFDRYLNIVHATQLYRRGPPARVTL
TCLAVWGLCLLFALPDFIFLSAHHDERLNATHCQYNFPQVGRTALRVLQLVAGFLLP
LLVMAYCYAHILAVLLVSRGQRRLRAMRLVVVVVVAFALCWTPYHLVVLVDILMD
LGALARNCGRESRVDVAKSVTSGLGYMHCCLNPLLYAFVGVKFRERMWMLLLRL
GCPNQRGLQRQPSSSRRDSSWSETSEASYSGL
Exemplary CXCR4 polypeptide
(SEQ ID NO: 26)
MEGISIYTSDNYTEEMGSGDYDSMKEPCFREENANFNKIFLPTIYSIIFLTGIVGNGLVI
LVMGYQKKLRSMTDKYRLHLSVADLLFVITLPFWAVDAVANWYFGNFLCKAVHVI
YTVNLYSSVLILAFISLDRYLAIVHATNSQRPRKLLAEKVVYVGVWIPALLLTIPDFIF
ANVSEADDRYICDRFYPNDLWVVVFQFQHIMVGLILPGIVILSCYCIIISKLSHSKGHQ
KRKALKTTVILILAFFACWLPYYIGISIDSFILLEIIKQGCEFENTVHKWISITEALAFFH
CCLNPILYAFLGAKFKTSAQHALTSVSRGSSLKILSKGKRGGHSSVSTESESSSFHSS
Exemplary CTLA4 polypeptide
(SEQ ID NO: 91)
MACLGFQRHKAQLNLATRTWPCTLLFFLLFIPVFCKAMHVAQPAVVLASSRGIASFV
CEYASPGKATEVRVTVLRQADSQVTEVCAATYMMGNELTFLDDSICTGTSSGNQVN
LTIQGLRAMDTGLYICKVELMYPPPYYLGIGNGTQIYVIDPEPCPDSDELLWILAAVS
SGLFFYSFLLTAVSLSKMLKKRSPLTTGVYVKMPPTEPECEKQFQPYFIPIN
Exemplary TLR3 polypeptide
(SEQ ID NO: 92)
MRQTLPCIYFWGGLLPFGMLCASSTTKCTVSHEVADCSHLKLTQVPDDLPTNITVLN
LTHNQLRRLPAANFTRYSQLTSLDVGFNTISKLEPELCQKLPMLKVLNLQHNELSQL
SDKTFAFCTNLTELHLMSNSIQKIKNNPFVKQKNLITLDLSHNGLSSTKLGTQVQLEN
LQELLLSNNKIQALKSEELDIFANSSLKKLELSSNQIKEFSPGCFHAIGRLFGLFLNNV
QLGPSLTEKLCLELANTSIRNLSLSNSQLSTTSNTTFLGLKWTNLTMLDLSYNNLNVV
GNDSFAWLPQLEYFFLEYNNIQHLFSHSLHGLFNVRYLNLKRSFTKQSISLASLPKID
DFSFQWLKCLEHLNMEDNDIPGIKSNMFTGLINLKYLSLSNSFTSLRTLTNETFVSLA
HSPLHILNLTKNKISKIESDAFSWLGHLEVLDLGLNEIGQELTGQEWRGLENIFEIYLS
YNKYLQLTRNSFALVPSLQRLMLRRVALKNVDSSPSPFQPLRNLTILDLSNNNIANIN
DDMLEGLEKLEILDLQHNNLARLWKHANPGGPIYFLKGLSHLHILNLESNGFDEIPVE
VFKDLFELKIIDLGLNNLNTLPASVFNNQVSLKSLNLQKNLITSVEKKVFGPAFRNLT
ELDMRFNPFDCTCESIAWFVNWINETHTNIPELSSHYLCNTPPHYHGFPVRLFDTSSC
KDSAPFELFFMINTSILLIFIFIVLLIHFEGWRISFYWNVSVHRVLGFKEIDRQTEQFEY
AAYIIHAYKDKDWVWEHFSSMEKEDQSLKFCLEERDFEAGVFELEAIVNSIKRSRKII
FVITHHLLKDPLCKRFKVHHAVQQAIEQNLDSIILVFLEEIPDYKLNHALCLRRGMFK
SHCILNWPVQKERIGAFRHKLQVALGSKNSVH
Exemplary TLR4 polypeptide
(SEQ ID NO: 93)
MMSASRLAGTLIPAMAFLSCVRPESWEPCVEVVPNITYQCMELNFYKIPDNLPFSTK
NLDLSFNPLRHLGSYSFFSFPELQVLDLSRCEIQTIEDGAYQSLSHLSTLILTGNPIQSL
ALGAFSGLSSLQKLVAVETNLASLENFPIGHLKTLKELNVAHNLIQSFKLPEYFSNLT
NLEHLDLSSNKIQSIYCTDLRVLHQMPLLNLSLDLSLNPMNFIQPGAFKEIRLHKLTL
RNNFDSLNVMKTCIQGLAGLEVHRLVLGEFRNEGNLEKFDKSALEGLCNLTIEEFRL
AYLDYYLDDIIDLFNCLTNVSSFSLVSVTIERVKDFSYNFGWQHLELVNCKFGQFPTL
KLKSLKRLTFTSNKGGNAFSEVDLPSLEFLDLSRNGLSFKGCCSQSDFGTTSLKYLDL
SFNGVITMSSNFLGLEQLEHLDFQHSNLKQMSEFSVFLSLRNLIYLDISHTHTRVAFN
GIFNGLSSLEVLKMAGNSFQENFLPDIFTELRNLTFLDLSQCQLEQLSPTAFNSLSSLQ
VLNMSHNNFFSLDTFPYKCLNSLQVLDYSLNHIMTSKKQELQHFPSSLAFLNLTQND
FACTCEHQSFLQWIKDQRQLLVEVERMECATPSDKQGMPVLSLNITCQMNKTIIGVS
VLSVLVVSVVAVLVYKFYFHLMLLAGCIKYGRGENIYDAFVIYSSQDEDWVRNELV
KNLEEGVPPFQLCLHYRDFIPGVAIAANIIHEGFHKSRKVIVVVSQHFIQSRWCIFEYEI
AQTWQFLSSRAGIIFIVLQKVEKTLLRQQVELYRLLSRNTYLEWEDSVLGRHIFWRR
LRKALLDGKSWNPEGTVGTGCNWQEATSI
Exemplary TLR9 polypeptide
(SEQ ID NO: 94)
MGFCRSALHPLSLLVQAIMLAMTLALGTLPAFLPCELQPHGLVNCNWLFLKSVPHFS
MAAPRGNVTSLSLSSNRIHHLHDSDFAHLPSLRHLNLKWNCPPVGLSPMHFPCHMTI
EPSTFLAVPTLEELNLSYNNIMTVPALPKSLISLSLSHTNILMLDSASLAGLHALRFLF
MDGNCYYKNPCRQALEVAPGALLGLGNLTHLSLKYNNLTVVPRNLPSSLEYLLLSY
NRIVKLAPEDLANLTALRVLDVGGNCRRCDHAPNPCMECPRHFPQLHPDTFSHLSRL
EGLVLKDSSLSWLNASWFRGLGNLRVLDLSENFLYKCITKTKAFQGLTQLRKLNLSF
NYQKRVSFAHLSLAPSFGSLVALKELDMHGIFFRSLDETTLRPLARLPMLQTLRLQM
NFINQAQLGIFRAFPGLRYVDLSDNRISGASELTATMGEADGGEKVWLQPGDLAPAP
VDTPSSEDFRPNCSTLNFTLDLSRNNLVTVQPEMFAQLSHLQCLRLSHNCISQAVNGS
QFLPLTGLQVLDLSHNKLDLYHEHSFTELPRLEALDLSYNSQPFGMQGVGHNFSFVA
HLRTLRHLSLAHNNIHSQVSQQLCSTSLRALDFSGNALGHMWAEGDLYLHFFQGLS
GLIWLDLSQNRLHTLLPQTLRNLPKSLQVLRLRDNYLAFFKWWSLHFLPKLEVLDLA
GNQLKALTNGSLPAGTRLRRLDVSCNSISFVAPGFFSKAKELRELNLSANALKTVDH
SWFGPLASALQILDVSANPLHCACGAAFMDFLLEVQAAVPGLPSRVKCGSPGQLQG
LSIFAQDLRLCLDEALSWDCFALSLLAVALGLGVPMLHHLCGWDLWYCFHLCLAW
LPWRGRQSGRDEDALPYDAFVVFDKTQSAVADWVYNELRGQLEECRGRWALRLC
LEERDWLPGKTLFENLWASVYGSRKTLFVLAHTDRVSGLLRASFLLAQQRLLEDRK
DVVVLVILSPDGRRSRYVRLRQRLCRQSVLLWPHQPSGQRSFWAQLGMALTRDNH
HFYNRNFCQGPTAE
Exemplary TNFR2 polypeptide
(SEQ ID NO: 95)
MAPVAVWAALAVGLELWAAAHALPAQVAFTPYAPEPGSTCRLREYYDQTAQMCC
SKCSPGQHAKVFCTKTSDTVCDSCEDSTYTQLWNWVPECLSCGSRCSSDQVETQAC
TREQNRICTCRPGWYCALSKQEGCRLCAPLRKCRPGFGVARPGTETSDVVCKPCAP
GTFSNTTSSTDICRPHQICNVVAIPGNASMDAVCTSTSPTRSMAPGAVHLPQPVSTRS
QHTQPTPEPSTAPSTSFLLPMGPSPPAEGSTGDFALPVGLIVGVTALGLLIIGVVNCVI
MTQVKKKPLCLQREAKVPHLPADKARGTQGPEQQHLLITAPSSSSSSLESSASALDR
RAPTRNQPQAPGVEASGAGEARASTGSSDSSPGGHGTQVNVTCIVNVCSSSDHSSQC
SSQASSTMGDTDSSPSESPKDEQVPFSKEECAFRSQLETPETLLGSTEEKPLPLGVPDA
GMKPS

### Example 3: Exemplary Nucleotide Sequences

Nucleic acids encoding exemplary scFvs
nucleic acid encoding an exemplary anti-Ecad scFv (also referred to as
anti-hmcECAD.6)
Heavy chain
(SEQ ID NO: 38)
GAGGTGCAGCTGGTGCAGTCTGGGGGAGGCTTGGTACAGCCTGGGGGGTCCCTG
AGACTCTCCTGTGCAGCCTCTGGATTCACCTTCAGTAGCTATAGCATGAACTGGG
TCCGCCAGGCTCCAGGGAAGGGGCTGGAGTGGGTTTCATACATTAGTAGTAGTA
GTAGTACCATATACTACGTAGACTCTGTGAAGGGCCGATTCACCATCTCCAGAGA
CAACGCCAAGAACTCACTGTATCTGCAAATGGACAGCCTGAGAGCCGAGGACAC
GGCTGTGTATTACTGTGCGAGAGGAGGTCGGGTGTTAGTGGGAGCTCTATTTGAC
TACTGGGGCCAGGGAACCCTGGTCACCGTCTCCTCA
Light chain
(SEQ ID NO: 39)
CTGCCTGTGCTGACTCAGCCACCCTCAGCGTCTGGGACCCCCGGGCAGAGGGTC
ACCATCTCTTGTTCTGGAAGCAGCTCCAACATCGGAAGTAATTATGTCTACTGGT
ACCAGCAACTCCCAGGAACGGCCCCCAAACTCCTCATCTATAGGAATAATCAGC
GGCCCTCAGGGGTCCCTGACCGATTCTCTGGCTCCAAGTCTGGCACCTCAGCCTC
CCTGGCCATCAGTGGGCTCCAGTCTGAGGATGAGGCTGATTATTATTGTGCATCA
TGGGATACCAGCCTGCGTGCCTGGGTGTTCGGCGGAGGGACCAAGCTGACCGTC
CTAGGT
nucleic acid encoding an exemplary anti-CD103 scFv
Heavy chain
(SEQ ID NO: 40)
CAGGTGCAGCTGCAGGAGTCGGGCCCAGGACTGGTGAAGCCTTCGGAGACCCTG
TCCCTCACCTGCACTGTCTCTGGTGGCTCCGTCAGTAGTTACTATTGGAGCTGGA
TCCGGCAGCCCCCAGGGAAGGGACTGGAGTGGATTGGCCATATCTATTACAGTG
GGAATACCAACTACAACCCCTCCCTCAAGAGTCGAGTCACCATATCAGTAGACA
CGTCCAAGAATCAGTTCTCCCTGAAACTGAGCTCTGTGACCGCTGCGGACACGG
CCGTGTATTTTTGTGCGAGAGATAGATGGAATTATTATGATAGTAGTCCCGGCTA
TTATTATTACTACGGTATGGACGTCTGGGGCCAAGGGACCACGGTCACCGTCAGC
TCA
Light chain
(SEQ ID NO: 41)
GACATCCAGATGACCCAGTCTCCATCCTCCCTGTCTGCATCTGTAGGAGACAGAG
TCACCATCACTTGCCGGGCGAGTCAGGGCATTAGAAATGATTTAGGCTGGTATCA
GCAAAAACCAGGGAAAGCCCCTAAGCGCCTAATCTTTGCTGCATCCCATTTGCA
AAGTGGAGTCCCTTCAAGGTTCAGCGGCAGTGGATCTGGGACAGAGTTCACTCT
CACAATCAGCAGCCTGCAGCCTGAAGATTTTGCAACTTATTACTGTCAACAGCAT
AATAGTTCCCCATTCACTTTCGGCCCTGGGACCAGAGTGGATATCAAA
nucleic acid encoding an exemplary anti-MOG scFv
Heavy chain
(SEQ ID NO: 42)
GAGGTGAAGCTGCACGAGAGCGGCGCAGGTCTGGTGAAGCCCGGCGCCAGCGT
GGAGATCAGCTGCAAGGCCACCGGCTACACCTTCAGCAGCTTCTGGATCGAGTG
GGTGAAGCAGAGACCCGGCCACGGCCTGGAGTGGATCGGCGAGATCCTGCCCGG
CAGAGGCAGAACCAACTACAACGAGAAGTTCAAGGGCAAGGCCACCTTCACCG
CCGAGACCAGCAGCAACACCGCCTACATGCAGCTGAGCAGCCTGACCAGCGAGG
ACAGCGCCGTGTACTACTGCGCCACCGGCAACACCATGGTGAACATGCCCTACT
GGGGCCAGGGCACCACCGTGACCGTGAGCAGCGGTGGAGGTGGTTCGGGAGGT
GGAGGTAGCGGAGGTGGTGGAAGC
Light chain
(SEQ ID NO: 43)
GATATTGAACTGACCCAGAGTCCCAGTAGCCTGGCCGTGAGTGCCGGCGAGAAA
GTGACCATGAGCTGCAAAAGCAGCCAGAGCCTGCTGAACAGCGGCAACCAGAA
AAACTACCTGGCCTGGTACCAGCAGAAACCCGGCCTGCCTCCTAAGCTGCTGAT
CTACGGCGCCAGCACCAGAGAAAGCGGCGTGCCCGATAGATTCACCGGCTCTGG
CTCTGGCACCGACTTCACCCTGACCATCAGCAGCGTGCAGGCCGAAGACCTGGC
TGTCTACTACTGCCAGAACGACCACAGCTACCCCCTGACCTTCGGCGCCGGCACC
AAGCTGGAGATCAAG
nucleic acid encoding an exemplary anti-HER2 scFv
(SEQ ID NO: 44)
GAAGTGCAGCTGGTGGAAAGCGGCGGCGGCCTGGTGCAGCCGGGCGGCAGCCT
GCGCCTGAGCTGCGCGGCGAGCGGCTTTAACATTAAAGATACCTATATTCATTGG
GTGCGCCAGGCGCCGGGCAAAGGCCTGGAATGGGTGGCGCGCATTTATCCGACC
AACGGCTATACCCGCTATGCGGATAGCGTGAAAGGCCGCTTTACCATTAGCGCG
GATACCAGCAAAAACACCGCGTATCTGCAGATGAACAGCCTGCGCGCGGAAGAT
ACCGCGGTGTATTATTGCAGCCGCTGGGGCGGCGATGGCTTTTATGCGATGGATT
ATTGGGGCCAGGGCACCCTGGTGACCGTGAGCAGCGCGAGCACCAAAGGCCCG
AGCGTGTTTCCGCTGGCGCCGAGCAGCAAAAGCACCAGCGGCGGCACCGCGGCG
CTGGGCTGCCTGGTGAAAGATTATTTTCCGGAACCGGTGACCGTGAGCTGGAAC
AGCGGCGCGCTGACCAGCGGCGTGCATACCTTTCCGGCGGTGCTGCAGAGCAGC
GGCCTGTATAGCCTGAGCAGCGTGGTGACCGTGCCGAGCAGCAGCCTGGGCACC
CAGACCTATATTTGCAACGTGAACCATAAACCGAGCAACACCAAAGTGGATAAA
AAAGTGGAACCG
nucleic acid encoding an exemplary anti-FAP scFv
Heavy chain
(SEQ ID NO: 45)
CAGGTGCAGCTCCAGCAGAGTGGCGCAGAGCTCGCTCGCCCAGGCGCTTCTGTG
AATCTGAGTTGTAAGGCCTCCGGATATACTTTTACGAACAACGGCATCAACTGGC
TGAAGCAGCGGACCGGCCAGGGCCTGGAGTGGATCGGCGAAATATACCCCCGGT
CCACAAACACTCTCTATAACGAGAAGTTTAAGGGCAAAGCAACTCTGACCGCGG
ACAGGTCCTCTAACACAGCCTATATGGAGCTGAGAAGCTTGACGAGTGAGGACT
CCGCTGTCTATTTTTGCGCCCGAACTCTGACCGCTCCTTTTGCTTTTTGGGGCCAG
GGCACGCTCGTGACCGTAAGTGCG
Light chain
(SEQ ID NO: 46)
CAGATCGTCCTGACGCAGTCTCCAGCCATCATGAGCGCCTCACCCGGCGAAAAG
GTGACCATGACCTGCTCAGCCTCTTCTGGTGTGAATTTCATGCACTGGTACCAGC
AAAAAAGTGGGACCTCCCCTAAAAGGTGGATCTTCGATACCAGCAAACTGGCTT
CTGGCGTTCCCGCAAGGTTTAGCGGCTCTGGTTCCGGCACATCATACAGCCTGAC
GATCAGCAGCATGGAGGCAGAAGACGCAGCTACCTATTACTGCCAGCAATGGAG
CTTTAACCCACCTACTTTCGGAGGAGGAACAAAGCTGGAAATAAAAAGA
nucleic acid encoding an exemplary anti-CS1 scFv
Heavy chain
(SEQ ID NO: 47)
ATGGGATGGAGCTCTATCATCCTCTTCTTGGTAGCAACAGCTACAGGTGTCCACT
CCCAGGTCCAACTGCAGCAGCCTGGGGCTGAGCTGGTGAGGCCTGGAGCTTCAG
TGAAGCTGTCCTGCAAGGCTTCGGGGTACTCCTTCACCACCTACTGGATGAACTG
GGTGAAGCAGAGGCCTGGACAAGGCCTTGAGTGGATTGGCATGATTCATCCTTC
CGATAGTGAAACTAGGTTAAATCAGAAGTTCAAGGACAAGGCCACATTGACTGT
AGACAAATCCTCCAGCACAGCC
Light chain
(SEQ ID NO: 48)
GACATTGTGATGACCCAGTCTCAGAAATCCATGTCCACATCAGTAGGAGACAGG
GTCAGCATCACCTGCAAGGCCAGTCAGGATGTTATTACTGGTGTAGCCTGGTATC
AACAGAAACCAGGGCAATCTCCTAAATTACTGATTTACTCGGCATCCTACCGGTA
CACTGGAGTCCCTGATCGCTTCACTGGCAGTGGATCTGGGACGGATTTCACTTTC
ACCATCAGCAATGTGCAGGCTGAAGACCTGGCAGTTTATTACTGTCAGCAACATT
ATAGTACTCCTCTCACTTTCGGTGCTGGGACCAAGCTGGAGCTGAAA
nucleic acid encoding an exemplary anti-AMPA scFv
Heavy chain
(SEQ ID NO: 49)
ATGAAACTGCCGGTGCTGCTGGTGGTGCTGCTGCTGTTTACCAGCCCGGCGAGCA
GCAGCGAAGTGCAGCTGCAGGAAAGCGGCCCGAGCCTGGTGAAACCGAGCCAG
ACCCTGAGCCTGACCTGCAGCGTGACCGGCGATAGCATTACCAGCGGCTTTTGG
AACTGGCTGCGCAAATTTCCGGGCAACAAACTGGAATATCTGGGCTATATTAACT
ATAGCGGCAGCACCTATTATAACCCGAGCCTGAAAAGCCGCATTAGCTTTACCC
GCGATACCAGCAAAAACCAGTATTATCTGCATCTGAACAGCGTGACCGCGGAAG
ATACCGCGACCTATTATTGCGCGAGCTGGGTGCTGCGCGATTGGGGCCAGGGCA
CCACCCTGACCGTGAGCAGCGCGCGCCCGACCGCGCCGAGCGTGTATCCGCTGG
CGCCGGTGTGCGGCGATACCACCGGCAGCAGCGTGACCCTGGGCTGCCTGGTGA
AAGGCTATTTTCCGGAACCGGTGACCCTGACCTGGAACAGCGGCAGCCTGAGCA
GCGGCGTGCATACCTTTCCGGCGGTGCTGCAGAGCGATCTGTATACCCTGAGCA
GCAGCGTGACCGTGACCAGCAGCACCTGGCCGAGCCAGAGCATTACCTGCAACG
TGGCGCATCCGGCGAGCAGCACCAAAGTGGATAAAAAAATTGAACCGCGCGGC
CCGACCATTAAACCGTGCCCGCCGTGCAAATGCCCGGCGCCGAACCTGCTGGGC
GGCCCGAGCGTGTTTATTTTTCCGCCGAAAATTAAAGATGTGCTGATGATTAGCC
TGAGCCCGATTGTGACCTGCGTGGTGGTGGATGTGAGCGAAGATGATCCGGATG
TGCAGATTAGCTGGTTTGTGAACAACGTGGAAGTGCATACCGCGCAGACCCAGA
CCCATCGCGAAGATTATAACAGCACCCTGCGCGTGGTGAGCGCGCTGCCGATTC
AGCATCAGGATTGGATGAGCGGCAAAGAATTTAAATGCAAAGTGAACAACAAA
GATCTGCCGGCGCCGATTGAACGCACCATTAGCAAACCGAAAGGCAGCGTGCGC
GCGCCGCAGGTGTATGTGCTGCCGCCGCCGGAAGAAGAAATGACCAAAAAACA
GGTGACCCTGACCTGCATGGTGACCGATTTTATGCCGGAAGATATTTATGTGGAA
TGGACCAACAACGGCAAAACCGAACTGAACTATAAAAACACCGAACCGGTGCT
GGATAGCGATGGCAGCTATTTTATGTATAGCAAACTGCGCGTGGAAAAAAAAAA
CTGGGTGGAACGCAACAGCTATAGCTGCAGCGTGGTGCATGAAGGCCTGCATAA
CCATCATACCACCAAAAGCTTTAGCCGCACCCCGGGCAAA
Light chain
(SEQ ID NO: 50)
ATGACCAGCACCCTGCCGTTTAGCCCGCAGGTGAGCACCCCGCGCAGCAAATTT
GCGACCATGGAATTTCAGACCCAGGTGCTGATGAGCCTGCTGCTGTGCATGAGC
GGCGCGGCGGCGGATGTGGTGATGACCCAGACCCCGCTGACCCTGAGCGTGACC
ATTGGCCAGCCGGCGAGCATTAGCTGCAAAAGCAGCCAGAGCCTGCTGGATAGC
GATGGCAAAACCTATCTGAACTGGCTGCTGCAGCGCCCGGGCCAGAGCCCGAAA
CGCCTGATTTATCTGGTGAGCAAACTGGATAGCGGCGTGCCGGATCGCTTTACCG
GCAGCGGCAGCGGCACCGATTTTACCCTGAAAATTAGCCGCGTGGAAGCGGAAG
ATCTGGGCGTGTATTATTGCTGGCAGGGCACCCATTTTCCGCAGACCTTTGGCGG
CGGCACCAAACTGGAAATTAAACGCGCGCGCGCGGATGCGGCGCCGACCGTGA
GCATTTTTCCGCCGAGCAGCGAACAGCTGACCAGCGGCGGCGCGAGCGTGGTGT
GCTTTCTGAACAACTTTTATCCGAAAGATATTAACGTGAAATGGAAAATTGATGG
CAGCGAACGCCAGAACGGCGTGCTGAACAGCTGGACCGATCAGGATAGCAAAG
ATAGCACCTATAGCATGAGCAGCACCCTGACCCTGACCAAAGATGAATATGAAC
GCCATAACAGCTATACCTGCGAAGCGACCCATAAAACCAGCACCAGCCCGATTG
TGAAAAGCTTTAACCGCAACGAATGC

### Nucleic Acids Encoding Exemplary Transmembrane Domains

nucleic acid encoding an exemplary CD28 transmembrane domain
(SEQ ID NO: 51)
TTTTGGGTGCTGGTGGTGGTTGGTGGAGTCCTGGCTTGCTATAGCTTGCTAGTAA
CAGTGGCCTTTATTATTTTCTGGGTG
nucleic acid encoding an exemplary CD8 transmembrane domain
(SEQ ID NO: 52)
ATCTACATCTGGGCGCCCTTGGCCGGGACTTGTGGGGTCCTTCTCCTGTCACTGG
TTATCACCCTTTACTGC
nucleic acid encoding an exemplary TLR3 transmembrane domain
(SEQ ID NO: 80)
TTTTTTATGATCAATACGTCTATTCTTCTCATATTTATTTTCATCGTTCTTCTGATT
CACTTT
nucleic acid encoding an exemplary TLR4 transmembrane domain
(SEQ ID NO: 81)
ATTGGGGTGTCTGTCCTAAGCGTGCTGGTTGTTTCCGTGGTTGCCGTTCTGGTATA
T
nucleic acid encoding an exemplary IFNGR1 transmembrane domain
(SEQ ID NO: 105)
TCTCTTTGGATTCCAGTTGTTGCTGCTTTACTACTCTTTCTAGTGCTTAGCCTGGT
ATTCATC
nucleic acid encoding an exemplary IL10RA transmembrane domain
(SEQ ID NO: 106)
GTCATCATATTCTTTGCCTTTGTTTTGCTGCTCTCCGGAGCGCTGGCTTACTGCCT
CGCGCTC
nucleic acid encoding an exemplary TLR9 transmembrane domain
(SEQ ID NO: 107)
TTTGCCCTCAGTCTTCTGGCTGTAGCTCTCGGGCTTGGCGTCCCTATGCTTCACCA
CCTGTGT
nucleic acid encoding an exemplary TNFR2 transmembrane domain
(SEQ ID NO: 108)
TTCGCTCTTCCAGTTGGACTGATTGTGGGTGTGACAGCCTTGGGTCTACTAATAA
TAGGAGTGGTGAACTGTGTCATCATGACCCAGGTG

### Nucleic Acids Encoding Exemplary Signaling Domains

nucleic acid encoding an exemplary CD28 signaling domain
(SEQ ID NO: 53)
AGGAGTAAGAGGAGCAGGCTCCTGCACAGTGACTACATGAACATGACTCCCCGC
CGCCCCGGGCCCACCCGCAAGCATTACCAGCCCTATGCCCCACCACGCGACTTC
GCAGCCTATCGCTCC
nucleic acid encoding an exemplary CD35 signaling domain
(SEQ ID NO: 54)
AGAGTGAAGTTCAGCAGGAGCGCAGACGCCCCCGCGTACAAGCAGGGCCAGAA
CCAGCTCTATAACGAGCTCAATCTAGGACGAAGAGAGGAGTACGATGTTTTGGA
CAAGAGACGTGGCCGGGACCCTGAGATGGGGGGAAAGCCGAGAAGGAAGAACC
CTCAGGAAGGCCTGTACAATGAACTGCAGAAAGATAAGATGGCGGAGGCCTACA
GTGAGATTGGGATGAAAGGCGAGCGCCGGAGGGGCAAGGGGCACGATGGCCTT
TACCAGGGTCTCAGTACAGCCACCAAGGACACCTACGACGCCCTTCACATGCAG
GCCCTGCCCCCTCGC
nucleic acid encoding an exemplary 4-1BB signaling domain
(SEQ ID NO: 82)
AAACGGGGCAGAAAGAAACTCCTGTATATATTCAAACAACCATTTATGAGACCA
GTACAAACTACTCAAGAGGAAGATGGCTGTAGCTGCCGATTTCCAGAAGAAGAA
GAAGGAGGATGTGAACTG
nucleic acid encoding an exemplary TLR3 signaling domain
(SEQ ID NO: 83)
GAGGGGTGGAGGATTTCCTTTTACTGGAATGTGTCTGTGCATAGGGTTTTGGGTT
TTAAGGAGATAGATCGCCAAACAGAGCAGTTCGAGTATGCAGCTTATATTATAC
ACGCGTACAAAGATAAGGACTGGGTATGGGAGCATTTCAGTTCCATGGAAAAAG
AAGACCAGTCCTTGAAGTTCTGCCTTGAAGAAAGGGACTTCGAGGCCGGGGTTT
TTGAGCTGGAAGCGATCGTTAATAGTATTAAGCGGTCACGCAAGATCATCTTCGT
AATAACACATCACCTCCTCAAGGATCCACTTTGCAAACGCTTCAAAGTTCACCAT
GCGGTGCAGCAAGCGATCGAACAGAACCTTGACTCCATAATCCTTGTCTTTCTGG
AAGAAATACCTGATTACAAGTTGAATCATGCTCTGTGTCTGCGGCGGGGAATGTT
TAAGTCTCATTGTATCCTTAACTGGCCTGTGCAGAAAGAGCGCATTGGGGCGTTT
AGGCATAAACTCCAGGTTGCGCTTGGAAGCAAAAATTCCGTTCAT
nucleic acid encoding an exemplary TIR3 signaling domain
(SEQ ID NO: 84)
TGGCGGATCTCTTTCTACTGGAACGTGTCAGTGCATAGAGTCCTCGGGTTCAAAG
AGATTGACAGGCAGACGGAACAATTTGAGTATGCAGCGTACATTATCCATGCCT
ACAAAGATAAAGACTGGGTTTGGGAACATTTCTCCTCCATGGAAAAGGAGGATC
AGTCTTTGAAGTTTTGTCTGGAGGAGCGGGACTTTGAGGCGGGAGTGTTCGAGCT
TGAGGCCATTGTAAACTCGATCAAGCGGTCCCGTAAAATCATCTTTGTGATAACA
CACCACCTGTTGAAAGACCCGCTCTGCAAGCGCTTTAAGGTGCATCATGCTGTCC
AGCAGGCCATCGAGCAGAATCTGGATTCAATTATTCTGGTGTTTTTAGAAGAGAT
CCCAGACTATAAGCTGAATCACGCTCTATGTTTGCGTCGAGGCATGTTCAAATCT
CATTGCATCTTGAATTGGCCGGTGCAAAAGGAAAGGATTGGTGCTTTTCGGCACA
AATTGCAGGTCGCCCTGGGCTCCAAAAACAGTGTTCACAGAGTGAAGTTCAGCA
GGAGCGCAGACGCCCCCGCGTACAAGCAGGGCCAGAACCAGCTCTATAACGAG
CTCAATCTAGGACGAAGAGAGGAGTACGATGTTTTGGACAAGAGACGTGGCCGG
GACCCTGAGATGGGGGGAAAGCCGAGAAGGAAGAACCCTCAGGAAGGCCTGTA
CAATGAACTGCAGAAAGATAAGATGGCGGAGGCCTACAGTGAGATTGGGATGA
AAGGCGAGCGCCGGAGGGGCAAGGGGCACGATGGCCTTTACCAGGGTCTCAGT
ACAGCCACCAAGGACACCTACGACGCCCTTCACATGCAGGCCCTGCCCCCTCGC
TAAG
nucleic acid encoding an exemplary TLR3/TIR3 signaling domain
(SEQ ID NO: 109)
GAAGGATGGCGAATCTCTTTCTACTGGAACGTGTCTGTTCATAGAGTTTTGGGAT
TCAAGGAAATAGATCGCCAAACCGAGCAATTCGAGTATGCCGCATATATCATAC
ATGCATATAAGGACAAGGACTGGGTATGGGAACACTTCTCCTCTATGGAGAAAG
AAGATCAGTCACTCAAATTCTGCTTGGAGGAACGAGATTTCGAAGCCGGTGTATT
TGAGCTTGAAGCAATAGTAAACTCTATAAAAAGAAGTCGAAAAATTATTTTCGT
CATCACACATCATCTGTTGAAGGACCCGCTTTGTAAGAGGTTTAAAGTGCATCAC
GCTGTGCAGCAAGCCATTGAACAAAATCTGGATAGCATCATTCTCGTTTTTCTCG
AAGAAATACCCGACTATAAGCTCAATCACGCTTTGTGCTTGCGACGGGGAATGTT
CAAGTCCCACTGCATTCTTAACTGGCCAGTTCAAAAGGAACGAATCGGGGCCTT
CAGACATAAATTGCAAGTTGCCCTGGGCTCAAAGAACTCAGTGCAC
nucleic acid encoding an exemplary TLR4 signaling domain
(SEQ ID NO: 85)
AAGTTCTATTTCCATCTGATGCTTCTCGCTGGCTGCATAAAGTACGGGAGGGGGG
AG
nucleic acid encoding an exemplary TIR4 signaling domain
(SEQ ID NO: 86)
AATATATATGACGCTTTCGTGATCTACTCGAGCCAGGATGAGGACTGGGTTCGCA
ACGAGCTAGTCAAGAATCTTGAAGAGGGCGTGCCTCCTTTCCAGCTCTGTCTGCA
TTACCGCGATTTTATTCCTGGGGTGGCCATCGCGGCCAACATCATCCACGAGGGC
TTCCATAAATCCAGAAAAGTGATTGTCGTTGTGAGCCAGCATTTCATCCAGTCCA
GGTGGTGCATTTTCGAATATGAGATAGCCCAGACCTGGCAGTTTCTTAGCAGTCG
GGCTGGGATTATTTTTATCGTGCTGCAGAAGGTTGAAAAGACCCTTTTGCGGCAA
CAGGTGGAACTGTACCGATTATTATCCCGTAACACTTACTTGGAATGGGAAGACT
CAGTTCTCGGACGCCACATTTTCTGGCGCCGGCTCAGGAAGGCCCTGCTGGATGG
TAAATCCTGGAACCCCGAGGGGACAGTGGGGACCGGATGTAACTGGCAAGAGG
CAACAAGTATA
nucleic acid encoding an exemplary TLR4/TIR4 signaling domain
(SEQ ID NO: 110)
AAGTTCTATTTCCATCTGATGCTTCTCGCTGGCTGCATAAAGTACGGGAGGGGGG
AGAATATATATGACGCTTTCGTGATCTACTCGAGCCAGGATGAGGACTGGGTTCG
CAACGAGCTAGTCAAGAATCTTGAAGAGGGCGTGCCTCCTTTCCAGCTCTGTCTG
CATTACCGCGATTTTATTCCTGGGGTGGCCATCGCGGCCAACATCATCCACGAGG
GCTTCCATAAATCCAGAAAAGTGATTGTCGTTGTGAGCCAGCATTTCATCCAGTC
CAGGTGGTGCATTTTCGAATATGAGATAGCCCAGACCTGGCAGTTTCTTAGCAGT
CGGGCTGGGATTATTTTTATCGTGCTGCAGAAGGTTGAAAAGACCCTTTTGCGGC
AACAGGTGGAACTGTACCGATTATTATCCCGTAACACTTACTTGGAATGGGAAG
ACTCAGTTCTCGGACGCCACATTTTCTGGCGCCGGCTCAGGAAGGCCCTGCTGGA
TGGTAAATCCTGGAACCCCGAGGGGACAGTGGGGACCGGATGTAACTGGCAAGA
GGCAACAAGTATA
nucleic acid encoding an exemplary IFNγ signaling domain
(SEQ ID NO: 87)
ATGAAGTACACTTCTTACATACTCGCCTTCCAGCTTTGTATAGTGTTGGGCAGTCT
GGGTTGCTATTGCCAAGATCCTTATGTGAAGGAAGCAGAAAATTTGAAGAAATA
CTTTAACGCGGGTCATTCTGACGTTGCAGATAATGGAACCTTGTTTTTGGGAATA
CTTAAAAATTGGAAGGAGGAAAGCGACCGCAAGATCATGCAGTCTCAAATCGTT
TCCTTTTATTTTAAACTTTTCAAGAATTTTAAGGACGATCAGTCCATACAGAAAT
CTGTAGAGACTATCAAAGAGGATATGAACGTAAAGTTTTTTAACAGCAACAAAA
AGAAAAGAGATGACTTTGAGAAACTTACGAATTATAGTGTCACCGATCTGAACG
TCCAACGCAAAGCAATCCACGAGTTGATTCAAGTTATGGCAGAGCTTTCCCCAG
CGGCGAAAACTGGAAAGCGCAAACGATCTCAAATGCTCTTCCGGGGT
nucleic acid encoding an exemplary IFNGR1 signaling domain
(SEQ ID NO: 111)
TGTTTTTATATTAAGAAAATTAATCCATTGAAGGAAAAAAGCATAATATTACCCA
AGTCCTTGATCTCTGTGGTAAGAAGTGCTACTTTAGAGACAAAACCTGAATCAAA
ATATGTATCACTCATCACGTCATACCAGCCATTTTCCTTAGAAAAGGAGGTGGTC
TGTGAAGAGCCGTTGTCTCCAGCAACAGTTCCAGGCATGCATACCGAAGACAAT
CCAGGAAAAGTGGAACATACAGAAGAACTTTCTAGTATAACAGAAGTGGTGACT
ACTGAAGAAAATATTCCTGACGTGGTCCCGGGCAGCCATCTGACTCCAATAGAG
AGAGAGAGTTCTTCACCTTTAAGTAGTAACCAGTCTGAACCTGGCAGCATCGCTT
TAAACTCGTATCACTCCAGAAATTGTTCTGAGAGTGATCACTCCAGAAATGGTTT
TGATACTGATTCCAGCTGTCTGGAATCACATAGCTCCTTATCTGACTCAGAATTT
CCCCCAAATAATAAAGGTGAAATAAAAACAGAAGGACAAGAGCTCATAACCGT
AATAAAAGCCCCCACCTCCTTTGGTTATGATAAACCACATGTGCTAGTGGATCTA
CTTGTGGATGATAGCGGTAAAGAGTCCTTGATTGGTTATAGACCAACAGAAGAT
TCCAAAGAATTTTCATGAGATCAGCTAAGTTGCACCAACTTTGAAGTCTGATTTT
CCTGGACAGTTTTCTGCTTTAATTTCATGAAAAGATTATGATCTCAGAAATTGTA
TCTTAGTTGGTATCAACCAAATGGAGTGACTTAGTGTACATGAAAGCGTAAAGA
GGATGTGTGGCATTTTCACTTTTGGCTTGTAAAGTACAGACTTTTTTTTTTTTTTA
AACAAAAAAAGCATTGTAACTTATGAACCTTTACATCCAGATAGGTTACCAGTA
ACGGAACAGTATCCAGTACTCCTGGTTCCTAGGTGAGCAGGTGATGCCCCAGGG
ACCTTTGTAGCCACTTCACTTTTTTTCTTTTCTCTGCCTTGGTATAGCATATGTTTT
TGTAAGTTTATGCATACAGTAATTTTAAGTAATTTCAGAAGAAATTCTGCAAGCT
TTTCAAAATTGGACTTAAAATCTAATTCAAACTAATAGAATTAATGGAATATGTA
AATAGAAACGTGTATATTTTTTATGAAACATTACAGTTAGAGATTTTTAAATAAA
GAATTTTAAAACTCGAAAAAAAAAAAAAAAAAA
nucleic acid encoding an exemplary IFNγ signaling domain
(SEQ ID NO: 112)
ATGAAGTACACTTCTTACATACTCGCCTTCCAGCTTTGTATAGTGTTGGGCAGTCT
GGGTTGCTATTGCCAAGATCCTTATGTGAAGGAAGCAGAAAATTTGAAGAAATA
CTTTAACGCGGGTCATTCTGACGTTGCAGATAATGGAACCTTGTTTTTGGGAATA
CTTAAAAATTGGAAGGAGGAAAGCGACCGCAAGATCATGCAGTCTCAAATCGTT
TCCTTTTATTTTAAACTTTTCAAGAATTTTAAGGACGATCAGTCCATACAGAAAT
CTGTAGAGACTATCAAAGAGGATATGAACGTAAAGTTTTTTAACAGCAACAAAA
AGAAAAGAGATGACTTTGAGAAACTTACGAATTATAGTGTCACCGATCTGAACG
TCCAACGCAAAGCAATCCACGAGTTGATTCAAGTTATGGCAGAGCTTTCCCCAG
CGGCGAAAACTGGAAAGCGCAAACGATCTCAAATGCTCTTCCGGGGT
nucleic acid encoding an exemplary IL10RA signaling domain
(SEQ ID NO: 113)
CAGCTCTATGTCAGAAGGAGAAAAAAGCTCCCGTCCGTGCTTCTTTTTAAGAAGC
CTTCCCCGTTCATCTTCATTAGTCAGCGACCGAGCCCTGAGACCCAGGACACGAT
CCATCCGCTTGATGAAGAGGCGTTTCTTAAGGTAAGCCCTGAACTGAAGAATCTC
GATCTGCACGGCAGCACCGACAGTGGATTCGGGTCCACGAAACCCTCACTCCAG
ACAGAAGAGCCTCAGTTTTTGTTGCCTGATCCTCACCCCCAGGCTGATAGAACGT
TGGGTAATCGCGAGCCGCCGGTACTTGGAGACAGCTGCTCATCTGGGTCAAGCA
ACTCAACGGATTCTGGGATATGTTTGCAGGAACCAAGCCTCTCACCTAGCACTGG
GCCAACCTGGGAGCAGCAAGTGGGAAGCAATTCACGGGGGCAGGACGACTCTG
GTATAGACCTGGTACAAAACTCTGAGGGACGGGCGGGAGATACTCAAGGTGGTA
GCGCCCTGGGACACCACAGCCCACCGGAACCAGAGGTCCCGGGCGAGGAGGAC
CCTGCAGCGGTGGCGTTTCAGGGATATTTGCGCCAAACCCGCTGTGCTGAGGAG
AAGGCTACTAAAACGGGCTGCTTGGAAGAAGAATCTCCGCTCACAGACGGGCTC
GGCCCGAAGTTCGGCCGATGTTTGGTGGACGAAGCGGGGCTTCATCCGCCGGCG
CTTGCTAAAGGGTACCTGAAGCAAGACCCCTTGGAGATGACCCTGGCCTCCTCC
GGGGCGCCTACTGGTCAGTGGAACCAGCCGACCGAGGAATGGTCTCTCCTCGCC
CTTTCCAGTTGCTCCGACCTCGGTATATCAGACTGGAGTTTCGCACACGACCTTG
CACCCCTCGGCTGTGTAGCCGCCCCAGGAGGCCTTCTTGGCTCATTTAACAGTGA
CCTCGTTACGCTCCCCCTCATTTCATCTCTGCAATCCTCTGAA
nucleic acid encoding an exemplary TLR9/TIR9 signaling domain
(SEQ ID NO: 114)
GGCTGGGACCTCTGGTACTGCTTCCACCTGTGCCTGGCCTGGCTTCCCTGGCGGG
GGCGGCAAAGTGGGCGAGATGAGGATGCCCTGCCCTACGATGCCTTCGTGGTCT
TCGACAAAACGCAGAGCGCAGTGGCAGACTGGGTGTACAACGAGCTTCGGGGG
CAGCTGGAGGAGTGCCGTGGGCGCTGGGCACTCCGCCTGTGCCTGGAGGAACGC
GACTGGCTGCCTGGCAAAACCCTCTTTGAGAACCTGTGGGCCTCGGTCTATGGCA
GCCGCAAGACGCTGTTTGTGCTGGCCCACACGGACCGGGTCAGTGGTCTCTTGCG
CGCCAGCTTCCTGCTGGCCCAGCAGCGCCTGCTGGAGGACCGCAAGGACGTCGT
GGTGCTGGTGATCCTGAGCCCTGACGGCCGCCGCTCCCGCTACGTGCGGCTGCGC
CAGCGCCTCTGCCGCCAGAGTGTCCTCCTCTGGCCCCACCAGCCCAGTGGTCAGC
GCAGCTTCTGGGCCCAGCTGGGCATGGCCCTGACCAGGGACAACCACCACTTCT
ATAACCGGAACTTCTGCCAGGGACCCACGGCCGAATAG
nucleic acid encoding an exemplary TNFR2 signaling domain
(SEQ ID NO: 115)
AAAAAGAAGCCCTTGTGCCTGCAGAGAGAAGCCAAGGTGCCTCACTTGCCTGCC
GATAAGGCCCGGGGTACACAGGGCCCCGAGCAGCAGCACCTGCTGATCACAGCG
CCGAGCTCCAGCAGCAGCTCCCTGGAGAGCTCGGCCAGTGCGTTGGACAGAAGG
GCGCCCACTCGGAACCAGCCACAGGCACCAGGCGTGGAGGCCAGTGGGGCCGG
GGAGGCCCGGGCCAGCACCGGGAGCTCAGATTCTTCCCCTGGTGGCCATGGGAC
CCAGGTCAATGTCACCTGCATCGTGAACGTCTGTAGCAGCTCTGACCACAGCTCA
CAGTGCTCCTCCCAAGCCAGCTCCACAATGGGAGACACAGATTCCAGCCCCTCG
GAGTCCCCGAAGGACGAGCAGGTCCCCTTCTCCAAGGAGGAATGTGCCTTTCGG
TCACAGCTGGAGACGCCAGAGACCCTGCTGGGGAGCACCGAAGAGAAGCCCCT
GCCCCTTGGAGTGCCTGATGCTGGGATGAAGCCCAGTTAACCAGGCCGGTGTGG
GCTGTGTCGTAGCCAAGGTGGGCTGAGCCCTGGCAGGATGACCCTGCGAAG
GGGCCCTGGTCCTTCCAGGCCCCCACCACTAGGACTCTGAGGCTCTTTCTGGGCC
AAGTTCCTCTAGTGCCCTCCACAGCCGCAGCCTCCCTCTGACCTGCAGGCCAAGA
GCAGAGGCAGCGAGTTGTGGAAAGCCTCTGCTGCCATGGCGTGTCCCTCTCGGA
AGGCTGGCTGGGCATGGACGTTCGGGGCATGCTGGGGCAAGTCCCTGACTCTCT
GTGACCTGCCCCGCCCAGCTGCACCTGCCAGCCTGGCTTCTGGAGCCCTTGGGTT
TTTTGTTTGTTTGTTTGTTTGTTTGTTTGTTTCTCCCCCTGGGCTCTGCCCCAGCTC
TGGCTTCCAGAAAACCCCAGCATCCTTTTCTGCAGAGGGGCTTTCTGGAGAGGAG
GGATGCTGCCTGAGTCACCCATGAAGACAGGACAGTGCTTCAGCCTGAGGCTGA
GACTGCGGGATGGTCCTGGGGCTCTGTGCAGGGAGGAGGTGGCAGCCCTGTAGG
GAACGGGGTCCTTCAAGTTAGCTCAGGAGGCTTGGAAAGCATCACCTCAGGCCA
GGTGCAGTGGCTCACGCCTATGATCCCAGCACTTTGGGAGGCTGAGGCGGGTGG
ATCACCTGAGGTTAGGAGTTCGAGACCAGCCTGGCCAACATGGTAAAACCCCAT
CTCTACTAAAAATACAGAAATTAGCCGGGCGTGGTGGCGGGCACCTATAGTCCC
AGCTACTCAGAAGCCTGAGGCTGGGAAATCGTTTGAACCCGGGAAGCGGAGGTT
GCAGGGAGCCGAGATCACGCCACTGCACTCCAGCCTGGGCGACAGAGCGAGAG
TCTGTCTCAAAAGAAAAAAAAAAGCACCGCCTCCAAATGCCAACTTGTCCTTTTG
TACCATGGTGTGAAAGTCAGATGCCCAGAGGGCCCAGGCAGGCCACCATATTCA
GTGCTGTGGCCTGGGCAAGATAACGCACTTCTAACTAGAAATCTGCCAATTTTTT
AAAAAAGTAAGTACCACTCAGGCCAACAAGCCAACGACAAAGCCAAACTCTGC
CAGCCACATCCAACCCCCCACCTGCCATTTGCACCCTCCGCCTTCACTCCGGTGT
GCCTGCAGCCCCGCGCCTCCTTCCTTGCTGTCCTAGGCCACACCATCTCCTTTCAG
GGAATTTCAGGAACTAGAGATGACTGAGTCCTCGTAGCCATCTCTCTACTCCTAC
CTCAGCCTAGACCCTCCTCCTCCCCCAGAGGGGGGGTTCCTCTTCCCCACTCCC
CACCTTCAATTCCTGGGCCCCAAACGGGCTGCCCTGCCACTTTGGTACATGGCCA
GTGTGATCCCAAGTGCCAGTCTTGTGTCTGCGTCTGTGTTGCGTGTCGTGGGTGT
GTGTAGCCAAGGTCGGTAAGTTGAATGGCCTGCCTTGAAGCCACTGAAGCTGGG
ATTCCTCCCCATTAGAGTCAGCCTTCCCCCTCCCAGGGCCAGGGCCCTGCAGAGG
GGAAACCAGTGTAGCCTTGCCCGGATTCTGGGAGGAAGCAGGTTGAGGGGCTCC
TGGAAAGGCTCAGTCTCAGGAGCATGGGGATAAAGGAGAAGGCATGAAATTGTC
TAGCAGAGCAGGGGCAGGGTGATAAATTGTTGATAAATTCCACTGGACTTGAGC
TTGGCAGCTGAACTATTGGAGGGTGGGAGAGCCCAGCCATTACCATGGAGACAA
GAAGGGTTTTCCACCCTGGAATCAAGATGTCAGACTGGCTGGCTGCAGTGACGT
GCACCTGTACTCAGGAGGCTGAGGGGAGGATCACTGGAGCCCAGGAGTTTGAGG
CTGCAGCGAGCTATGATCGCGCCACTACACTCCAGCCTGAGCAACAGAGTGAGA
CCCTGTCTCTTAAAGAAAAAAAAAGTCAGACTGCTGGGACTGGCCAGGTTTCTG
CCCACATTGGACCCACATGAGGACATGATGGAGCGCACCTGCCCCCTGGTGGAC
AGTCCTGGGAGAACCTCAGGCTTCCTTGGCATCACAGGGCAGAGCCGGGAAGCG
ATGAATTTGGAGACTCTGTGGGGCCTTGGTTCCCTTGTGTGTGTGTGTTGATCCC
AAGACAATGAAAGTTTGCACTGTATGCTGGACGGCATTCCTGCTTATCAATAAAC
CTGTTTGTTTTAA
micleic acid encoding an exemplary BDNF signaling domain
(SEQ ID NO: 55)
AAACTGGCGCGCCATAGCAAATTTGGCATGAAAGGCCCGGCGAGCGTGATTAGC
AACGATGATGATAGCGCGAGCCCGCTGCATCATATTAGCAACGGCAGCAACACC
CCGAGCAGCAGCGAAGGCGGCCCGGATGCGGTGATTATTGGCATGACCAAAATT
CCGGTGATTGAAAACCCGCAGTATTTTGGCATTACCAACAGCCAGCTGAAACCG
GATACCTTTGTGCAGCATATTAAACGCCATAACATTGTGCTGAAACGCGAACTGG
GCGAAGGCGCGTTTGGCAAAGTGTTTCTGGCGGAATGCTATAACCTGTGCCCGG
AACAGGATAAAATTCTGGTGGCGGTGAAAACCCTGAAAGATGCGAGCGATAACG
CGCGCAAAGATTTTCATCGCGAAGCGGAACTGCTGACCAACCTGCAGCATGAAC
ATATTGTGAAATTTTATGGCGTGTGCGTGGAAGGCGATCCGCTGATTATGGTGTT
TGAATATATGAAACATGGCGATCTGAACAAATTTCTGCGCGCGCATGGCCCGGA
TGCGGTGCTGATGGCGGAAGGCAACCCGCCGACCGAACTGACCCAGAGCCAGAT
GCTGCATATTGCGCAGCAGATTGCGGCGGGCATGGTGTATCTGGCGAGCCAGCA
TTTTGTGCATCGCGATCTGGCGACCCGCAACTGCCTGGTGGGCGAAAACCTGCTG
GTGAAAATTGGCGATTTTGGCATGAGCCGCGATGTGTATAGCACCGATTATTATC
GCGTGGGCGGCCATACCATGCTGCCGATTCGCTGGATGCCGCCGGAAAGCATTA
TGTATCGCAAATTTACCACCGAAAGCGATGTGTGGAGCCTGGGCGTGGTGCTGT
GGGAAATTTTTACCTATGGCAAACAGCCGTGGTATCAGCTGAGCAACAACGAAG
TGATTGAATGCATTACCCAGGGCCGCGTGCTGCAGCGCCCGCGCACCTGCCCGC
AGGAAGTGTATGAACTGATGCTGGGCTGCTGGCAGCGCGAACCGCATATGCGCA
AAAACATTAAAGGCATTCATACCCTGCTGCAGAACCTGGCGAAAGCGAGCCCGG
TGTATCTGGATATTCTGGGC
nucleic acid encoding an exemplary VEGFR2 signaling domain
(SEQ ID NO: 88)
CTGAAGCTGGGCAAGCCCCTGGGCAGGGGCGCCTTCGGCCAGGTGATCGAGGCC
GACGCCTTCGGCATCGACAAGACCGCCACCTGCAGGACCGTGGCCGTGAAGATG
CTGAAGGAGGGCGCCACCCACAGCGAGCACAGGGCCCTGATGAGCGAGCTGAA
GATCCTGATCCACATCGGCCACCACCTGAACGTGGTGAACCTGCTGGGCGCCTG
CACCAAGCCCGGCGGCCCCCTGATGGTGATCGTGGAGTTCTGCAAGTTCGGCAA
CCTGAGCACCTACCTGAGGAGCAAGAGGAACGAGTTCGTGCCCTACAAGACCAA
GGGCGCCAGGTTCAGGCAGGGCAAGGACTACGTGGGCGCCATCCCCGTGGACCT
GAAGAGGAGGCTGGACAGCATCACCAGCAGCCAGAGCAGCGCCAGCAGCGGCT
TCGTGGAGGAGAAGAGCCTGAGCGACGTGGAGGAGGAGGAGGCCCCCGAGGAC
CTGTACAAGGACTTCCTGACCCTGGAGCACCTGATCTGCTACAGCTTCCAGGTGG
CCAAGGGCATGGAGTTCCTGGCCAGCAGGAAGTGCATCCACAGGGACCTGGCCG
CCAGGAACATCCTGCTGAGCGAGAAGAACGTGGTGAAGATCTGCGACTTCGGCC
TGGCCAGGGACATCTACAAGGACCCCGACTACGTGAGGAAGGGCGACGCCAGG
CTGCCCCTGAAGTGGATGGCCCCCGAGACCATCTTCGACAGGGTGTACACCATC
CAGAGCGACGTGTGGAGCTTCGGCGTGCTGCTGTGGGAGATCTTCAGCCTGGGC
GCCAGCCCCTACCCCGGCGTGAAGATCGACGAGGAGTTCTGCAGGAGGCTGAAG
GAGGGCACCAGGATGAGGGCCCCCGACTACACCACCCCCGAGATGTACCAGACC
ATGCTGGACTGCTGGCACGGCGAGCCCAGCCAGAGGCCCACCTTCAGCGAGCTG
GTGGAGCACCTGGGCAAC

### Nucleic Acids Encoding Exemplary Hinge Domains

nucleic acid encoding an exemplary CD28 hinge
domain
(SEQ ID NO: 56)
CTCGAGCCCAAATCTTGTGACAAAACTCACACATGCCCACCGTGCCCGG
ATCCCAAA
nucleic acid encoding an exemplary CD8 hinge
domain
(SEQ ID NO: 57)
ACCACGACGCCAGCGCCGCGACCACCAACACCGGCGCCCACCATCGCGT
CGCACCCCTGTCCCTGCGCCCAGAGGCGTGCCGGCCAGCGGCGGGGGGC
GCAGTGCAACGAGGGGGCTGGACTTCGCCTGTGAT

### Nucleic Acids Encoding Exemplary Polypeptides

nucleic acid encoding an exemplary NFKB1 polypeptide
(SEQ ID NO: 58)
ATGGCAGAAGATGATCCATATTTGGGAAGGCCTGAACAAATGTTTCATTTGGATC
CTTCTTTGACTCATACAATATTTAATCCAGAAGTATTTCAACCACAGATGGCACT
GCCAACAGCAGATGGCCCATACCTTCAAATATTAGAGCAACCTAAACAGAGAGG
ATTTCGTTTCCGTTATGTATGTGAAGGCCCATCCCATGGTGGACTACCTGGTGCC
TCTAGTGAAAAGAACAAGAAGTCTTACCCTCAGGTCAAAATCTGCAACTATGTG
GGACCAGCAAAGGTTATTGTTCAGTTGGTCACAAATGGAAAAAATATCCACCTG
CATGCCCACAGCCTGGTGGGAAAACACTGTGAGGATGGGATCTGCACTGTAACT
GCTGGACCCAAGGACATGGTGGTCGGCTTCGCAAACCTGGGTATACTTCATGTG
ACAAAGAAAAAAGTATTTGAAACACTGGAAGCACGAATGACAGAGGCGTGTAT
AAGGGGCTATAATCCTGGACTCTTGGTGCACCCTGACCTTGCCTATTTGCAAGCA
GAAGGTGGAGGGGACCGGCAGCTGGGAGATCGGGAAAAAGAGCTAATCCGCCA
AGCAGCTCTGCAGCAGACCAAGGAGATGGACCTCAGCGTGGTGCGGCTCATGTT
TACAGCTTTTCTTCCGGATAGCACTGGCAGCTTCACAAGGCGCCTGGAACCCGTG
GTATCAGACGCCATCTATGACAGTAAAGCCCCCAATGCATCCAACTTGAAAATT
GTAAGAATGGACAGGACAGCTGGATGTGTGACTGGAGGGGAGGAAATTTATCTT
CTTTGTGACAAAGTTCAGAAAGATGACATCCAGATTCGATTTTATGAAGAGGAA
GAAAATGGTGGAGTCTGGGAAGGATTTGGAGATTTTTCCCCCACAGATGTTCATA
GACAATTTGCCATTGTCTTCAAAACTCCAAAGTATAAAGATATTAATATTACAAA
ACCAGCCTCTGTGTTTGTCCAGCTTCGGAGGAAATCTGACTTGGAAACTAGTGAA
CCAAAACCTTTCCTCTACTATCCTGAAATCAAAGATAAAGAAGAAGTGCAGAGG
AAACGTCAGAAGCTCATGCCCAATTTTTCGGATAGTTTCGGCGGTGGTAGTGGTG
CCGGAGCTGGAGGCGGAGGCATGTTTGGTAGTGGCGGTGGAGGAGGGGGCACT
GGAAGTACAGGTCCAGGGTATAGCTTCCCACACTATGGATTTCCTACTTATGGTG
GGATTACTTTCCATCCTGGAACTACTAAATCTAATGCTGGGATGAAGCATGGAAC
CATGGACACTGAATCTAAAAAGGACCCTGAAGGTTGTGACAAAAGTGATGACAA
AAACACTGTAAACCTCTTTGGGAAAGTTATTGAAACCACAGAGCAAGATCAGGA
GCCCAGCGAGGCCACCGTTGGGAATGGTGAGGTCACTCTAACGTATGCAACAGG
AACAAAAGAAGAGAGTGCTGGAGTTCAGGATAACCTCTTTCTAGAGAAGGCTAT
GCAGCTTGCAAAGAGGCATGCCAATGCCCTTTTCGACTACGCGGTGACAGGAGA
CGTGAAGATGCTGCTGGCCGTCCAGCGCCATCTCACTGCTGTGCAGGATGAGAA
TGGGGACAGTGTCTTACACTTAGCAATCATCCACCTTCATTCTCAACTTGTGAGG
GATCTACTAGAAGTCACATCTGGTTTGATTTCTGATGACATTATCAACATGAGAA
ATGATCTGTACCAGACGCCCTTGCACTTGGCAGTGATCACTAAGCAGGAAGATG
TGGTGGAGGATTTGCTGAGGGCTGGGGCCGACCTGAGCCTTCTGGACCGCTTGG
GTAACTCTGTTTTGCACCTAGCTGCCAAAGAAGGACATGATAAAGTTCTCAGTAT
CTTACTCAAGCACAAAAAGGCAGCACTACTTCTTGACCACCCCAACGGGGACGG
TCTGAATGCCATTCATCTAGCCATGATGAGCAATAGCCTGCCATGTTTGCTGCTG
CTGGTGGCCGCTGGGGCTGACGTCAATGCTCAGGAGCAGAAGTCCGGGCGCACA
GCACTGCACCTGGCTGTGGAGCACGACAACATCTCATTGGCAGGCTGCCTGCTCC
TGGAGGGTGATGCCCATGTGGACAGTACTACCTACGATGGAACCACACCCCTGC
ATATAGCAGCTGGGAGAGGGTCCACCAGGCTGGCAGCTCTTCTCAAAGCAGCAG
GAGCAGATCCCCTGGTGGAGAACTTTGAGCCTCTCTATGACCTGGATGACTCTTG
GGAAAATGCAGGAGAGGATGAAGGAGTTGTGCCTGGAACCACGCCTCTAGATAT
GGCCACCAGCTGGCAGGTATTTGACATATTAAATGGGAAACCATATGAGCCAGA
GTTTACATCTGATGATTTACTAGCACAAGGAGACATGAAACAGCTGGCTGAAGA
TGTGAAGCTGCAGCTGTATAAGTTACTAGAAATTCCTGATCCAGACAAAAACTG
GGCTACTCTGGCGCAGAAATTAGGTCTGGGGATACTTAATAATGCCTTCCGGCTG
AGTCCTGCTCCTTCCAAAACACTTATGGACAACTATGAGGTCTCTGGGGGTACAG
TCAGAGAGCTGGTGGAGGCCCTGAGACAAATGGGCTACACCGAAGCAATTGAAG
TGATCCAGGCAGCCTCCAGCCCAGTGAAGACCACCTCTCAGGCCCACTCGCTGC
CTCTCTCGCCTGCCTCCACAAGGCAGCAAATAGACGAGCTCCGAGACAGTGACA
GTGTCTGCGACAGCGGCGTGGAGACATCCTTCCGCAAACTCAGCTTTACCGAGTC
TCTGACCAGTGGTGCCTCACTGCTAACTCTCAACAAAATGCCCCATGATTATGGG
CAGGAAGGACCTCTAGAAGGCAAAATTTAG
nucleic acid encoding an exemplary JUN polypeptide
(SEQ ID NO: 59)
CGTGAAGTGACGGACTGTTCTATGACTGCAAAGATGGAAACGACCTTCTATGAC
GATGCCCTCAACGCCTCGTTCCTCCCGTCCGAGAGCGGACCTTATGGCTACAGTA
ACCCCAAGATCCTGAAACAGAGCATGACCCTGAACCTGGCCGACCCAGTGGGGA
GCCTGAAGCCGCACCTCCGCGCCAAGAACTCGGACCTCCTCACCTCGCCCGACG
TGGGGCTGCTCAAGCTGGCGTCGCCCGAGCTGGAGCGCCTGATAATCCAGTCCA
GCAACGGGCACATCACCACCACGCCGACCCCCACCCAGTTCCTGTGCCCCAAGA
ACGTGACAGATGAGCAGGAGGGCTTCGCCGAGGGCTTCGTGCGCGCCCTGGCCG
AACTGCACAGCCAGAACACGCTGCCCAGCGTCACGTCGGCGGCGCAGCCGGTCA
ACGGGGCAGGCATGGTGGCTCCCGCGGTAGCCTCGGTGGCAGGGGGCAGCGGC
AGCGGCGGCTTCAGCGCCAGCCTGCACAGCGAGCCGCCGGTCTACGCAAACCTC
AGCAACTTCAACCCAGGCGCGCTGAGCAGCGGCGGCGGGGCGCCCTCCTACGGC
GCGGCCGGCCTGGCCTTTCCCGCGCAACCCCAGCAGCAGCAGCAGCCGCCGCAC
CACCTGCCCCAGCAGATGCCCGTGCAGCACCCGCGGCTGCAGGCCCTGAAGGAG
GAGCCTCAGACAGTGCCCGAGATGCCCGGCGAGACACCGCCCCTGTCCCCCATC
GACATGGAGTCCCAGGAGCGGATCAAGGCGGAGAGGAAGCGCATGAGGAACCG
CATCGCTGCCTCCAAGTGCCGAAAAAGGAAGCTGGAGAGAATCGCCCGGCTGGA
GGAAAAAGTGAAAACCTTGAAAGCTCAGAACTCGGAGCTGGCGTCCACGGCCA
ACATGCTCAGGGAACAGGTGGCACAGCTTAAACAGAAAGTCATGAACCACGTTA
ACAGTGGGTGCCAACTCATGCTAACGCAGCAGTTGCAAACATTTTGA
nucleic acid encoding an exemplary RELB polypeptide
(SEQ ID NO: 60)
ATGCTTCGGTCTGGGCCAGCCTCTGGGCCGTCCGTCCCCACTGGCCGGGCCATGC
CGAGTCGCCGCGTCGCCAGACCGCCGGCTGCGCCGGAGCTGGGGGCCTTAGGGT
CCCCCGACCTCTCCTCACTCTCGCTCGCCGTTTCCAGGAGCACAGATGAATTGGA
GATCATCGACGAGTACATCAAGGAGAACGGCTTCGGCCTGGACGGGGGACAGCC
GGGCCCGGGCGAGGGGCTGCCACGCCTGGTGTCTCGCGGGGCTGCGTCCCTGAG
CACGGTCACCCTGGGCCCTGTGGCGCCCCCAGCCACGCCGCCGCCTTGGGGCTG
CCCCCTGGGCCGACTAGTGTCCCCAGCGCCGGGCCCGGGCCCGCAGCCGCACCT
GGTCATCACGGAGCAGCCCAAGCAGCGCGGCATGCGCTTCCGCTACGAGTGCGA
GGGCCGCTCGGCCGGCAGCATCCTTGGGGAGAGCAGCACCGAGGCCAGCAAGA
CGCTGCCCGCCATCGAGCTCCGGGATTGTGGAGGGCTGCGGGAGGTGGAGGTGA
CTGCCTGCCTGGTGTGGAAGGACTGGCCTCACCGAGTCCACCCCCACAGCCTCGT
GGGGAAAGACTGCACCGACGGCATCTGCAGGGTGCGGCTCCGGCCTCACGTCAG
CCCCCGGCACAGTTTTAACAACCTGGGCATCCAGTGTGTGAGGAAGAAGGAGAT
TGAGGCTGCCATTGAGCGGAAGATTCAACTGGGCATTGACCCCTACAACGCTGG
GTCCCTGAAGAACCATCAGGAAGTAGACATGAATGTGGTGAGGATCTGCTTCCA
GGCCTCATATCGGGACCAGCAGGGACAGATGCGCCGGATGGATCCTGTGCTTTC
CGAGCCCGTCTATGACAAGAAATCCACAAACACATCAGAGCTGCGGATTTGCCG
AATTAACAAGGAAAGCGGGCCGTGCACCGGTGGCGAGGAGCTCTACTTGCTCTG
CGACAAGGTGCAGAAAGAGGACATATCAGTGGTGTTCAGCAGGGCCTCCTGGGA
AGGTCGGGCTGACTTCTCCCAGGCCGACGTGCACCGCCAGATTGCCATTGTGTTC
AAGACGCCGCCCTACGAGGACCTGGAGATTGTCGAGCCCGTGACAGTCAACGTC
TTCCTGCAGCGGCTCACCGATGGGGTCTGCAGCGAGCCATTGCCTTTCACGTACC
TGCCTCGCGACCATGACAGCTACGGCGTGGACAAGAAGCGGAAACGGGGGATG
CCCGACGTCCTTGGGGAGCTGAACAGCTCTGACCCCCATGGCATCGAGAGCAAA
CGGCGGAAGAAAAAGCCGGCCATCCTGGACCACTTCCTGCCCAACCACGGCTCA
GGCCCGTTCCTCCCGCCGTCAGCCCTGCTGCCAGACCCTGACTTCTTCTCTGGCA
CCGTGTCCCTGCCCGGCCTGGAGCCCCCTGGCGGGCCTGACCTCCTGGACGATGG
CTTTGCCTACGACCCTACGGCCCCCACACTCTTCACCATGCTGGACCTGCTGCCC
CCGGCACCGCCACACGCTAGCGCTGTTGTGTGCAGCGGAGGTGCCGGGGCCGTG
GTTGGGGAGACCCCCGGCCCTGAACCACTGACACTGGACTCGTACCAGGCCCCG
GGCCCCGGGGATGGAGGCACCGCCAGCCTTGTGGGCAGCAACATGTTCCCCAAT
CATTACCGCGAGGCGGCCTTTGGGGGCGGCCTCCTATCCCCGGGGCCTGAAGCC
ACG
nucleic acid encoding an exemplary IRF1 polypeptide
(SEQ ID NO: 61)
TACCCATACGATGTTCCTGACTATGCCGGTACCGAGCTCGGATCCACCATGGCTA
GCTGGAGCCACCCGCAGTTCGAGAAAGGTGGAGGTTCCGGAGGTGGATCGGGAG
GTGGATCGTGGAGCCACCCGCAGTTCGAAAAAGCGGCCGATATCACAAGTTTGT
ACAAAAAAGCAGGCTCCACCATGCCCATCACTCGGATGCGCATGAGACCCTGGC
TAGAGATGCAGATTAATTCCAACCAAATCCCGGGGCTCATCTGGATTAATAAAG
AGGAGATGATCTTCCAGATCCCATGGAAGCATGCTGCCAAGCATGGCTGGGACA
TCAACAAGGATGCCTGTTTGTTCCGGAGCTGGGCCATTCACACAGGCCGATACA
AAGCAGGGGAAAAGGAGCCAGATCCCAAGACGTGGAAGGCCAACTTTCGCTGT
GCCATGAACTCCCTGCCAGATATCGAGGAGGTGAAAGACCAGAGCAGGAACAA
GGGCAGCTCAGCTGTGCGAGTGTACCGGATGCTTCCACCTCTCACCAAGAACCA
GAGAAAAGAAAGAAAGTCGAAGTCCAGCCGAGATGCTAAGAGCAAGGCCAAGA
GGAAGTCATGTGGGGATTCCAGCCCTGATACCTTCTCTGATGGACTCAGCAGCTC
CACTCTGCCTGATGACCACAGCAGCTACACAGTTCCAGGCTACATGCAGGACTT
GGAGGTGGAGCAGGCCCTGACTCCAGCACTGTCGCCATGTGCTGTCAGCAGCAC
TCTCCCCGACTGGCACATCCCAGTGGAAGTIGTGCCGGACAGCACCAGTGATCT
GTACAACTTCCAGGTGTCACCCATGCCCTCCACCTCTGAAGCTACAACAGATGAG
GATGAGGAAGGGAAATTACCTGAGGACATCATGAAGCTCTTGGAGCAGTCGGAG
TGGCAGCCAACAAACGTGGATGGGAAGGGGTACCTACTCAATGAACCTGGAGTC
CAGCCCACCTCTGTCTATGGAGACTTTAGCTGTAAGGAGGAGCCAGAAATTGAC
AGCCCAGGGGGGGATATTGGGCTGAGTCTACAGCGTGTCTTCACAGATCTGAAG
AACATGGATGCCACCTGGCTGGACAGCCTGCTGACCCCAGTCCGGTTGCCCTCCA
TCCAGGCCATTCCCTGTGCACCGTTGGACCCAGCTTTCTTGTACAAAGTGGTGAC
GTAA
nucleic acid encoding an exemplary TNFα polypeptide
(SEQ ID NO: 62)
ATGTCTACCGAGTCTATGATTAGGGACGTGGAACTGGCTGAGGAGGCACTGCCC
AAAAAAACCGGCGGACCACAGGGCTCTAGGAGATGTCTGTTTCTGTCTCTGTTCT
CTTTTCTCATCGTGGCTGGCGCTACAACACTCTTCTGTCTGCTCCATTTCGGCGTG
ATTGGACCACAGCGAGAGGAATTTCCCCGGGATCTGTCACTCATTTCACCACTGG
CACAGGCTGTCCGATCTTCATCTCGGACTCCATCCGACAAACCTGTCGCCCATGT
CGTCGCCAACCCACAGGCCGAGGGCCAGCTCCAGTGGCTCAATAGGAGGGCAAA
CGCTCTGCTCGCCAATGGCGTGGAACTCCGGGATAACCAGCTCGTCGTGCCTAGT
GAGGGACTGTACCTCATCTACTCCCAGGTGCTGTTTAAGGGCCAGGGATGTCCTT
CTACACATGTGCTGCTCACACACACAATTTCACGGATCGCCGTGTCTTACCAGAC
TAAAGTCAATCTGCTCTCTGCCATCAAATCCCCATGTCAGCGGGAAACACCTGAG
GGCGCTGAGGCTAAACCTTGGTACGAACCCATCTACCTCGGAGGCGTGTTCCAG
CTGGAGAAGGGCGATAGACTGA
nucleic acid encoding an exemplary IL-10 polypeptide
(SEQ ID NO: 63)
ATGCACAGCTCAGCACTGCTCTGTTGCCTGGTCCTTCTGACAGGCGTAAGGGCGT
CACCTGGCCAAGGAACACAGTCAGAGAACAGCTGTACACATTTCCCCGGCAACT
TGCCCAATATGCTTAGGGATCTTCGCGATGCCTTCTCACGAGTGAAGACATTTTT
TCAGATGAAGGATCAGTTGGATAACCTTTTGCTGAAGGAAAGCCTGCTCGAGGA
CTTCAAGGGATACCTCGGATGCCAGGCACTGAGCGAAATGATACAGTTCTACCT
GGAAGAGGTAATGCCTCAGGCAGAAAATCAGGACCCCGATATTAAAGCTCATGT
GAACTCTCTGGGTGAGAACCTGAAAACTCTGAGGCTGAGGCTGCGGAGGTGTCA
CAGATTCCTGCCATGCGAGAACAAATCAAAAGCCGTCGAACAGGTGAAGAACGC
CTTTAACAAACTGCAGGAGAAAGGCATCTATAAAGCGATGAGCGAGTTCGATAT
TTTCATCAACTACATTGAGGCATACATGACGATGAAAATCCGAAATTAG
nucleic acid encoding an exemplary FGF-2 polypeptide
(SEQ ID NO: 64)
GGCGGCTGGCTCTATCACCACTCTGCCGGCCCTGCCGGAAGATGGCGGCAGCGG
CGCGTTCCCGCCGGGCCATTTTAAGGATCCGAAGCGCCTGTACTGCAAAAACGG
CGGCTTTTTCCTGCGCATTCACCCGGATGGCCGTGTGGACGGTGTGCGTGAGAAA
AGCGACCCGCACATCAAACTGCAACTGCAAGCGGAGGAACGTGGTGTAGTGAGC
ATCAAGGGTGTATGCGCTAACCGTTATCTGGCGATGAAAGAAGACGGCCGCCTG
CTGGCGAGCAAATGCGTCACCGACGAATGTTTCTTCTTCGAACGCCTGGAGTCCA
ACAACTACAACACGTATCGCTCCCGCAAATACACCTCTTGGTACGTGGCTCTGAA
ACGCACCGGCCAGTACAAACTGGGCTCCAAAACCGGCCCTGGCCAGAAAGCGAT
CCTGTTCCTGCCGATGTCCGCGAAATCCTAA
nucleic acid encoding an exemplary G-CSF polypeptide
(SEQ ID NO: 65)
ATGACTCCTTTGGGTCCAGCTTCTTCCTTGCCTCAATCCTTCTTGTTGAAGTGTTT
GGAGCAGGTTAGAAAGATCCAGGGTGATGGTGCTGCTTTGCAAGAGAAGTTGTG
TGCTACTTACAAGTTGTGTCACCCAGAAGAGTTGGTTTTGTTGGGTCACTCCTTG
GGTATTCCTTGGGCTCCATTGTCCTCTTGTCCATCCCAAGCTTTGCAATTGGCTGG
TTGTTTGTCCCAATTGCACTCCGGTTTGTTCTTGTACCAGGGTTTGTTGCAAGCTT
TGGAGGGTATTTCTCCAGAGTTGGGTCCAACTTTGGACACATTGCAGTTGGACGT
TGCTGACTTCGCTACTACTATCTGGCAACAGATGGAAGAATTGGGTATGGCTCCA
GCTTTGCAGCCAACTCAAGGTGCTATGCCAGCTTTTGCTTCTGCTTTCCAGAGAA
GAGCTGGTGGTGTTTTGGTTGCTTCTCACTTGCAGTCTTTCTTGGAGGTTTCCTAC
AGAGTTTTGAGACACTTGGCTCAACCATAA
nucleic acid encoding an exemplary GM-CSF polypeptide
(SEQ ID NO: 66)
ATGTGGCTGCAGAGCCTGCTGCTCTTGGGCACTGTGGCCTGCAGCATCTCTGCAC
CCGCCCGCTCGCCCAGCCCCAGCACGCAGCCCTGGGAGCATGTGAATGCCATCC
AGGAGGCCCGGCGTCTCCTGAACCTGAGTAGAGACACTGCTGCTGAGATGAATG
AAACAGTAGAAGTCATCTCAGAAATGTTTGACCTCCAGGAGCCGACCTGCCTAC
AGACCCGCCTGGAGCTGTACAAGCAGGGCCTGCGGGGCAGCCTCACCAAGCTCA
AGGGCCCCTTGACCATGATGGCCAGCCACTACAAGCAGCACTGCCCTCCAACCC
CGGAAACTTCCTGTGCAACCCAGATTATCACCTTTGAAAGTTTCAAAGAGAACCT
GAAGGACTTTCTGCTTGTCATCCCCTTTGACTGCTGGGAGCCAGTCCAGGAGTTG
CCAACTTTCTTGTACAAAGTGGTTGGCTCCACCAGCGGCAGCGGCAAGCCAGGC
TCCGGCGAAGGCAGCACCAAAGGC
nucleic acid encoding an exemplary Gal-9 polypeptide
(SEQ ID NO: 67)
GCCTTCAGCGGTTCCCAGGCTCCCTACCTGAGTCCAGCTGTCCCCTTTTCTGGGA
CTATTCAAGGAGGTCTCCAGGACGGACTTCAGATCACTGTCAATGGGACCGTTCT
CAGCTCCAGTGGAACCAGGTTTGCTGTGAACTTTCAGACTGGCTTCAGTGGAAAT
GACATTGCCTTCCACTTCAACCCTCGGTTTGAAGATGGAGGGTACGTGGTGTGCA
ACACGAGGCAGAACGGAAGCTGGGGGCCCGAGGAGAGGAAGACACACATGCCT
TTCCAGAAGGGGATGCCCTTTGACCTCTGCTTCCTGGTGCAGAGCTCAGATTTCA
AGGTGATGGTGAACGGGATCCTCTTCGTGCAGTACTTCCACCGCGTGCCCTTCCA
CCGTGTGGACACCATCTCCGTCAATGGCTCTGTGCAGCTGTCCTACATCAGCTTC
CAGCCTCCCGGCGTGTGGCCTGCCAACCCGGCTCCCATTACCCAGACAGTCATCC
ACACAGTGCAGAGCGCCCCTGGACAGATGTTCTCTACTCCCGCCATCCCACCTAT
GATGTACCCCCACCCCGCCTATCCGATGCCTTTCATCACCACCATTCTGGGAGGG
CTGTACCCATCCAAGTCCATCCTCCTGTCAGGCACTGTCCTGCCCAGTGCTCAGA
GGTTCCACATCAACCTGTGCTCTGGGAACCACATCGCCTTCCACCTGAACCCCCG
TTTTGATGAGAATGCTGTGGTCCGCAACACCCAGATCGACAACTCCTGGGGGTCT
GAGGAGCGAAGTCTGCCCCGAAAAATGCCCTTCGTCCGTGGCCAGAGCTTCTCA
GTGTGGATCTTGTGTGAAGCTCACTGCCTCAAGGTGGCCGTGGATGGTCAGCACC
TGTTTGAATACTACCATCGCCTGAGGAACCTGCCCACCATCAACAGACTGGAAG
TGGGGGGCGACATCCAGCTGACCCATGTGCAGACATAG
nucleic acid encoding an exemplary PD1 polypeptide
(SEQ ID NO: 68)
ATGCAGATCCCACAGGCGCCCTGGCCAGTCGTCTGGGCGGTGCTACAACTGGGC
TGGCGGCCAGGATGGTTCTTAGACTCCCCAGACAGGCCCTGGAACCCCCCCACC
TTCTCCCCAGCCCTGCTCGTGGTGACCGAAGGGGACAACGCCACCTTCACCTGCA
GCTTCTCCAACACATCGGAGAGCTTCGTGCTAAACTGGTACCGCATGAGCCCCA
GCAACCAGACGGACAAGCTGGCCGCTTTCCCCGAGGACCGCAGCCAGCCCGGCC
AGGACTGCCGCTTCCGTGTCACACAACTGCCCAACGGGCGTGACTTCCACATGA
GCGTGGTCAGGGCCCGGCGCAATGACAGCGGCACCTACCTCTGTGGGGCCATCT
CCCTGGCCCCCAAGGCGCAGATCAAAGAGAGCCTGCGGGCAGAGCTCAGGGTG
ACAGAGAGAAGGGCAGAAGTGCCCACAGCCCACCCCAGCCCCTCACCCAGGCC
AGCCGGCCAGTTCCAAACCCTGGTGGTTGGTGTCGTGGGCGGCCTGCTGGGCAG
CCTGGTGCTGCTAGTCTGGGTCCTGGCCGTCATCTGCTCCCGGGCCGCACGAGGG
ACAATAGGAGCCAGGCGCACCGGCCAGCCCCTGAAGGAGGACCCCTCAGCCGTG
CCTGTGTTCTCTATTGTTTATGCTTCCCTGGATTTCCAGTGGCGAGAGAAGACCCC
GGAGCCCCCCGTGCCCTGTGTCCCTGAGCAGACGGAGTATGCCACCATTGTCTTT
CCTAGCGGAATGGGCACCTCATCCCCCGCCCGCAGGGGCTCAGCCGACGGCCCT
CGGAGTGCCCAGCCACTGAGGCCTGAGGATGGACACTGCTCTTGGCCCCTGACG
CGTCTGCAGGATATCAAGCTTGCGGTACCGCGGGCCCGGGATCCACCGGTCGCC
ACC
nucleic acid encoding an exemplary TIM-3 polypeptide
(SEQ ID NO: 69)
ATGTTTTCACATCTTCCCTTTGACTGTGTCCTGCTGCTGCTGCTGCTACTACTTAC
AAGGTCCTCAGAAGTGGAATACAGAGCGGAGGTCGGTCAGAATGCCTATCTGCC
CTGCTTCTACACCCCAGCCGCCCCAGGGAACCTCGTGCCCGTCTGCTGGGGCAAA
GGAGCCTGTCCTGTGTTTGAATGTGGCAACGTGGTGCTCAGGACTGATGAAAGG
GATGTGAATTATTGGACATCCAGATACTGGCTAAATGGGGATTTCCGCAAAGGA
GATGTGTCCCTGACCATAGAGAATGTGACTCTAGCAGACAGTGGGATCTACTGCT
GCCGGATCCAAATCCCAGGCATAATGAATGATGAAAAATTTAACCTGAAGTTGG
TCATCAAACCAGCCAAGGTCACCCCTGCACCGACTCTGCAGAGAGACTTCACTG
CAGCCTTTCCAAGGATGCTTACCACCAGGGGACATGGCCCAGCAGAGACACAGA
CACTGGGGAGCCTCCCTGATATAAATCTAACACAAATATCCACATTGGCCAATG
AGTTACGGGACTCTAGATTGGCCAATGACTTACGGGACTCTGGAGCAACCATCA
GAATAGGCATCTACATCGGAGCAGGGATCTGTGCTGGGCTGGCTCTGGCTCTTAT
CTTCGGCGCTTTAATTTTCAAATGGTATTCTCATAGCAAAGAGAAGATACAGAAT
TTAAGCCTCATCTCTTTGGCCAACCTCCCTCCCTCAGGATTGGCAAATGCAGTAG
CAGAGGGAATTCGCTCAGAAGAAAACATCTATACCATTGAAGAGAACGTATATG
AAGTGGAGGAGCCCAATGAGTATTATTGCTATGTCAGCAGCAGGCAGCAACCCT
CACAACCTTTGGGTTGTCGCTTTGCAATGCCATAG
nucleic acid encoding an exemplary CXCR3 polypeptide
(SEQ ID NO: 70)
ATGGTTCTGGAAGTTTCCGACCACCAGGTGCTTAATGATGCGGAAGTTGCGGCAC
TGCTGGAAAACTTTAGCTCATCCTATGACTACGGCGAGAACGAGTCAGACTCTTG
CTGTACCTCCCCTCCTTGCCCCCAAGACTTTAGCTTGAATTTCGATCGGGCCTTCC
TGCCCGCACTTTACTCTCTGCTGTTCCTGTTGGGACTGTTGGGCAACGGGGCCGT
GGCAGCAGTACTGTTGAGTCGAAGGACTGCCCTGTCCAGTACCGATACATTTCTG
CTGCACCTCGCCGTGGCTGACACACTGCTGGTTCTGACCCTGCCCCTTTGGGCAG
TCGATGCAGCTGTCCAGTGGGTTTTCGGGAGTGGACTTTGTAAGGTGGCAGGGG
CGCTGTTCAATATCAACTTCTACGCCGGAGCACTGCTGCTGGCTTGTATAAGCTT
CGACCGATATCTGAACATTGTGCACGCCACGCAACTCTATAGAAGAGGCCCTCC
AGCAAGGGTTACACTCACCTGTCTTGCAGTTTGGGGGCTGTGCTTGCTTTTCGCA
TTGCCCGACTTCATTTTCCTGAGTGCTCACCATGACGAGCGGCTGAACGCCACTC
ATTGTCAATACAACTTCCCTCAGGTGGGGAGAACAGCGCTGCGAGTGCTGCAGT
TGGTGGCCGGATTCCTGCTGCCTTTGCTGGTGATGGCGTACTGCTACGCGCATAT
TTTGGCGGTCTTGTTGGTTTCTAGAGGGCAAAGGCGGCTGAGGGCTATGCGACTG
GTCGTTGTGGTGGTTGTCGCCTTCGCGCTCTGCTGGACCCCATACCACCTGGTGG
TGCTGGTTGATATACTGATGGATTTGGGAGCCCTGGCGAGGAATTGCGGTAGAG
AAAGCCGGGTCGACGTTGCAAAAAGCGTCACCTCAGGGCTGGGCTACATGCACT
GTTGTCTGAACCCCCTTCTGTACGCATTCGTGGGGGTGAAGTTCCGGGAACGCAT
GTGGATGTTGTTGCTGCGACTGGGTTGCCCTAATCAGCGCGGTCTGCAGCGACAG
CCCAGTTCTTCTCGAAGGGACTCATCATGGTCCGAAACCTCAGAGGCTAGTTATT
CTGGCCTC
nucleic acid encoding an exemplary CXCR4 polypeptide
(SEQ ID NO: 71)
ATGGAGGGGATATCAATCTACACATCAGATAATTACACGGAAGAGATGGGTTCC
GGCGATTACGACTCTATGAAAGAGCCGTGTTTTAGAGAGGAAAACGCGAACTTT
AACAAGATCTTTCTCCCCACCATCTACAGCATCATCTTTCTCACAGGCATCGTAG
GGAACGGCCTGGTCATCCTGGTGATGGGATACCAAAAGAAGCTGAGGTCAATGA
CCGACAAGTATAGGCTCCATCTGTCCGTGGCCGACCTCCTGTTTGTGATTACCCT
GCCTTTTTGGGCAGTTGACGCTGTCGCTAATTGGTACTTCGGCAACTTCCTCTGTA
AGGCAGTGCACGTTATCTACACTGTGAATCTTTATAGTTCCGTCTTGATCTTGGCC
TTTATCAGTCTCGACAGGTATTTGGCGATTGTGCACGCTACCAACTCACAACGAC
CTAGAAAACTCCTGGCTGAGAAGGTGGTTTACGTTGGTGTGTGGATTCCAGCTCT
CCTGTTGACAATACCAGACTTCATTTTCGCTAATGTGAGCGAGGCCGATGACAGA
TACATTTGTGACCGATTTTACCCAAACGATCTGTGGGTAGTGGTATTTCAGTTCC
AACACATTATGGTCGGGCTGATCTTGCCCGGCATTGTCATACTGTCTTGCTACTG
CATCATTATTTCTAAGCTGTCACACTCAAAAGGCCACCAAAAGAGGAAGGCTCT
GAAAACAACGGTGATCCTGATACTGGCCTTCTTCGCATGTTGGCTGCCCTACTAT
ATCGGCATCAGCATTGACTCATTTATACTCCTGGAAATTATCAAGCAGGGCTGCG
AGTTCGAGAACACCGTTCATAAGTGGATTTCTATAACCGAGGCCCTCGCCTTCTT
TCACTGTTGTTTGAATCCGATTCTCTACGCGTTTCTTGGCGCCAAATTTAAAACAA
GCGCCCAACATGCACTGACATCAGTGTCTAGGGGGAGCTCTCTGAAAATCCTCTC
CAAGGGAAAACGAGGCGGACATAGCAGTGTCAGCACTGAGTCCGAATCCAGCTC
ATTTCATAGCTCT
nucleic acid encoding an exemplary CTLA4 polypeptide
(SEQ ID NO: 116)
ATGGCTTGCCTTGGATTTCAGCGGCACAAGGCTCAGCTGAACCTGGCTACCAGG
ACCTGGCCCTGCACTCTCCTGTTTTTTCTTCTCTTCATCCCTGTCTTCTGCAAAGC
AATGCACGTGGCCCAGCCTGCTGTGGTACTGGCCAGCAGCCGAGGCATCGCCAG
CTTTGTGTGTGAGTATGCATCTCCAGGCAAAGCCACTGAGGTCCGGGTGACAGTG
CTTCGGCAGGCTGACAGCCAGGTGACTGAAGTCTGTGCGGCAACCTACATGATG
GGGAATGAGTTGACCTTCCTAGATGATTCCATCTGCACGGGCACCTCCAGTGGAA
ATCAAGTGAACCTCACTATCCAAGGACTGAGGGCCATGGACACGGGACTCTACA
TCTGCAAGGTGGAGCTCATGTACCCACCGCCATACTACCTGGGCATAGGCAACG
GAACCCAGATTTATGTAATTGATCCAGAACCGTGCCCAGATTCTGACTTCCTCC
TCTGGATCCTTGCAGCAGTTAGTTCGGGGTTGTTTTTTTATAGCTTTCTCCTCACA
GCTGTTTCTTTGAGCAAAATGCTAAAGAAAAGAAGCCCTCTTACAACAGGGGTC
TATGTGAAAATGCCCCCAACAGAGCCAGAATGTGAAAAGCAATTTCAGCCTTAT
TTTATTCCCATCAATTGA
nucleic acid encoding an exemplary TLR3 polypeptide
(SEQ ID NO: 117)
ATGAGACAGACTTTGCCTTGTATCTACTTTTGGGGGGGCCTTTTGCCCTTTGGGAT
GCTGTGTGCATCCTCCACCACCAAGTGCACTGTTAGCCATGAAGTTGCTGACTGC
AGCCACCTGAAGTTGACTCAGGTACCCGATGATCTACCCACAAACATAACAGTG
TTGAACCTTACCCATAATCAACTCAGAAGATTACCAGCCGCCAACTTCACAAGGT
ATAGCCAGCTAACTAGCTTGGATGTAGGATTTAACACCATCTCAAAACTGGAGC
CAGAATTGTGCCAGAAACTTCCCATGTTAAAAGTTTTGAACCTCCAGCACAATGA
GCTATCTCAACTTTCTGATAAAACCTTTGCCTTCTGCACGAATTTGACTGAACTCC
ATCTCATGTCCAACTCAATCCAGAAAATTAAAAATAATCCCTTTGTCAAGCAGAA
GAATTTAATCACATTAGATCTGTCTCATAATGGCTTGTCATCTACAAAATTAGGA
ACTCAGGTTCAGCTGGAAAATCTCCAAGAGCTTCTATTATCAAACAATAAAATTC
AAGCGCTAAAAAGTGAAGAACTGGATATCTTTGCCAATTCATCTTTAAAAAAATT
AGAGTTGTCATCGAATCAAATTAAAGAGTTTTCTCCAGGGTGTTTTCACGCAATT
GGAAGATTATTTGGCCTCTTTCTGAACAATGTCCAGCTGGGTCCCAGCCTTACAG
AGAAGCTATGTTTGGAATTAGCAAACACAAGCATTCGGAATCTGTCTCTGAGTA
ACAGCCAGCTGTCCACCACCAGCAATACAACTTTCTTGGGACTAAAGTGGACAA
ATCTCACTATGCTCGATCTTTCCTACAACAACTTAAATGTGGTTGGTAACGATTC
CTTTGCTTGGCTTCCACAACTAGAATATTTCTTCCTAGAGTATAATAATATACAG
CATTTGTTTTCTCACTCTTTGCACGGGCTTTTCAATGTGAGGTACCTGAATTTGAA
ACGGTCTTTTACTAAACAAAGTATTTCCCTTGCCTCACTCCCCAAGATTGATGATT
TTTCTTTTCAGTGGCTAAAATGTTTGGAGCACCTTAACATGGAAGATAATGATAT
TCCAGGCATAAAAAGCAATATGTTCACAGGATTGATAAACCTGAAATACTTAAG
TCTATCCAACTCCTTTACAAGTTTGCGAACTTTGACAAATGAAACATTTGTATCA
CTTGCTCATTCTCCCTTACACATACTCAACCTAACCAAGAATAAAATCTCAAAAA
TAGAGAGTGATGCTTTCTCTTGGTTGGGCCACCTAGAAGTACTTGACCTGGGCCT
TAATGAAATTGGGCAAGAACTCACAGGCCAGGAATGGAGAGGTCTAGAAAATAT
TTTCGAAATCTATCTTTCCTACAACAAGTACCTGCAGCTGACTAGGAACTCCTTT
GCCTTGGTCCCAAGCCTTCAACGACTGATGCTCCGAAGGGTGGCCCTTAAAAAT
GTGGATAGCTCTCCTTCACCATTCCAGCCTCTTCGTAACTTGACCATTCTGGATCT
AAGCAACAACAACATAGCCAACATAAATGATGACATGTTGGAGGGTCTTGAGAA
ACTAGAAATTCTCGATTTGCAGCATAACAACTTAGCACGGCTCTGGAAACACGC
AAACCCTGGTGGTCCCATTTATTTCCTAAAGGGTCTGTCTCACCTCCACATCCTTA
ACTTGGAGTCCAACGGCTTTGACGAGATCCCAGTTGAGGTCTTCAAGGATTTATT
TGAACTAAAGATCATCGATTTAGGATTGAATAATTTAAACACACTTCCAGCATCT
GTCTTTAATAATCAGGTGTCTCTAAAGTCATTGAACCTTCAGAAGAATCTCATAA
CATCCGTTGAGAAGAAGGTTTTCGGGCCAGCTTTCAGGAACCTGACTGAGTTAG
ATATGCGCTTTAATCCCTTTGATTGCACGTGTGAAAGTATTGCCTGGTTTGTTAAT
TGGATTAACGAGACCCATACCAACATCCCTGAGCTGTCAAGCCACTACCTTTGCA
ACACTCCACCTCACTATCATGGGTTCCCAGTGAGACTTTTTGATACATCATCTTGC
AAAGACAGTGCCCCCTTTGAACTCTTTTTCATGATCAATACCAGTATCCTGTTGA
TTTTTATCTTTATTGTACTTCTCATCCACTTTGAGGGCTGGAGGATATCTTTTTATT
GGAATGTTTCAGTACATCGAGTTCTTGGTTTCAAAGAAATAGACAGACAGACAG
AACAGTTTGAATATGCAGCATATATAATTCATGCCTATAAAGATAAGGATTGGGT
CTGGGAACATTTCTCTTCAATGGAAAAGGAAGACCAATCTCTCAAATTTTGTCTG
GAAGAAAGGGACTTTGAGGCGGGTGTTTTTGAACTAGAAGCAATTGTTAACAGC
ATCAAAAGAAGCAGAAAAATTATTTTTGTTATAACACACCATCTATTAAAAGAC
CCATTATGCAAAAGATTCAAGGTACATCATGCAGTTCAACAAGCTATTGAACAA
AATCTGGATTCCATTATATTGGTTTTCCTTGAGGAGATTCCAGATTATAAACTGA
ACCATGCACTCTGTTTGCGAAGAGGAATGTTTAAATCTCACTGCATCTTGAACTG
GCCAGTTCAGAAAGAACGGATAGGTGCCTTTCGTCATAAATTGCAAGTAGCACT
TGGATCCAAAAACTCTGTACATTAA
nucleic acid encoding an exemplary TLR4 polypeptide
(SEQ ID NO: 118)
ATGATGTCTGCCTCGCGCCTGGCTGGGACTCTGATCCCAGCCATGGCCTTCCTCT
CCTGCGTGAGACCAGAAAGCTGGGAGCCCTGCGTGGAGGTGGTTCCTAATATTA
CTTATCAATGCATGGAGCTGAATTTCTACAAAATCCCCGACAACCTCCCCTTCTC
AACCAAGAACCTGGACCTGAGCTTTAATCCCCTGAGGCATTTAGGCAGCTATAG
CTTCTTCAGTTTCCCAGAACTGCAGGTGCTGGATTTATCCAGGTGTGAAATCCAG
ACAATTGAAGATGGGGCATATCAGAGCCTAAGCCACCTCTCTACCTTAATATTGA
CAGGAAACCCCATCCAGAGTTTAGCCCTGGGAGCCTTTTCTGGACTATCAAGTTT
ACAGAAGCTGGTGGCTGTGGAGACAAATCTAGCATCTCTAGAGAACTTCCCCAT
TGGACATCTCAAAACTTTGAAAGAACTTAATGTGGCTCACAATCTTATCCAATCT
TTCAAATTACCTGAGTATTTTTCTAATCTGACCAATCTAGAGCACTTGGACCTTTC
CAGCAACAAGATTCAAAGTATTTATTGCACAGACTTGCGGGTTCTACATCAAATG
CCCCTACTCAATCTCTCTTTAGACCTGTCCCTGAACCCTATGAACTTTATCCAACC
AGGTGCATTTAAAGAAATTAGGCTTCATAAGCTGACTTTAAGAAATAATTTTGAT
AGTTTAAATGTAATGAAAACTTGTATTCAAGGTCTGGCTGGTTTAGAAGTCCATC
GTTTGGTTCTGGGAGAATTTAGAAATGAAGGAAACTTGGAAAAGTTTGACAAAT
CTGCTCTAGAGGGCCTGTGCAATTTGACCATTGAAGAATTCCGATTAGCATACTT
AGACTACTACCTCGATGATATTATTGACTTATTTAATTGTTTGACAAATGTTTCTT
CATTTTCCCTGGTGAGTGTGACTATTGAAAGGGTAAAAGACTTTTCTTATAATTT
CGGATGGCAACATTTAGAATTAGTTAACTGTAAATTTGGACAGTTTCCCACATTG
AAACTCAAATCTCTCAAAAGGCTTACTTTCACTTCCAACAAAGGTGGGAATGCTT
TTTCAGAAGTTGATCTACCAAGCCTTGAGTTTCTAGATCTCAGTAGAAATGGCTT
GAGTTTCAAAGGTTGCTGTTCTCAAAGTGATTTTGGGACAACCAGCCTAAAGTAT
TTAGATCTGAGCTTCAATGGTGTTATTACCATGAGTTCAAACTTCTTGGGCTTAG
AACAACTAGAACATCTGGATTTCCAGCATTCCAATTTGAAACAAATGAGTGAGTT
TTCAGTATTCCTATCACTCAGAAACCTCATTTACCTTGACATTTCTCATACTCACA
CCAGAGTTGCTTTCAATGGCATCTTCAATGGCTTGTCCAGTCTCGAAGTCTTGAA
AATGGCTGGCAATTCTTTCCAGGAAAACTTCCTTCCAGATATCTTCACAGAGCTG
AGAAACTTGACCTTCCTGGACCTCTCTCAGTGTCAACTGGAGCAGTTGTCTCCAA
CAGCATTTAACTCACTCTCCAGTCTTCAGGTACTAAATATGAGCCACAACAACTT
CTTTTCATTGGATACGTTTCCTTATAAGTGTCTGAACTCCCTCCAGGTTCTTGATT
ACAGTCTCAATCACATAATGACTTCCAAAAAACAGGAACTACAGCATTTTCCAA
GTAGTCTAGCTTTCTTAAATCTTACTCAGAATGACTTTGCTTGTACTTGTGAACAC
CAGAGTTTCCTGCAATGGATCAAGGACCAGAGGCAGCTCTTGGTGGAAGTTGAA
CGAATGGAATGTGCAACACCTTCAGATAAGCAGGGCATGCCTGTGCTGAGTTTG
AATATCACCTGTCAGATGAATAAGACCATCATTGGTGTGTCGGTCCTCAGTGTGC
TTGTAGTATCTGTTGTAGCAGTTCTGGTCTATAAGTTCTATTTTCACCTGATGCTT
CTTGCTGGCTGCATAAAGTATGGTAGAGGTGAAAACATCTATGATGCCTTTGTTA
TCTACTCAAGCCAGGATGAGGACTGGGTAAGGAATGAGCTAGTAAAGAATTTAG
AAGAAGGGGTGCCTCCATTTCAGCTCTGCCTTCACTACAGAGACTTTATTCCCGG
TGTGGCCATTGCTGCCAACATCATCCATGAAGGTTTCCATAAAAGCCGAAAGGT
GATTGTTGTGGTGTCCCAGCACTTCATCCAGAGCCGCTGGTGTATCTTTGAATAT
GAGATTGCTCAGACCTGGCAGTTTCTGAGCAGTCGTGCTGGTATCATCTTCATTG
TCCTGCAGAAGGTGGAGAAGACCCTGCTCAGGCAGCAGGTGGAGCTGTACCGCC
TTCTCAGCAGGAACACTTACCTGGAGTGGGAGGACAGTGTCCTGGGGCGGCACA
TCTTCTGGAGACGACTCAGAAAAGCCCTGCTGGATGGTAAATCATGGAATCCAG
AAGGAACAGTGGGTACAGGATGCAATTGGCAGGAAGCAACATCTATCTGA
nucleic acid encoding an exemplary TLR9 polypeptide
(SEQ ID NO: 119)
ATGGGTTTCTGCCGCAGCGCCCTGCACCCGCTGTCTCTCCTGGTGCAGGCCATCA
TGCTGGCCATGACCCTGGCCCTGGGTACCTTGCCTGCCTTCCTACCCTGTGAGCT
CCAGCCCCACGGCCTGGTGAACTGCAACTGGCTGTTCCTGAAGTCTGTGCCCCAC
TTCTCCATGGCAGCACCCCGTGGCAATGTCACCAGCCTTTCCTTGTCCTCCAACC
GCATCCACCACCTCCATGATTCTGACTTTGCCCACCTGCCCAGCCTGCGGCATCT
CAACCTCAAGTGGAACTGCCCGCCGGTTGGCCTCAGCCCCATGCACTTCCCCTGC
CACATGACCATCGAGCCCAGCACCTTCTTGGCTGTGCCCACCCTGGAAGAGCTA
AACCTGAGCTACAACAACATCATGACTGTGCCTGCGCTGCCCAAATCCCTCATAT
CCCTGTCCCTCAGCCATACCAACATCCTGATGCTAGACTCTGCCAGCCTCGCCGG
CCTGCATGCCCTGCGCTTCCTATTCATGGACGGCAACTGTTATTACAAGAACCCC
TGCAGGCAGGCACTGGAGGTGGCCCCGGGTGCCCTCCTTGGCCTGGGCAACCTC
ACCCACCTGTCACTCAAGTACAACAACCTCACTGTGGTGCCCCGCAACCTGCCTT
CCAGCCTGGAGTATCTGCTGTTGTCCTACAACCGCATCGTCAAACTGGCGCCTGA
GGACCTGGCCAATCTGACCGCCCTGCGTGTGCTCGATGTGGGCGGAAATTGCCG
CCGCTGCGACCACGCTCCCAACCCCTGCATGGAGTGCCCTCGTCACTTCCCCCAG
CTACATCCCGATACCTTCAGCCACCTGAGCCGTCTTGAAGGCCTGGTGTTGAAGG
ACAGTTCTCTCTCCTGGCTGAATGCCAGTTGGTTCCGTGGGCTGGGAAACCTCCG
AGTGCTGGACCTGAGTGAGAACTTCCTCTACAAATGCATCACTAAAACCAAGGC
CTTCCAGGGCCTAACACAGCTGCGCAAGCTTAACCTGTCCTTCAATTACCAAAAG
AGGGTGTCCTTTGCCCACCTGTCTCTGGCCCCTTCCTTCGGGAGCCTGGTCGCCCT
GAAGGAGCTGGACATGCACGGCATCTTCTTCCGCTCACTCGATGAGACCACGCT
CCGGCCACTGGCCCGCCTGCCCATGCTCCAGACTCTGCGTCTGCAGATGAACTTC
ATCAACCAGGCCCAGCTCGGCATCTTCAGGGCCTTCCCTGGCCTGCGCTACGTGG
ACCTGTCGGACAACCGCATCAGCGGAGCTTCGGAGCTGACAGCCACCATGGGGG
AGGCAGATGGAGGGGAGAAGGTCTGGCTGCAGCCTGGGGACCTTGCTCCGGCCC
CAGTGGACACTCCCAGCTCTGAAGACTTCAGGCCCAACTGCAGCACCCTCAACTT
CACCTTGGATCTGTCACGGAACAACCTGGTGACCGTGCAGCCGGAGATGTTTGC
CCAGCTCTCGCACCTGCAGTGCCTGCGCCTGAGCCACAACTGCATCTCGCAGGCA
GTCAATGGCTCCCAGTTCCTGCCGCTGACCGGTCTGCAGGTGCTAGACCTGTCCC
ACAATAAGCTGGACCTCTACCACGAGCACTCATTCACGGAGCTACCGCGACTGG
AGGCCCTGGACCTCAGCTACAACAGCCAGCCCTTTGGCATGCAGGGCGTGGGCC
ACAACTTCAGCTTCGTGGCTCACCTGCGCACCCTGCGCCACCTCAGCCTGGCCCA
CAACAACATCCACAGCCAAGTGTCCCAGCAGCTCTGCAGTACGTCGCTGCGGGC
CCTGGACTTCAGCGGCAATGCACTGGGCCATATGTGGGCCGAGGGAGACCTCTA
TCTGCACTTCTTCCAAGGCCTGAGCGGTTTGATCTGGCTGGACTTGTCCCAGAAC
CGCCTGCACACCCTCCTGCCCCAAACCCTGCGCAACCTCCCCAAGAGCCTACAG
GTGCTGCGTCTCCGTGACAATTACCTGGCCTTCTTTAAGTGGTGGAGCCTCCACT
TCCTGCCCAAACTGGAAGTCCTCGACCTGGCAGGAAACCAGCTGAAGGCCCTGA
CCAATGGCAGCCTGCCTGCTGGCACCCGGCTCCGGAGGCTGGATGTCAGCTGCA
ACAGCATCAGCTTCGTGGCCCCCGGCTTCTTTTCCAAGGCCAAGGAGCTGCGAG
AGCTCAACCTTAGCGCCAACGCCCTCAAGACAGTGGACCACTCCTGGTTTGGGC
CCCTGGCGAGTGCCCTGCAAATACTAGATGTAAGCGCCAACCCTCTGCACTGCG
CCTGTGGGGCGGCCTTTATGGACTTCCTGCTGGAGGTGCAGGCTGCCGTGCCCGG
TCTGCCCAGCCGGGTGAAGTGTGGCAGTCCGGGCCAGCTCCAGGGCCTCAGCAT
CTTTGCACAGGACCTGCGCCTCTGCCTGGATGAGGCCCTCTCCTGGGACTGTTTC
GCCCTCTCGCTGCTGGCTGTGGCTCTGGGCCTGGGTGTGCCCATGCTGCATCACC
TCTGTGGCTGGGACCTCTGGTACTGCTTCCACCTGTGCCTGGCCTGGCTTCCCTG
GCGGGGGCGGCAAAGTGGGCGAGATGAGGATGCCCTGCCCTACGATGCCTTCGT
GGTCTTCGACAAAACGCAGAGCGCAGTGGCAGACTGGGTGTACAACGAGCTTCG
GGGGCAGCTGGAGGAGTGCCGTGGGCGCTGGGCACTCCGCCTGTGCCTGGAGGA
ACGCGACTGGCTGCCTGGCAAAACCCTCTTTGAGAACCTGTGGGCCTCGGTCTAT
GGCAGCCGCAAGACGCTGTTTGTGCTGGCCCACACGGACCGGGTCAGTGGTCTC
TTGCGCGCCAGCTTCCTGCTGGCCCAGCAGCGCCTGCTGGAGGACCGCAAGGAC
GTCGTGGTGCTGGTGATCCTGAGCCCTGACGGCCGCCGCTCCCGCTACGTGCGGC
TGCGCCAGCGCCTCTGCCGCCAGAGTGTCCTCCTCTGGCCCCACCAGCCCAGTGG
TCAGCGCAGCTTCTGGGCCCAGCTGGGCATGGCCCTGACCAGGGACAACCACCA
CTTCTATAACCGGAACTTCTGCCAGGGACCCACGGCCGAATAG
nucleic acid encoding an exemplary TNFR2 polypeptide
(SEQ ID NO: 120)
ATGGCGCCCGTCGCCGTCTGGGCCGCGCTGGCCGTCGGACTGGAGCTCTGGGCT
GCGGCGCACGCCTTGCCCGCCCAGGTGGCATTTACACCCTACGCCCCGGAGCCC
GGGAGCACATGCCGGCTCAGAGAATACTATGACCAGACAGCTCAGATGTGCTGC
AGCAAATGCTCGCCGGGCCAACATGCAAAAGTCTTCTGTACCAAGACCTCGGAC
ACCGTGTGTGACTCCTGTGAGGACAGCACATACACCCAGCTCTGGAACTGGGTT
CCCGAGTGCTTGAGCTGTGGCTCCCGCTGTAGCTCTGACCAGGTGGAAACTCAA
GCCTGCACTCGGGAACAGAACCGCATCTGCACCTGCAGGCCCGGCTGGTACTGC
GCGCTGAGCAAGCAGGAGGGGTGCCGGCTGTGCGCGCCGCTGCGCAAGTGCCGC
CCGGGCTTCGGCGTGGCCAGACCAGGAACTGAAACATCAGACGTGGTGTGCAAG
CCCTGTGCCCCGGGGACGTTCTCCAACACGACTTCATCCACGGATATTTGCAGGC
CCCACCAGATCTGTAACGTGGTGGCCATCCCTGGGAATGCAAGCATGGATGCAG
TCTGCACGTCCACGTCCCCCACCCGGAGTATGGCCCCAGGGGCAGTACACTTACC
CCAGCCAGTGTCCACACGATCCCAACACACGCAGCCAACTCCAGAACCCAGCAC
TGCTCCAAGCACCTCCTTCCTGCTCCCAATGGGCCCCAGCCCCCCAGCTGAAGGG
AGCACTGGCGACTTCGCTCTTCCAGTTGGACTGATTGTGGGTGTGACAGCCTTGG
GTCTACTAATAATAGGAGTGGTGAACTGTGTCATCATGACCCAGGTGAAAAAGA
AGCCCTTGTGCCTGCAGAGAGAAGCCAAGGTGCCTCACTTGCCTGCCGATAAGG
CCCGGGGTACACAGGGCCCCGAGCAGCAGCACCTGCTGATCACAGCGCCGAGCT
CCAGCAGCAGCTCCCTGGAGAGCTCGGCCAGTGCGTTGGACAGAAGGGCGCCCA
CTCGGAACCAGCCACAGGCACCAGGCGTGGAGGCCAGTGGGGCCGGGGAGGCC
CGGGCCAGCACCGGGAGCTCAGATTCTTCCCCTGGTGGCCATGGGACCCAGGTC
AATGTCACCTGCATCGTGAACGTCTGTAGCAGCTCTGACCACAGCTCACAGTGCT
CCTCCCAAGCCAGCTCCACAATGGGAGACACAGATTCCAGCCCCTCGGAGTCCC
CGAAGGACGAGCAGGTCCCCTTCTCCAAGGAGGAATGTGCCTTTCGGTCACAGC
TGGAGACGCCAGAGACCCTGCTGGGGAGCACCGAAGAGAAGCCCCTGCCCCTTG
GAGTGCCTGATGCTGGGATGAAGCCCAGTTAA

### Example 4: Bioengineering Mesenchymal Stromal Cells with Chimeric Antigen Receptors for Enhanced Immunosuppressive Treatment Efficacy

This Example describes the design and engineering of MSCs to express one or more chimeric antigen receptors (CARs-MSCs) and describes how such CAR-MSCs can enhance immunosuppression at antigen-specific inflammatory target sites.

The results in this Example re-present and expand on at least some of the results provided in other Examples.

### Methods

### Cell Lines, Primary Peripheral Blood Mononuclear Cells (PBMCs), Primary T Cells, and Primary Mesenchymal Stromal Cells (MSCs)

Primary human adipose-derived MSCs were obtained and cultured with StemXVivo Mesenchymal Stem Cell Expansion Media (R&D Systems, Minneapolis, MN). Human embryonic kidney 293 (HEK-293T) and human epithelial breast cancer cell line (MCF7) were obtained from ATCC (HTB-22, Manassas, VA, USA) and cultured in D10 (DMEM Gibco, Gaithersburg, MD, US) with 10% (v/v) fetal bovine serum (FBS, Millipore Sigma, Ontario, Canada) and 1% (v/v) penicillin-streptomycin-glutamine (PSG, Gibco, Gaithersburg, MD, US). HEK-293T were used for lentiviral production. MCF7 was irradiated and confirmed for E-cadherin (Ecad) positivity by flow cytometry for use as a form of CAR-based single chain variable fragment (scFv) stimulation within in vivo tumor models as described.

Normal Human Primary Epidermal Keratinocytes were derived from the epidermis of juvenile or adult foreskin from single or pooled donors and cultured in Keratinocyte growth media (PromoCell, Heidelberg, Germany). Normal Human Primary Bronchial Epithelial Cells were isolated from the epithelial lining of airways above bifurcation of the lungs (Lonza, Cohasset, MN). These cells were subsequently cultured in Airway epithelial cell basal medium expansion media (PromoCell, Heidelberg, Germany).

Acute lymphoblastic leukemia cell line NALM6 and mantle cell lymphoma cell line JeKo-1 were also purchased from ATCC (CRL-3273 and CRL-3006, respectively, Manassas, VA, USA). These cell lines were cultured in R20 and R10 (RPMI 1640, Gibco, Gaithersburg, MD, US) with 20% (v/v) or 10% (v/v) FBS (Millipore Sigma, Ontario, Canada), respectively, and 1% (v/v) PSG (Gibco, Gaithersburg, MD, US). For in vivo experiment use, cell lines were transduced with luciferase-ZsGreen lentivirus (Addgene, Cambridge, MA, USA). In addition, NALM6 and/or luciferase⁺ NALM6 were transduced with human Ecad-encoding lentivirus as a form of cell-based Ecad stimulation (GeneCopoeia, Rockville, MD). Cell lines were cultured up to 20 passages, and fresh aliquots were thawed every 7-8 weeks. Cell lines were authenticated by the manufacturer and routinely checked for phenotype by flow cytometry. Cell lines were tested monthly for mycoplasma. PBMCs were isolated from de-identified normal donor blood apheresis cones using SepMate tubes (STEMCELL Technologies, Vancouver, Canada). T cells were separated with negative selection magnetic beads using EasySep™ Human T Cell 80 Isolation Kit (STEMCELL Technologies, Vancouver, Canada).

Primary T cells and PBMCs were cultured in T cell medium containing X-VIVO 15 (Lonza, Walkersville, MD, USA), 10% (v/v) human serum albumin (Innovative Research, Novi, MI, USA), and 1% (v/v) PSG (Gibco, Gaithersburg, MD, USA) before selection for in vitro coculture. Freshly isolated human PBMCs were injected via i.v. administration for in vivo experiments.

scFv Phage Display Library

hmcECAD.6 scFv sequence was generated and optimized for binding to Ecad protein through phage display library selection. A naive human scFv library of approximately 2×10⁹ diversity was used to screen against recombinant Ecad protein (Biomolecular Discovery, Rochester, NY). The hmcECAD.6 scFv clone was identified after two rounds of screening against mouse Ecad followed by another round of panning on human Ecad Fc chimeric protein (both from BioLegend, San Diego, CA). This clone was shown to react with mouse Ecad by phage ELISA. Relative affinity versus the FLAG antibody estimated the hmcECAD.6 clone to be approximately 5 nM. Final hmcECAD.6 sequence (SEQ ID NO: 90) was codon optimized for cloning of heavy and light chain into EcCAR plasmid.

### Chimeric Antigen Receptor (CAR) Design and Virus Production

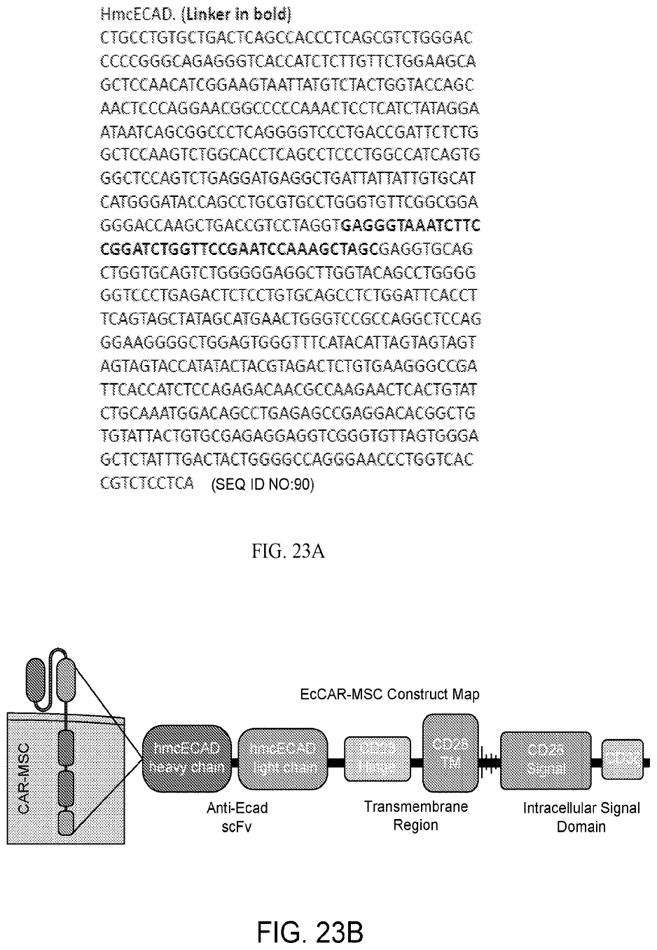

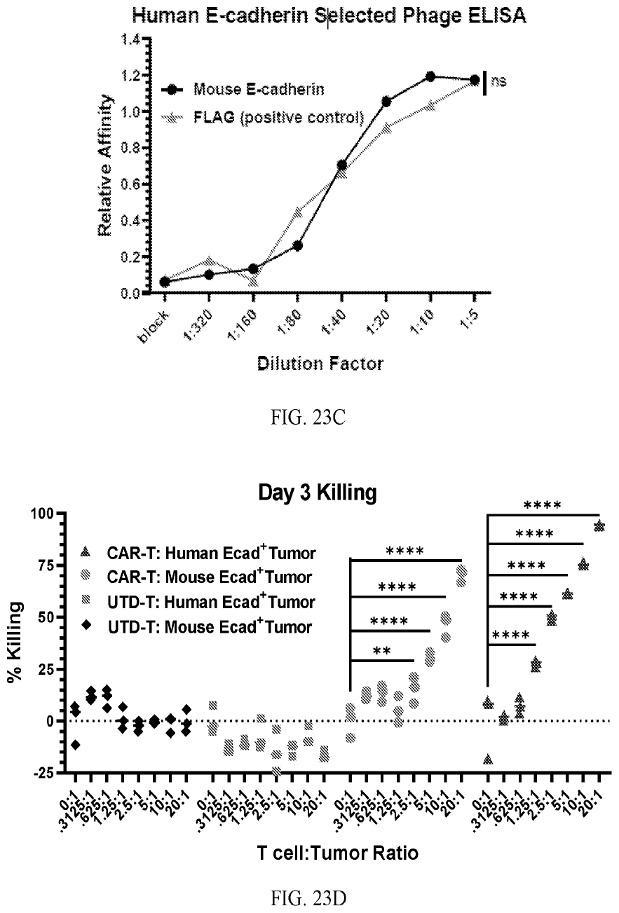

MSCs were transduced via lentiviral vectors encoding specifically designed CAR constructs under EF-1 alpha promoter. Optimization of transduction was performed with CD19-targeted CAR (CAR19) construct. See, for example, June et al., Science 359, 1361-1365 (2018). CAR19 is composed of a CD19-directed scFv derived from clone FMC-63 and fused to 4-1BB and CD3ζ signaling domains (FMC63-41BB-ζ). A VSV-g-pseudotyped second-generation lentivirus loaded with CAR transgene was generated via lipofectamine transfection of HEK-293T cells, followed by standard procedures for harvesting, concentration, and functional titration of lentivirus. See, for example, Sterneret al. Blood 133, 697-709 (2019) and Sakemura et al. Blood 139 (26): 3708-3721 (2022). CAR constructs were designed to include an scFv against human/canine Ecad (FIG. 23A) with CD28ζ intracellular signaling domain to induce MSC immunomodulatory activation (FIG. 23B). CAR plasmid was generated and sequence validated. Pre-seeded MSCs (concentration of 250,000 cells/well) were transduced in a 6-well plate with lentiviral particles at a multiplicity of infection (MOI) of 3 with various concentrations of protamine sulfate (25, 50, 100 ug/uL) for optimization of CAR expression and MSC proliferation.

### T Cell Suppression Assay

To test the ability of CAR-MSCs to inhibit activated T cells, a T cell suppression assay was used. Briefly, untransduced (UTD)-MSCs or EcCAR-MSCs (suppressors) were co-cultured with activated T cells (effectors) in the presence or absence of 1) soluble Ecad (stimulator) or 2) a matched Ecad⁺ and Ecad⁻ NALM6 cell line to provide both soluble and cell-based antigen-specific stimulation to the CAR-MSCs. T cells were isolated from PBMCs of normal donors using negative selection magnetic beads. See, for example, Sterner et al. Blood 133, 697-709 (2019). Isolated T cells were non-specifically activated using CD3/CD28 stimulating beads (Dynabeads, Invitrogen, Waltham, MA, USA) at a 1:1 bead to T cell ratio in T cell medium containing X-VIVO 15 (Lonza, Walkersville, MD, USA), 10% (v/v) human serum albumin (Innovative Research, Novi, MI, USA), and 1% (v/v) PSG (Gibco, Gaithersburg, MD, USA). MSCs were preincubated with or without soluble Ecad protein (250 ng-1000 ng/ml) or matched Ecad⁺ and Ecad⁻ cell lines (1:1 MSC to Ecad⁺ cell ratio) (stimulator) to activate EcCAR-MSCs specifically through the CAR in StemXVivo serum-free Mesenchymal Stem Cell Expansion media (R&D Systems, Minneapolis, MN, USA). After 24 hours, stimulated T cells (effectors) were co-cultured with UTD-MSCs or EcCAR-MSCs (suppressors). Cells were co-cultured at a 1:5 MSC to T cell ratio with 250,000 T cells per 6-well plate. Cells were harvested and analyzed by flow cytometry after different coculture time points as indicated in individual experiments.

### Multiparametric Flow Cytometry

Staining for flow cytometry was performed. Briefly, MSCs were isolated and grown in culture, then transduced with CAR to create CAR-MSCs to be cocultured with T cells/PBMCs in 96-well plates for 24-hour assays and 6-well plates for longer culture. Following desired coculture durations, adherent MSCs were detached with Accutase (STEMCELL Technologies, Vancouver, Canada) at 10 mL per 75 cm² surface area and incubated for 10-15 minutes at 37° C. Following detachment, all well contents were spun and washed in flow buffer (Phosphate-buffered saline (PBS), 2% (v/v) FBS, and 1% (v/v) sodium azide) and stained with desired antibodies for 15 minutes in the dark at room temperature. Following final wash steps, cells were analyzed for desired surface markers, and positivity was determined through negative gating by fluorescence minus one (FMO) control wells. Absolute cell count numbers were obtained using volumetric measurement. Cells were gated on SSC vs. FSC plots for cell separation by size and complexity, FSC-H vs. FSC-A plots to exclude doublets, and live/dead Aqua staining (Cat #L34966, Thermo Fisher Scientific, Waltham, MA, USA) to exclude dead cells, followed by cell subset characterization based on predesigned antibody panels which were optimized and used to stain samples.

Anti-human antibodies were purchased from Biolegend, eBioscience, or BD Biosciences (San Diego, CA, USA). Samples were prepared for flow cytometry. See, for example, Sakemura et al. Blood 139 (26): 3708-3721 (2022). All antibodies used to stain are listed in (Table 4). Flow cytometry was performed on three-laser CytoFLEX (Beckman Coulter, Chaska, MN, USA) where cells were gated by singlet discrimination and live cells were determined by live/dead aqua staining (Cat #L34966, Thermo Fisher Scientific, Waltham, MA, USA). All gating analyses were performed with FlowJo X10.0.7r2 software (Becton Dickenson, Ashland, OR, USA) or Kaluza Analysis software (Beckman Coulter, Indianapolis, Indiana, USA).

TABLE 4
List of antibodies used for flow cytometry, clone, vendor, and catalog numbers.

| Antibody Name | Clone | Vendor | Catalog # |
|---|---|---|---|
| PE/Cyanine7 anti-human Galectin-9 Antibody | 9M1-3 | BioLegend | 348916 |
| PE anti-human CD105 Antibody | 43A3 | BioLegend | 323205 |
| PerCP/Cyanine5.5 anti-human CD90 (Thy1) | 5E10 | BioLegend | 328117 |
| Antibody | | | |
| CD73 Monoclonal Antibody (AD2), PE, | AD2 | Invitrogen | 25073942 |
| eBioscience ™ | | | |
| APC/Cyanine7 anti-human CD183 (CXCR3) | G025H7 | BioLegend | 353722 |
| Antibody | | | |
| CD184 (CXCR4) Monoclonal Antibody (2B11), | 2B11 | Life | 17999182 |
| APC, eBioscience ™ | | Technologies | |
| Brilliant Violet 650 ™ anti-human CD3 Antibody | OKT3 | BioLegend | 317324 |
| CD279 (PD-1) Monoclonal Antibody (MIH4), | MIH4 | eBioscience | 11996942 |
| FITC, eBioscience ™ | | | |
| APC anti-human CD4 Antibody | OKT4 | BioLegend | 317416 |
| Brilliant Violet 421 ™ anti-human CD45 | HI30 | BioLegend | 304032 |
| Antibody | | | |
| APC/Cyanine7 anti-mouse CD45 Antibody | 30-F11 | BioLegend | 103116 |
| PE/Cyanine7 CD152 (CTLA-4) Antibody(BNI3) | BNI3 | BioLegend | 369614 |
| Brilliant Violet 605 ™ anti-human CD25 | BC96 | BioLegend | 302632 |
| Antibody | | | |
| FITC Mouse Anti-Human CD127 | HIL7RM21 | BD Biosciences | 561697 |
| CD279 (PD-1) Monoclonal Antibody (eBioJ105 | eBioJ105 | Life | 25279942 |
| (J105)), PE-Cyanine7, eBioscience ™ | | Technologies | |
| Goat anti-Mouse IgG (H + L) Cross-Adsorbed | | Invitrogen | A21235 |
| Secondary Antibody, Alexa Fluor ™ 647 | | | |
| Biotin-Protein L | | GenScript | M00097 |
| PE Streptavidin | | BioLegend | 405203 |

### In Vivo Mouse Studies

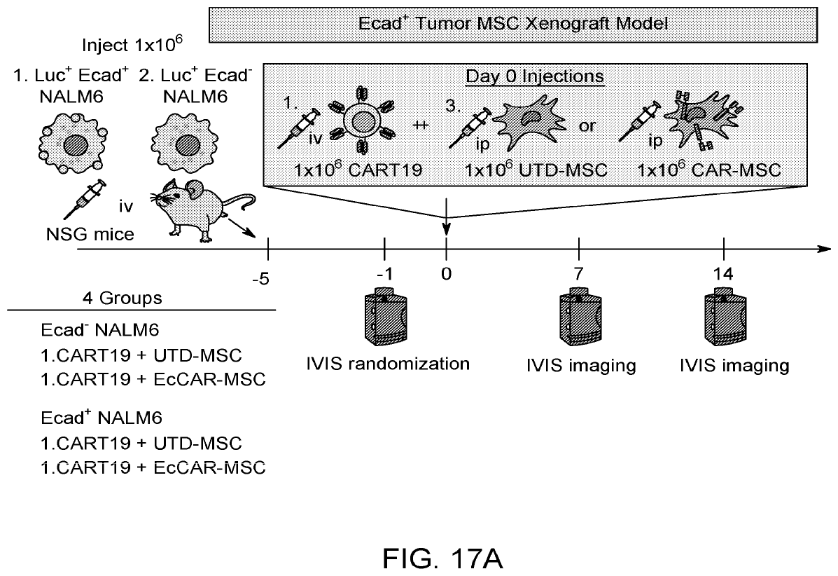

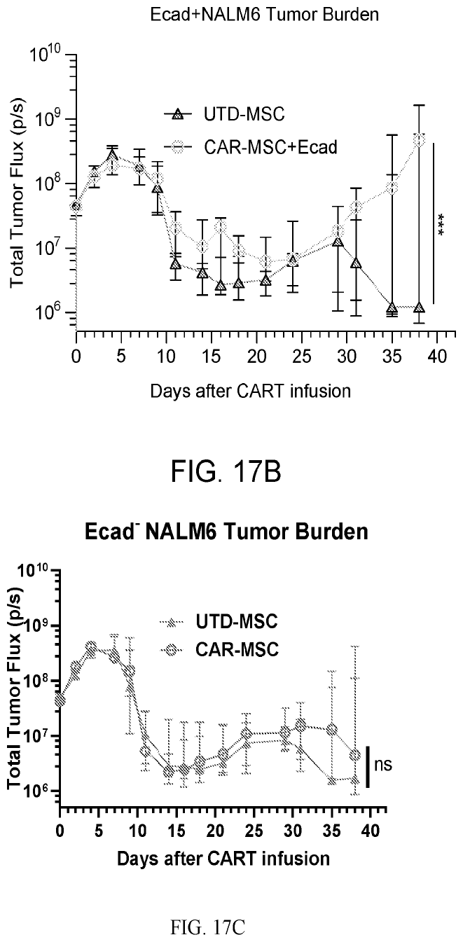

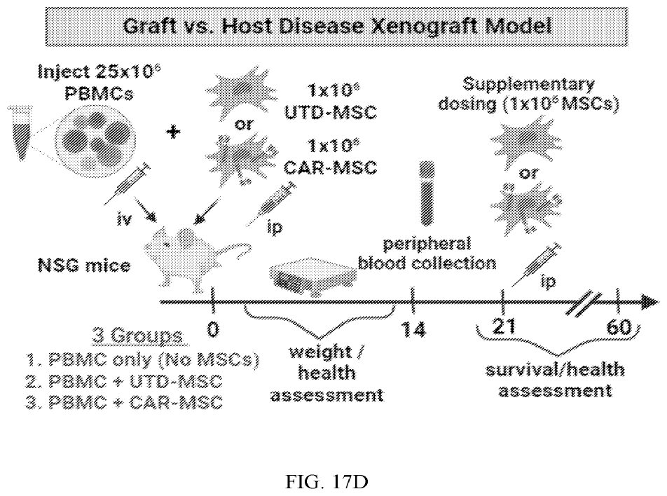

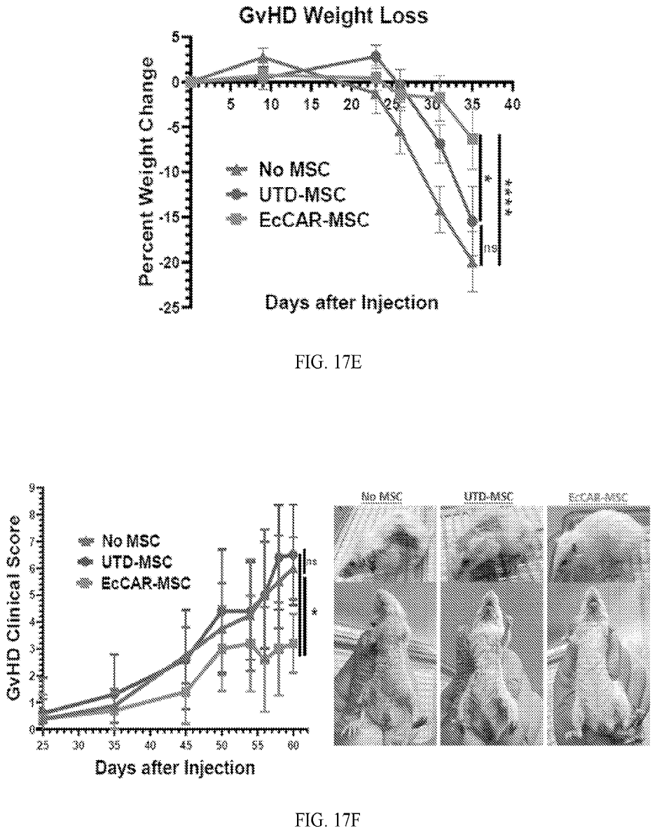

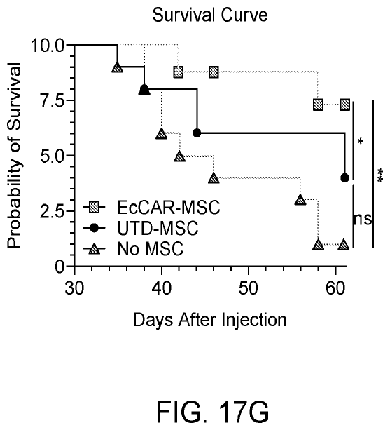

Female and male immunocompromised NOD-SCID-γ^{-/-} (NSG) mice were obtained from Jackson Laboratories at 6-8 weeks old and housed in BSL2+ animal facilities. All cells were injected in 100-200 uL of PBS via syringe through tail vein i.v. or i.p. injection. Mice were imaged with bioluminescent imaging (BLI) using an IVIS® Lumina S5 Imaging System (PerkinElmer, Hopkinton, MA, USA) to confirm engraftment of luciferase⁺CD19⁺ NALM6/JeKo-1 cells in the cancer xenograft model or luciferase⁺ CAR-MSCs in the MSC persistence xenograft model. Imaging was performed 10 minutes after the i.p. injection of 10 μL/g D-luciferin (15 mg/mL, Gold Biotechnology, St. Louis, MO, USA). In Ecad tumor xenograft models, NSG mice were engrafted with Ecad⁺ or Ecad⁻ luciferase⁺ CD19⁺ NALM6 (1×10⁶ cells given i.v.). Engraftment was confirmed by BLI 5 days after injection, and mice were randomized to receive either UTD-MSCs or EcCAR-MSCs (1×10⁶ cells given i.p.) along with CD19-targeted CART cells (CART19) (1×10⁶ cells given i.v. as a strategy to treat the CD19⁺ tumor) (FIG. 17A). Upon efficient CART19 killing of NALM6 tumor burden, mice were rechallenged to again receive Ecad⁺ or Ecad⁻ luciferase⁺CD19⁺ NALM6 (1×10⁶ cells given i.v.) and UTD-MSCs or EcCAR-MSCs (1×10⁶ cells given i.p.). Serial BLI was subsequently performed to assess residual disease and determine antitumor activity of CART19 cells on Ecad⁺ NALM6 and Ecad⁻ NALM6.

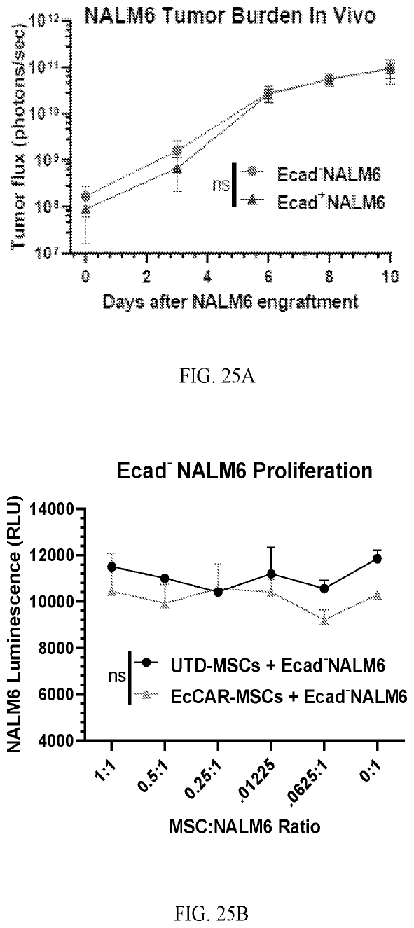

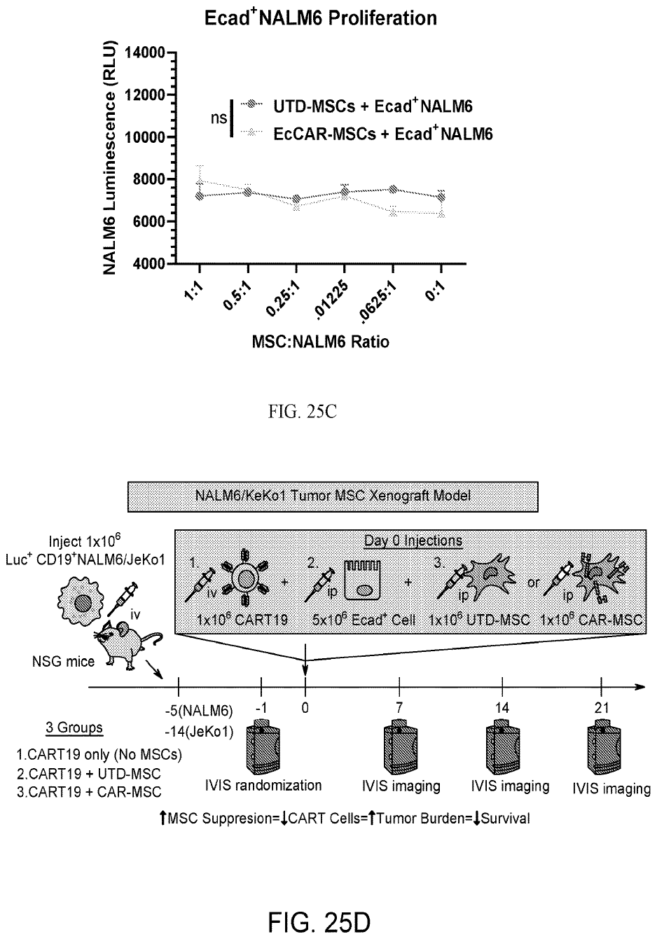

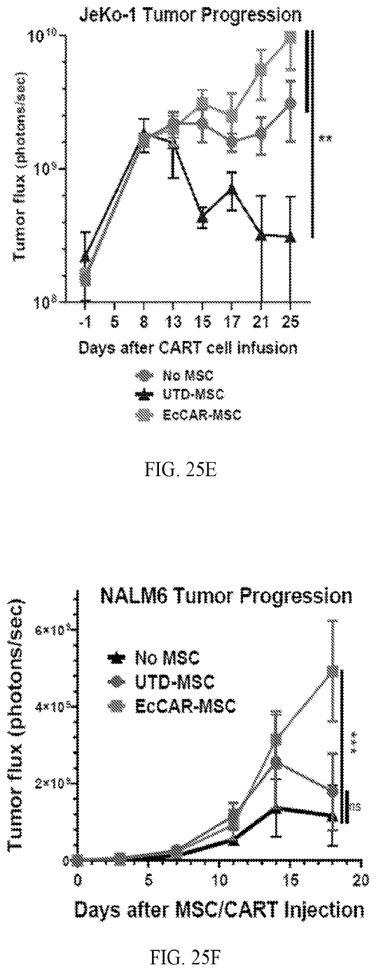

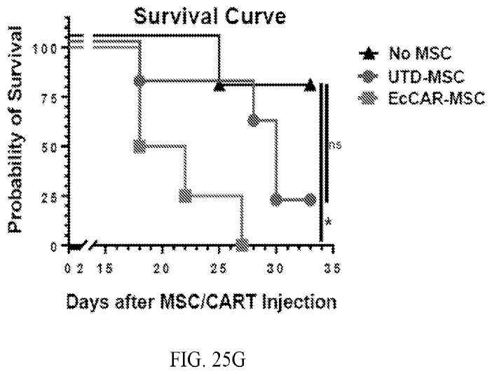

In supplementary tumor models, NSG mice were engrafted with luciferase⁺CD19⁺ JeKo-1 or NALM6 (1×10⁶ cells given i.v.). Engraftment was confirmed by BLI 1-2 weeks or 5 days after injection depending on tumor subtype. All mice then received irradiated Ecad⁺ MCF7 cells (5×10⁶ cells given i.p. to stimulate the EcCAR-MSCs) and CART19 (1×10⁶ cells given i.v. as a strategy to treat the CD19⁻ tumor). Mice were then randomized based on BLI as a measure of tumor burden to receive UTD-MSCs, EcCAR-MSCs, or no additional treatment (FIG. 25B). Serial BLI was subsequently performed to assess residual disease and determine antitumor activity of CART19 cells.

In Graft vs. Host disease (GVHD) models, GVHD was induced in NSG mice with allogeneic human PBMCs (20-30×10⁶ cells i.v.) and treated with UTD-MSCs (1×10⁶ cells i.p. every 3 weeks) or EcCAR-MSCs (1×10⁶ cells i.p. every 3 weeks) (FIGS. 17D-17G). Body weight and clinical GVHD scoring (based on body weight, posture, diarrhea, activity, fur condition, and skin integrity, Table 5) were monitored in all experiments for GVHD progression in each experimental group.

TABLE 5
GVHD clinical scoring system.

| | Weight | | | | Fur | Skin |
|---|---|---|---|---|---|---|
| Grade | Loss | Diarrhea | Posture | Activity | Texture | Integrity |
| 0 | <10% | No | Normal | Normal | Normal | Normal |
| 1 | 10% to | Yes | Hunched | Reduced | Rufflings | Flaked |
| | <25% | | | | | |
| 2 | ≥25% | Yes | Severely | Inactive | Severe | Obviously |
| | | | hunched | | Rufflings | denuded |

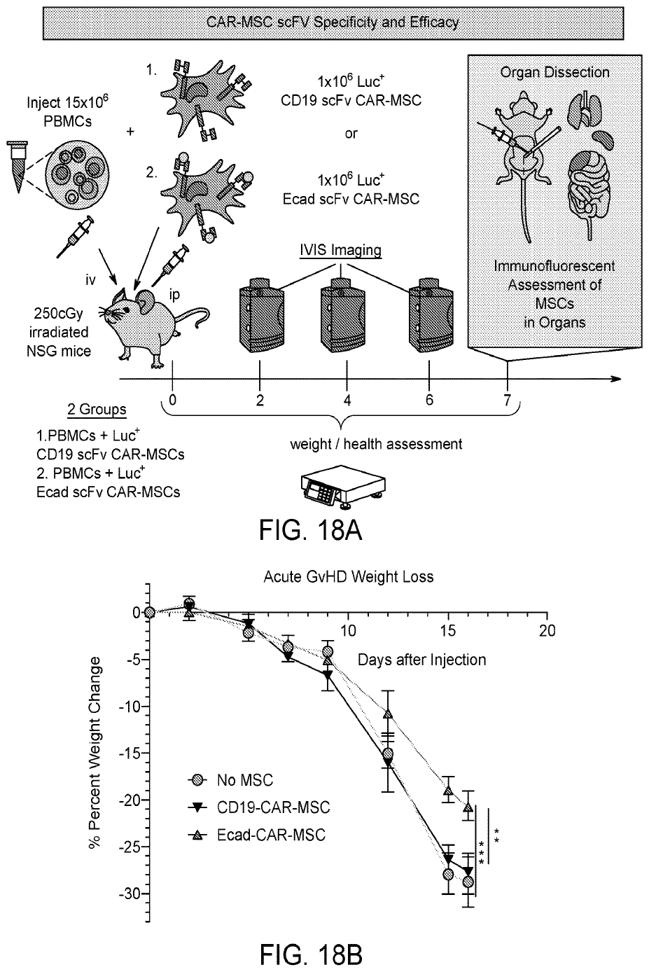

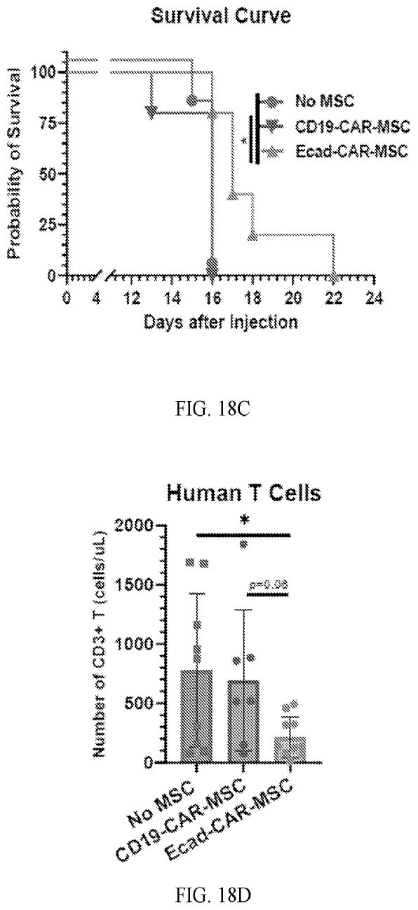

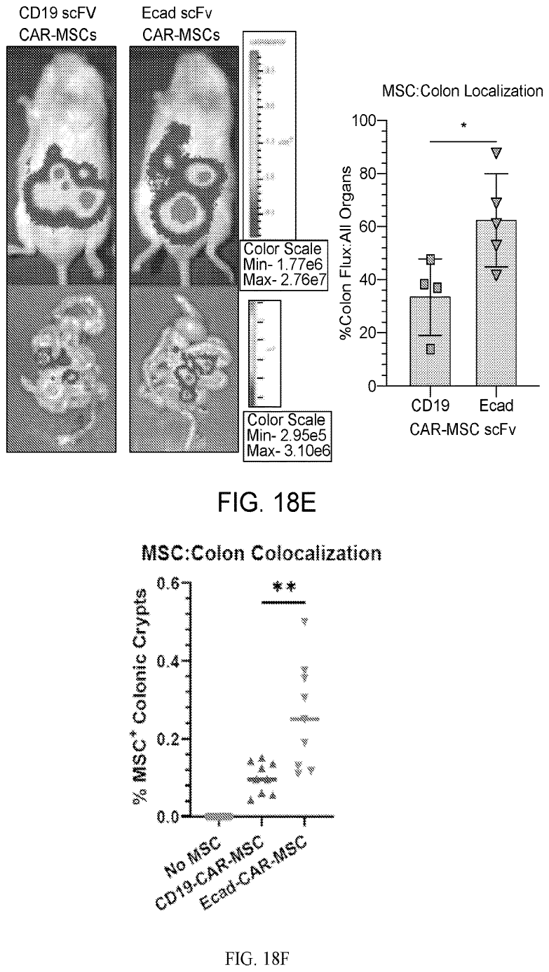

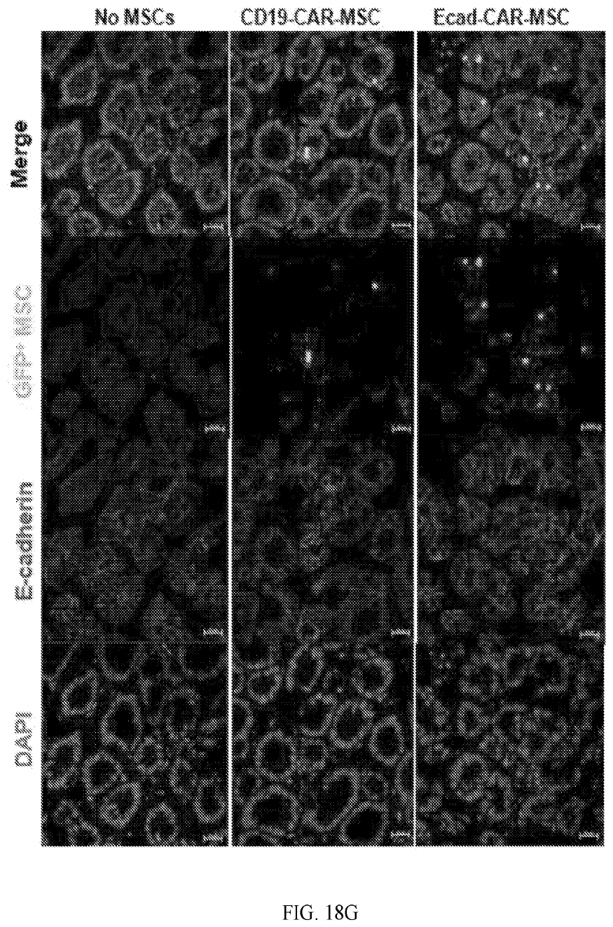

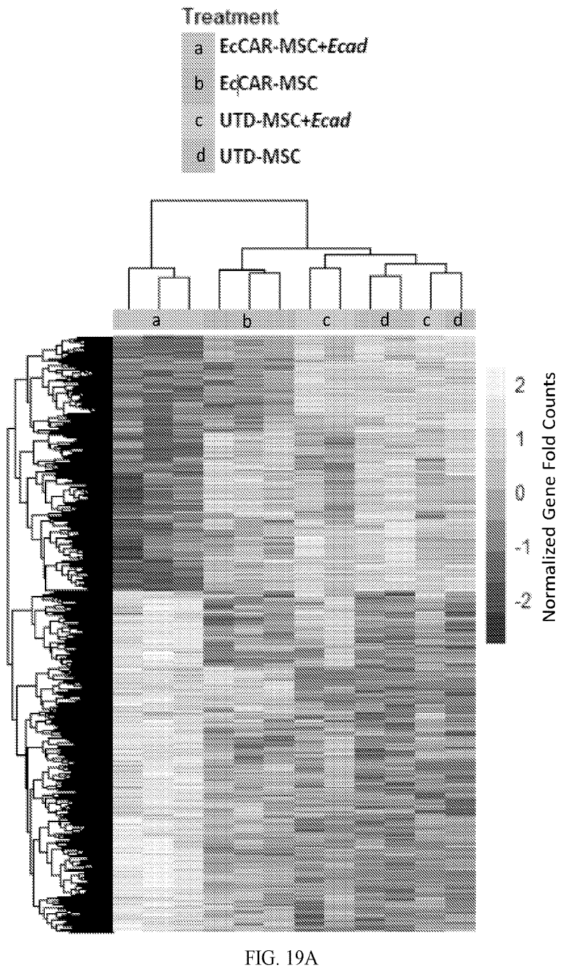

In acute GVHD models, GVHD was more rapidly induced in NSG mice by first irradiating the mice at a dose of 250cGy. Allogeneic human PBMCs (10-15×10⁶ cells i.v.) were then given to irradiated mice, and groups were subsequently randomized by weight to receive UTD- or CAR-MSCs (FIG. 18A). Luciferase⁺ GFP⁺ CD19-CAR-MSCs were utilized as a control for luciferase⁺ GFP⁺ EcCAR-MSC treatment. MSC localization was detected in the organs by BLI and immunofluorescent staining. Allogeneic PBMCs were B-cell depleted using CD19 Pan B Cell Dynabeads (Invitrogen, Waltham, MA, USA) to ensure no human CD19⁺ cells were present in the mice to stimulate CD19CAR-MSCs. Body weight and clinical GVHD scoring were monitored in all experiments for GVHD progression in each experimental group with the same criteria as listed (above).

To assess trafficking and localization of luciferase⁺ anti-Ecad CAR-MSCs and luciferase⁺ anti-CD19 CAR-MSC, mice were also imaged with bioluminescent imaging (BLI) using an IVIS® Lumina S5 Imaging System (PerkinElmer, Hopkinton, MA, USA). One week following MSCs injection in acute GVHD xenograft models, satellite mice were randomly selected for euthanasia and subsequent organ flux assessment. Here, organ imaging was performed 10-20 minutes after a 250 uL i.p. injection of 50 mg/mL D-luciferin (Gold Biotechnology, St. Louis, MO, USA) to detect luciferase⁺ MSCs in the colon, kidneys, liver, lung and spleen relative to whole body BLI detection.

For all in vivo experiments, mouse blood was collected by tail vein bleeding (approximately 100 μL), with 70 μL of blood used for flow cytometry analysis. Red blood cell (RBC) lysis was applied using 1:10 BD FACS Lyse buffer (BD Biosciences, San Jose, CA, USA). Cells were then washed in flow buffer and incubated with their specific antibody mix at room temperature in the dark before flow analysis using CytoFLEX (Beckman Coulter, Chaska, MN, USA) was performed. The remainder of the blood was centrifuged at 4° C., 13,000 rpm for 10 minutes to separate serum for cytokine analysis.

### Immunofluorescent Staining

In the acute GVHD mouse xenograft model, satellite mice were sacrificed, and organs were harvested and preserved, including colon, liver, lungs, and heart 7 days following MSC injection. Organs were fixed in 4% paraformaldehyde for 48 hours and promptly frozen in Tissue-Tek O.C.T Compound (Sakura, Hayward, CA) for frozen sectioning and slide preparation. Tissue slides were next thawed from −80° C. storage and washed in PBS, slides were incubated 0.1% Sudan Black (Sigma-Aldrich, Burlington, MA) solution and solubilized in blocking buffer containing 0.3% Triton-X-100 (Thermo Fisher Scientific, Waltham, MA, USA) and 5% bovine serum albumin (Sigma-Aldrich, Burlington, MA). Antigen retrieval was performed through boiling in unmasking solution for 15 minutes. Tissues were incubated with primary antibodies for 1 hour at room temperature to stain for Ecad (cat. 144725, Cell Signaling Technology, Danvers, MA) and GFP⁺ CAR-MSCs (cat. Ab183734, Abcam, Boston, MA). Secondary antibodies (cat. A11001, cat. A11012, Thermo Fisher Scientific, Waltham, MA, USA) were next incubated over slides for 1 hour at room temperature. Slides were imaged on a Zeiss LSM 980 Confocal Microscope (Zeiss Group, Oberkochen, Germany) and analyzed using ZEN 2.3 Microscopy Software (Zeiss Group, Oberkochen, Germany). MSC localization to Ecad⁺ colonic crypts were compared across EcCAR-MSC,

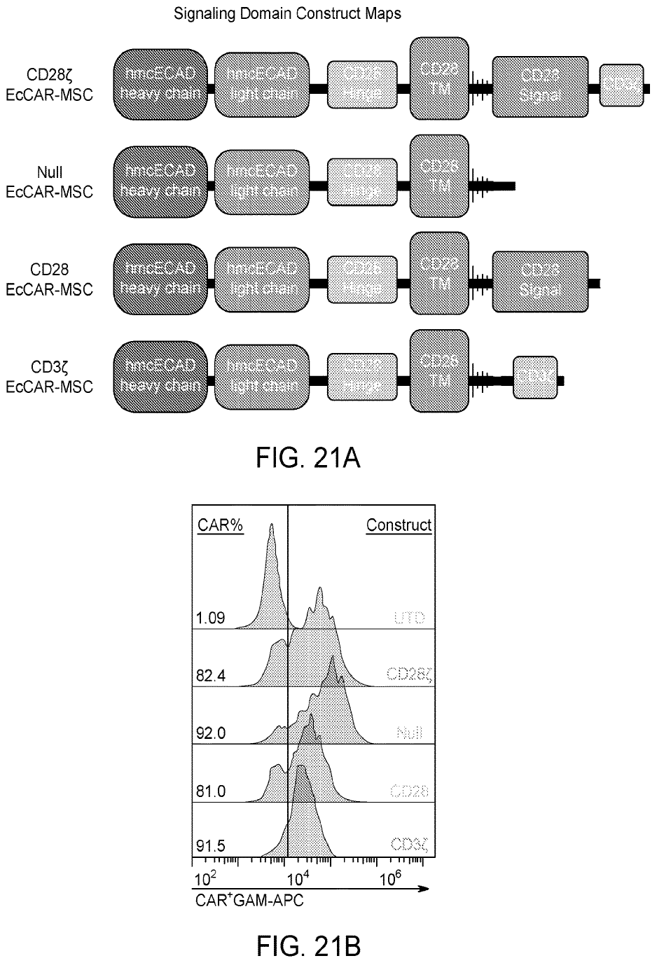

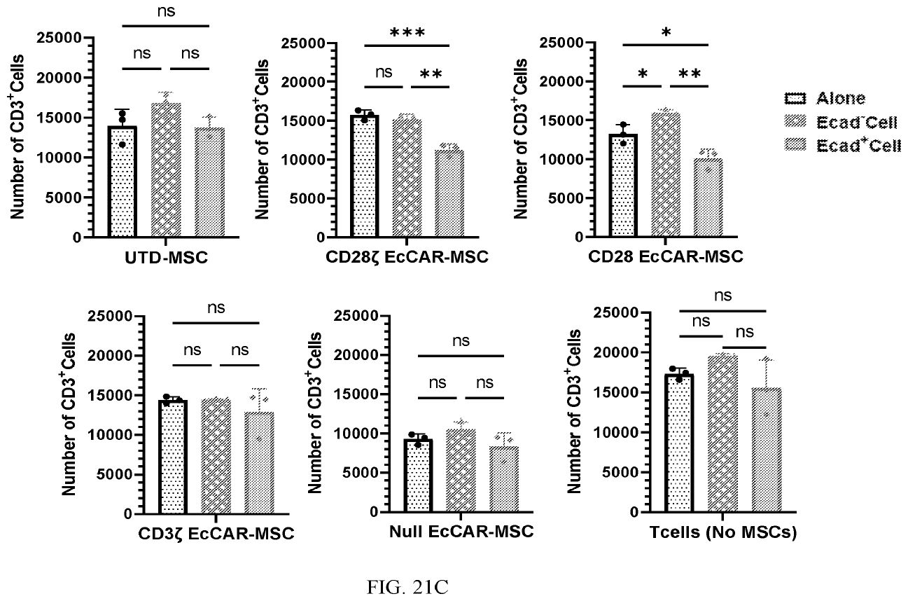

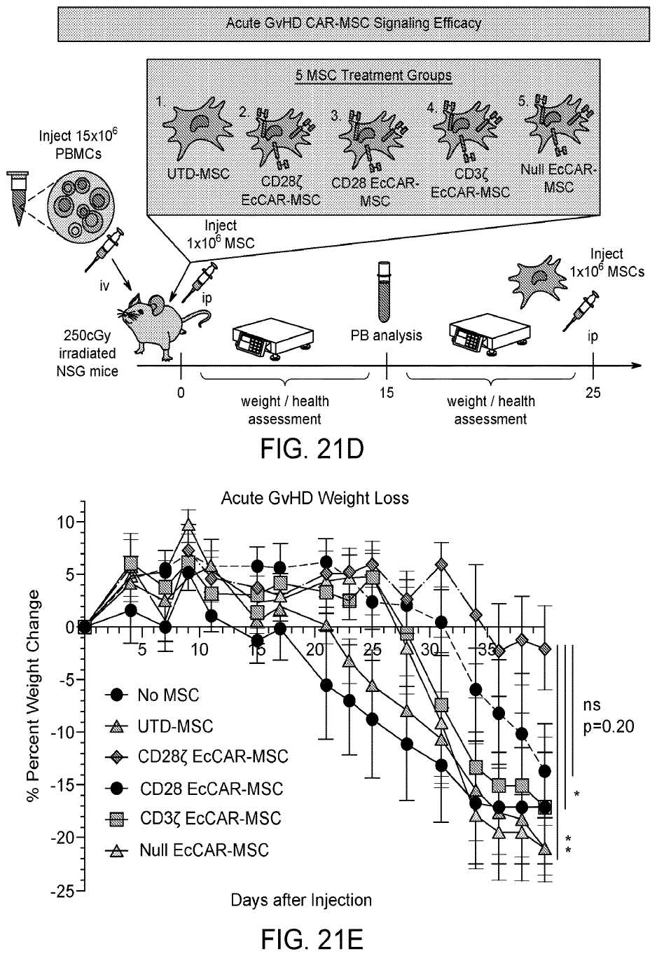

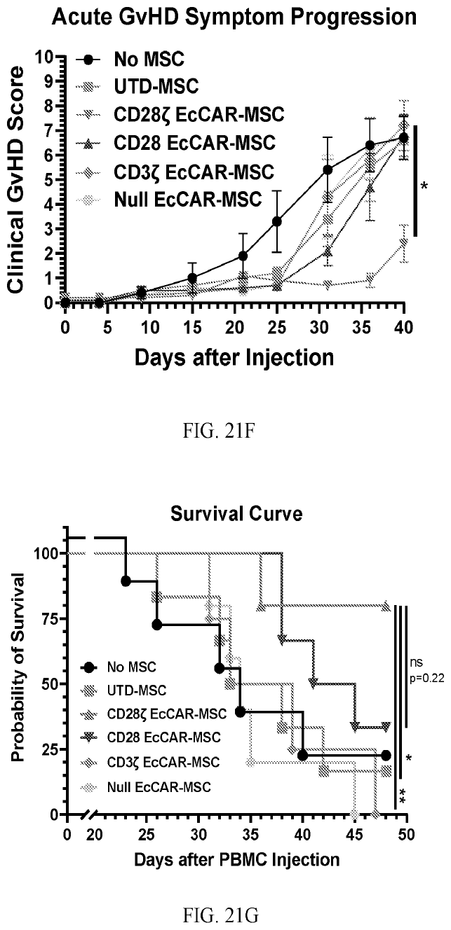

CD19CAR-MSC, and no MSCs (negative control) groups. Localization was calculated by quantification of colonic crypts containing GFP⁺ MSCs over an average of five 40x magnified microscopic fields across each condition (FIGS. 18F-18G and FIGS. 21D and 21F).

### Canine Studies

Ecad-targeted CAR expressed on human MSCs in all experiments was based on a human/mouse/canine cross-reactive scFv. This scFv was generated using phage display, and cross-reactivity with both dog and human Ecad was confirmed (FIG. 23A-23D). EcCAR-MSCs in healthy beagles. Subjects were injected i.p. with EcCAR-MSCs (2×10⁶ cells/kg). Subjects were followed daily, weights were monitored, and serial blood sampling was performed. Colon biopsies were performed on Day 3 and examined by hematoxylin and eosin (H&E) to assess tissue integrity.

### RNA Isolation, Sequencing, and Analysis

EcCAR-MSCs or UTD-MSCs were stimulated accordingly in culture with or without Recombinant Human E-cadherin Fc chimera (BioLegend, San Diego, CA, USA) at 250 ng/ml as a form of CAR specific stimulation for 24 hours. Following in vitro culture, MSCs were detached, and RNA was isolated using a QIAGEN RNeasy Plus Mini Kit (Cat #74134, QIAGEN, Germantown, MD, USA). Bulk RNA sequencing was performed on MSCs from 3 different biological donor replicates in both UTD-MSC and EcCAR-MSC groups to ensure rigor of results.

Total RNA was prepped with a SMARTer stranded total RNA-seq kit v2, Pico input mammalian (Takara, Mountain View, CA, USA). Total RNA (three samples per lane) was sequenced on an Illumina HiSeq 4000 (Illumina, San Diego, CA, USA). Library preparation and sequencing were performed by the Medical Genome Facility Genome Analysis Core (Mayo Clinic, Rochester, MN, USA). Quality check was performed with FastQC v0.11.8 on sample generated fastqc files. Cutadapt v1.18 was used to trim and remove adapter sequences. Output files were confirmed for adaptor removal and quality using FastQC v0.11.8.

Paired end reads from trimmed fastq files were mapped to the latest human reference genome (GRCh38) downloaded from NCBI (ncbi.nlm.nih.gov/). Genome index files were built and aligned with STAR v2.5.4b. Generic expression counts for each gene were generated with HTSeq (Python 3.6.5). Gene counts were normalized (geometric mean) and differential expression analysis was calculated with DESeq2 (R v3.6.1) using adjusted p values≤0.05 as statistical cut offs. Heatmaps were created using pheatmap with PCAs generated with ggplot2 and Prism Graph Pad (La Jolla, CA,). Gene set enrichment analysis for cell phenotypes were performed using Enrichr Cell Augmented Gene set. Activated and inhibited canonical pathways, molecules, and protein-protein interaction networks were generated using Ingenuity Pathway Analysis (QIAGEN, Redwood City, CA, USA) with stringent p value and fold change cut offs (p≤0.01, ±1 fold change) across differential gene expression comparisons.

### Multiplex Cytokine Assays

Cytokine assays were performed on mouse serum samples collected 2 weeks following MSC or control treatment. In vitro cytokine assays were performed on supernatant collected 24 following coculture with MSCs and stimulation source. Debris were removed from serum or supernatant by centrifugation at 10,000×g for 5 minutes. Serum and/or supernatant was diluted 1:2 with serum matrix before plating and following the manufacturer's protocol for Milliplex Human Cytokine/Chemokine MAGNETIC BEAD Premixed 38 Plex Kit (HCYTMAG-60K-PX38, Millipore Sigma, Ontario, Canada). Data were collected using a Luminex (Millipore Sigma, Ontario, Canada) and analyzed with Belysa Immunoassay Curve Fitting Software (Millipore Sigma, Ontario, Canada) and Microsoft Excel (Microsoft, Redmond, WA, USA). Significant findings were determined and reported with Prism Graph Pad (La Jolla, CA, USA).

### Statistical Analysis and Figures

In vitro and in vivo experiments were performed using technical and biological replicates for appropriate statistical analyses. 2-way or 1-way ANOVA were used to evaluate the differences between in vitro EcCAR-MSC and UTD-MSC surface marker expression by % expression and T cell suppression capabilities by absolute T cell count evaluations. ANOVA were also used to determine significant differences between in vivo EcCAR-MSC-treated mice and UTD-MSC-treated mice through IVIS imaging of luciferase⁺ tumors and luciferase⁺ MSCs, weight comparisons, blood composition by flow, and GVHD clinical score comparisons. Kaplan Meier survival analyses with Cox Regression were used to determine significance of differences and adjust for confounders (e.g., sex) in survival outcomes in tumor and GVHD xenograft models. To make multiple comparisons between each individual group in previously described analyses, Tukey's multiple comparisons test supplemented ANOVA analyses. For comparison between two groups, a two tailed unpaired Student's t-test was used in place of ANOVA.

RNA-seq data was processed with DeSeq2 program where raw counts were normalized across the samples (geometric mean), and Benjamini-Hochberg procedure was used for multiple hypothesis correction. All relevant statistically significant comparisons were demarked with asterisks corresponding to levels of significance below p≤0.05 indicating a 95% confidence interval (where ns=p≥0.05, *p≤0.05, **p≤0.01, ***p≤0.001, and ****p≤0.0001). End data points without asterisks represent not significant (ns) differences between groups. Relevant data depict the mean of all data points with either standard deviation (SD) or standard error of the mean (SEM) used to determine error bars. Prism Graph Pad (La Jolla, CA, USA) and Microsoft Excel (Microsoft, Redmond, WA, USA) were used to analyze and create the experimental data figures. Finally, canonical pathway enrichments and molecular networks were generated with significant findings (*padj≤0.05) reported by Ingenuity Pathway Analysis (QIAGEN, Redwood City, CA, USA).

### Results

MSCs are Successfully Transduced with Stable CAR Expression

Using lentiviral vectors augmented with polycation enhancer, efficient transduction of CAR onto MSCs was established. The delivery of CAR-loaded lentivirus was first optimized with protamine sulfate yielding ≥70% transduction efficiency on healthy donor adipose-derived MSCs (FIG. 15A). Following optimization using a standardized CAR vector, alternative CAR constructs were developed for CAR-MSCs immune-mediated disease therapy. The primary CAR-MSC construct was directed to Ecad, a ligand expressed on inflamed intestinal epithelial cells involved in Graph versus Host Disease (GVHD). GVHD results from donor T cells attacking host epithelial tissues in part through the interaction of T cell integrins with E-cadherin (Ecad) expressed on the gastrointestinal tract. To redirect MSCs to Ecad⁺ tissue susceptible to immune attack, several anti-Ecad scFv clones were generated for CAR incorporation by phage display. An optimized scFv sequence (hmcECAD.6 (SEQ ID NO:90), FIGS. 23A-23B) was selected with human, mouse (FIGS. 23C-23D), and canine cross-reactivity to enable preclinical testing in animal models.

To determine if the inclusion of CAR signaling domains would enhance MSC immunosuppressive function without risking cytotoxic T cell conversion, CAR constructs containing CD28ζ signaling domains were tested. hmcECAD.6 was incorporated into a CAR CD28ζ backbone (FIG. 23B) to generate EcCAR-MSCs (FIG. 15B). Stable EcCAR expression was maintained through multiple ex vivo passages with ≥90% transduction efficiency across various MSC donors (FIG. 15C-15D and Table 5).

EcCAR-MSCs Retain their Stemness

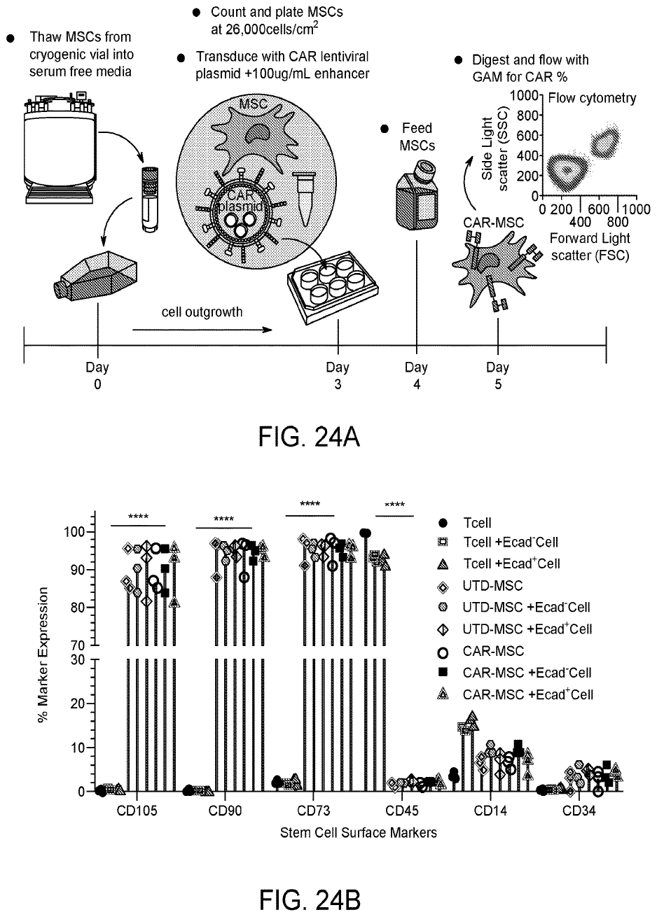

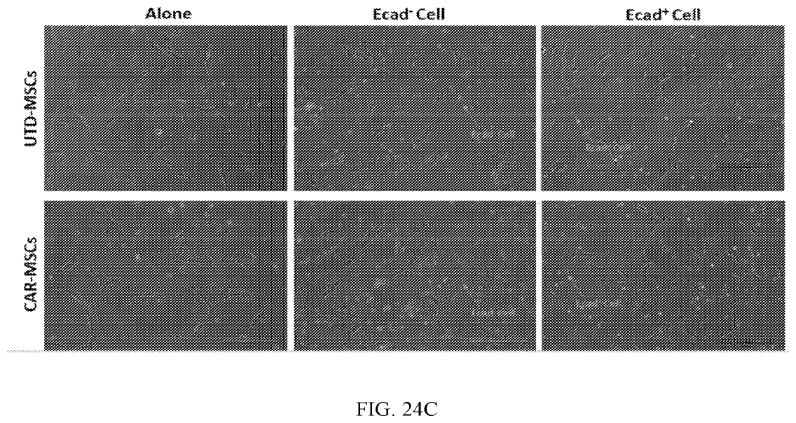

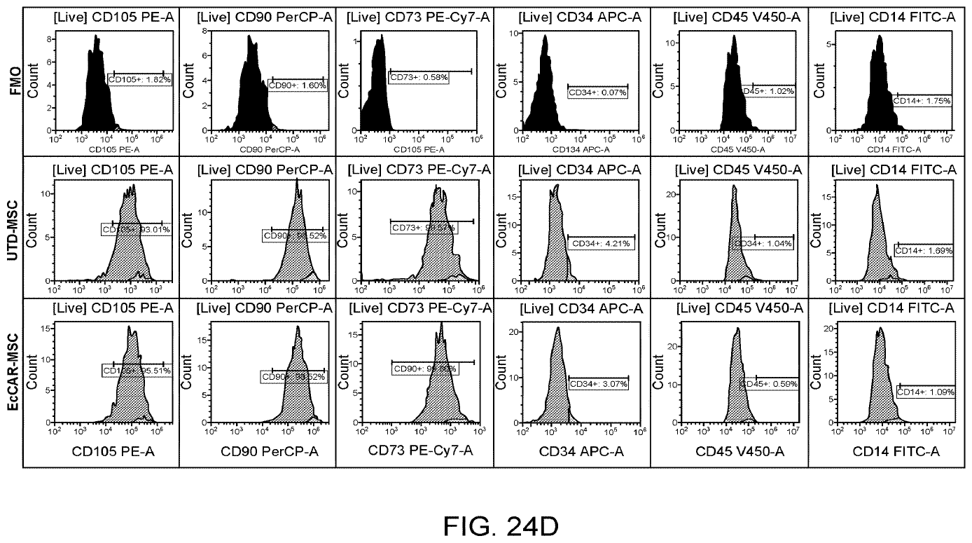

To rule out any unwanted differentiation induced by CAR-MSC engineering, EcCAR-MSC stem phenotype, morphology, and gene expression were determined. Based on MSC stem maintenance criteria set by the International Society for Cell & Gene Therapy, EcCAR-MSCs maintained their stem phenotype and morphology, similar to untransduced (UTD)-MSCs (FIG. 15E and FIGS. 24A-24C). Bulk RNA sequencing (RNAseq) between

EcCAR-MSCs and UTD-MSCs was also used to identify phenotypic gene set enrichments. Adipose-derived stem cell phenotypes were enriched in upregulated EcCAR-MSC genes, while differentiated cell lineages were enriched in downregulated genes (FIG. 15F). These results support maintained stemness following MSC engineering with limited differentiation induced by CAR transduction.

EcCAR-MSCs Exhibit Superior Antigen-Specific Immunosuppression with Maintained Stemness

The ability of CAR-MSCs to suppress T cells was next tested in vitro. Briefly, EcCAR-MSCs or UTD-MSCs (suppressors) were cocultured for 24-hours with activated T cells (effectors), in the presence or absence of Ecad protein as a strategy to stimulate CAR (stimulator). Both soluble Ecad protein and NALM6 cell lines engineered to stably overexpress Ecad (FIG. 16A) were used as sources for CAR stimulation. EcCAR-MSC stimulation by soluble Ecad led to a dose-dependent, superior suppression of T cells as compared to UTD-MSC controls (FIG. 16B). MSC stem phenotypes were notably maintained following stimulation through soluble Ecad (FIG. 16C). Additionally, EcCAR-MSCs stimulated by Ecad⁺ cell line also displayed antigen-specific immunosuppression. Superior T cell suppression was achieved following cocultures of EcCAR-MSCs with Ecad⁺ cells, but not Ecad cells, across multiple MSC donors (FIGS. 16D-16F). Stimulation by Ecad⁺ or Ecad⁻ cell lines also did not affect the stemness properties or morphology of EcCAR-MSCs (FIG. 16G and FIGS. 24B-24C).

### EcCAR-MSCs Suppress the Antitumor Activity of CART19 in Tumor Xenograft Models

Based on these findings in vitro, and to determine if CAR-MSCs suppress CART19 and enhance tumor growth in an antigen-specific context, CAR-MSC immunosuppression was evaluated in vivo through tumor and GVHD xenograft models. In tumor models, the ability of MSCs to suppress the potent anti-tumor activities of CD19-targeted T cells (CART19) was measured (FIG. 17A). To provide antigen-specific stimulation for EcCAR-MSCs, Ecad⁺ NALM6 cells and Ecad⁻ NALM6 cells were used as controls (FIG. 16A). Tumor models were established by intravenous (i.v.) administration of luciferase⁺ Ecad⁺ NALM6 or luciferase⁺ Ecad⁻ NALM6 tumor cells into immunocompromised NOD-SCID-γ^{-/-} (NSG) mice (FIG. 17A). It was determined that the growth of Ecad⁻ NALM6 and Ecad NALM6 in immunocompromised mice without additional treatment was similar (FIG. 25A).

Following engraftment, mice were randomized based on tumor burden as determined by bioluminescent imaging (BLI) to treatment with CART19 plus UTD-MSCs, or CART19 plus EcCAR-MSCs with tumor burdens assessed through biweekly BLI. Results indicated an Ecad dependent activation of EcCAR-MSCs in vivo. Treatment of Ecad⁺ NALM6 (FIG. 17B), but not Ecad⁻ NALM6 (FIG. 17C) xenograft mice with EcCAR-MSCs resulted in dampened antitumor activity, as compared to treatment with UTD-MSCs. EcCAR-MSCs did not directly promote leukemic growth alone in NALM6 cocultures (FIGS. 25B-25C).

It was also found that EcCAR-MSC treatment significantly impairs the antitumor activity and decreases survival in tumor xenograft models when coinjected with irradiated Ecad⁺ MCF7 as an alternative strategy to stimulate EcCAR-MSCs (FIGS. 25D-25G). These results highlight the potent immunosuppressive potential of EcCAR-MSCs in the presence of Ecad antigen in vivo.

### EcCAR-MSCs Improve Therapeutic Outcomes and Display Antigen-Specific Trafficking to Ecad⁺ Target Sites in GVHD Xenograft Models

The therapeutic efficacy of EcCAR-MSCs was tested in GVHD xenograft models. GVHD xenograft models were generated through the i.v. administration of human peripheral blood mononuclear cells (PBMCs) into NSG mice. See, for example, Ehx et al. Front Immunol 9, 1943 (2018). Mice received intraperitoneal (i.p.) doses of PBS control, UTD-MSCs, or EcCAR-MSCs as a form of GVHD treatment (FIG. 17D). GVHD progression was recorded through bodyweight measurements, clinical GVHD symptom scoring (Table 4), and survival outcomes. EcCAR-MSC treatment prevented bodyweight loss (FIG. 17E), ameliorated clinical GVHD symptoms (including fur, skin integrity, posture, and activity) (FIG. 17F), and improved the overall survival (FIG. 17G) as compared to mice receiving UTD-MSCs or no MSC treatment.

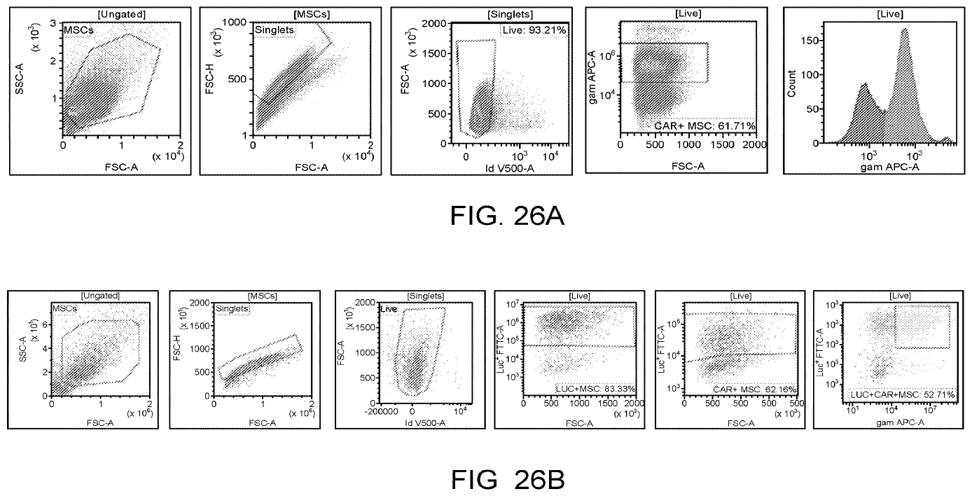

To evaluate the impact of the anti-Ecad scFv contained within EcCAR-MSCs, the efficacy of EcCAR-MSCs was compared to CAR-MSCs containing an scFv of irrelevant specificity to mouse tissue (anti-CD19 CAR-MSC). Specifically, antigen-specific stimulation and trafficking of EcCAR-MSCs to Ecad⁺ epithelia within the colon of GVHD xenograft mice was assessed. GFP⁺ luciferase⁺ EcCAR-MSCs and GFP⁺ luciferase⁺ control anti-CD19 CAR-MSCs were generated (FIGS. 26A-26B). Trafficking and efficacy of the GFP⁺ luciferase⁺ EcCAR-MSCs and GFP⁺ luciferase⁺ control anti-CD19 CAR-MSCs in a GVHD xenograft model (FIG. 18A). In this model, mice were irradiated prior to PBMC and MSC injection to induce a more severe GVHD phenotype (which will be referred to as ‘acute GVHD model’) in which mice develop acute GVHD within one week of PBMC injection. See, for example, Schroeder and DiPersio. Dis. Model Mech. 4, 318-333 (2011). In this acute GVHD model, EcCAR-MSC treatment again led to a significant reduction in weight loss (FIG. 18B) and prolonged survival (FIG. 18C) of mice as compared to control CD19-CAR-MSCs or no MSCs. To elucidate the mechanism underlying increased EcCAR-MSC efficacy in this model, mouse peripheral blood was also collected two weeks following MSC injections and analyzed for human T cells. The administration of EcCAR-MSCs led to an increased suppression of human T cells in the peripheral blood as compared to CD19-CAR-MSC and no MSC treated control mice (FIG. 18D). One week following CAR-MSC injection, satellite mice from these experiments were euthanized, and organs were harvested and analyzed for the presence of CAR-MSCs both by BLI and immunofluorescence. Both BLI and immunofluorescent assessment of mouse colonic tissues following MSC treatment demonstrated superior infiltration of EcCAR-MSCs within Ecad⁺ colon tissues as compared to CD19-CAR-MSCs (FIGS. 18E-18G). Collectively, these results indicate enhanced immunosuppressive efficacy and target tissue trafficking by EcCAR-MSCs in the GVHD models.

### EcCAR-MSC Stimulation Induces an Immunosuppressive Phenotype Through the Activation of Signaling Pathways, Suppressive Cytokines, and Surface Receptors

To evaluate the mechanisms of immunosuppressive enhancement in EcCAR-MSCs, gene expression, cytokine secretion, and surface marker profiles of stimulated EcCAR-MSCs were surveyed. Gene expression profiles were first analyzed through bulk RNAseq across the following conditions: UTD-MSCs (nonstimulated), UTD-MSCs (stimulated with soluble Ecad as a control), EcCAR-MSCs (nonstimulated), and EcCAR-MSCs (stimulated with soluble Ecad to test antigen-specific CAR stimulation). Pathway enrichments were identified between conditions through predictive networks generated in Ingenuity Pathway Analysis (IPA) machine learning software.

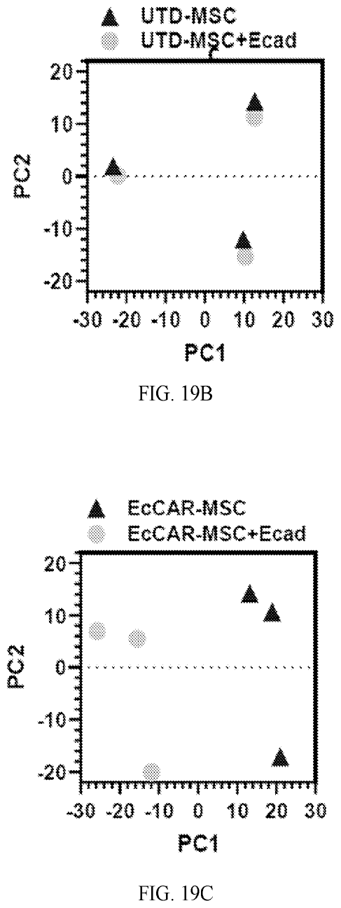

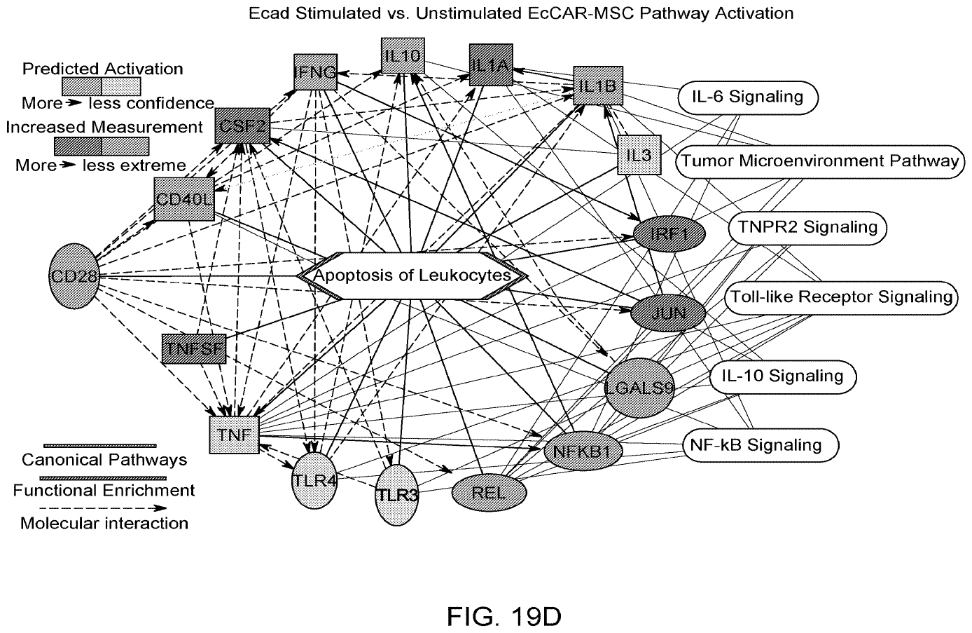

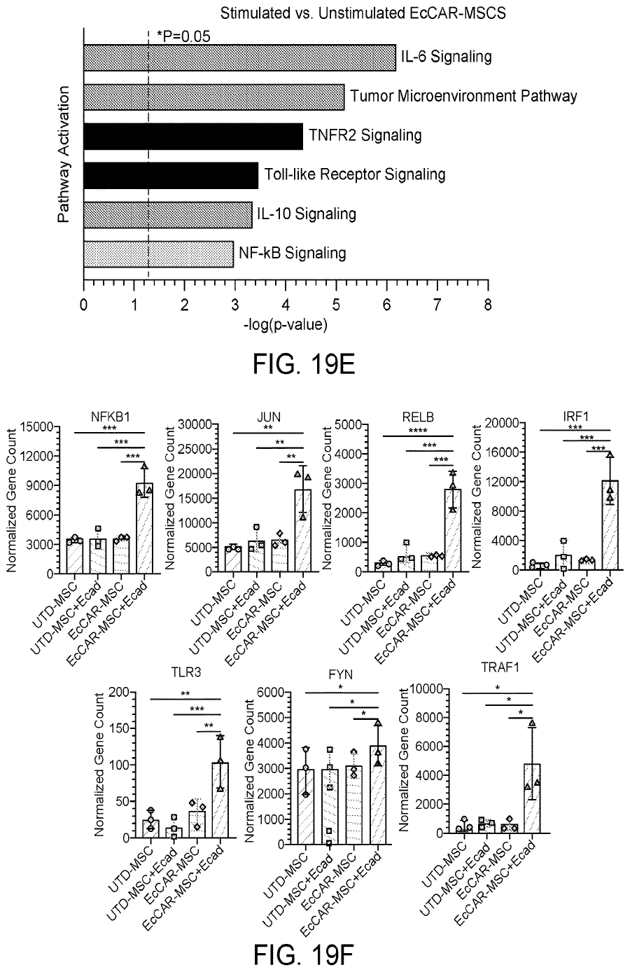

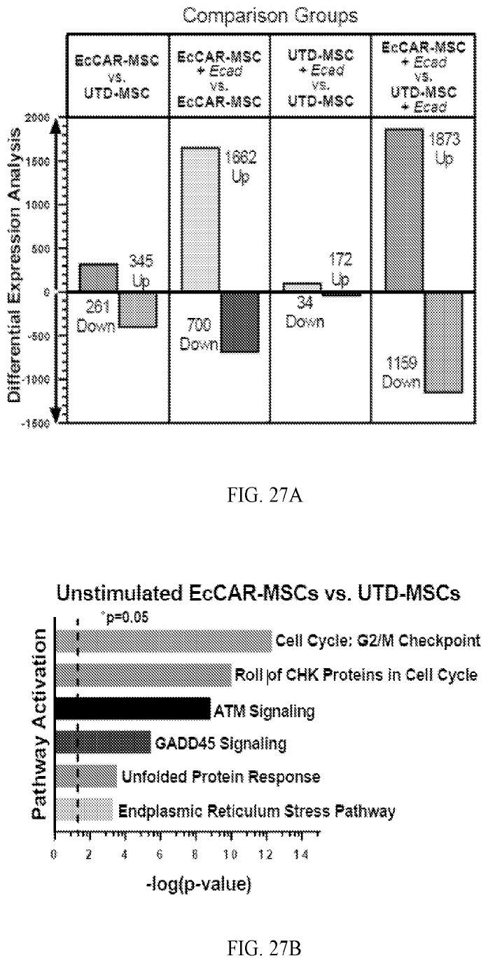

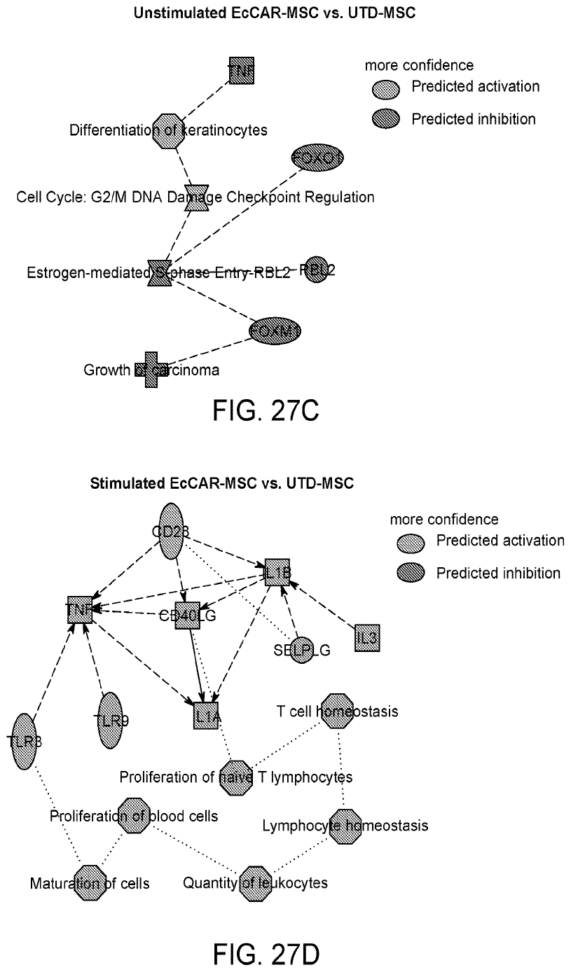

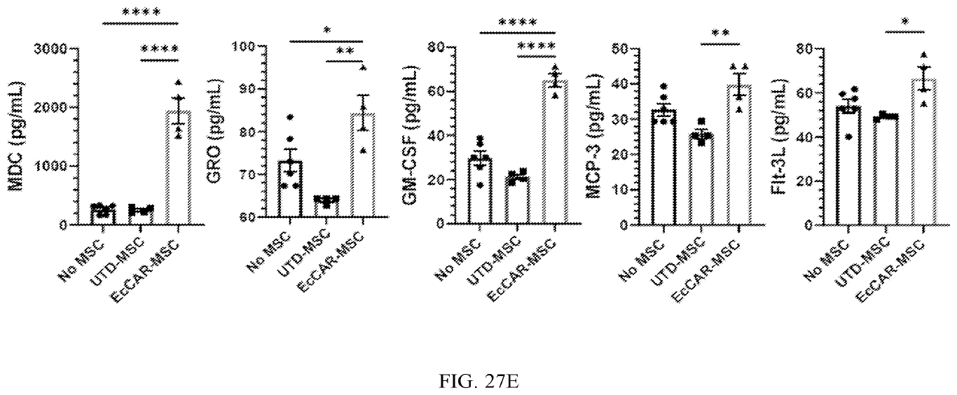

To characterize the impact of CAR transduction on MSCs, nonstimulated EcCAR-MSC vs. UTD-MSC samples were compared. While 331 genes were significantly upregulated between groups, gene enrichments were mostly attributed to cellular stress response pathways that are commonly induced by lentiviral transduction (FIGS. 27A-27C). Notably, these enrichments become insignificant between stimulated EcCAR-MSCs vs. UTD-MSCs, instead revealing predominant homeostatic pathway enrichments (FIG. 27D). Unbiased hierarchical gene clustering across all conditions indicated that the most evident changes to gene expression were driven by antigen-specific stimulation of EcCAR-MSCs(+Ecad) creating a cluster distinguished from all other conditions (FIG. 19A).

To characterize the impact of CAR antigen-specific CD28ζ stimulation, Ecad-stimulated EcCAR-MSC samples were compared to nonstimulated EcCAR-MSC samples. Activation of 1662 upregulated genes was identified (FIG. 27A), supporting that while CAR transduction modifies MSC gene expression, CAR stimulation results in more striking changes to gene activation (FIG. 19A). Principal component analyses of gene expression across Ecad-stimulated and nonstimulated conditions revealed a propensity for UTD-MSC samples to cluster by their derived MSC biological donor (FIG. 19B), while EcCAR-MSC samples instead clustered by Ecad stimulation status (FIG. 19C). Canonical pathways significantly upregulated by CAR stimulation were robustly linked to MSC anti-inflammatory interleukins (IL-6 and IL-10), NFκB, tumor necrosis factor receptor 2 (TNFR2), and toll-like-receptor (TLR) signaling pathways (FIGS. 19D-19E). These pathways each contribute to MSC immunosuppressive function through either cytokine-mediated T cell suppression, the induction of Tregs, or direct receptor interactions.

Gene counts of transcription factors (NFκB1, JUN, RELB, IRF1) and concomitant regulatory factors (TRAF1, TLR3, and FYN) were robustly elevated in Ecad-stimulated EcCAR-MSC samples (FIG. 19F). Altogether, these transcription factors, among others identified following CAR stimulation, were also identified as direct downstream targets of CD28 stimulatory molecule (FIG. 19D), and as regulators of MSC immunosuppressive function. Apoptosis of leukocytes was identified as a significant functional enrichment between all signaling molecules contained in the predictive network for EcCAR-MSC stimulation (FIG. 19D).

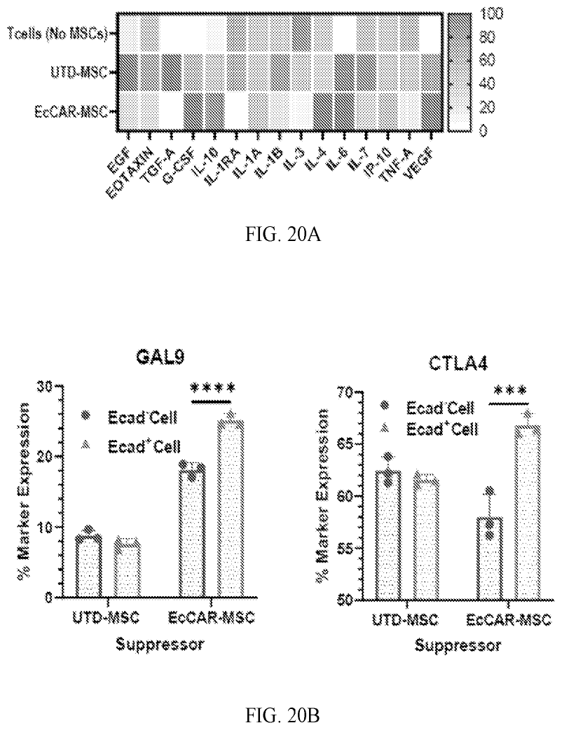

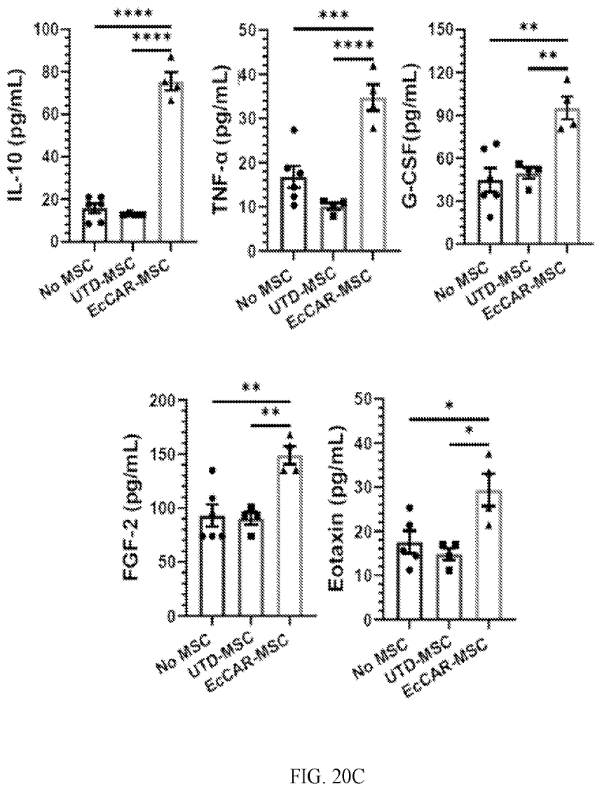

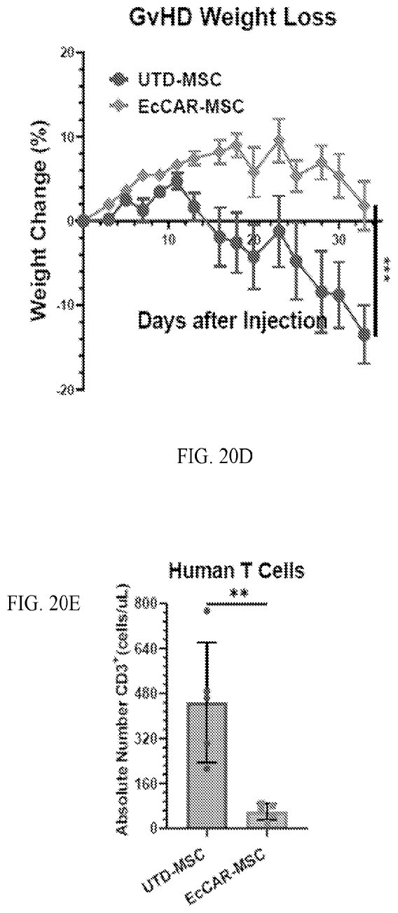

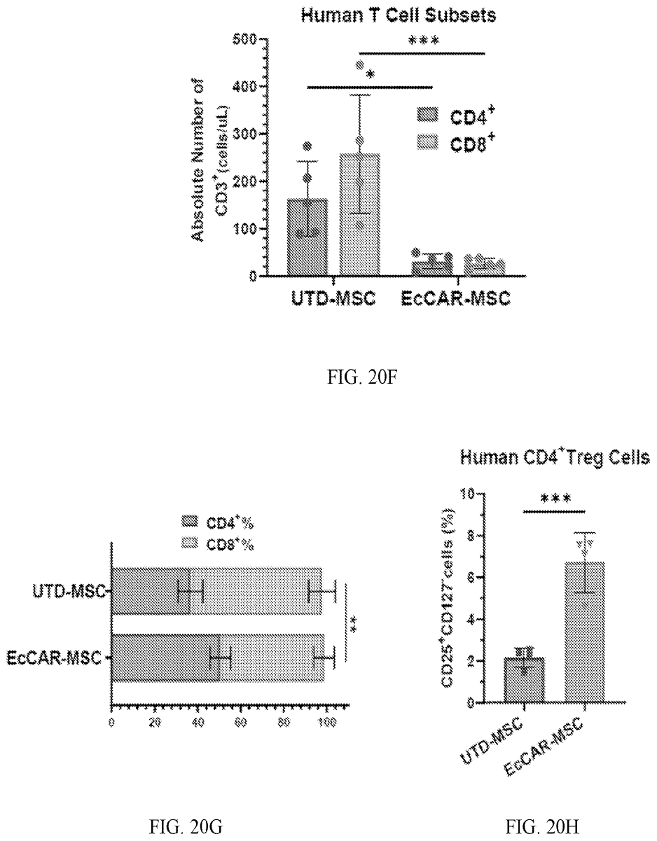

To understand how gene activation in EcCAR-MSCs may translate to functional immunosuppression, the expression of inhibitory receptors and human cytokine production following antigen specific stimulation of EcCAR-MSCs were measured. Antigen-specific stimulation of EcCAR-MSCs (with Ecad⁺ cell lines) resulted in increased secretion of suppressive cytokines in vitro, such as interleukin 10 (IL-10), interleukin 4 (IL-4), granulocyte-colony stimulating factor (G-CSF), and vascular endothelial growth factor (VEGF) (FIG. 20A), as compared to co-culture of EcCAR-MSC with activated T cells in the presence of Ecad cell lines. Additionally, there was an antigen-dependent increase in critical inhibitory surface markers galectin-9 (Gal-9) and cytotoxic T-lymphocyte-associated protein 4 (CTLA4) in stimulated EcCAR-MSCs (FIG. 20B). Human cytokine production was measured in the serum of mice 2 weeks after EcCAR-MSC or UTD-MSC treatment in the tumor model (FIG. 17A). Elevation of IL-10, G-CSF, and eotaxin, among other cytokines in EcCAR-MSC-treated mice were found (FIG. 20C and FIG. 27E).

The peripheral blood was also assessed for human CD³⁺ T cell subtypes in GVHD xenograft mice treated with EcCAR-MSCs compared to UTD-MSCs. In correlation with a significant prevention of weight loss (FIGS. 20D-20H), mice treated with EcCAR-MSCs demonstrated a significantly suppressed absolute numbers of human CD³⁺ T cells circulating in the peripheral blood (FIG. 20E). Although both human CD4⁺ and CD8⁺ T cells were suppressed in EcCAR-MSC-treated mice (FIG. 20F), an increase in the proportion of human CD4⁺ T cells was identified 2 weeks post-treatment in EcCAR-MSC-treated mice (FIG. 20G). Analysis of peripheral blood 4 weeks post-treatment also indicated an enrichment of Tregs (human CD4⁺, CD25⁺, CD127⁻) in EcCAR-MSC-treated mice compared to UTD-MSC-treated mice (FIG. 20H).

Altogether, these findings indicated enhanced T cell suppression after antigen specific stimulation of EcCAR-MSCs, and demonstrated an association with activation of immunosuppressive pathways, increased production of inhibitory cytokines and upregulation of inhibitory receptors, as compared to unstimulated EcCAR-MSCs.

### Incorporation of CD28ζ Signaling Domain in CAR-MSCs is Required for Enhanced Immunosuppressive Activity

To determine which components of the CD28ζ signaling domains are required to drive EcCAR-MSC-mediated enhanced immunosuppression following their antigen specific stimulation, CD28-stimulated EcCAR-MSCs (CD28 EcCAR-MSC), CD3ζ-stimulated EcCAR-MSCs (CD3ζ EcCAR-MSC), and EcCAR-MSCs lacking intracellular domains (Null EcCAR-MSC) were generated and compared to CD28-CD3ζ-stimulated EcCAR-MSCs (CD28ζ EcCAR-MSC) (FIG. 21A-21B). CD28ζ EcCAR-MSCs and CD28 EcCAR-MSCs showed antigen-specific suppression of activated T cells, while CD3ζ EcCAR-MSCs, Null EcCAR-MSCs, and UTD-MSCs did not (FIG. 21C), indicating a role for CD28 signaling in CAR-MSC immunosuppressive properties. The impact of CAR signaling domain incorporation was also tested in the acute GVHD xenograft model (FIG. 21D). In this model, treatment with CD28ζ EcCAR-MSCs resulted in the prevention of weight loss (FIG. 21E), the lowest clinical GVHD symptom severity score (FIG. 17F), and the longest overall survival (FIG. 17G) when compared to mice treated with CD28, CD3ζ, or null signaling domain EcCAR-MSCs. CD28 EcCAR-MSCs treatment also resulted in ameliorated weight loss and overall survival that was numerically, but not statistically, inferior to CD28ζ EcCAR-MSC. These data suggest that while CD28 alone confers immunosuppressive capabilities in EcCAR-MSCs, CD28ζ incorporated in EcCAR-MSCs results in enhanced immunosuppressive treatment outcomes.

### EcCAR-MSCs are Safe in Animal Models

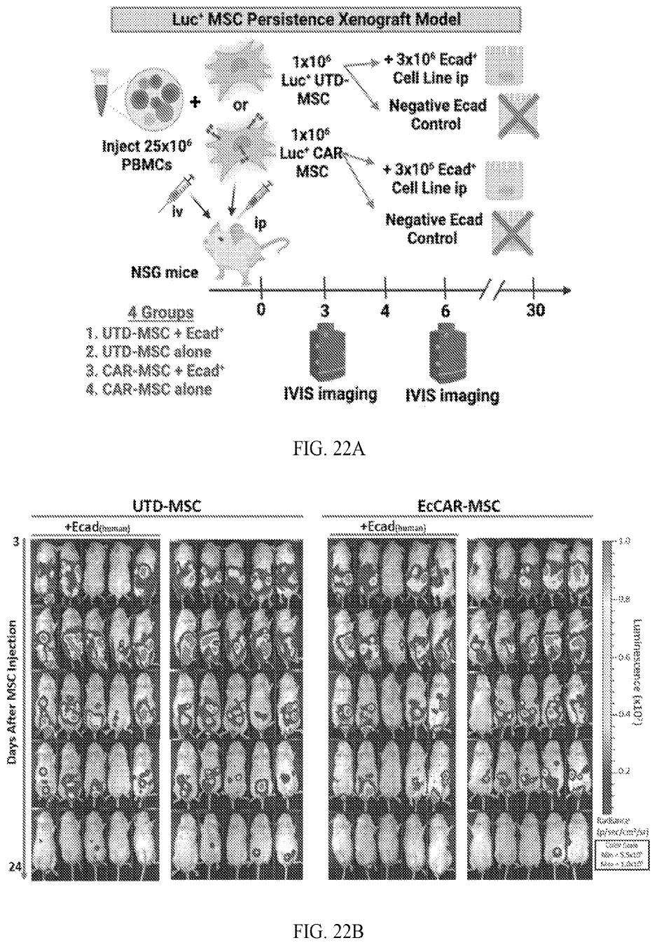

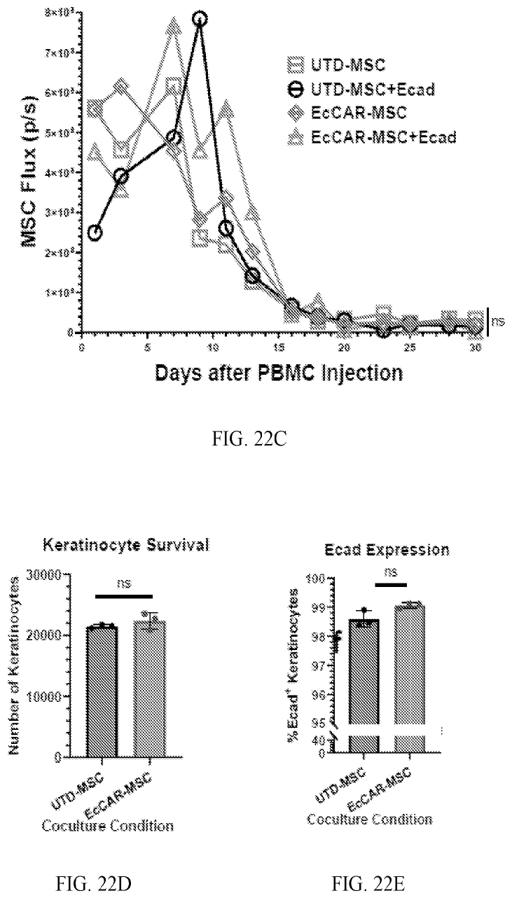

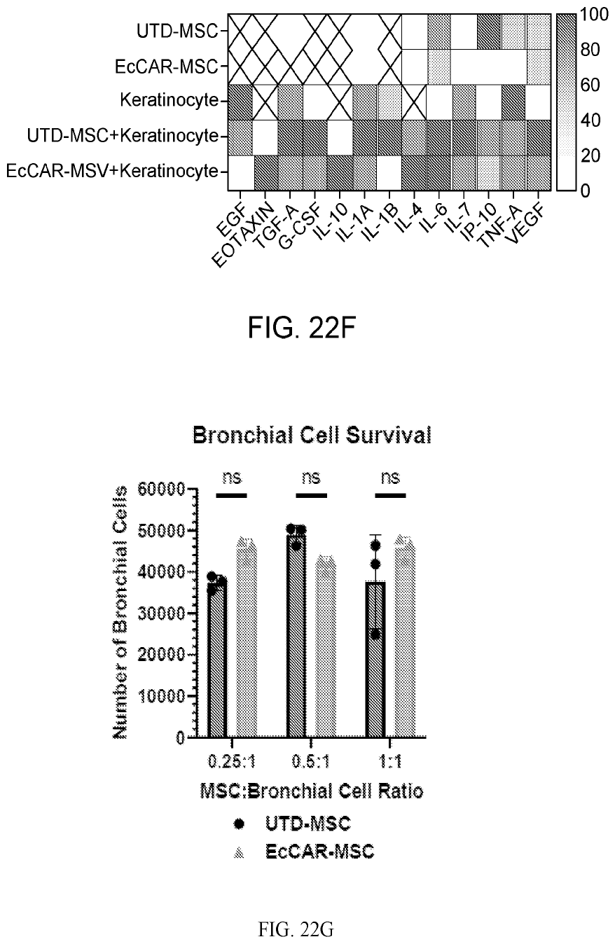

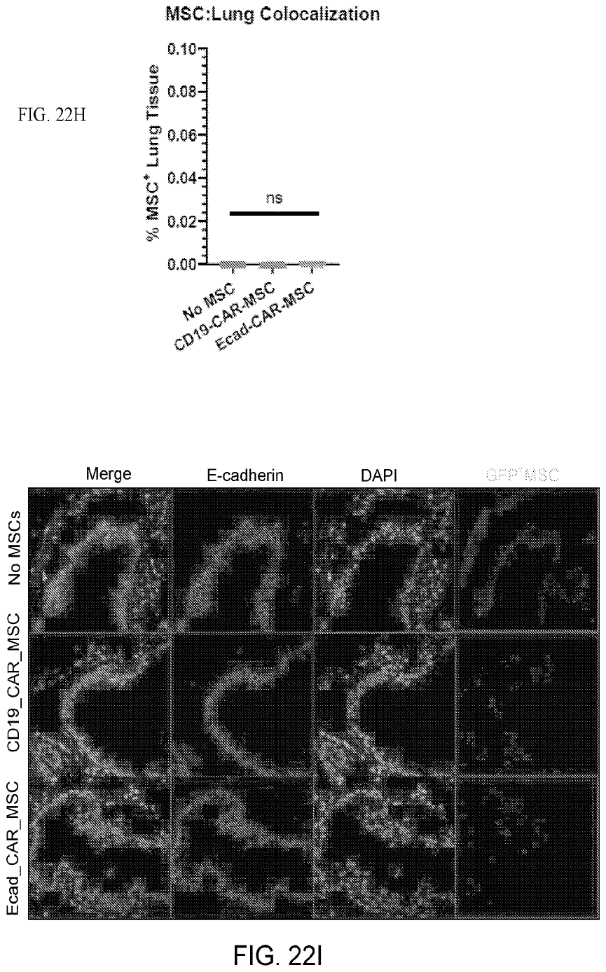

Following functional interrogation, studies were performed to ensure allogeneic EcCAR-MSC safety. First, EcCAR-MSC expansion and clearance was defined in NSG mice, using luciferase⁺ EcCAR-MSCs and UTD-MSCs (FIG. 22A and FIG. 26A). MSC persistence was monitored in mice with or without irradiated human Ecad⁺ MCF7 cells to understand the impact of antigen-specific stimulation on MSC clearance. Mice were treated with 1) luciferase⁺ UTD-MSCs alone, 2) luciferase⁺ UTD-MSCs plus human Ecad⁺ cells, 3) luciferase⁺ EcCAR-MSCs alone, or 4) luciferase⁺ EcCAR-MSCs plus human Ecad⁺ cells (FIG. 22A). Serial BLIs were performed every 2-3 days for MSC quantification. These studies revealed no significant difference between the clearance time (~30 days) of UTD-MSCs and EcCAR-MSCs with or without additional target antigen stimulation, further supporting the safety profile of EcCAR-MSCs (FIGS. 22B-22C).

Then, the toxicity of EcCAR-MSCs on normal epithelial tissues expressing Ecad was determined. First, the effect of EcCAR-MSCs on Ecad⁺ keratinocytes derived from healthy donors was determined. Co-cultures of EcCAR-MSCs with keratinocytes resulted in no significant differences in survival of keratinocytes as compared to co-cultures with UTD-MSCs (FIG. 22D). These cocultures also did not appear to significantly impact expression of Ecad on keratinocyte target cells (FIG. 22E). Evaluation of coculture supernatants indicate antigen specific stimulation of EcCAR-MSC as evident by upregulation in cytokines including IL-10, IL-4, and eotaxin (FIG. 22F).

Second, the toxicity of EcCAR-MSCs on Ecad⁺ bronchial cells was determined. Cocultures of Ecad⁺ bronchial cells with EcCAR-MSCs had no apparent impact on bronchial cell survival as compared to cocultures with UTD-MSCs (FIG. 22G). Additionally, no trafficking of EcCAR-MSCs to the lungs was observed as determined by immunofluorescence assessment of lungs harvested 1 week following EcCAR-MSC treatment in the GVHD xenograft model (FIGS. 22H-221).

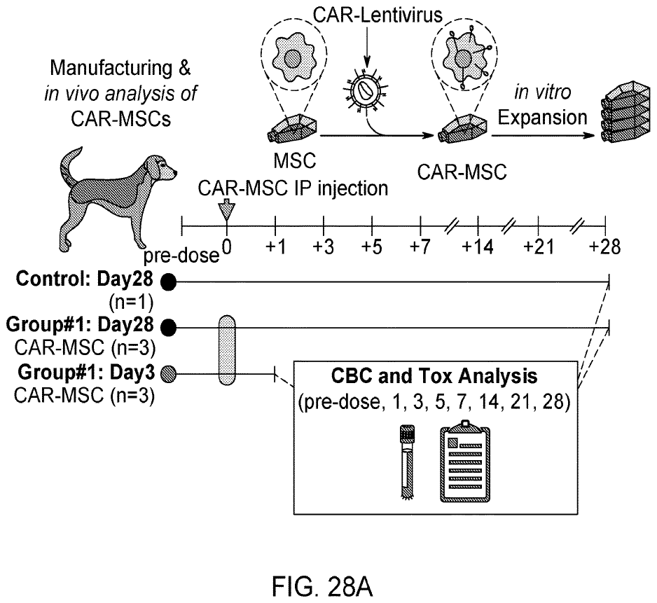

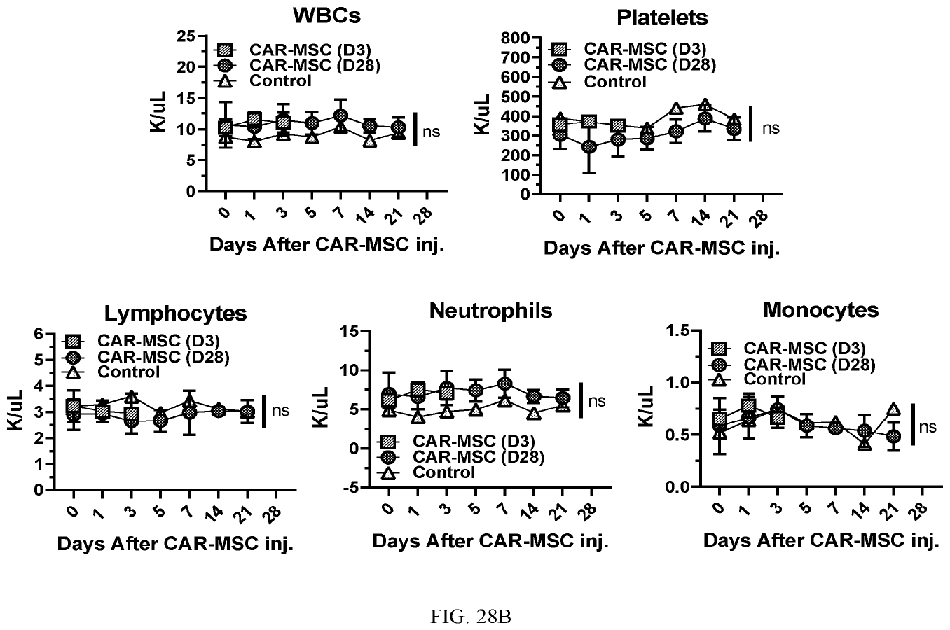

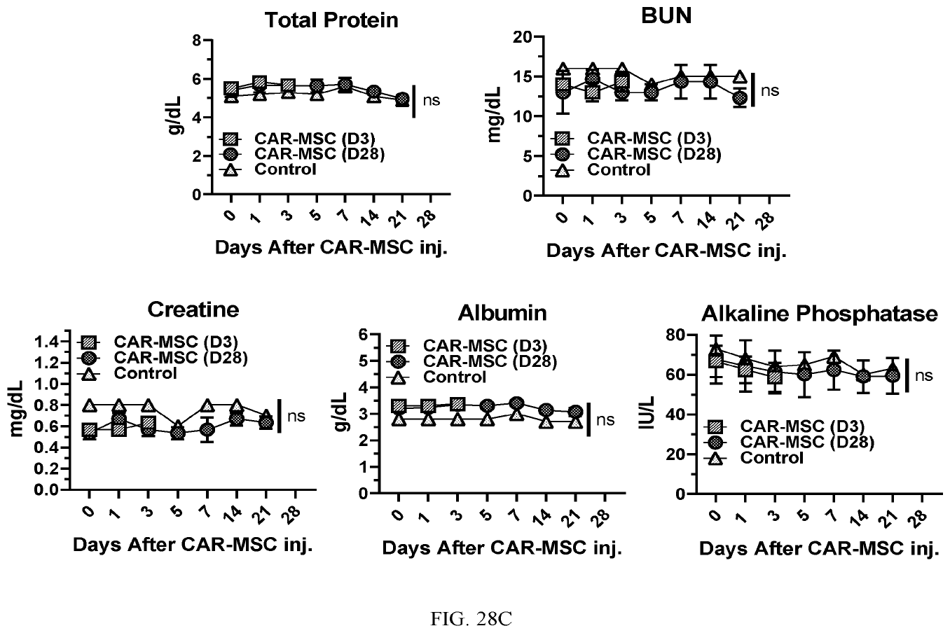

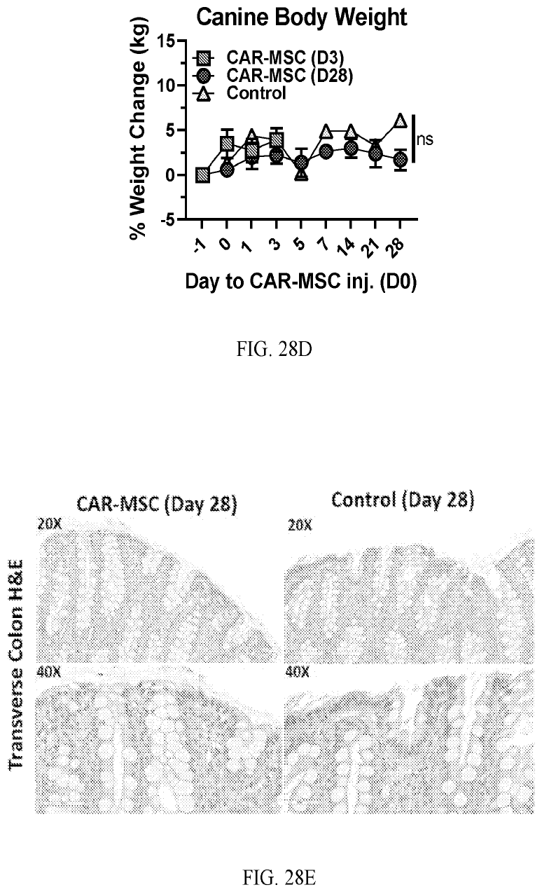

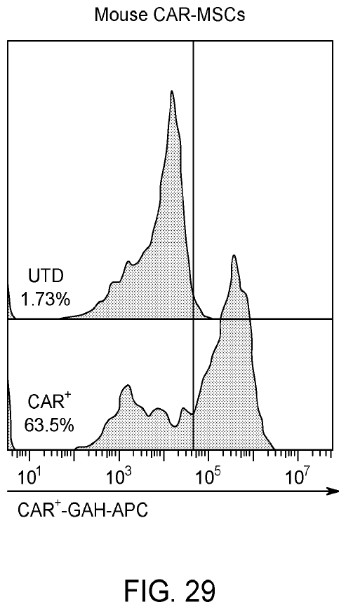

Canine models were used to assess if any toxicities were associated with administration of human EcCAR-MSCs containing canine cross-reactive scFv (FIG. 23A). Healthy canines received EcCAR-MSCs by i.p. injection and were monitored for safety outcomes and tissue integrity for 28 days. (FIG. 28A). Toxicity was assessed by serial measurements of complete blood counts, liver, and kidney functions. EcCAR-MSCs were not associated with hematopoietic toxicity (FIG. 28B), organ toxicity (FIG. 28C), weight loss (FIG. 28D), or tissue damage (FIG. 28E), indicating a strong safety profile and lack of toxicity induced by EcCAR-MSC administration.

Together, these results demonstrate that CAR-MSC generation provides antigen-specific immunosuppressive activation designed to augment MSC therapy safely.

### Example 5: Treating GVHD

Ecad-CAR-MSCs engineered to express one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides are administered to a human identified as having or as being at risk of developing GVHD. The CAR-MSCs engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides are administered using intravenous injection. After the administration of the one or more CAR-MSCs engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides, the number of activated T cells within the human is reduced. After the administration of the one or more CAR-MSCs engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides, one or more symptoms of GVHD within the human are reduced.

### Example 6: Treating GVHD

MSCs engineered to express a CAR targeting an epithelial specific antigen and engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides are administered to a human identified as having or as being at risk of developing GVHD. The MSC engineered to express a CAR targeting an epithelial specific antigen and engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides are administered using intravenous injection. After the administration of the one or more MSC engineered to express a CAR targeting an epithelial specific antigen and engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides, the number of activated T cells within the human is reduced. After the administration of the one or more MSC engineered to express a CAR targeting an epithelial specific antigen and engineered to express an elevated level of one or more polypeptides selected from NFκB1 polypeptides, JUN polypeptides, RELB polypeptides, IRF1 polypeptides, TNFα polypeptides, IL-10 polypeptides, FGF-2 polypeptides, PD-1, polypeptides, G-CSF polypeptides, GM-CSF polypeptides, eotaxin polypeptides, Gal-9 polypeptides, PD-1 polypeptides, TIM-3 polypeptides, CXCR3 polypeptides, and CXCR4 polypeptides, one or more symptoms of GVHD within the human are reduced.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A mesenchymal stromal cell (MSC) comprising:
(1) exogenous nucleic acid encoding a chimeric antigen receptor (CAR) targeting an epithelial-specific antigen, wherein said MSC expresses said CAR, and
(2a) an elevated level of a polypeptide selected from the group consisting of a nuclear factor kappa B subunit 1 (NFκB1) polypeptide, a Jun proto-oncogene (JUN) polypeptide, a transcription factor RelB (RELB) polypeptide, an interferon regulatory factor 1 (IRF1) polypeptide, a tumor necrosis factor (TNF) a polypeptide, an interleukin (IL)-10 polypeptide, a fibroblast growth factor (FGF)-2 polypeptide, a granulocyte colony stimulating factor (G-CSF) polypeptide, a granulocyte-macrophage colony-stimulating factor (GM-CSF) polypeptide, an eotaxin polypeptide, a galectin 9 (Gal-9) polypeptide, a programmed cell death protein 1 (PD-1) polypeptide, a T-cell immunoglobin mucin-3 (TIM-3) polypeptide, a CXC chemokine receptor (CXCR) 3 polypeptide, and a CXCR4 polypeptide, or
(2b) an elevated level of a polypeptide selected from the group consisting of a NFκB1 polypeptide, a JUN polypeptide, a RELB polypeptide, an IRF1 polypeptide, a TNFα polypeptide, a cytotoxic T-lymphocyte-associated-protein 4 (CTLA4), a toll-like receptor 3 (TLR3), a toll-like receptor 4 (TLR4), a toll-like receptor 9 (TLR9), a tumor necrosis factor receptor 2 (TNFR2) polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, and a CXCR4 polypeptide.

2. The MSC of claim 1, wherein said MSC is a human MSC.

3. The MSC of claim 1, wherein said MSC is an adipose derived-MSC.

4. The MSC of claim 1, wherein said epithelial-specific antigen is E-cadherin (Ecad).

5. The MSC of claim 1, wherein said MSC comprises said (2a).

6. The MSC of claim 5, wherein said MSC comprises exogenous nucleic acid encoding said polypeptide of said (2a), wherein said MSC expresses said polypeptide of said (2a).

7-9. (canceled)

10. The MSC of claim 1, wherein said MSC comprises said (2b).

11. The MSC of claim 10, wherein said MSC comprises exogenous nucleic acid encoding said polypeptide of said (2b), wherein said MSC expresses said polypeptide of said (2b).

12-24. (canceled)

25. A composition comprising a MSC of comprising:
(1) exogenous nucleic acid encoding a CAR targeting an epithelial-specific antigen, wherein said MSC expresses said CAR, and
(2a) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR 3 polypeptide, and a CXCR4 polypeptide, or
(2b) an elevated level of a polypeptide selected from the group consisting of a NFκB1 polypeptide, a JUN polypeptide, a RELB polypeptide, an IRF1 polypeptide, a TNFα polypeptide, a CTLA4, a TLR3, a TLR4, a TLR9, a TNFR2 polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, and a CXCR4 polypeptide.

26. A method for (a) treating a mammal having graft versus host disease (GVHD), (b) suppressing an immune response within a mammal, or (c) reducing the number of activated T cells within a mammal, wherein said method comprises administering to said mammal a MSC or a composition comprising said MSC, wherein said MSC comprises:
(1) exogenous nucleic acid encoding a CAR targeting an epithelial-specific antigen, wherein said MSC expresses said CAR, and
(2a) an elevated level of a polypeptide selected from the group consisting of a NFκB1 polypeptide, a JUN polypeptide, a RELB polypeptide, an IRF1 polypeptide, a TNFα polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR 3 polypeptide, and a CXCR4 polypeptide, or
(2b) an elevated level of a polypeptide selected from the group consisting of a NFκB1 polypeptide, a JUN polypeptide, a RELB polypeptide, an IRF1 polypeptide, a TNFα polypeptide, a CTLA4, a TLR3, a TLR4, a TLR9, a TNFR2 polypeptide, an IL-10 polypeptide, a FGF-2 polypeptide, a G-CSF polypeptide, a GM-CSF polypeptide, an eotaxin polypeptide, a Gal-9 polypeptide, a PD-1 polypeptide, a TIM-3 polypeptide, a CXCR3 polypeptide, and a CXCR4 polypeptide.

27. The method of claim 26, wherein said mammal is a human.

28. The method of claim 31, wherein a symptom of said GVHD is reduced at least 10 percent.

29. The method of claim 31, wherein a number of regulatory T cells (Tregs) within the mammal is increased at least 10 percent.

30. The method of claim 26, wherein said method comprises is suppressing said immune response within said mammal.

31. The method of claim 26, wherein said method comprises treating said mammal having GVHD.

32. The method of claim 30, wherein a number of activated T cells within the mammal is reduced at least 10 percent.

33. The method of claim 30, wherein a number of Tregs within the mammal is increased at least 10 percent.

34. The method of claim 26, wherein said method comprises reducing the number of activated T cells within said mammal.

35. (canceled)

36. The method of claim 34, wherein the number of activated T cells within the mammal is reduced at least 10 percent.

37. The method of claim 34, wherein a number of Tregs within the mammal is increased at least 10 percent.

38-70. (canceled)


SEQUENCE LISTING (120 sequences)

SEQ ID NO: 1 - protein, 128 aa, synthetic construct
QVQLQESGPGLVKPSETLSLTCTVSGGSVSSYYWSWIRQPPGKGLEWIGHIYYSGNTNYNPSLKSRVTISVDTSKNQFSLKLSSVTAADTAVYFCARDRWNYYDSSPGYYYYYGMDVWGQGTTVTVSS

SEQ ID NO: 2 - protein, 111 aa, synthetic construct
LPVLTQPPSASGTPGQRVTISCSGSSSNIGSNYVYWYQQLPGTAPKLLIYRNNQRPSGVPDRFSGSKSGTSASLAISGLQSEDEADYYCASWDTSLRAWVFGGGTKLTVLG

SEQ ID NO: 3 - protein, 128 aa, synthetic construct; identical to SEQ ID NO: 1

SEQ ID NO: 4 - protein, 107 aa, synthetic construct
DIQMTQSPSSLSASVGDRVTITCRASQGIRNDLGWYQQKPGKAPKRLIFAASHLQSGVPSRFSGSGSGTEFTLTISSLQPEDFATYYCQQHNSSPFTFGPGTRVDIK

SEQ ID NO: 5 - protein, 133 aa, synthetic construct
EVKLHESGAGLVKPGASVEISCKATGYTFSSFWIEWVKQRPGHGLEWIGEILPGRGRTNYNEKFKGKATFTAETSSNTAYMQLSSLTSEDSAVYYCATGNTMVNMPYWGQGTTVTVSSGGGGSGGGGSGGGGS

SEQ ID NO: 6 - protein, 113 aa, synthetic construct
DIELTQSPSSLAVSAGEKVTMSCKSSQSLLNSGNQKNYLAWYQQKPGLPPKLLIYGASTRESGVPDRFTGSGSGTDFTLTISSVQAEDLAVYYCQNDHSYPLTFGAGTKLEIK

SEQ ID NO: 7 - protein, 463 aa, synthetic construct
MKLPVLLVVLLLFTSPASSSEVQLQESGPSLVKPSQTLSLTCSVTGDSITSGFWNWLRKFPGNKLEYLGYINYSGSTYYNPSLKSRISFTRDTSKNQYYLHLNSVTAEDTATYYCASWVLRDWGQGTTLTVSSARPTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 8 - protein, 261 aa, synthetic construct
MTSTLPFSPQVSTPRSKFATMEFQTQVLMSLLLCMSGAAADVVMTQTPLTLSVTIGQPASISCKSSQSLLDSDGKTYLNWLLQRPGQSPKRLIYLVSKLDSGVPDRFTGSGSGTDFTLKISRVEAEDLGVYYCWQGTHFPQTFGGGTKLEIKRARADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 9 - protein, 27 aa, synthetic construct
FWVLVVVGGVLACYSLLVTVAFIIFWV

SEQ ID NO: 10 - protein, 41 aa, synthetic construct
RSKRSRLLHSDYMNMTPRRPGPTRKHYQPYAPPRDFAAYRS

SEQ ID NO: 11 - protein, 112 aa, synthetic construct
RVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 12 - protein, 969 aa, synthetic construct
MAEDDPYLGRPEQMFHLDPSLTHTIFNPEVFQPQMALPTADGPYLQILEQPKQRGFRFRYVCEGPSHGGLPGASSEKNKKSYPQVKICNYVGPAKVIVQLVTNGKNIHLHAHSLVGKHCEDGICTVTAGPKDMVVGFANLGILHVTKKKVFETLEARMTEACIRGYNPGLLVHPDLAYLQAEGGGDRQLGDREKELIRQAALQQTKEMDLSVVRLMFTAFLPDSTGSFTRRLEPVVSDAIYDSKAPNASNLKIVRMDRTAGCVTGGEEIYLLCDKVQKDDIQIRFYEEEENGGVWEGFGDFSPTDVHRQFAIVFKTPKYKDINITKPASVFVQLRRKSDLETSEPKPFLYYPEIKDKEEVQRKRQKLMPNFSDSFGGGSGAGAGGGGMFGSGGGGGGTGSTGPGYSFPHYGFPTYGGITFHPGTTKSNAGMKHGTMDTESKKDPEGCDKSDDKNTVNLFGKVIETTEQDQEPSEATVGNGEVTLTYATGTKEESAGVQDNLFLEKAMQLAKRHANALFDYAVTGDVKMLLAVQRHLTAVQDENGDSVLHLAIIHLHSQLVRDLLEVTSGLISDDIINMRNDLYQTPLHLAVITKQEDVVEDLLRAGADLSLLDRLGNSVLHLAAKEGHDKVLSILLKHKKAALLLDHPNGDGLNAIHLAMMSNSLPCLLLLVAAGADVNAQEQKSGRTALHLAVEHDNISLAGCLLLEGDAHVDSTTYDGTTPLHIAAGRGSTRLAALLKAAGADPLVENFEPLYDLDDSWENAGEDEGVVPGTTPLDMATSWQVFDILNGKPYEPEFTSDDLLAQGDMKQLAEDVKLQLYKLLEIPDPDKNWATLAQKLGLGILNNAFRLSPAPSKTLMDNYEVSGGTVRELVEALRQMGYTEAIEVIQAASSPVKTTSQAHSLPLSPASTRQQIDELRDSDSVCDSGVETSFRKLSFTESLTSGASLLTLNKMPHDYGQEGPLEGKI

SEQ ID NO: 13 - protein, 969 aa, synthetic construct; identical to SEQ ID NO: 12

SEQ ID NO: 14 - protein, 579 aa, synthetic construct
MLRSGPASGPSVPTGRAMPSRRVARPPAAPELGALGSPDLSSLSLAVSRSTDELEIIDEYIKENGFGLDGGQPGPGEGLPRLVSRGAASLSTVTLGPVAPPATPPPWGCPLGRLVSPAPGPGPQPHLVITEQPKQRGMRFRYECEGRSAGSILGESSTEASKTLPAIELRDCGGLREVEVTACLVWKDWPHRVHPHSLVGKDCTDGICRVRLRPHVSPRHSFNNLGIQCVRKKEIEAAIERKIQLGIDPYNAGSLKNHQEVDMNVVRICFQASYRDQQGQMRRMDPVLSEPVYDKKSTNTSELRICRINKESGPCTGGEELYLLCDKVQKEDISVVFSRASWEGRADFSQADVHRQIAIVFKTPPYEDLEIVEPVTVNVFLQRLTDGVCSEPLPFTYLPRDHDSYGVDKKRKRGMPDVLGELNSSDPHGIESKRRKKKPAILDHFLPNHGSGPFLPPSALLPDPDFFSGTVSLPGLEPPGGPDLLDDGFAYDPTAPTLFTMLDLLPPAPPHASAVVCSGGAGAVVGETPGPEPLTLDSYQAPGPGDGGTASLVGSNMFPNHYREAAFGGGLLSPGPEAT

SEQ ID NO: 15 - protein, 397 aa, synthetic construct
YPYDVPDYAGTELGSTMASWSHPQFEKGGGSGGGSGGGSWSHPQFEKAADITSLYKKAGSTMPITRMRMRPWLEMQINSNQIPGLIWINKEEMIFQIPWKHAAKHGWDINKDACLFRSWAIHTGRYKAGEKEPDPKTWKANFRCAMNSLPDIEEVKDQSRNKGSSAVRVYRMLPPLTKNQRKERKSKSSRDAKSKAKRKSCGDSSPDTFSDGLSSSTLPDDHSSYTVPGYMQDLEVEQALTPALSPCAVSSTLPDWHIPVEVVPDSTSDLYNFQVSPMPSTSEATTDEDEEGKLPEDIMKLLEQSEWQPTNVDGKGYLLNEPGVQPTSVYGDFSCKEEPEIDSPGGDIGLSLQRVFTDLKNMDATWLDSLLTPVRLPSIQAIPCAPLDPAFLYKVVT

SEQ ID NO: 16 - protein, 208 aa, synthetic construct
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRL

SEQ ID NO: 17 - protein, 178 aa, synthetic construct
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 18 - protein, 152 aa, synthetic construct
GGWLYHHSAGPAGRWRQRRVPAGPFGSEAPVLQKRRLFPAHSPGWPCGRCAEKRPAHQTATASGGTWCSEHQGCMRPLSGDERRRPPAGEQMRHRRMFLLRTPGVQQLQHVSLPQIHLLVRGSETHRPVQTGLQNRPWPESDPVPADVREIL

SEQ ID NO: 19 - protein, 175 aa, synthetic construct
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 20 - protein, 171 aa, synthetic construct
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQELPTFLYKVVGSTSGSGKPGSGEGSTKG

SEQ ID NO: 21 - protein, 97 aa, synthetic construct
MKVSAALLWLLLIAAAFSPQGLAGPASVPTTCCFNLANRKIPLQRLESYRRITSGKCPQKAVIFKTKLAKDICADPKKKWVQDSMKYLDQKSPTPKP

SEQ ID NO: 22 - protein, 322 aa, synthetic construct
AFSGSQAPYLSPAVPFSGTIQGGLQDGLQITVNGTVLSSSGTRFAVNFQTGFSGNDIAFHFNPRFEDGGYVVCNTRQNGSWGPEERKTHMPFQKGMPFDLCFLVQSSDFKVMVNGILFVQYFHRVPFHRVDTISVNGSVQLSYISFQPPGVWPANPAPITQTVIHTVQSAPGQMFSTPAIPPMMYPHPAYPMPFITTILGGLYPSKSILLSGTVLPSAQRFHINLCSGNHIAFHLNPRFDENAVVRNTQIDNSWGSEERSLPRKMPFVRGQSFSVWILCEAHCLKVAVDGQHLFEYYHRLRNLPTINRLEVGGDIQLTHVQT

SEQ ID NO: 23 - protein, 308 aa, synthetic construct
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSIVYASLDFQWREKTPEPPVPCVPEQTEYATIVFPSGMGTSSPARRGSADGPRSAQPLRPEDGHCSWPLTRLQDIKLAVPRARDPPVAT

SEQ ID NO: 24 - protein, 301 aa, synthetic construct
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTLQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 25 - protein, 368 aa, synthetic construct
MVLEVSDHQVLNDAEVAALLENFSSSYDYGENESDSCCTSPPCPQDFSLNFDRAFLPALYSLLFLLGLLGNGAVAAVLLSRRTALSSTDTFLLHLAVADTLLVLTLPLWAVDAAVQWVFGSGLCKVAGALFNINFYAGALLLACISFDRYLNIVHATQLYRRGPPARVTLTCLAVWGLCLLFALPDFIFLSAHHDERLNATHCQYNFPQVGRTALRVLQLVAGFLLPLLVMAYCYAHILAVLLVSRGQRRLRAMRLVVVVVVAFALCWTPYHLVVLVDILMDLGALARNCGRESRVDVAKSVTSGLGYMHCCLNPLLYAFVGVKFRERMWMLLLRLGCPNQRGLQRQPSSSRRDSSWSETSEASYSGL

SEQ ID NO: 26 - protein, 352 aa, synthetic construct
MEGISIYTSDNYTEEMGSGDYDSMKEPCFREENANFNKIFLPTIYSIIFLTGIVGNGLVILVMGYQKKLRSMTDKYRLHLSVADLLFVITLPFWAVDAVANWYFGNFLCKAVHVIYTVNLYSSVLILAFISLDRYLAIVHATNSQRPRKLLAEKVVYVGVWIPALLLTIPDFIFANVSEADDRYICDRFYPNDLWVVVFQFQHIMVGLILPGIVILSCYCIIISKLSHSKGHQKRKALKTTVILILAFFACWLPYYIGISIDSFILLEIIKQGCEFENTVHKWISITEALAFFHCCLNPILYAFLGAKFKTSAQHALTSVSRGSSLKILSKGKRGGHSSVSTESESSSFHSS

SEQ ID NO: 27 - protein, 220 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEP

SEQ ID NO: 28 - protein, 117 aa, synthetic construct
QVQLQQSGAELARPGASVNLSCKASGYTFTNNGINWLKQRTGQGLEWIGEIYPRSTNTLYNEKFKGKATLTADRSSNTAYMELRSLTSEDSAVYFCARTLTAPFAFWGQGTLVTVSA

SEQ ID NO: 29 - protein, 107 aa, synthetic construct
QIVLTQSPAIMSASPGEKVTMTCSASSGVNFMHWYQQKSGTSPKRWIFDTSKLASGVPARFSGSGSGTSYSLTISSMEAEDAATYYCQQWSFNPPTFGGGTKLEIKR

SEQ ID NO: 30 - protein, 98 aa, synthetic construct
MGWSSIILFLVATATGVHSQVQLQQPGAELVRPGASVKLSCKASGYSFTTYWMNWVKQRPGQGLEWIGMIHPSDSETRLNQKFKDKATLTVDKSSSTA

SEQ ID NO: 31 - protein, 107 aa, synthetic construct
DIVMTQSQKSMSTSVGDRVSITCKASQDVITGVAWYQQKPGQSPKLLIYSASYRYTGVPDRFTGSGSGTDFTFTISNVQAEDLAVYYCQQHYSTPLTFGAGTKLELK

SEQ ID NO: 32 - protein, 24 aa, synthetic construct
IYIWAPLAGTCGVLLLSLVITLYC

SEQ ID NO: 33 - protein, 368 aa, synthetic construct
KLARHSKFGMKGPASVISNDDDSASPLHHISNGSNTPSSSEGGPDAVIIGMTKIPVIENPQYFGITNSQLKPDTFVQHIKRHNIVLKRELGEGAFGKVFLAECYNLCPEQDKILVAVKTLKDASDNARKDFHREAELLTNLQHEHIVKFYGVCVEGDPLIMVFEYMKHGDLNKFLRAHGPDAVLMAEGNPPTELTQSQMLHIAQQIAAGMVYLASQHFVHRDLATRNCLVGENLLVKIGDFGMSRDVYSTDYYRVGGHTMLPIRWMPPESIMYRKFTTESDVWSLGVVLWEIFTYGKQPWYQLSNNEVIECITQGRVLQRPRTCPQEVYELMLGCWQREPHMRKNIKGIHTLLQNLAKASPVYLDILG

SEQ ID NO: 34 - protein, 45 aa, synthetic construct
TTTPAPRPPTPAPTIASQPLSLRPEACRPAAGGAVHTRGLDFACD

SEQ ID NO: 35 - protein, 19 aa, synthetic construct
LEPKSCDKTHTCPPCPDPK

SEQ ID NO: 36 - protein, 21 aa, synthetic construct
MALPVTALLLPLALLLHAARP

SEQ ID NO: 37 - protein, 14 aa, synthetic construct
EGKSSGSGSESKAS

SEQ ID NO: 38 - DNA, 363 bp, synthetic construct
  source 1..363: mol_type = other DNA
gaggtgcagctggtgcagtctgggggaggcttggtacagcctggggggtccctgagactctcctgtgcagcctctggattcaccttcagtagctatagcatgaactgggtccgccaggctccagggaaggggctggagtgggtttcatacattagtagtagtagtagtaccatatactacgtagactctgtgaagggccgattcaccatctccagagacaacgccaagaactcactgtatctgcaaatggacagcctgagagccgaggacacggctgtgtattactgtgcgagaggaggtcgggtgttagtgggagctctatttgactactggggccagggaaccctggtcaccgtctcctca

SEQ ID NO: 39 - DNA, 333 bp, synthetic construct
  source 1..333: mol_type = other DNA
ctgcctgtgctgactcagccaccctcagcgtctgggacccccgggcagagggtcaccatctcttgttctggaagcagctccaacatcggaagtaattatgtctactggtaccagcaactcccaggaacggcccccaaactcctcatctataggaataatcagcggccctcaggggtccctgaccgattctctggctccaagtctggcacctcagcctccctggccatcagtgggctccagtctgaggatgaggctgattattattgtgcatcatgggataccagcctgcgtgcctgggtgttcggcggagggaccaagctgaccgtcctaggt

SEQ ID NO: 40 - DNA, 384 bp, synthetic construct
  source 1..384: mol_type = other DNA
caggtgcagctgcaggagtcgggcccaggactggtgaagccttcggagaccctgtccctcacctgcactgtctctggtggctccgtcagtagttactattggagctggatccggcagcccccagggaagggactggagtggattggccatatctattacagtgggaataccaactacaacccctccctcaagagtcgagtcaccatatcagtagacacgtccaagaatcagttctccctgaaactgagctctgtgaccgctgcggacacggccgtgtatttttgtgcgagagatagatggaattattatgatagtagtcccggctattattattactacggtatggacgtctggggccaagggaccacggtcaccgtcagctca

SEQ ID NO: 41 - DNA, 321 bp, synthetic construct
  source 1..321: mol_type = other DNA
gacatccagatgacccagtctccatcctccctgtctgcatctgtaggagacagagtcaccatcacttgccgggcgagtcagggcattagaaatgatttaggctggtatcagcaaaaaccagggaaagcccctaagcgcctaatctttgctgcatcccatttgcaaagtggagtcccttcaaggttcagcggcagtggatctgggacagagttcactctcacaatcagcagcctgcagcctgaagattttgcaacttattactgtcaacagcataatagttccccattcactttcggccctgggaccagagtggatatcaaa

SEQ ID NO: 42 - DNA, 399 bp, synthetic construct
  source 1..399: mol_type = other DNA
gaggtgaagctgcacgagagcggcgcaggtctggtgaagcccggcgccagcgtggagatcagctgcaaggccaccggctacaccttcagcagcttctggatcgagtgggtgaagcagagacccggccacggcctggagtggatcggcgagatcctgcccggcagaggcagaaccaactacaacgagaagttcaagggcaaggccaccttcaccgccgagaccagcagcaacaccgcctacatgcagctgagcagcctgaccagcgaggacagcgccgtgtactactgcgccaccggcaacaccatggtgaacatgccctactggggccagggcaccaccgtgaccgtgagcagcggtggaggtggttcgggaggtggaggtagcggaggtggtggaagc

SEQ ID NO: 43 - DNA, 339 bp, synthetic construct
  source 1..339: mol_type = other DNA
gatattgaactgacccagagtcccagtagcctggccgtgagtgccggcgagaaagtgaccatgagctgcaaaagcagccagagcctgctgaacagcggcaaccagaaaaactacctggcctggtaccagcagaaacccggcctgcctcctaagctgctgatctacggcgccagcaccagagaaagcggcgtgcccgatagattcaccggctctggctctggcaccgacttcaccctgaccatcagcagcgtgcaggccgaagacctggctgtctactactgccagaacgaccacagctaccccctgaccttcggcgccggcaccaagctggagatcaag

SEQ ID NO: 44 - DNA, 660 bp, synthetic construct
  source 1..660: mol_type = other DNA
gaagtgcagctggtggaaagcggcggcggcctggtgcagccgggcggcagcctgcgcctgagctgcgcggcgagcggctttaacattaaagatacctatattcattgggtgcgccaggcgccgggcaaaggcctggaatgggtggcgcgcatttatccgaccaacggctatacccgctatgcggatagcgtgaaaggccgctttaccattagcgcggataccagcaaaaacaccgcgtatctgcagatgaacagcctgcgcgcggaagataccgcggtgtattattgcagccgctggggcggcgatggcttttatgcgatggattattggggccagggcaccctggtgaccgtgagcagcgcgagcaccaaaggcccgagcgtgtttccgctggcgccgagcagcaaaagcaccagcggcggcaccgcggcgctgggctgcctggtgaaagattattttccggaaccggtgaccgtgagctggaacagcggcgcgctgaccagcggcgtgcatacctttccggcggtgctgcagagcagcggcctgtatagcctgagcagcgtggtgaccgtgccgagcagcagcctgggcacccagacctatatttgcaacgtgaaccataaaccgagcaacaccaaagtggataaaaaagtggaaccg

SEQ ID NO: 45 - DNA, 351 bp, synthetic construct
  source 1..351: mol_type = other DNA
caggtgcagctccagcagagtggcgcagagctcgctcgcccaggcgcttctgtgaatctgagttgtaaggcctccggatatacttttacgaacaacggcatcaactggctgaagcagcggaccggccagggcctggagtggatcggcgaaatatacccccggtccacaaacactctctataacgagaagtttaagggcaaagcaactctgaccgcggacaggtcctctaacacagcctatatggagctgagaagcttgacgagtgaggactccgctgtctatttttgcgcccgaactctgaccgctccttttgctttttggggccagggcacgctcgtgaccgtaagtgcg

SEQ ID NO: 46 - DNA, 321 bp, synthetic construct
  source 1..321: mol_type = other DNA
cagatcgtcctgacgcagtctccagccatcatgagcgcctcacccggcgaaaaggtgaccatgacctgctcagcctcttctggtgtgaatttcatgcactggtaccagcaaaaaagtgggacctcccctaaaaggtggatcttcgataccagcaaactggcttctggcgttcccgcaaggtttagcggctctggttccggcacatcatacagcctgacgatcagcagcatggaggcagaagacgcagctacctattactgccagcaatggagctttaacccacctactttcggaggaggaacaaagctggaaataaaaaga

SEQ ID NO: 47 - DNA, 294 bp, synthetic construct
  source 1..294: mol_type = other DNA
atgggatggagctctatcatcctcttcttggtagcaacagctacaggtgtccactcccaggtccaactgcagcagcctggggctgagctggtgaggcctggagcttcagtgaagctgtcctgcaaggcttcggggtactccttcaccacctactggatgaactgggtgaagcagaggcctggacaaggccttgagtggattggcatgattcatccttccgatagtgaaactaggttaaatcagaagttcaaggacaaggccacattgactgtagacaaatcctccagcacagcc

SEQ ID NO: 48 - DNA, 321 bp, synthetic construct
  source 1..321: mol_type = other DNA
gacattgtgatgacccagtctcagaaatccatgtccacatcagtaggagacagggtcagcatcacctgcaaggccagtcaggatgttattactggtgtagcctggtatcaacagaaaccagggcaatctcctaaattactgatttactcggcatcctaccggtacactggagtccctgatcgcttcactggcagtggatctgggacggatttcactttcaccatcagcaatgtgcaggctgaagacctggcagtttattactgtcagcaacattatagtactcctctcactttcggtgctgggaccaagctggagctgaaa

SEQ ID NO: 49 - DNA, 1389 bp, synthetic construct
  source 1..1389: mol_type = other DNA
atgaaactgccggtgctgctggtggtgctgctgctgtttaccagcccggcgagcagcagcgaagtgcagctgcaggaaagcggcccgagcctggtgaaaccgagccagaccctgagcctgacctgcagcgtgaccggcgatagcattaccagcggcttttggaactggctgcgcaaatttccgggcaacaaactggaatatctgggctatattaactatagcggcagcacctattataacccgagcctgaaaagccgcattagctttacccgcgataccagcaaaaaccagtattatctgcatctgaacagcgtgaccgcggaagataccgcgacctattattgcgcgagctgggtgctgcgcgattggggccagggcaccaccctgaccgtgagcagcgcgcgcccgaccgcgccgagcgtgtatccgctggcgccggtgtgcggcgataccaccggcagcagcgtgaccctgggctgcctggtgaaaggctattttccggaaccggtgaccctgacctggaacagcggcagcctgagcagcggcgtgcatacctttccggcggtgctgcagagcgatctgtataccctgagcagcagcgtgaccgtgaccagcagcacctggccgagccagagcattacctgcaacgtggcgcatccggcgagcagcaccaaagtggataaaaaaattgaaccgcgcggcccgaccattaaaccgtgcccgccgtgcaaatgcccggcgccgaacctgctgggcggcccgagcgtgtttatttttccgccgaaaattaaagatgtgctgatgattagcctgagcccgattgtgacctgcgtggtggtggatgtgagcgaagatgatccggatgtgcagattagctggtttgtgaacaacgtggaagtgcataccgcgcagacccagacccatcgcgaagattataacagcaccctgcgcgtggtgagcgcgctgccgattcagcatcaggattggatgagcggcaaagaatttaaatgcaaagtgaacaacaaagatctgccggcgccgattgaacgcaccattagcaaaccgaaaggcagcgtgcgcgcgccgcaggtgtatgtgctgccgccgccggaagaagaaatgaccaaaaaacaggtgaccctgacctgcatggtgaccgattttatgccggaagatatttatgtggaatggaccaacaacggcaaaaccgaactgaactataaaaacaccgaaccggtgctggatagcgatggcagctattttatgtatagcaaactgcgcgtggaaaaaaaaaactgggtggaacgcaacagctatagctgcagcgtggtgcatgaaggcctgcataaccatcataccaccaaaagctttagccgcaccccgggcaaa

SEQ ID NO: 50 - DNA, 783 bp, synthetic construct
  source 1..783: mol_type = other DNA
atgaccagcaccctgccgtttagcccgcaggtgagcaccccgcgcagcaaatttgcgaccatggaatttcagacccaggtgctgatgagcctgctgctgtgcatgagcggcgcggcggcggatgtggtgatgacccagaccccgctgaccctgagcgtgaccattggccagccggcgagcattagctgcaaaagcagccagagcctgctggatagcgatggcaaaacctatctgaactggctgctgcagcgcccgggccagagcccgaaacgcctgatttatctggtgagcaaactggatagcggcgtgccggatcgctttaccggcagcggcagcggcaccgattttaccctgaaaattagccgcgtggaagcggaagatctgggcgtgtattattgctggcagggcacccattttccgcagacctttggcggcggcaccaaactggaaattaaacgcgcgcgcgcggatgcggcgccgaccgtgagcatttttccgccgagcagcgaacagctgaccagcggcggcgcgagcgtggtgtgctttctgaacaacttttatccgaaagatattaacgtgaaatggaaaattgatggcagcgaacgccagaacggcgtgctgaacagctggaccgatcaggatagcaaagatagcacctatagcatgagcagcaccctgaccctgaccaaagatgaatatgaacgccataacagctatacctgcgaagcgacccataaaaccagcaccagcccgattgtgaaaagctttaaccgcaacgaatgc

SEQ ID NO: 51 - DNA, 81 bp, synthetic construct
  source 1..81: mol_type = other DNA
ttttgggtgctggtggtggttggtggagtcctggcttgctatagcttgctagtaacagtggcctttattattttctgggtg

SEQ ID NO: 52 - DNA, 72 bp, synthetic construct
  source 1..72: mol_type = other DNA
atctacatctgggcgcccttggccgggacttgtggggtccttctcctgtcactggttatcaccctttactgc

SEQ ID NO: 53 - DNA, 123 bp, synthetic construct
  source 1..123: mol_type = other DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 54 - DNA, 336 bp, synthetic construct
  source 1..336: mol_type = other DNA
agagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 55 - DNA, 1104 bp, synthetic construct
  source 1..1104: mol_type = other DNA
aaactggcgcgccatagcaaatttggcatgaaaggcccggcgagcgtgattagcaacgatgatgatagcgcgagcccgctgcatcatattagcaacggcagcaacaccccgagcagcagcgaaggcggcccggatgcggtgattattggcatgaccaaaattccggtgattgaaaacccgcagtattttggcattaccaacagccagctgaaaccggatacctttgtgcagcatattaaacgccataacattgtgctgaaacgcgaactgggcgaaggcgcgtttggcaaagtgtttctggcggaatgctataacctgtgcccggaacaggataaaattctggtggcggtgaaaaccctgaaagatgcgagcgataacgcgcgcaaagattttcatcgcgaagcggaactgctgaccaacctgcagcatgaacatattgtgaaattttatggcgtgtgcgtggaaggcgatccgctgattatggtgtttgaatatatgaaacatggcgatctgaacaaatttctgcgcgcgcatggcccggatgcggtgctgatggcggaaggcaacccgccgaccgaactgacccagagccagatgctgcatattgcgcagcagattgcggcgggcatggtgtatctggcgagccagcattttgtgcatcgcgatctggcgacccgcaactgcctggtgggcgaaaacctgctggtgaaaattggcgattttggcatgagccgcgatgtgtatagcaccgattattatcgcgtgggcggccataccatgctgccgattcgctggatgccgccggaaagcattatgtatcgcaaatttaccaccgaaagcgatgtgtggagcctgggcgtggtgctgtgggaaatttttacctatggcaaacagccgtggtatcagctgagcaacaacgaagtgattgaatgcattacccagggccgcgtgctgcagcgcccgcgcacctgcccgcaggaagtgtatgaactgatgctgggctgctggcagcgcgaaccgcatatgcgcaaaaacattaaaggcattcataccctgctgcagaacctggcgaaagcgagcccggtgtatctggatattctgggc

SEQ ID NO: 56 - DNA, 57 bp, synthetic construct
  source 1..57: mol_type = other DNA
ctcgagcccaaatcttgtgacaaaactcacacatgcccaccgtgcccggatcccaaa

SEQ ID NO: 57 - DNA, 135 bp, synthetic construct
  source 1..135: mol_type = other DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 58 - DNA, 2910 bp, synthetic construct
  source 1..2910: mol_type = other DNA
atggcagaagatgatccatatttgggaaggcctgaacaaatgtttcatttggatccttctttgactcatacaatatttaatccagaagtatttcaaccacagatggcactgccaacagcagatggcccataccttcaaatattagagcaacctaaacagagaggatttcgtttccgttatgtatgtgaaggcccatcccatggtggactacctggtgcctctagtgaaaagaacaagaagtcttaccctcaggtcaaaatctgcaactatgtgggaccagcaaaggttattgttcagttggtcacaaatggaaaaaatatccacctgcatgcccacagcctggtgggaaaacactgtgaggatgggatctgcactgtaactgctggacccaaggacatggtggtcggcttcgcaaacctgggtatacttcatgtgacaaagaaaaaagtatttgaaacactggaagcacgaatgacagaggcgtgtataaggggctataatcctggactcttggtgcaccctgaccttgcctatttgcaagcagaaggtggaggggaccggcagctgggagatcgggaaaaagagctaatccgccaagcagctctgcagcagaccaaggagatggacctcagcgtggtgcggctcatgtttacagcttttcttccggatagcactggcagcttcacaaggcgcctggaacccgtggtatcagacgccatctatgacagtaaagcccccaatgcatccaacttgaaaattgtaagaatggacaggacagctggatgtgtgactggaggggaggaaatttatcttctttgtgacaaagttcagaaagatgacatccagattcgattttatgaagaggaagaaaatggtggagtctgggaaggatttggagatttttcccccacagatgttcatagacaatttgccattgtcttcaaaactccaaagtataaagatattaatattacaaaaccagcctctgtgtttgtccagcttcggaggaaatctgacttggaaactagtgaaccaaaacctttcctctactatcctgaaatcaaagataaagaagaagtgcagaggaaacgtcagaagctcatgcccaatttttcggatagtttcggcggtggtagtggtgccggagctggaggcggaggcatgtttggtagtggcggtggaggagggggcactggaagtacaggtccagggtatagcttcccacactatggatttcctacttatggtgggattactttccatcctggaactactaaatctaatgctgggatgaagcatggaaccatggacactgaatctaaaaaggaccctgaaggttgtgacaaaagtgatgacaaaaacactgtaaacctctttgggaaagttattgaaaccacagagcaagatcaggagcccagcgaggccaccgttgggaatggtgaggtcactctaacgtatgcaacaggaacaaaagaagagagtgctggagttcaggataacctctttctagagaaggctatgcagcttgcaaagaggcatgccaatgcccttttcgactacgcggtgacaggagacgtgaagatgctgctggccgtccagcgccatctcactgctgtgcaggatgagaatggggacagtgtcttacacttagcaatcatccaccttcattctcaacttgtgagggatctactagaagtcacatctggtttgatttctgatgacattatcaacatgagaaatgatctgtaccagacgcccttgcacttggcagtgatcactaagcaggaagatgtggtggaggatttgctgagggctggggccgacctgagccttctggaccgcttgggtaactctgttttgcacctagctgccaaagaaggacatgataaagttctcagtatcttactcaagcacaaaaaggcagcactacttcttgaccaccccaacggggacggtctgaatgccattcatctagccatgatgagcaatagcctgccatgtttgctgctgctggtggccgctggggctgacgtcaatgctcaggagcagaagtccgggcgcacagcactgcacctggctgtggagcacgacaacatctcattggcaggctgcctgctcctggagggtgatgcccatgtggacagtactacctacgatggaaccacacccctgcatatagcagctgggagagggtccaccaggctggcagctcttctcaaagcagcaggagcagatcccctggtggagaactttgagcctctctatgacctggatgactcttgggaaaatgcaggagaggatgaaggagttgtgcctggaaccacgcctctagatatggccaccagctggcaggtatttgacatattaaatgggaaaccatatgagccagagtttacatctgatgatttactagcacaaggagacatgaaacagctggctgaagatgtgaagctgcagctgtataagttactagaaattcctgatccagacaaaaactgggctactctggcgcagaaattaggtctggggatacttaataatgccttccggctgagtcctgctccttccaaaacacttatggacaactatgaggtctctgggggtacagtcagagagctggtggaggccctgagacaaatgggctacaccgaagcaattgaagtgatccaggcagcctccagcccagtgaagaccacctctcaggcccactcgctgcctctctcgcctgcctccacaaggcagcaaatagacgagctccgagacagtgacagtgtctgcgacagcggcgtggagacatccttccgcaaactcagctttaccgagtctctgaccagtggtgcctcactgctaactctcaacaaaatgccccatgattatgggcaggaaggacctctagaaggcaaaatttag

SEQ ID NO: 59 - DNA, 1017 bp, synthetic construct
  source 1..1017: mol_type = other DNA
cgtgaagtgacggactgttctatgactgcaaagatggaaacgaccttctatgacgatgccctcaacgcctcgttcctcccgtccgagagcggaccttatggctacagtaaccccaagatcctgaaacagagcatgaccctgaacctggccgacccagtggggagcctgaagccgcacctccgcgccaagaactcggacctcctcacctcgcccgacgtggggctgctcaagctggcgtcgcccgagctggagcgcctgataatccagtccagcaacgggcacatcaccaccacgccgacccccacccagttcctgtgccccaagaacgtgacagatgagcaggagggcttcgccgagggcttcgtgcgcgccctggccgaactgcacagccagaacacgctgcccagcgtcacgtcggcggcgcagccggtcaacggggcaggcatggtggctcccgcggtagcctcggtggcagggggcagcggcagcggcggcttcagcgccagcctgcacagcgagccgccggtctacgcaaacctcagcaacttcaacccaggcgcgctgagcagcggcggcggggcgccctcctacggcgcggccggcctggcctttcccgcgcaaccccagcagcagcagcagccgccgcaccacctgccccagcagatgcccgtgcagcacccgcggctgcaggccctgaaggaggagcctcagacagtgcccgagatgcccggcgagacaccgcccctgtcccccatcgacatggagtcccaggagcggatcaaggcggagaggaagcgcatgaggaaccgcatcgctgcctccaagtgccgaaaaaggaagctggagagaatcgcccggctggaggaaaaagtgaaaaccttgaaagctcagaactcggagctggcgtccacggccaacatgctcagggaacaggtggcacagcttaaacagaaagtcatgaaccacgttaacagtgggtgccaactcatgctaacgcagcagttgcaaacattttga

SEQ ID NO: 60 - DNA, 1737 bp, synthetic construct
  source 1..1737: mol_type = other DNA
atgcttcggtctgggccagcctctgggccgtccgtccccactggccgggccatgccgagtcgccgcgtcgccagaccgccggctgcgccggagctgggggccttagggtcccccgacctctcctcactctcgctcgccgtttccaggagcacagatgaattggagatcatcgacgagtacatcaaggagaacggcttcggcctggacgggggacagccgggcccgggcgaggggctgccacgcctggtgtctcgcggggctgcgtccctgagcacggtcaccctgggccctgtggcgcccccagccacgccgccgccttggggctgccccctgggccgactagtgtccccagcgccgggcccgggcccgcagccgcacctggtcatcacggagcagcccaagcagcgcggcatgcgcttccgctacgagtgcgagggccgctcggccggcagcatccttggggagagcagcaccgaggccagcaagacgctgcccgccatcgagctccgggattgtggagggctgcgggaggtggaggtgactgcctgcctggtgtggaaggactggcctcaccgagtccacccccacagcctcgtggggaaagactgcaccgacggcatctgcagggtgcggctccggcctcacgtcagcccccggcacagttttaacaacctgggcatccagtgtgtgaggaagaaggagattgaggctgccattgagcggaagattcaactgggcattgacccctacaacgctgggtccctgaagaaccatcaggaagtagacatgaatgtggtgaggatctgcttccaggcctcatatcgggaccagcagggacagatgcgccggatggatcctgtgctttccgagcccgtctatgacaagaaatccacaaacacatcagagctgcggatttgccgaattaacaaggaaagcgggccgtgcaccggtggcgaggagctctacttgctctgcgacaaggtgcagaaagaggacatatcagtggtgttcagcagggcctcctgggaaggtcgggctgacttctcccaggccgacgtgcaccgccagattgccattgtgttcaagacgccgccctacgaggacctggagattgtcgagcccgtgacagtcaacgtcttcctgcagcggctcaccgatggggtctgcagcgagccattgcctttcacgtacctgcctcgcgaccatgacagctacggcgtggacaagaagcggaaacgggggatgcccgacgtccttggggagctgaacagctctgacccccatggcatcgagagcaaacggcggaagaaaaagccggccatcctggaccacttcctgcccaaccacggctcaggcccgttcctcccgccgtcagccctgctgccagaccctgacttcttctctggcaccgtgtccctgcccggcctggagccccctggcgggcctgacctcctggacgatggctttgcctacgaccctacggcccccacactcttcaccatgctggacctgctgcccccggcaccgccacacgctagcgctgttgtgtgcagcggaggtgccggggccgtggttggggagacccccggccctgaaccactgacactggactcgtaccaggccccgggccccggggatggaggcaccgccagccttgtgggcagcaacatgttccccaatcattaccgcgaggcggcctttgggggcggcctcctatccccggggcctgaagccacg

SEQ ID NO: 61 - DNA, 1194 bp, synthetic construct
  source 1..1194: mol_type = other DNA
tacccatacgatgttcctgactatgccggtaccgagctcggatccaccatggctagctggagccacccgcagttcgagaaaggtggaggttccggaggtggatcgggaggtggatcgtggagccacccgcagttcgaaaaagcggccgatatcacaagtttgtacaaaaaagcaggctccaccatgcccatcactcggatgcgcatgagaccctggctagagatgcagattaattccaaccaaatcccggggctcatctggattaataaagaggagatgatcttccagatcccatggaagcatgctgccaagcatggctgggacatcaacaaggatgcctgtttgttccggagctgggccattcacacaggccgatacaaagcaggggaaaaggagccagatcccaagacgtggaaggccaactttcgctgtgccatgaactccctgccagatatcgaggaggtgaaagaccagagcaggaacaagggcagctcagctgtgcgagtgtaccggatgcttccacctctcaccaagaaccagagaaaagaaagaaagtcgaagtccagccgagatgctaagagcaaggccaagaggaagtcatgtggggattccagccctgataccttctctgatggactcagcagctccactctgcctgatgaccacagcagctacacagttccaggctacatgcaggacttggaggtggagcaggccctgactccagcactgtcgccatgtgctgtcagcagcactctccccgactggcacatcccagtggaagttgtgccggacagcaccagtgatctgtacaacttccaggtgtcacccatgccctccacctctgaagctacaacagatgaggatgaggaagggaaattacctgaggacatcatgaagctcttggagcagtcggagtggcagccaacaaacgtggatgggaaggggtacctactcaatgaacctggagtccagcccacctctgtctatggagactttagctgtaaggaggagccagaaattgacagcccagggggggatattgggctgagtctacagcgtgtcttcacagatctgaagaacatggatgccacctggctggacagcctgctgaccccagtccggttgccctccatccaggccattccctgtgcaccgttggacccagctttcttgtacaaagtggtgacgtaa

SEQ ID NO: 62 - DNA, 625 bp, synthetic construct
  source 1..625: mol_type = other DNA
atgtctaccgagtctatgattagggacgtggaactggctgaggaggcactgcccaaaaaaaccggcggaccacagggctctaggagatgtctgtttctgtctctgttctcttttctcatcgtggctggcgctacaacactcttctgtctgctccatttcggcgtgattggaccacagcgagaggaatttccccgggatctgtcactcatttcaccactggcacaggctgtccgatcttcatctcggactccatccgacaaacctgtcgcccatgtcgtcgccaacccacaggccgagggccagctccagtggctcaataggagggcaaacgctctgctcgccaatggcgtggaactccgggataaccagctcgtcgtgcctagtgagggactgtacctcatctactcccaggtgctgtttaagggccagggatgtccttctacacatgtgctgctcacacacacaatttcacggatcgccgtgtcttaccagactaaagtcaatctgctctctgccatcaaatccccatgtcagcgggaaacacctgagggcgctgaggctaaaccttggtacgaacccatctacctcggaggcgtgttccagctggagaagggcgatagactga

SEQ ID NO: 63 - DNA, 537 bp, synthetic construct
  source 1..537: mol_type = other DNA
atgcacagctcagcactgctctgttgcctggtccttctgacaggcgtaagggcgtcacctggccaaggaacacagtcagagaacagctgtacacatttccccggcaacttgcccaatatgcttagggatcttcgcgatgccttctcacgagtgaagacattttttcagatgaaggatcagttggataaccttttgctgaaggaaagcctgctcgaggacttcaagggatacctcggatgccaggcactgagcgaaatgatacagttctacctggaagaggtaatgcctcaggcagaaaatcaggaccccgatattaaagctcatgtgaactctctgggtgagaacctgaaaactctgaggctgaggctgcggaggtgtcacagattcctgccatgcgagaacaaatcaaaagccgtcgaacaggtgaagaacgcctttaacaaactgcaggagaaaggcatctataaagcgatgagcgagttcgatattttcatcaactacattgaggcatacatgacgatgaaaatccgaaattag

SEQ ID NO: 64 - DNA, 466 bp, synthetic construct
  source 1..466: mol_type = other DNA
ggcggctggctctatcaccactctgccggccctgccggaagatggcggcagcggcgcgttcccgccgggccattttaaggatccgaagcgcctgtactgcaaaaacggcggctttttcctgcgcattcacccggatggccgtgtggacggtgtgcgtgagaaaagcgacccgcacatcaaactgcaactgcaagcggaggaacgtggtgtagtgagcatcaagggtgtatgcgctaaccgttatctggcgatgaaagaagacggccgcctgctggcgagcaaatgcgtcaccgacgaatgtttcttcttcgaacgcctggagtccaacaactacaacacgtatcgctcccgcaaatacacctcttggtacgtggctctgaaacgcaccggccagtacaaactgggctccaaaaccggccctggccagaaagcgatcctgttcctgccgatgtccgcgaaatcctaa

SEQ ID NO: 65 - DNA, 528 bp, synthetic construct
  source 1..528: mol_type = other DNA
atgactcctttgggtccagcttcttccttgcctcaatccttcttgttgaagtgtttggagcaggttagaaagatccagggtgatggtgctgctttgcaagagaagttgtgtgctacttacaagttgtgtcacccagaagagttggttttgttgggtcactccttgggtattccttgggctccattgtcctcttgtccatcccaagctttgcaattggctggttgtttgtcccaattgcactccggtttgttcttgtaccagggtttgttgcaagctttggagggtatttctccagagttgggtccaactttggacacattgcagttggacgttgctgacttcgctactactatctggcaacagatggaagaattgggtatggctccagctttgcagccaactcaaggtgctatgccagcttttgcttctgctttccagagaagagctggtggtgttttggttgcttctcacttgcagtctttcttggaggtttcctacagagttttgagacacttggctcaaccataa

SEQ ID NO: 66 - DNA, 513 bp, synthetic construct
  source 1..513: mol_type = other DNA
atgtggctgcagagcctgctgctcttgggcactgtggcctgcagcatctctgcacccgcccgctcgcccagccccagcacgcagccctgggagcatgtgaatgccatccaggaggcccggcgtctcctgaacctgagtagagacactgctgctgagatgaatgaaacagtagaagtcatctcagaaatgtttgacctccaggagccgacctgcctacagacccgcctggagctgtacaagcagggcctgcggggcagcctcaccaagctcaagggccccttgaccatgatggccagccactacaagcagcactgccctccaaccccggaaacttcctgtgcaacccagattatcacctttgaaagtttcaaagagaacctgaaggactttctgcttgtcatcccctttgactgctgggagccagtccaggagttgccaactttcttgtacaaagtggttggctccaccagcggcagcggcaagccaggctccggcgaaggcagcaccaaaggc

SEQ ID NO: 67 - DNA, 969 bp, synthetic construct
  source 1..969: mol_type = other DNA
gccttcagcggttcccaggctccctacctgagtccagctgtccccttttctgggactattcaaggaggtctccaggacggacttcagatcactgtcaatgggaccgttctcagctccagtggaaccaggtttgctgtgaactttcagactggcttcagtggaaatgacattgccttccacttcaaccctcggtttgaagatggagggtacgtggtgtgcaacacgaggcagaacggaagctgggggcccgaggagaggaagacacacatgcctttccagaaggggatgccctttgacctctgcttcctggtgcagagctcagatttcaaggtgatggtgaacgggatcctcttcgtgcagtacttccaccgcgtgcccttccaccgtgtggacaccatctccgtcaatggctctgtgcagctgtcctacatcagcttccagcctcccggcgtgtggcctgccaacccggctcccattacccagacagtcatccacacagtgcagagcgcccctggacagatgttctctactcccgccatcccacctatgatgtacccccaccccgcctatccgatgcctttcatcaccaccattctgggagggctgtacccatccaagtccatcctcctgtcaggcactgtcctgcccagtgctcagaggttccacatcaacctgtgctctgggaaccacatcgccttccacctgaacccccgttttgatgagaatgctgtggtccgcaacacccagatcgacaactcctgggggtctgaggagcgaagtctgccccgaaaaatgcccttcgtccgtggccagagcttctcagtgtggatcttgtgtgaagctcactgcctcaaggtggccgtggatggtcagcacctgtttgaatactaccatcgcctgaggaacctgcccaccatcaacagactggaagtggggggcgacatccagctgacccatgtgcagacatag

SEQ ID NO: 68 - DNA, 924 bp, synthetic construct
  source 1..924: mol_type = other DNA
atgcagatcccacaggcgccctggccagtcgtctgggcggtgctacaactgggctggcggccaggatggttcttagactccccagacaggccctggaacccccccaccttctccccagccctgctcgtggtgaccgaaggggacaacgccaccttcacctgcagcttctccaacacatcggagagcttcgtgctaaactggtaccgcatgagccccagcaaccagacggacaagctggccgctttccccgaggaccgcagccagcccggccaggactgccgcttccgtgtcacacaactgcccaacgggcgtgacttccacatgagcgtggtcagggcccggcgcaatgacagcggcacctacctctgtggggccatctccctggcccccaaggcgcagatcaaagagagcctgcgggcagagctcagggtgacagagagaagggcagaagtgcccacagcccaccccagcccctcacccaggccagccggccagttccaaaccctggtggttggtgtcgtgggcggcctgctgggcagcctggtgctgctagtctgggtcctggccgtcatctgctcccgggccgcacgagggacaataggagccaggcgcaccggccagcccctgaaggaggacccctcagccgtgcctgtgttctctattgtttatgcttccctggatttccagtggcgagagaagaccccggagccccccgtgccctgtgtccctgagcagacggagtatgccaccattgtctttcctagcggaatgggcacctcatcccccgcccgcaggggctcagccgacggccctcggagtgcccagccactgaggcctgaggatggacactgctcttggcccctgacgcgtctgcaggatatcaagcttgcggtaccgcgggcccgggatccaccggtcgccacc

SEQ ID NO: 69 - DNA, 906 bp, synthetic construct
  source 1..906: mol_type = other DNA
atgttttcacatcttccctttgactgtgtcctgctgctgctgctgctactacttacaaggtcctcagaagtggaatacagagcggaggtcggtcagaatgcctatctgccctgcttctacaccccagccgccccagggaacctcgtgcccgtctgctggggcaaaggagcctgtcctgtgtttgaatgtggcaacgtggtgctcaggactgatgaaagggatgtgaattattggacatccagatactggctaaatggggatttccgcaaaggagatgtgtccctgaccatagagaatgtgactctagcagacagtgggatctactgctgccggatccaaatcccaggcataatgaatgatgaaaaatttaacctgaagttggtcatcaaaccagccaaggtcacccctgcaccgactctgcagagagacttcactgcagcctttccaaggatgcttaccaccaggggacatggcccagcagagacacagacactggggagcctccctgatataaatctaacacaaatatccacattggccaatgagttacgggactctagattggccaatgacttacgggactctggagcaaccatcagaataggcatctacatcggagcagggatctgtgctgggctggctctggctcttatcttcggcgctttaattttcaaatggtattctcatagcaaagagaagatacagaatttaagcctcatctctttggccaacctccctccctcaggattggcaaatgcagtagcagagggaattcgctcagaagaaaacatctataccattgaagagaacgtatatgaagtggaggagcccaatgagtattattgctatgtcagcagcaggcagcaaccctcacaacctttgggttgtcgctttgcaatgccatag

SEQ ID NO: 70 - DNA, 1104 bp, synthetic construct
  source 1..1104: mol_type = other DNA
atggttctggaagtttccgaccaccaggtgcttaatgatgcggaagttgcggcactgctggaaaactttagctcatcctatgactacggcgagaacgagtcagactcttgctgtacctcccctccttgcccccaagactttagcttgaatttcgatcgggccttcctgcccgcactttactctctgctgttcctgttgggactgttgggcaacggggccgtggcagcagtactgttgagtcgaaggactgccctgtccagtaccgatacatttctgctgcacctcgccgtggctgacacactgctggttctgaccctgcccctttgggcagtcgatgcagctgtccagtgggttttcgggagtggactttgtaaggtggcaggggcgctgttcaatatcaacttctacgccggagcactgctgctggcttgtataagcttcgaccgatatctgaacattgtgcacgccacgcaactctatagaagaggccctccagcaagggttacactcacctgtcttgcagtttgggggctgtgcttgcttttcgcattgcccgacttcattttcctgagtgctcaccatgacgagcggctgaacgccactcattgtcaatacaacttccctcaggtggggagaacagcgctgcgagtgctgcagttggtggccggattcctgctgcctttgctggtgatggcgtactgctacgcgcatattttggcggtcttgttggtttctagagggcaaaggcggctgagggctatgcgactggtcgttgtggtggttgtcgccttcgcgctctgctggaccccataccacctggtggtgctggttgatatactgatggatttgggagccctggcgaggaattgcggtagagaaagccgggtcgacgttgcaaaaagcgtcacctcagggctgggctacatgcactgttgtctgaacccccttctgtacgcattcgtgggggtgaagttccgggaacgcatgtggatgttgttgctgcgactgggttgccctaatcagcgcggtctgcagcgacagcccagttcttctcgaagggactcatcatggtccgaaacctcagaggctagttattctggcctc

SEQ ID NO: 71 - DNA, 1056 bp, synthetic construct
  source 1..1056: mol_type = other DNA
atggaggggatatcaatctacacatcagataattacacggaagagatgggttccggcgattacgactctatgaaagagccgtgttttagagaggaaaacgcgaactttaacaagatctttctccccaccatctacagcatcatctttctcacaggcatcgtagggaacggcctggtcatcctggtgatgggataccaaaagaagctgaggtcaatgaccgacaagtataggctccatctgtccgtggccgacctcctgtttgtgattaccctgcctttttgggcagttgacgctgtcgctaattggtacttcggcaacttcctctgtaaggcagtgcacgttatctacactgtgaatctttatagttccgtcttgatcttggcctttatcagtctcgacaggtatttggcgattgtgcacgctaccaactcacaacgacctagaaaactcctggctgagaaggtggtttacgttggtgtgtggattccagctctcctgttgacaataccagacttcattttcgctaatgtgagcgaggccgatgacagatacatttgtgaccgattttacccaaacgatctgtgggtagtggtatttcagttccaacacattatggtcgggctgatcttgcccggcattgtcatactgtcttgctactgcatcattatttctaagctgtcacactcaaaaggccaccaaaagaggaaggctctgaaaacaacggtgatcctgatactggccttcttcgcatgttggctgccctactatatcggcatcagcattgactcatttatactcctggaaattatcaagcagggctgcgagttcgagaacaccgttcataagtggatttctataaccgaggccctcgccttctttcactgttgtttgaatccgattctctacgcgtttcttggcgccaaatttaaaacaagcgcccaacatgcactgacatcagtgtctagggggagctctctgaaaatcctctccaagggaaaacgaggcggacatagcagtgtcagcactgagtccgaatccagctcatttcatagctct

SEQ ID NO: 72 - protein, 21 aa, synthetic construct
FFMINTSILLIFIFIVLLIHF

SEQ ID NO: 73 - protein, 19 aa, synthetic construct
IGVSVLSVLVVSVVAVLVY

SEQ ID NO: 74 - protein, 179 aa, synthetic construct
EGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 75 - protein, 289 aa, synthetic construct
WRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVHRVKFSRSADAPAYKQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 76 - protein, 19 aa, synthetic construct
KFYFHLMLLAGCIKYGRGE

SEQ ID NO: 77 - protein, 168 aa, synthetic construct
NIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 78 - protein, 161 aa, synthetic construct
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRG

SEQ ID NO: 79 - protein, 329 aa, synthetic construct
LKLGKPLGRGAFGQVIEADAFGIDKTATCRTVAVKMLKEGATHSEHRALMSELKILIHIGHHLNVVNLLGACTKPGGPLMVIVEFCKFGNLSTYLRSKRNEFVPYKTKGARFRQGKDYVGAIPVDLKRRLDSITSSQSSASSGFVEEKSLSDVEEEEAPEDLYKDFLTLEHLICYSFQVAKGMEFLASRKCIHRDLAARNILLSEKNVVKICDFGLARDIYKDPDYVRKGDARLPLKWMAPETIFDRVYTIQSDVWSFGVLLWEIFSLGASPYPGVKIDEEFCRRLKEGTRMRAPDYTTPEMYQTMLDCWHGEPSQRPTFSELVEHLGN

SEQ ID NO: 80 - DNA, 63 bp, synthetic construct
  source 1..63: mol_type = other DNA
ttttttatgatcaatacgtctattcttctcatatttattttcatcgttcttctgattcacttt

SEQ ID NO: 81 - DNA, 57 bp, synthetic construct
  source 1..57: mol_type = other DNA
attggggtgtctgtcctaagcgtgctggttgtttccgtggttgccgttctggtatat

SEQ ID NO: 82 - DNA, 126 bp, synthetic construct
  source 1..126: mol_type = other DNA
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 83 - DNA, 537 bp, synthetic construct
  source 1..537: mol_type = other DNA
gaggggtggaggatttccttttactggaatgtgtctgtgcatagggttttgggttttaaggagatagatcgccaaacagagcagttcgagtatgcagcttatattatacacgcgtacaaagataaggactgggtatgggagcatttcagttccatggaaaaagaagaccagtccttgaagttctgccttgaagaaagggacttcgaggccggggtttttgagctggaagcgatcgttaatagtattaagcggtcacgcaagatcatcttcgtaataacacatcacctcctcaaggatccactttgcaaacgcttcaaagttcaccatgcggtgcagcaagcgatcgaacagaaccttgactccataatccttgtctttctggaagaaatacctgattacaagttgaatcatgctctgtgtctgcggcggggaatgtttaagtctcattgtatccttaactggcctgtgcagaaagagcgcattggggcgtttaggcataaactccaggttgcgcttggaagcaaaaattccgttcat

SEQ ID NO: 84 - DNA, 871 bp, synthetic construct
  source 1..871: mol_type = other DNA
tggcggatctctttctactggaacgtgtcagtgcatagagtcctcgggttcaaagagattgacaggcagacggaacaatttgagtatgcagcgtacattatccatgcctacaaagataaagactgggtttgggaacatttctcctccatggaaaaggaggatcagtctttgaagttttgtctggaggagcgggactttgaggcgggagtgttcgagcttgaggccattgtaaactcgatcaagcggtcccgtaaaatcatctttgtgataacacaccacctgttgaaagacccgctctgcaagcgctttaaggtgcatcatgctgtccagcaggccatcgagcagaatctggattcaattattctggtgtttttagaagagatcccagactataagctgaatcacgctctatgtttgcgtcgaggcatgttcaaatctcattgcatcttgaattggccggtgcaaaaggaaaggattggtgcttttcggcacaaattgcaggtcgccctgggctccaaaaacagtgttcacagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgctaag

SEQ ID NO: 85 - DNA, 57 bp, synthetic construct
  source 1..57: mol_type = other DNA
aagttctatttccatctgatgcttctcgctggctgcataaagtacgggaggggggag

SEQ ID NO: 86 - DNA, 504 bp, synthetic construct
  source 1..504: mol_type = other DNA
aatatatatgacgctttcgtgatctactcgagccaggatgaggactgggttcgcaacgagctagtcaagaatcttgaagagggcgtgcctcctttccagctctgtctgcattaccgcgattttattcctggggtggccatcgcggccaacatcatccacgagggcttccataaatccagaaaagtgattgtcgttgtgagccagcatttcatccagtccaggtggtgcattttcgaatatgagatagcccagacctggcagtttcttagcagtcgggctgggattatttttatcgtgctgcagaaggttgaaaagacccttttgcggcaacaggtggaactgtaccgattattatcccgtaacacttacttggaatgggaagactcagttctcggacgccacattttctggcgccggctcaggaaggccctgctggatggtaaatcctggaaccccgaggggacagtggggaccggatgtaactggcaagaggcaacaagtata

SEQ ID NO: 87 - DNA, 483 bp, synthetic construct
  source 1..483: mol_type = other DNA
atgaagtacacttcttacatactcgccttccagctttgtatagtgttgggcagtctgggttgctattgccaagatccttatgtgaaggaagcagaaaatttgaagaaatactttaacgcgggtcattctgacgttgcagataatggaaccttgtttttgggaatacttaaaaattggaaggaggaaagcgaccgcaagatcatgcagtctcaaatcgtttccttttattttaaacttttcaagaattttaaggacgatcagtccatacagaaatctgtagagactatcaaagaggatatgaacgtaaagttttttaacagcaacaaaaagaaaagagatgactttgagaaacttacgaattatagtgtcaccgatctgaacgtccaacgcaaagcaatccacgagttgattcaagttatggcagagctttccccagcggcgaaaactggaaagcgcaaacgatctcaaatgctcttccggggt

SEQ ID NO: 88 - DNA, 987 bp, synthetic construct
  source 1..987: mol_type = other DNA
ctgaagctgggcaagcccctgggcaggggcgccttcggccaggtgatcgaggccgacgccttcggcatcgacaagaccgccacctgcaggaccgtggccgtgaagatgctgaaggagggcgccacccacagcgagcacagggccctgatgagcgagctgaagatcctgatccacatcggccaccacctgaacgtggtgaacctgctgggcgcctgcaccaagcccggcggccccctgatggtgatcgtggagttctgcaagttcggcaacctgagcacctacctgaggagcaagaggaacgagttcgtgccctacaagaccaagggcgccaggttcaggcagggcaaggactacgtgggcgccatccccgtggacctgaagaggaggctggacagcatcaccagcagccagagcagcgccagcagcggcttcgtggaggagaagagcctgagcgacgtggaggaggaggaggcccccgaggacctgtacaaggacttcctgaccctggagcacctgatctgctacagcttccaggtggccaagggcatggagttcctggccagcaggaagtgcatccacagggacctggccgccaggaacatcctgctgagcgagaagaacgtggtgaagatctgcgacttcggcctggccagggacatctacaaggaccccgactacgtgaggaagggcgacgccaggctgcccctgaagtggatggcccccgagaccatcttcgacagggtgtacaccatccagagcgacgtgtggagcttcggcgtgctgctgtgggagatcttcagcctgggcgccagcccctaccccggcgtgaagatcgacgaggagttctgcaggaggctgaaggagggcaccaggatgagggcccccgactacaccacccccgagatgtaccagaccatgctggactgctggcacggcgagcccagccagaggcccaccttcagcgagctggtggagcacctgggcaac

SEQ ID NO: 89 - protein, 42 aa, synthetic construct
KRGRKKLLYIFKQPFMRPVQTTQEEDGCSCRFPEEEEGGCEL

SEQ ID NO: 90 - protein, 738 aa, synthetic construct
CTGCCTGTGCTGACTCAGCCACCCTCAGCGTCTGGGACCCCCGGGCAGAGGGTCACCATCTCTTGTTCTGGAAGCAGCTCCAACATCGGAAGTAATTATGTCTACTGGTACCAGCAACTCCCAGGAACGGCCCCCAAACTCCTCATCTATAGGAATAATCAGCGGCCCTCAGGGGTCCCTGACCGATTCTCTGGCTCCAAGTCTGGCACCTCAGCCTCCCTGGCCATCAGTGGGCTCCAGTCTGAGGATGAGGCTGATTATTATTGTGCATCATGGGATACCAGCCTGCGTGCCTGGGTGTTCGGCGGAGGGACCAAGCTGACCGTCCTAGGTGAGGGTAAATCTTCCGGATCTGGTTCCGAATCCAAAGCTAGCGAGGTGCAGCTGGTGCAGTCTGGGGGAGGCTTGGTACAGCCTGGGGGGTCCCTGAGACTCTCCTGTGCAGCCTCTGGATTCACCTTCAGTAGCTATAGCATGAACTGGGTCCGCCAGGCTCCAGGGAAGGGGCTGGAGTGGGTTTCATACATTAGTAGTAGTAGTAGTACCATATACTACGTAGACTCTGTGAAGGGCCGATTCACCATCTCCAGAGACAACGCCAAGAACTCACTGTATCTGCAAATGGACAGCCTGAGAGCCGAGGACACGGCTGTGTATTACTGTGCGAGAGGAGGTCGGGTGTTAGTGGGAGCTCTATTTGACTACTGGGGCCAGGGAACCCTGGTCACCGTCTCCTCA

SEQ ID NO: 91 - protein, 223 aa, synthetic construct
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 92 - protein, 904 aa, synthetic construct
MRQTLPCIYFWGGLLPFGMLCASSTTKCTVSHEVADCSHLKLTQVPDDLPTNITVLNLTHNQLRRLPAANFTRYSQLTSLDVGFNTISKLEPELCQKLPMLKVLNLQHNELSQLSDKTFAFCTNLTELHLMSNSIQKIKNNPFVKQKNLITLDLSHNGLSSTKLGTQVQLENLQELLLSNNKIQALKSEELDIFANSSLKKLELSSNQIKEFSPGCFHAIGRLFGLFLNNVQLGPSLTEKLCLELANTSIRNLSLSNSQLSTTSNTTFLGLKWTNLTMLDLSYNNLNVVGNDSFAWLPQLEYFFLEYNNIQHLFSHSLHGLFNVRYLNLKRSFTKQSISLASLPKIDDFSFQWLKCLEHLNMEDNDIPGIKSNMFTGLINLKYLSLSNSFTSLRTLTNETFVSLAHSPLHILNLTKNKISKIESDAFSWLGHLEVLDLGLNEIGQELTGQEWRGLENIFEIYLSYNKYLQLTRNSFALVPSLQRLMLRRVALKNVDSSPSPFQPLRNLTILDLSNNNIANINDDMLEGLEKLEILDLQHNNLARLWKHANPGGPIYFLKGLSHLHILNLESNGFDEIPVEVFKDLFELKIIDLGLNNLNTLPASVFNNQVSLKSLNLQKNLITSVEKKVFGPAFRNLTELDMRFNPFDCTCESIAWFVNWINETHTNIPELSSHYLCNTPPHYHGFPVRLFDTSSCKDSAPFELFFMINTSILLIFIFIVLLIHFEGWRISFYWNVSVHRVLGFKEIDRQTEQFEYAAYIIHAYKDKDWVWEHFSSMEKEDQSLKFCLEERDFEAGVFELEAIVNSIKRSRKIIFVITHHLLKDPLCKRFKVHHAVQQAIEQNLDSIILVFLEEIPDYKLNHALCLRRGMFKSHCILNWPVQKERIGAFRHKLQVALGSKNSVH

SEQ ID NO: 93 - protein, 839 aa, synthetic construct
MMSASRLAGTLIPAMAFLSCVRPESWEPCVEVVPNITYQCMELNFYKIPDNLPFSTKNLDLSFNPLRHLGSYSFFSFPELQVLDLSRCEIQTIEDGAYQSLSHLSTLILTGNPIQSLALGAFSGLSSLQKLVAVETNLASLENFPIGHLKTLKELNVAHNLIQSFKLPEYFSNLTNLEHLDLSSNKIQSIYCTDLRVLHQMPLLNLSLDLSLNPMNFIQPGAFKEIRLHKLTLRNNFDSLNVMKTCIQGLAGLEVHRLVLGEFRNEGNLEKFDKSALEGLCNLTIEEFRLAYLDYYLDDIIDLFNCLTNVSSFSLVSVTIERVKDFSYNFGWQHLELVNCKFGQFPTLKLKSLKRLTFTSNKGGNAFSEVDLPSLEFLDLSRNGLSFKGCCSQSDFGTTSLKYLDLSFNGVITMSSNFLGLEQLEHLDFQHSNLKQMSEFSVFLSLRNLIYLDISHTHTRVAFNGIFNGLSSLEVLKMAGNSFQENFLPDIFTELRNLTFLDLSQCQLEQLSPTAFNSLSSLQVLNMSHNNFFSLDTFPYKCLNSLQVLDYSLNHIMTSKKQELQHFPSSLAFLNLTQNDFACTCEHQSFLQWIKDQRQLLVEVERMECATPSDKQGMPVLSLNITCQMNKTIIGVSVLSVLVVSVVAVLVYKFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 94 - protein, 1032 aa, synthetic construct
MGFCRSALHPLSLLVQAIMLAMTLALGTLPAFLPCELQPHGLVNCNWLFLKSVPHFSMAAPRGNVTSLSLSSNRIHHLHDSDFAHLPSLRHLNLKWNCPPVGLSPMHFPCHMTIEPSTFLAVPTLEELNLSYNNIMTVPALPKSLISLSLSHTNILMLDSASLAGLHALRFLFMDGNCYYKNPCRQALEVAPGALLGLGNLTHLSLKYNNLTVVPRNLPSSLEYLLLSYNRIVKLAPEDLANLTALRVLDVGGNCRRCDHAPNPCMECPRHFPQLHPDTFSHLSRLEGLVLKDSSLSWLNASWFRGLGNLRVLDLSENFLYKCITKTKAFQGLTQLRKLNLSFNYQKRVSFAHLSLAPSFGSLVALKELDMHGIFFRSLDETTLRPLARLPMLQTLRLQMNFINQAQLGIFRAFPGLRYVDLSDNRISGASELTATMGEADGGEKVWLQPGDLAPAPVDTPSSEDFRPNCSTLNFTLDLSRNNLVTVQPEMFAQLSHLQCLRLSHNCISQAVNGSQFLPLTGLQVLDLSHNKLDLYHEHSFTELPRLEALDLSYNSQPFGMQGVGHNFSFVAHLRTLRHLSLAHNNIHSQVSQQLCSTSLRALDFSGNALGHMWAEGDLYLHFFQGLSGLIWLDLSQNRLHTLLPQTLRNLPKSLQVLRLRDNYLAFFKWWSLHFLPKLEVLDLAGNQLKALTNGSLPAGTRLRRLDVSCNSISFVAPGFFSKAKELRELNLSANALKTVDHSWFGPLASALQILDVSANPLHCACGAAFMDFLLEVQAAVPGLPSRVKCGSPGQLQGLSIFAQDLRLCLDEALSWDCFALSLLAVALGLGVPMLHHLCGWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 95 - protein, 461 aa, synthetic construct
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGDFALPVGLIVGVTALGLLIIGVVNCVIMTQVKKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 96 - protein, 21 aa, synthetic construct
SLWIPVVAALLLFLVLSLVFI

SEQ ID NO: 97 - protein, 21 aa, synthetic construct
VIIFFAFVLLLSGALAYCLAL

SEQ ID NO: 98 - protein, 21 aa, synthetic construct
FALSLLAVALGLGVPMLHHLC

SEQ ID NO: 99 - protein, 30 aa, synthetic construct
FALPVGLIVGVTALGLLIIGVVNCVIMTQV

SEQ ID NO: 100 - protein, 187 aa, synthetic construct
KFYFHLMLLAGCIKYGRGENIYDAFVIYSSQDEDWVRNELVKNLEEGVPPFQLCLHYRDFIPGVAIAANIIHEGFHKSRKVIVVVSQHFIQSRWCIFEYEIAQTWQFLSSRAGIIFIVLQKVEKTLLRQQVELYRLLSRNTYLEWEDSVLGRHIFWRRLRKALLDGKSWNPEGTVGTGCNWQEATSI

SEQ ID NO: 101 - protein, 223 aa, synthetic construct
CFYIKKINPLKEKSIILPKSLISVVRSATLETKPESKYVSLITSYQPFSLEKEVVCEEPLSPATVPGMHTEDNPGKVEHTEELSSITEVVTTEENIPDVVPGSHLTPIERESSSPLSSNQSEPGSIALNSYHSRNCSESDHSRNGFDTDSSCLESHSSLSDSEFPPNNKGEIKTEGQELITVIKAPTSFGYDKPHVLVDLLVDDSGKESLIGYRPTEDSKEFS

SEQ ID NO: 102 - protein, 322 aa, synthetic construct
QLYVRRRKKLPSVLLFKKPSPFIFISQRPSPETQDTIHPLDEEAFLKVSPELKNLDLHGSTDSGFGSTKPSLQTEEPQFLLPDPHPQADRTLGNREPPVLGDSCSSGSSNSTDSGICLQEPSLSPSTGPTWEQQVGSNSRGQDDSGIDLVQNSEGRAGDTQGGSALGHHSPPEPEVPGEEDPAAVAFQGYLRQTRCAEEKATKTGCLEEESPLTDGLGPKFGRCLVDEAGLHPPALAKGYLKQDPLEMTLASSGAPTGQWNQPTEEWSLLALSSCSDLGISDWSFAHDLAPLGCVAAPGGLLGSFNSDLVTLPLISSLQSSE

SEQ ID NO: 103 - protein, 193 aa, synthetic construct
GWDLWYCFHLCLAWLPWRGRQSGRDEDALPYDAFVVFDKTQSAVADWVYNELRGQLEECRGRWALRLCLEERDWLPGKTLFENLWASVYGSRKTLFVLAHTDRVSGLLRASFLLAQQRLLEDRKDVVVLVILSPDGRRSRYVRLRQRLCRQSVLLWPHQPSGQRSFWAQLGMALTRDNHHFYNRNFCQGPTAE

SEQ ID NO: 104 - protein, 174 aa, synthetic construct
KKKPLCLQREAKVPHLPADKARGTQGPEQQHLLITAPSSSSSSLESSASALDRRAPTRNQPQAPGVEASGAGEARASTGSSDSSPGGHGTQVNVTCIVNVCSSSDHSSQCSSQASSTMGDTDSSPSESPKDEQVPFSKEECAFRSQLETPETLLGSTEEKPLPLGVPDAGMKPS

SEQ ID NO: 105 - DNA, 63 bp, synthetic construct
  source 1..63: mol_type = other DNA
tctctttggattccagttgttgctgctttactactctttctagtgcttagcctggtattcatc

SEQ ID NO: 106 - DNA, 63 bp, synthetic construct
  source 1..63: mol_type = other DNA
gtcatcatattctttgcctttgttttgctgctctccggagcgctggcttactgcctcgcgctc

SEQ ID NO: 107 - DNA, 63 bp, synthetic construct
  source 1..63: mol_type = other DNA
tttgccctcagtcttctggctgtagctctcgggcttggcgtccctatgcttcaccacctgtgt

SEQ ID NO: 108 - DNA, 90 bp, synthetic construct
  source 1..90: mol_type = other DNA
ttcgctcttccagttggactgattgtgggtgtgacagccttgggtctactaataataggagtggtgaactgtgtcatcatgacccaggtg

SEQ ID NO: 109 - DNA, 537 bp, synthetic construct
  source 1..537: mol_type = other DNA
gaaggatggcgaatctctttctactggaacgtgtctgttcatagagttttgggattcaaggaaatagatcgccaaaccgagcaattcgagtatgccgcatatatcatacatgcatataaggacaaggactgggtatgggaacacttctcctctatggagaaagaagatcagtcactcaaattctgcttggaggaacgagatttcgaagccggtgtatttgagcttgaagcaatagtaaactctataaaaagaagtcgaaaaattattttcgtcatcacacatcatctgttgaaggacccgctttgtaagaggtttaaagtgcatcacgctgtgcagcaagccattgaacaaaatctggatagcatcattctcgtttttctcgaagaaatacccgactataagctcaatcacgctttgtgcttgcgacggggaatgttcaagtcccactgcattcttaactggccagttcaaaaggaacgaatcggggccttcagacataaattgcaagttgccctgggctcaaagaactcagtgcac

SEQ ID NO: 110 - DNA, 561 bp, synthetic construct
  source 1..561: mol_type = other DNA
aagttctatttccatctgatgcttctcgctggctgcataaagtacgggaggggggagaatatatatgacgctttcgtgatctactcgagccaggatgaggactgggttcgcaacgagctagtcaagaatcttgaagagggcgtgcctcctttccagctctgtctgcattaccgcgattttattcctggggtggccatcgcggccaacatcatccacgagggcttccataaatccagaaaagtgattgtcgttgtgagccagcatttcatccagtccaggtggtgcattttcgaatatgagatagcccagacctggcagtttcttagcagtcgggctgggattatttttatcgtgctgcagaaggttgaaaagacccttttgcggcaacaggtggaactgtaccgattattatcccgtaacacttacttggaatgggaagactcagttctcggacgccacattttctggcgccggctcaggaaggccctgctggatggtaaatcctggaaccccgaggggacagtggggaccggatgtaactggcaagaggcaacaagtata

SEQ ID NO: 111 - DNA, 1237 bp, synthetic construct
  source 1..1237: mol_type = other DNA
tgtttttatattaagaaaattaatccattgaaggaaaaaagcataatattacccaagtccttgatctctgtggtaagaagtgctactttagagacaaaacctgaatcaaaatatgtatcactcatcacgtcataccagccattttccttagaaaaggaggtggtctgtgaagagccgttgtctccagcaacagttccaggcatgcataccgaagacaatccaggaaaagtggaacatacagaagaactttctagtataacagaagtggtgactactgaagaaaatattcctgacgtggtcccgggcagccatctgactccaatagagagagagagttcttcacctttaagtagtaaccagtctgaacctggcagcatcgctttaaactcgtatcactccagaaattgttctgagagtgatcactccagaaatggttttgatactgattccagctgtctggaatcacatagctccttatctgactcagaatttcccccaaataataaaggtgaaataaaaacagaaggacaagagctcataaccgtaataaaagcccccacctcctttggttatgataaaccacatgtgctagtggatctacttgtggatgatagcggtaaagagtccttgattggttatagaccaacagaagattccaaagaattttcatgagatcagctaagttgcaccaactttgaagtctgattttcctggacagttttctgctttaatttcatgaaaagattatgatctcagaaattgtatcttagttggtatcaaccaaatggagtgacttagtgtacatgaaagcgtaaagaggatgtgtggcattttcacttttggcttgtaaagtacagacttttttttttttttaaacaaaaaaagcattgtaacttatgaacctttacatccagataggttaccagtaacggaacagtatccagtactcctggttcctaggtgagcaggtgatgccccagggacctttgtagccacttcactttttttcttttctctgccttggtatagcatatgtttttgtaagtttatgcatacagtaattttaagtaatttcagaagaaattctgcaagcttttcaaaattggacttaaaatctaattcaaactaatagaattaatggaatatgtaaatagaaacgtgtatattttttatgaaacattacagttagagatttttaaataaagaattttaaaactcgaaaaaaaaaaaaaaaaaa

SEQ ID NO: 112 - DNA, 483 bp, synthetic construct; identical to SEQ ID NO: 87
  source 1..483: mol_type = other DNA

SEQ ID NO: 113 - DNA, 966 bp, synthetic construct
  source 1..966: mol_type = other DNA
cagctctatgtcagaaggagaaaaaagctcccgtccgtgcttctttttaagaagccttccccgttcatcttcattagtcagcgaccgagccctgagacccaggacacgatccatccgcttgatgaagaggcgtttcttaaggtaagccctgaactgaagaatctcgatctgcacggcagcaccgacagtggattcgggtccacgaaaccctcactccagacagaagagcctcagtttttgttgcctgatcctcacccccaggctgatagaacgttgggtaatcgcgagccgccggtacttggagacagctgctcatctgggtcaagcaactcaacggattctgggatatgtttgcaggaaccaagcctctcacctagcactgggccaacctgggagcagcaagtgggaagcaattcacgggggcaggacgactctggtatagacctggtacaaaactctgagggacgggcgggagatactcaaggtggtagcgccctgggacaccacagcccaccggaaccagaggtcccgggcgaggaggaccctgcagcggtggcgtttcagggatatttgcgccaaacccgctgtgctgaggagaaggctactaaaacgggctgcttggaagaagaatctccgctcacagacgggctcggcccgaagttcggccgatgtttggtggacgaagcggggcttcatccgccggcgcttgctaaagggtacctgaagcaagaccccttggagatgaccctggcctcctccggggcgcctactggtcagtggaaccagccgaccgaggaatggtctctcctcgccctttccagttgctccgacctcggtatatcagactggagtttcgcacacgaccttgcacccctcggctgtgtagccgccccaggaggccttcttggctcatttaacagtgacctcgttacgctccccctcatttcatctctgcaatcctctgaa

SEQ ID NO: 114 - DNA, 582 bp, synthetic construct
  source 1..582: mol_type = other DNA
ggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatag

SEQ ID NO: 115 - DNA, 2726 bp, synthetic construct
  source 1..2726: mol_type = other DNA
aaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaaccaggccggtgtgggctgtgtcgtagccaaggtgggctgagccctggcaggatgaccctgcgaaggggccctggtccttccaggcccccaccactaggactctgaggctctttctgggccaagttcctctagtgccctccacagccgcagcctccctctgacctgcaggccaagagcagaggcagcgagttgtggaaagcctctgctgccatggcgtgtccctctcggaaggctggctgggcatggacgttcggggcatgctggggcaagtccctgactctctgtgacctgccccgcccagctgcacctgccagcctggcttctggagcccttgggttttttgtttgtttgtttgtttgtttgtttgtttctccccctgggctctgccccagctctggcttccagaaaaccccagcatccttttctgcagaggggctttctggagaggagggatgctgcctgagtcacccatgaagacaggacagtgcttcagcctgaggctgagactgcgggatggtcctggggctctgtgcagggaggaggtggcagccctgtagggaacggggtccttcaagttagctcaggaggcttggaaagcatcacctcaggccaggtgcagtggctcacgcctatgatcccagcactttgggaggctgaggcgggtggatcacctgaggttaggagttcgagaccagcctggccaacatggtaaaaccccatctctactaaaaatacagaaattagccgggcgtggtggcgggcacctatagtcccagctactcagaagcctgaggctgggaaatcgtttgaacccgggaagcggaggttgcagggagccgagatcacgccactgcactccagcctgggcgacagagcgagagtctgtctcaaaagaaaaaaaaaagcaccgcctccaaatgccaacttgtccttttgtaccatggtgtgaaagtcagatgcccagagggcccaggcaggccaccatattcagtgctgtggcctgggcaagataacgcacttctaactagaaatctgccaattttttaaaaaagtaagtaccactcaggccaacaagccaacgacaaagccaaactctgccagccacatccaaccccccacctgccatttgcaccctccgccttcactccggtgtgcctgcagccccgcgcctccttccttgctgtcctaggccacaccatctcctttcagggaatttcaggaactagagatgactgagtcctcgtagccatctctctactcctacctcagcctagaccctcctcctcccccagaggggtgggttcctcttccccactccccaccttcaattcctgggccccaaacgggctgccctgccactttggtacatggccagtgtgatcccaagtgccagtcttgtgtctgcgtctgtgttgcgtgtcgtgggtgtgtgtagccaaggtcggtaagttgaatggcctgccttgaagccactgaagctgggattcctccccattagagtcagccttccccctcccagggccagggccctgcagaggggaaaccagtgtagccttgcccggattctgggaggaagcaggttgaggggctcctggaaaggctcagtctcaggagcatggggataaaggagaaggcatgaaattgtctagcagagcaggggcagggtgataaattgttgataaattccactggacttgagcttggcagctgaactattggagggtgggagagcccagccattaccatggagacaagaagggttttccaccctggaatcaagatgtcagactggctggctgcagtgacgtgcacctgtactcaggaggctgaggggaggatcactggagcccaggagtttgaggctgcagcgagctatgatcgcgccactacactccagcctgagcaacagagtgagaccctgtctcttaaagaaaaaaaaagtcagactgctgggactggccaggtttctgcccacattggacccacatgaggacatgatggagcgcacctgccccctggtggacagtcctgggagaacctcaggcttccttggcatcacagggcagagccgggaagcgatgaatttggagactctgtggggccttggttcccttgtgtgtgtgtgttgatcccaagacaatgaaagtttgcactgtatgctggacggcattcctgcttatcaataaacctgtttgttttaa

SEQ ID NO: 116 - DNA, 672 bp, synthetic construct
  source 1..672: mol_type = other DNA
atggcttgccttggatttcagcggcacaaggctcagctgaacctggctaccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgatggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 117 - DNA, 2715 bp, synthetic construct
  source 1..2715: mol_type = other DNA
atgagacagactttgccttgtatctacttttgggggggccttttgccctttgggatgctgtgtgcatcctccaccaccaagtgcactgttagccatgaagttgctgactgcagccacctgaagttgactcaggtacccgatgatctacccacaaacataacagtgttgaaccttacccataatcaactcagaagattaccagccgccaacttcacaaggtatagccagctaactagcttggatgtaggatttaacaccatctcaaaactggagccagaattgtgccagaaacttcccatgttaaaagttttgaacctccagcacaatgagctatctcaactttctgataaaacctttgccttctgcacgaatttgactgaactccatctcatgtccaactcaatccagaaaattaaaaataatccctttgtcaagcagaagaatttaatcacattagatctgtctcataatggcttgtcatctacaaaattaggaactcaggttcagctggaaaatctccaagagcttctattatcaaacaataaaattcaagcgctaaaaagtgaagaactggatatctttgccaattcatctttaaaaaaattagagttgtcatcgaatcaaattaaagagttttctccagggtgttttcacgcaattggaagattatttggcctctttctgaacaatgtccagctgggtcccagccttacagagaagctatgtttggaattagcaaacacaagcattcggaatctgtctctgagtaacagccagctgtccaccaccagcaatacaactttcttgggactaaagtggacaaatctcactatgctcgatctttcctacaacaacttaaatgtggttggtaacgattcctttgcttggcttccacaactagaatatttcttcctagagtataataatatacagcatttgttttctcactctttgcacgggcttttcaatgtgaggtacctgaatttgaaacggtcttttactaaacaaagtatttcccttgcctcactccccaagattgatgatttttcttttcagtggctaaaatgtttggagcaccttaacatggaagataatgatattccaggcataaaaagcaatatgttcacaggattgataaacctgaaatacttaagtctatccaactcctttacaagtttgcgaactttgacaaatgaaacatttgtatcacttgctcattctcccttacacatactcaacctaaccaagaataaaatctcaaaaatagagagtgatgctttctcttggttgggccacctagaagtacttgacctgggccttaatgaaattgggcaagaactcacaggccaggaatggagaggtctagaaaatattttcgaaatctatctttcctacaacaagtacctgcagctgactaggaactcctttgccttggtcccaagccttcaacgactgatgctccgaagggtggcccttaaaaatgtggatagctctccttcaccattccagcctcttcgtaacttgaccattctggatctaagcaacaacaacatagccaacataaatgatgacatgttggagggtcttgagaaactagaaattctcgatttgcagcataacaacttagcacggctctggaaacacgcaaaccctggtggtcccatttatttcctaaagggtctgtctcacctccacatccttaacttggagtccaacggctttgacgagatcccagttgaggtcttcaaggatttatttgaactaaagatcatcgatttaggattgaataatttaaacacacttccagcatctgtctttaataatcaggtgtctctaaagtcattgaaccttcagaagaatctcataacatccgttgagaagaaggttttcgggccagctttcaggaacctgactgagttagatatgcgctttaatccctttgattgcacgtgtgaaagtattgcctggtttgttaattggattaacgagacccataccaacatccctgagctgtcaagccactacctttgcaacactccacctcactatcatgggttcccagtgagactttttgatacatcatcttgcaaagacagtgccccctttgaactctttttcatgatcaataccagtatcctgttgatttttatctttattgtacttctcatccactttgagggctggaggatatctttttattggaatgtttcagtacatcgagttcttggtttcaaagaaatagacagacagacagaacagtttgaatatgcagcatatataattcatgcctataaagataaggattgggtctgggaacatttctcttcaatggaaaaggaagaccaatctctcaaattttgtctggaagaaagggactttgaggcgggtgtttttgaactagaagcaattgttaacagcatcaaaagaagcagaaaaattatttttgttataacacaccatctattaaaagacccattatgcaaaagattcaaggtacatcatgcagttcaacaagctattgaacaaaatctggattccattatattggttttccttgaggagattccagattataaactgaaccatgcactctgtttgcgaagaggaatgtttaaatctcactgcatcttgaactggccagttcagaaagaacggataggtgcctttcgtcataaattgcaagtagcacttggatccaaaaactctgtacattaa

SEQ ID NO: 118 - DNA, 2520 bp, synthetic construct
  source 1..2520: mol_type = other DNA
atgatgtctgcctcgcgcctggctgggactctgatcccagccatggccttcctctcctgcgtgagaccagaaagctgggagccctgcgtggaggtggttcctaatattacttatcaatgcatggagctgaatttctacaaaatccccgacaacctccccttctcaaccaagaacctggacctgagctttaatcccctgaggcatttaggcagctatagcttcttcagtttcccagaactgcaggtgctggatttatccaggtgtgaaatccagacaattgaagatggggcatatcagagcctaagccacctctctaccttaatattgacaggaaaccccatccagagtttagccctgggagccttttctggactatcaagtttacagaagctggtggctgtggagacaaatctagcatctctagagaacttccccattggacatctcaaaactttgaaagaacttaatgtggctcacaatcttatccaatctttcaaattacctgagtatttttctaatctgaccaatctagagcacttggacctttccagcaacaagattcaaagtatttattgcacagacttgcgggttctacatcaaatgcccctactcaatctctctttagacctgtccctgaaccctatgaactttatccaaccaggtgcatttaaagaaattaggcttcataagctgactttaagaaataattttgatagtttaaatgtaatgaaaacttgtattcaaggtctggctggtttagaagtccatcgtttggttctgggagaatttagaaatgaaggaaacttggaaaagtttgacaaatctgctctagagggcctgtgcaatttgaccattgaagaattccgattagcatacttagactactacctcgatgatattattgacttatttaattgtttgacaaatgtttcttcattttccctggtgagtgtgactattgaaagggtaaaagacttttcttataatttcggatggcaacatttagaattagttaactgtaaatttggacagtttcccacattgaaactcaaatctctcaaaaggcttactttcacttccaacaaaggtgggaatgctttttcagaagttgatctaccaagccttgagtttctagatctcagtagaaatggcttgagtttcaaaggttgctgttctcaaagtgattttgggacaaccagcctaaagtatttagatctgagcttcaatggtgttattaccatgagttcaaacttcttgggcttagaacaactagaacatctggatttccagcattccaatttgaaacaaatgagtgagttttcagtattcctatcactcagaaacctcatttaccttgacatttctcatactcacaccagagttgctttcaatggcatcttcaatggcttgtccagtctcgaagtcttgaaaatggctggcaattctttccaggaaaacttccttccagatatcttcacagagctgagaaacttgaccttcctggacctctctcagtgtcaactggagcagttgtctccaacagcatttaactcactctccagtcttcaggtactaaatatgagccacaacaacttcttttcattggatacgtttccttataagtgtctgaactccctccaggttcttgattacagtctcaatcacataatgacttccaaaaaacaggaactacagcattttccaagtagtctagctttcttaaatcttactcagaatgactttgcttgtacttgtgaacaccagagtttcctgcaatggatcaaggaccagaggcagctcttggtggaagttgaacgaatggaatgtgcaacaccttcagataagcagggcatgcctgtgctgagtttgaatatcacctgtcagatgaataagaccatcattggtgtgtcggtcctcagtgtgcttgtagtatctgttgtagcagttctggtctataagttctattttcacctgatgcttcttgctggctgcataaagtatggtagaggtgaaaacatctatgatgcctttgttatctactcaagccaggatgaggactgggtaaggaatgagctagtaaagaatttagaagaaggggtgcctccatttcagctctgccttcactacagagactttattcccggtgtggccattgctgccaacatcatccatgaaggtttccataaaagccgaaaggtgattgttgtggtgtcccagcacttcatccagagccgctggtgtatctttgaatatgagattgctcagacctggcagtttctgagcagtcgtgctggtatcatcttcattgtcctgcagaaggtggagaagaccctgctcaggcagcaggtggagctgtaccgccttctcagcaggaacacttacctggagtgggaggacagtgtcctggggcggcacatcttctggagacgactcagaaaagccctgctggatggtaaatcatggaatccagaaggaacagtgggtacaggatgcaattggcaggaagcaacatctatctga

SEQ ID NO: 119 - DNA, 3099 bp, synthetic construct
  source 1..3099: mol_type = other DNA
atgggtttctgccgcagcgccctgcacccgctgtctctcctggtgcaggccatcatgctggccatgaccctggccctgggtaccttgcctgccttcctaccctgtgagctccagccccacggcctggtgaactgcaactggctgttcctgaagtctgtgccccacttctccatggcagcaccccgtggcaatgtcaccagcctttccttgtcctccaaccgcatccaccacctccatgattctgactttgcccacctgcccagcctgcggcatctcaacctcaagtggaactgcccgccggttggcctcagccccatgcacttcccctgccacatgaccatcgagcccagcaccttcttggctgtgcccaccctggaagagctaaacctgagctacaacaacatcatgactgtgcctgcgctgcccaaatccctcatatccctgtccctcagccataccaacatcctgatgctagactctgccagcctcgccggcctgcatgccctgcgcttcctattcatggacggcaactgttattacaagaacccctgcaggcaggcactggaggtggccccgggtgccctccttggcctgggcaacctcacccacctgtcactcaagtacaacaacctcactgtggtgccccgcaacctgccttccagcctggagtatctgctgttgtcctacaaccgcatcgtcaaactggcgcctgaggacctggccaatctgaccgccctgcgtgtgctcgatgtgggcggaaattgccgccgctgcgaccacgctcccaacccctgcatggagtgccctcgtcacttcccccagctacatcccgataccttcagccacctgagccgtcttgaaggcctggtgttgaaggacagttctctctcctggctgaatgccagttggttccgtgggctgggaaacctccgagtgctggacctgagtgagaacttcctctacaaatgcatcactaaaaccaaggccttccagggcctaacacagctgcgcaagcttaacctgtccttcaattaccaaaagagggtgtcctttgcccacctgtctctggccccttccttcgggagcctggtcgccctgaaggagctggacatgcacggcatcttcttccgctcactcgatgagaccacgctccggccactggcccgcctgcccatgctccagactctgcgtctgcagatgaacttcatcaaccaggcccagctcggcatcttcagggccttccctggcctgcgctacgtggacctgtcggacaaccgcatcagcggagcttcggagctgacagccaccatgggggaggcagatggaggggagaaggtctggctgcagcctggggaccttgctccggccccagtggacactcccagctctgaagacttcaggcccaactgcagcaccctcaacttcaccttggatctgtcacggaacaacctggtgaccgtgcagccggagatgtttgcccagctctcgcacctgcagtgcctgcgcctgagccacaactgcatctcgcaggcagtcaatggctcccagttcctgccgctgaccggtctgcaggtgctagacctgtcccacaataagctggacctctaccacgagcactcattcacggagctaccgcgactggaggccctggacctcagctacaacagccagccctttggcatgcagggcgtgggccacaacttcagcttcgtggctcacctgcgcaccctgcgccacctcagcctggcccacaacaacatccacagccaagtgtcccagcagctctgcagtacgtcgctgcgggccctggacttcagcggcaatgcactgggccatatgtgggccgagggagacctctatctgcacttcttccaaggcctgagcggtttgatctggctggacttgtcccagaaccgcctgcacaccctcctgccccaaaccctgcgcaacctccccaagagcctacaggtgctgcgtctccgtgacaattacctggccttctttaagtggtggagcctccacttcctgcccaaactggaagtcctcgacctggcaggaaaccagctgaaggccctgaccaatggcagcctgcctgctggcacccggctccggaggctggatgtcagctgcaacagcatcagcttcgtggcccccggcttcttttccaaggccaaggagctgcgagagctcaaccttagcgccaacgccctcaagacagtggaccactcctggtttgggcccctggcgagtgccctgcaaatactagatgtaagcgccaaccctctgcactgcgcctgtggggcggcctttatggacttcctgctggaggtgcaggctgccgtgcccggtctgcccagccgggtgaagtgtggcagtccgggccagctccagggcctcagcatctttgcacaggacctgcgcctctgcctggatgaggccctctcctgggactgtttcgccctctcgctgctggctgtggctctgggcctgggtgtgcccatgctgcatcacctctgtggctgggacctctggtactgcttccacctgtgcctggcctggcttccctggcgggggcggcaaagtgggcgagatgaggatgccctgccctacgatgccttcgtggtcttcgacaaaacgcagagcgcagtggcagactgggtgtacaacgagcttcgggggcagctggaggagtgccgtgggcgctgggcactccgcctgtgcctggaggaacgcgactggctgcctggcaaaaccctctttgagaacctgtgggcctcggtctatggcagccgcaagacgctgtttgtgctggcccacacggaccgggtcagtggtctcttgcgcgccagcttcctgctggcccagcagcgcctgctggaggaccgcaaggacgtcgtggtgctggtgatcctgagccctgacggccgccgctcccgctacgtgcggctgcgccagcgcctctgccgccagagtgtcctcctctggccccaccagcccagtggtcagcgcagcttctgggcccagctgggcatggccctgaccagggacaaccaccacttctataaccggaacttctgccagggacccacggccgaatag

SEQ ID NO: 120 - DNA, 1386 bp, synthetic construct
  source 1..1386: mol_type = other DNA
atggcgcccgtcgccgtctgggccgcgctggccgtcggactggagctctgggctgcggcgcacgccttgcccgcccaggtggcatttacaccctacgccccggagcccgggagcacatgccggctcagagaatactatgaccagacagctcagatgtgctgcagcaaatgctcgccgggccaacatgcaaaagtcttctgtaccaagacctcggacaccgtgtgtgactcctgtgaggacagcacatacacccagctctggaactgggttcccgagtgcttgagctgtggctcccgctgtagctctgaccaggtggaaactcaagcctgcactcgggaacagaaccgcatctgcacctgcaggcccggctggtactgcgcgctgagcaagcaggaggggtgccggctgtgcgcgccgctgcgcaagtgccgcccgggcttcggcgtggccagaccaggaactgaaacatcagacgtggtgtgcaagccctgtgccccggggacgttctccaacacgacttcatccacggatatttgcaggccccaccagatctgtaacgtggtggccatccctgggaatgcaagcatggatgcagtctgcacgtccacgtcccccacccggagtatggccccaggggcagtacacttaccccagccagtgtccacacgatcccaacacacgcagccaactccagaacccagcactgctccaagcacctccttcctgctcccaatgggccccagccccccagctgaagggagcactggcgacttcgctcttccagttggactgattgtgggtgtgacagccttgggtctactaataataggagtggtgaactgtgtcatcatgacccaggtgaaaaagaagcccttgtgcctgcagagagaagccaaggtgcctcacttgcctgccgataaggcccggggtacacagggccccgagcagcagcacctgctgatcacagcgccgagctccagcagcagctccctggagagctcggccagtgcgttggacagaagggcgcccactcggaaccagccacaggcaccaggcgtggaggccagtggggccggggaggcccgggccagcaccgggagctcagattcttcccctggtggccatgggacccaggtcaatgtcacctgcatcgtgaacgtctgtagcagctctgaccacagctcacagtgctcctcccaagccagctccacaatgggagacacagattccagcccctcggagtccccgaaggacgagcaggtccccttctccaaggaggaatgtgcctttcggtcacagctggagacgccagagaccctgctggggagcaccgaagagaagcccctgccccttggagtgcctgatgctgggatgaagcccagttaa